(19) United States
(12) Patent Application Publication
(10) Pub. No.: US 2021/0154188 A1
(43) Pub. Date: May 27, 2021

(54) ALK5 INHIBITORS, CONJUGATES, AND USES THEREOF

(71) Applicant: SILVERBACK THERAPEUTICS, INC., Seattle, WA (US)

(72) Inventors: Sean Wesley Smith, Seattle, WA (US); Craig Alan Coburn, Seattle, WA (US); Robert Finley Dubose, Seattle, WA (US)

(21) Appl. No.: 17/045,151

(22) PCT Filed: Apr. 2, 2019
(86) PCT No.: PCT/US2019/025370

§ 371 Date: Oct. 2, 2020

Related U.S. Application Data
(60) Provisional application No. 62/651,561, filed on Apr. 2, 2018.

(51) Int. Cl. A61K 31/4725; C07D 471/04; C07F 5/02; A61K 31/444; A61K 31/69; A61K 31/4709; A61K 31/496; A61K 31/4545; A61K 47/68

(52) U.S. Cl. CPC A61K 31/4725; C07D 471/04; C07F 5/027; A61K 31/444; A61K 31/69; A61K 47/6803; A61K 31/496; A61K 31/4545; A61K 47/6889; A61K 47/6851; A61K 31/4709

(57) ABSTRACT

Compounds, conjugates and pharmaceutical compositions for use in the treatment of disease, such as cancer, are disclosed herein. The disclosed compounds may be useful, among other things, in treating of cancer and inhibiting ALKS. Compounds incorporated into a conjugate with an antibody construct are also described herein.

### CROSS-REFERENCE TO RELATED APPLICATION

This application claims the benefit of U.S. Provisional Application No. 62/651,561 filed Apr. 2, 2018, the entire contents of which are incorporated herein by reference.

### BACKGROUND OF THE INVENTION

One of the leading causes of death in the United States is cancer. The conventional methods of cancer treatment, like chemotherapy, surgery, or radiation therapy, tend to be either highly toxic or nonspecific to a cancer, or both, resulting in limited efficacy and harmful side effects. However, the immune system has the potential to be a powerful, specific tool in fighting cancers. In many cases tumors can specifically express genes whose products are required for inducing or maintaining the malignant state. These proteins may serve as antigen markers for the development and establishment of more specific anti-cancer immune response. The boosting of this specific immune response has the potential to be a powerful anti-cancer treatment that can be more effective than conventional methods of cancer treatment and can have fewer side effects.

### SUMMARY OF THE INVENTION

The present disclosure provides, inter alia, compounds represented by the structure of Formula (I):

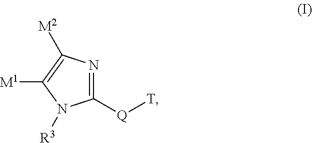

or a salt thereof, wherein M¹, M², R³, Q, and T are as described herein.

Also provided are compounds of the present invention as compound-linkers. Exemplary compounds of the present invention can be bound to a linker L³. In some embodiments, L³ is covalently bound to a substitutable nitrogen or substitutable oxygen of the compound. In some embodiments, L³ is covalently bound to a substitutable nitrogen of the compound. L³ can be a cleavable or a non-cleavable linker. The compound-linker can be further covalently attached to an antibody construct or to a targeting moiety, optionally through the linker. In some embodiments, the targeting moiety or antibody construct specifically binds to a tumor antigen. In some embodiments, the antibody construct or targeting moiety further comprises a target binding domain. Also provided are compounds of the present invention as conjugates. In one aspect, the present disclosure provides a conjugate represented by the formula:

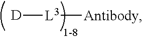

wherein Antibody is an antibody construct, D is a compound or salt of any one of Formulas (I), (Ia), (Ib), (Ic), (Id) and (Ie), and L³ is a linker moiety. Also provided are methods for preparing such antibody conjugates.

The present disclosure provides pharmaceutical compositions comprising the compounds or conjugates of the present invention and at least one pharmaceutically acceptable excipient.

In another aspect, the present disclosure provides a method of killing tumor cells in vivo, or a methot for treatment comprising contacting a tumor cell population with a conjugate or compound as described herein.

In a further aspect, the present disclosure provides a method for treating diseases including, but not limited to, cancer and fibrosis. The methods can comprise administering to a subject in need thereof a conjugate or compound as described herein. In some aspects, a method for treating fibrosis comprises administering to a subject in need thereof a compound or a conjugate as described herein. In some embodiments, the targeting moiety or antibody construct specifically binds to LRRC15. In some embodiments, the targeting moiety or antibody construct specifically binds to a tumor antigen on non-cancerous cells associated with fibrosis, autoimmune disease or inflammatory disease. In some embodiments, the targeting moiety or antibody construct is used for the treatment of fibrosis, automimmune disease, or inflammatory disease. In some embodiments, the antibody construct comprises a chimeric, human or humanized antibody.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The present disclosure provides compounds, conjugates and pharmaceutical compositions for use in the treatment of disease. In certain embodiments, the compounds of the disclosure are activin receptor-like kinase 5 (ALK5) inhibitors. Activin receptor-like kinase 5 (ALK5), which is also commonly known as transforming growth factor beta receptor 1 (TGF-βR¹⁰), is a serine/threonine kinase transmembrane receptor. It is a part of the TGFβ signaling pathway and is involved in signal transduction from the cell surface to the cytoplasm. The TGFβ signaling pathway regulates gene expression of genes involved in cellular processes such as differentiation, apoptosis, wound healing, and cell growth. ALK5 and TGF-βR1 can be used interchangeably.

In the absence of TGFβ ligands, ALK5 remains a homodimeric cell surface receptor. However, ligand binding to type II TGFβ receptor (TGFβR2) induces the formation of the TGFβR1/TGFβR2 complex, which leads to phosphorylation of Mothers Against Decapentaplegic homolog 2 (Smad2) and Mothers Against Decapentaplegic homolog 3 (Smad3) and subsequent modulation of a number of downstream signaling targets involved in the regulation of gene expression. As such, inhibitors of ALK5 may be useful in altering or modulating the expression of genes involved in cancer, and thus, may be useful in treating and preventing cancer.

The compounds of the present disclosure may act as ALK5 inhibitors. The compounds, salts, and conjugates of the present disclosure may be useful for treatment and/or prevention, e.g., vaccination, of cancer, autoimmune diseases, inflammation, fibrosis, sepsis, allergy, asthma, graft rejection, graft-versus-host disease, immunodeficiencies, and infectious diseases.

In certain embodiments, the compounds, salts, and conjugates have utility in the treatment of cancer either as single agents or in combination therapy. In certain embodiments, the compounds, salts, and conjugates have utility as single agent immunomodulators, vaccine adjuvants and in combination with conventional cancer therapies. In certain embodiments, the compounds and salts are incorporated into a conjugate that can be utilized, for example, to enhance an immune response. In certain embodiments, the disclosure provides conjugates including a compound or salt described herein and an antibody construct.

In some aspects, an ALK5 inhibitor has an IC₅₀ value of between 0.1 nM and 1000 nM, between 0.1 nm and 100 nM, or between 0.1 nM and 80 nM in an ALK5 enzyme inhibition assay. An exemplary ALK5 enzyme inhibition assay is as set forth in the example section.

In some aspects, ALK5 inhibitor has an IC₅₀ value of between 0.1 nM and 1000 nM, between 0.1 nm and 100 nM, between 0.1 nM and 80 nM, or between 0.1nM and 10 nM in a TGF-βR1 reporter assay. An exemplary TGF-βR1 reporter assay is as set forth in the example section.

In some aspects, an ALK5 inhibitor has an IC₅₀ value of between 0.1 nM and 1000 nM, between 0.1 nm and 100 nM, or between 0.1 nM and 80 nM in a ALK5 enzyme inhibition assay and an IC₅₀ value of between 0.1 nM and 1000 nM , between 0.1 nm and 100 nM, between 0.1 nM and 80 nM, or between 0.1nM and 10 nM in a TGF-βR1 reporter assay.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

As used herein, “sequence identity” refers to the identity between a DNA, RNA, nucleotide, amino acid, or protein sequence to another DNA, RNA, nucleotide, amino acid, or protein sequence, respectively, according to context. Sequence identity can be expressed in terms of a percentage of sequence identity of a first sequence to a second sequence. Percent (%) sequence identity with respect to a reference DNA sequence is the percentage of DNA nucleotides in a candidate sequence that are identical with the DNA nucleotides in the reference DNA sequence after aligning the sequences and introducing gaps, as necessary. Percent (%) sequence identity with respect to a reference amino acid sequence is the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity.

As used herein, the term “antibody” refers to an immunoglobulin molecule that specifically binds to, or is immunologically reactive toward, a specific antigen. Antibody can include, for example, polyclonal, monoclonal, genetically engineered, and antigen binding fragments thereof. An antibody can be, for example, murine, chimeric, humanized, heteroconjugate, bispecific, a diabody, a triabody, or a tetrabody. The antigen binding fragment can include, for example, Fab′, F(ab′)₂, Fab, Fv, rIgG, and scFv.

As used herein, the abbreviations for the natural L-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); and valine (V, Val). Unless otherwise specified, X can indicate any amino acid. In some aspects, X can be asparagine (N), glutamine (Q), histidine (H), lysine (K), or arginine (R).

As used herein, an “antigen binding domain” refers to a region of a molecule that binds to an antigen. An antigen binding domain may be a domain that can specifically bind to an antigen. An antigen binding domain can be an antigen-binding portion of an antibody or an antibody fragment. An antigen binding domain can be one or more fragments of an antibody that can retain the ability to specifically bind to an antigen. An antigen binding domain can be an antigen binding fragment. An antigen binding domain can recognize a single antigen.

As used herein, an “antibody construct” refers to a molecule, e.g., a protein, peptide, antibody or portion thereof, that contains an antigen binding domain and an Fc domain. An antibody construct can recognize, for example, one antigen or multiple antigens.

“Conjugate”, as used herein, refers to an antibody construct that is linked, e.g., covalently linked, either directly or through a linker to a compound described herein, e.g., a compound or salt of any one of Formulas (I), (Ia), (Ib), (Ic), and (Id).

As used herein, an “Fc domain” can be an Fc domain from an antibody or from a non-antibody that can bind to an Fc receptor.

As used herein, “recognize” and “specifically bind” with regard to antibody or antibody construct interactions refer to the specific association or binding between an antigen binding domain of an antibody or portion thereof and an antigen, as compared with the binding of the antibody or antibody construct to a non-antigen.

As used herein, a “target binding domain” refers to a construct that contains an antigen binding domain from an antibody or from a non-antibody that can specifically bind to the antigen.

As used herein, a “tumor antigen” refers to an antigenic substance associated with a tumor or cancer cell, and can trigger an immune response in a host.

The phrase “targeting moiety” refers to a structure that has a selective affinity for a target molecule relative to other non-target molecules. The targeting moiety binds to a target molecule. A targeting moiety may include, for example, an antibody, a peptide, a ligand, a receptor, or a binding portion thereof. The target biological molecule may be a biological receptor or other structure of a cell such as a tumor antigen.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The term “C_{x-y}” when used in conjunction with a chemical moiety, such as alkyl, alkenyl, or alkynyl, is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkynylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons, respectively. In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons, respectively. In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons, respectively. In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

“Heteroalkylene” refers to a straight divalent hydrocarbon chain including at least one heteroatom in the chain, containing no unsaturation, and preferably having from one to twelve carbon atoms and from one to 6 heteroatoms, e.g., —O—, —NH—, —S—, and —B—. The heteroalkylene chain is attached to the rest of the molecule through a single bond and to the radical group through a single bond. The points of attachment of the heteroalkylene chain to the rest of the molecule and to the radical group are through the terminal atoms of the chain. In other embodiments, a heteroalkylene comprises one to five carbon atoms and from one to three heteroatoms. In other embodiments, a heteroalkylene comprises one to four carbon atoms and from one to three heteroatoms. In other embodiments, a heteroalkylene comprises one to three carbon atoms and from one to two heteroatoms. In other embodiments, a heteroalkylene comprises one to two carbon atoms and from one to two heteroatoms. In other embodiments, a heteroalkylene comprises one carbon atom and from one to two heteroatoms. In other embodiments, a heteroalkylene comprises five to eight carbon atoms and from one to four heteroatoms. In other embodiments, a heteroalkylene comprises two to five carbon atoms and from one to three heteroatoms. In other embodiments, a heteroalkylene comprises three to five carbon atoms and from one to three heteroatoms. Unless stated otherwise specifically in the specification, a heteroalkylene chain is optionally substituted by one or more substituents such as those substituents described herein.

“Heteroalkenylene” refers to a straight divalent hydrocarbon chain including at least one heteroatom in the chain, containing at least one carbon-carbon double bond, and preferably having from one to twelve carbon atoms and from one to 6 heteroatoms, e.g., —O—, —NH—, —S—, and —B—. The heteroalkenylene chain is attached to the rest of the molecule through a single bond and to the radical group through a single bond. The points of attachment of the heteroalkenylene chain to the rest of the molecule and to the radical group are through the terminal atoms of the chain. In certain embodiments, a heteroalkenylene comprises two to five carbon atoms and from one to three heteroatoms. In other embodiments, a heteroalkenylene comprises two to four carbon atoms and from one to three heteroatoms. In certain embodiments, a heteroalkenylene comprises two to three carbon atoms and from one to two heteroatoms. In certain embodiments, a heteroalkenylene comprises two carbon atoms and from one to two heteroatoms. In certain embodiments, a heteroalkenylene comprises five to eight carbon atoms and from one to four heteroatoms. In certain embodiments, a heteroalkenylene comprises two to five carbon atoms and from one to three heteroatoms. In other embodiments, a heteroalkenylene comprises three to five carbon atoms and from one to three heteroatoms. Unless stated otherwise specifically in the specification, a heteroalkenylene chain is optionally substituted by one or more substituents such as those substituents described herein.

The term “carbocycle” as used herein refers to a saturated, unsaturated, or aromatic ring or ring system in which each atom of the ring(s) is carbon. Carbocycle includes 3- to 10-membered monocyclic rings, 4- to 12-membered bicyclic rings (e.g., 6- to 12-membered bicyclic rings), and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated, and aromatic bicyclic rings, as valence permits. A bicyclic carbocycle includes any combination of ring sizes such as 4-5 fused ring systems, 5-5 fused ring systems, 5-6 fused ring systems, and 6-6 fused ring systems. Exemplary carbocycles include cyclopropyl, cyclobutyl, cyclopentyl, cyclohexyl, cyclohexenyl, adamantyl, phenyl, indanyl, and naphthyl. The term “saturated carbocycle” refers to carbocycles including no multiple bonds (e.g., double or triple bonds). Examples of saturated carbocycles include cyclopropane, cyclobutane, cyclopentane, and cyclohexane. The term “unsaturated carbocycle” refers to carbocycles with at least one degree of unsaturation and excluding aromatic carbocycles. Examples of unsaturated carbocycles include cyclohexadiene, cyclohexene, and cyclopentene. Further examples of carbocycles include, but are not limited to, cyclopropane, cyclobutane, cyclopentane, cyclopentadiene, cyclohexane, cycloheptane, cycloheptene, naphthalene, and adamantine.

The term “heterocycle” as used herein refers to a saturated, unsaturated, or aromatic ring or ring system including one or more heteroatoms. Exemplary heteroatoms include N, O, Si, P, B, and S atoms. Heterocycles include, for example, 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, and 6-6 fused ring systems. The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine. Additional examples of heterocycles include, but are not limited to, indole, benzothiophene, benzthiazole, benzoxazole, benzimidazole, oxazolopyridine, imidazopyridine, thiazolopyridine, furan, oxazole, pyrrole, pyrazole, imidazole, thiophene, thiazole, isothiazole, and isoxazole.

The term “aryl” includes aromatic carbocycles with single ring structures or polycyclic structures. For a polycyclic aryl group, at least one of the rings of the polycycle is aromatic. Examples of aryl include phenyl, naphthyl, and dihydronaphthyl.

The term “heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more cyclic rings in which two or more atoms are common to two adjoining rings wherein at least one of the rings is heteroaromatic, e.g., the other cyclic rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OC(O)-R^{a}, —R^{b}—OC(O)——OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OC(O)—R^{a}, —R^{b}—OC(O)—OR^{a}, —R^{b}—OC(O)—N(R^{a})₂, —R^{b}—N(R^{a})₂, —R^{b}-C(O)R^{a}, —R^{b}—C(O)OR^{a}, —R^{b}—C(O)N(R^{a})₂, —R^{b}—O—R^{a}—C(O)N(R^{a})₂, —R^{b}—N(R^{a})C(O)OR^{a}, —R^{b}-N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants, unless specified otherwise.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E-, and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

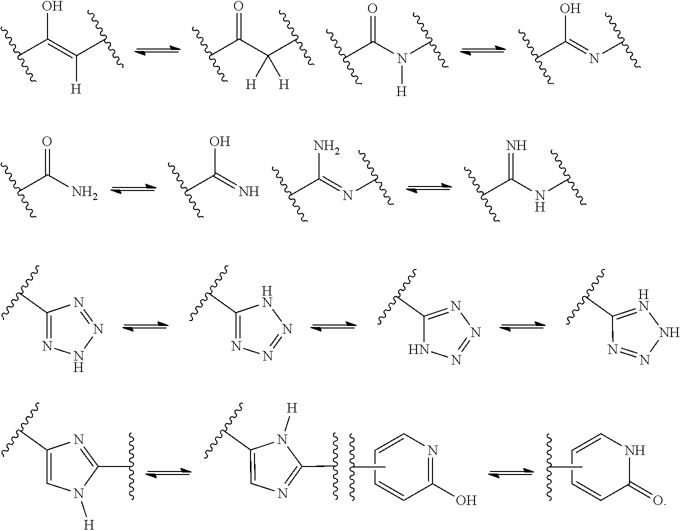

The compounds disclosed herein, in some embodiments, are used in different enriched isotopic forms, e.g., enriched in the content of ²H, ³H, ¹¹C ¹³C and/or ¹⁴C. In certain embodiments, the compound is deuterated in at least one position. Such deuterated forms can be made by the procedure described in, for example, U.S. Pat. Nos. 5,846,514 and 6,334,997. As described in therein, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C) Isotopic substitution with ²H, ¹¹C, ¹³_{C,} ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. Methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, a moiety described herein includes the symbol [symbol] which indicates the point of attachment, e.g., the point of attachment of a chemical moiety to the compound, the point of attachment of a linker to a compound of the disclosure, or the point of attachment of a linker to an antibody construct, as described herein.

### Antibody Construct

Disclosed herein are targeting moieties and antibody constructs that may be used together with compounds of the disclosure. In certain embodiments, compounds of the disclosure are linked, e.g., covalently linked, either directly or through a linker to an antibody construct or targeting moiety, thereby forming conjugates. In certain embodiments, conjugates are represented by:

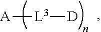

wherein A is an antibody construct; L³ is a linker; D is a compound or salt of any one of Formulas (I), (Ia), (Ib), (Ic), (Id) and (Ie); and n is from 1 to 20. In certain embodiments, n is from 1 to 10, such as from 1 to 9, such as from 1 to 8, such as from 2 to 8, such as from 1 to 6, such as from 3 to 5 or such as from 1 to 3.

In certain embodiments, a compound or salt of the disclosure, e.g., a compound or salt of Formula (I), (Ia), (Ib), (Ic), (Id) and (Ie), may be referred to herein as a drug, D, an ALK5 inhibitor, a TGF-βR1 inhibitor, or a payload, particularly when referenced as part of a conjugate. “LP”, “linker-payload”, “D-L³”, or “compound-linker” may be used herein to refer to a compound or salt of the disclosure bound to a linker.

An antibody construct may contain, for example, two, three, four, five, six, seven, eight, nine, ten, or more antigen binding domains. An antibody construct may contain two antigen binding domains in which each antigen binding domain can recognize the same antigen. An antibody construct may contain two antigen binding domains in which each antigen binding domain can recognize different antigens. An antigen binding domain may be in a scaffold, in which a scaffold is a supporting framework for the antigen binding domain. An antigen binding domain may be in a non-antibody scaffold. An antigen binding domain may be in an antibody scaffold. An antibody construct may comprise an antigen binding domain in a scaffold. The antibody construct may comprise a Fc fusion protein. In some embodiments, the antibody construct is a Fc fusion protein. In some embodiments, an antigen binding domain may specifically bind to a tumor antigen. An antigen binding domain may specifically bind to an antigen that is at least 80%, at least 90%, at least 95%, at least 99%, or 100% homologous to a tumor antigen. In some embodiments, an antigen binding domain may specifically bind to an antigen on an immune cell, such as an antigen presenting cell (APC). An antigen binding domain may specifically bind to an antigen that is at least 80%, at least 90%, at least 95%, at least 99%, or 100% homologous to an antigen on an immune cell, such as an antigen presenting cell (APC). In some embodiments, an antigen binding domain may specifically bind to an antigen on non-cancerous cells associated with fibrosis, autoimmune disease or inflammatory disease, such as stellate cells, myofibroblasts, synovial fibroblasts, epithelial cells, or podocytes. An antigen binding domain may specifically bind to an antigen that is at least 80%, at least 90%, at least 95%, at least 99%, or 100% homologous to an antigen on non-cancerous cells associated with fibrosis, autoimmune disease or inflammatory disease, such as stellate cells, myofibroblasts, synovial fibroblasts, epithelial cells, or podocytes.

An antigen binding domain of an antibody may comprise one or more light chain (LC) CDRs and one or more heavy chain (HC) CDRs. For example, an antigen binding domain of an antibody may comprise one or more of the following: a light chain complementary determining region 1 (LC CDR¹⁰), a light chain complementary determining region 2 (LC CDR2), or a light chain complementary determining region 3 (LC CDR3). For another example, an antigen binding domain may comprise one or more of the following: a heavy chain complementary determining region 1 (HC CDR¹⁰), a heavy chain complementary determining region 2 (HC CDR2), or a heavy chain complementary determining region 3 (HC CDR3). As an additional example, an antigen binding domain of an antibody may comprise one or more of the following: LC CDR1, LC CDR2, LC CDR3, HC CDR1, HC CDR2, and HC CDR3. In further embodiments, an antigen binding domain of an antibody may comprise all six of the following: LC CDR1, LC CDR2, LC CDR3, HC CDR1, HC CDR2, and HC CDR3.

The antigen binding domain of an antibody construct may be selected from any domain that binds the antigen including, but not limited to, from a monoclonal antibody, a polyclonal antibody, a recombinant antibody, or an antigen binding fragment thereof, for example, a heavy chain variable domain (V_{H}) and a light chain variable domain (V_{L}), or a DARPin, an affimer, an avimer, a knottin, a monobody, an affinity clamp, an ectodomain, a receptor ectodomain, a receptor, a T cell receptor, or a recombinant T cell receptor.

The antigen binding domain of an antibody construct may be at least 80% homologous to an antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor.

In certain embodiments, an antibody construct comprises an Fc domain that may further comprise an Fc region, in which the Fc domain may be the part of an Fc region that interacts with Fc receptors. The Fc domain of an antibody construct may interact with Fc-receptors (FcRs) found on immune cells. The Fc domain may also mediate the interaction between effector molecules and cells, which can lead to activation of the immune system. The Fc domain may be derived from IgG, IgA, or IgD antibody isotypes, and may comprise two identical protein fragments, which are derived from the second and third constant domains of the antibody's heavy chains. In an Fc domain derived from an IgG antibody isotype, the Fc domain may comprise a highly-conserved N-glycosylation site, which may be essential for FcR-mediated downstream effects. The Fc domain may be derived from IgM or IgE antibody isotypes, in which the Fc region may comprise three heavy chain constant domains.

An Fc domain may interact with different types of FcRs. The different types of FcRs may include, for example, FcγRI, FcγRIIA, FcγRIIB, FcγRIIIA, FcγRIIIB, FcαRI, FcμR, FcεRI, FcεRII, and FcRn. FcRs may be located on the membrane of certain immune cells including, for example, B lymphocytes, natural killer cells, macrophages, monocytes, neutrophils, follicular dendritic cells, eosinophils, basophils, platelets, and mast cells. Once the FcR is engaged by the Fc domain, the FcR may initiate functions including, for example, clearance of an antigen-antibody complex via receptor-mediated endocytosis, antibody-dependent cell-mediated cytotoxicity (ADCC), antibody dependent cell-mediated phagocytosis (ADCP), and ligand-triggered transmission of signals across the plasma membrane that can result in alterations in secretion, exocytosis, and cellular metabolism. FcRs may deliver signals when FcRs are aggregated by antibodies and multivalent antigens at the cell surface. The aggregation of FcRs with immunoreceptor tyrosine-based activation motifs (ITAMs) may sequentially activate SRC family tyrosine kinases and SYK family tyrosine kinases. ITAM comprises a twice-repeated YxxL sequence flanking seven variable residues. The SRC and SYK kinases may connect the transduced signals with common activation pathways.

In some embodiments, an Fc domain or region can exhibit reduced binding affinity to one or more Fc receptors. In some embodiments, an Fc domain or region can exhibit reduced binding affinity to one or more Fcgamma receptors. In some embodiments, an Fc domain or region can exhibit reduced binding affinity to FcRn receptors. In some embodiments, an Fc domain or region can exhibit reduced binding affinity to Fcgamm and FcRn receptors. In some embodiments, an Fc domain is an Fc null domain or region. As used herein, an “Fc null” refers to a domain that exhibits weak to no binding to any of the Fcgamma receptors. In some embodiments, an Fc null domain or region exhibits a reduction in binding affinity (e.g., increase in Kd) to Fc gamma receptors of at least 1000-fold.

The Fc domain may have one or more, two or more, three or more, or four or more amino acid substitutions that decrease binding of the Fc domain to an Fc receptor. In certain embodiments, an Fc domain exhibits decreased binding to FcγRI (CD64), FcγRIIA (CD32), FcγRIIIA (CD16a), FcγRIIIB (CD16b), or any combination thereof. In order to decrease binding affinity of an Fc domain or region to an Fc receptor, the Fc domain or region may comprise one or more substitutions that has the effect of reducing the affinity of the Fc domain or region to an Fc receptor. In certain embodiments, the one or more substitutions comprise any one or more of IgG1 heavy chain mutations corresponding to E233P, L234V, L234A, L235A, L235E, ΔG236, G237A, E318A, K320A, K322A, A327G, A330S, or P331S according to the EU index of Kabat numbering.

In some embodiments, the Fc domain or region can comprise a sequence of the IgG1 isoform that has been modified from the wild-type IgG1 sequence. A modification can comprise a substitution at more than one amino acid residue, such as at 5 different amino acid residues including L235V/F243L/R292P/Y300L/P396L (IgG1VLPLL) according to the EU index of Kabat numbering. A modification can comprise a substitution at more than one amino acid residue such as at 2 different amino acid residues including S239D/I332E (IgG1DE) according to the EU index of Kabat numbering. A modification can comprise a substitution at more than one amino acid residue such as at 3 different amino acid residues including S298A/E333A/K334A (IgG1AAA) according to the EU index of Kabat numbering.

An antibody may consist of two identical light chains and two identical heavy chains, all held together covalently by disulfide linkages. The N-terminal regions of the light and heavy chains together may form the antigen recognition site of an antibody. Structurally, various functions of an antibody may be confined to discrete protein domains (e.g., regions). The sites that can recognize and can bind antigen may consist of three complementarities determining regions (CDRs) that may lie within the variable heavy chain region and variable light chain region at the N-terminal end of the heavy chain and the light chain. The constant domains may provide the general framework of the antibody and may not be involved directly in binding the antibody to an antigen, but may be involved in various effector functions, such as participation of the antibody in antibody-dependent cellular cytotoxicity, and may bind Fc receptors. The constant domains may be in an Fc region. The constant domains may include an Fc domain. The domains of natural light and heavy chains may have the same general structures, and each domain may comprise four framework regions, whose sequences can be somewhat conserved, connected by three hyper-variable regions or CDRs. The four framework regions (FR) may largely adopt a β-sheet conformation and the CDRs can form loops connecting, and in some aspects forming part of, the β-sheet structure. The CDRs in each chain may be held in close proximity by the framework regions and, with the CDRs from the other chain, may contribute to the formation of the antigen binding site.

An antibody construct may comprise a light chain of an amino acid sequence having at least one, two, three, four, five, six, seven, eight, nine or ten modifications and in certain embodiments, not more than 40, 35, 30, 25, 20, 15 or 10 modifications of the amino acid sequence relative to the natural or original amino acid sequence. An antibody construct may comprise a heavy chain of an amino acid sequence having at least one, two, three, four, five, six, seven, eight, nine or ten modifications and in certain embodiments, not more than 40, 35, 30, 25, 20, 15 or 10 modifications of the amino acid sequence relative to the natural or original amino acid sequence.

An antibody of an antibody construct may include an antibody of any type, which may be assigned to different classes of immunoglobulins, e.g., IgA, IgD, IgE, IgG, and IgM. Several different classes may be further divided into isotypes, e.g., IgG1, IgG2, IgG3, IgG4, IgA1, and IgA2. An antibody may further comprise a light chain and a heavy chain, often more than one chain. The heavy-chain constant regions (Fc) that corresponds to the different classes of immunoglobulins may be α, δ, ε, γ, and μ, respectively. The light chains may be one of either kappa (κ) or lambda (λ), based on the amino acid sequences of the constant domains. The Fc domain may comprise an Fc region. An Fc receptor may bind an Fc domain. Antibody constructs may also include any fragment or recombinant forms thereof, including but not limited to, single chain variable fragments (scFvs), ‘T-bodies’, anti-calins, centyrins, affibodies, domain antibodies, or peptibodies.

An antibody construct may comprise an antigen binding antibody fragment. An antibody fragment may include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; and (iii) a Fv fragment consisting of the V_{L} and V_{H} domains of a single arm of an antibody. Although the two domains of the Fv fragment, V_{L} and V_{H}, may be coded for by separate genes, they may be linked by a synthetic linker to be made as a single protein chain in which the V_{L} and V_{H} regions pair to form monovalent molecules.

F(ab′)₂ and Fab′ moieties may be recombinantly produced or produced by treating immunoglobulin (e.g., monoclonal antibody) with a protease such as pepsin and papain, and may include an antibody fragment generated by digesting immunoglobulin near the disulfide bonds existing between the hinge regions in each of the two H chains. The Fab fragment may also contain the constant domain of the light chain and the first constant domain (C_{H1}) of the heavy chain. Fab′ fragments may differ from Fab fragments by the addition of a few residues at the carboxyl terminus of the heavy chain C_{H1} domain including one or more cysteine(s) from the antibody hinge region.

An Fv may be the minimum antibody fragment which contains a complete antigen-recognition and antigen-binding site. This region may consist of a dimer of one heavy chain and one light chain variable domain in tight, non-covalent association. In this configuration, the CDRs of each variable domain may interact to define an antigen-binding site on the surface of the V_{H}-V_{L} dimer. A single variable domain (or half of an Fv comprising only three hypervariable regions or CDRs specific for an antigen) may recognize and bind antigen, although the binding can be at a lower affinity than the affinity of the entire binding site.

An antibody may include an Fc domain comprising an Fc region. The Fc domain of an antibody may interact with FcRs found on immune cells. The Fc domain may also mediate the interaction between effector molecules and cells, which may lead to activation of the immune system. In the IgG, IgA, and IgD antibody isotypes, the Fc domain or region may comprise two identical protein fragments, which can be derived from the second and third constant domains of the antibody's heavy chains. In the IgM and IgE antibody isotypes, the Fc regions may comprise three heavy chain constant domains. In the IgG antibody isotype, the Fc regions may comprise a highly-conserved N-glycosylation site, which may be important for FcR-mediated downstream effects.

An antibody used herein may be “chimeric” or “humanized.” Chimeric and humanized forms of non-human (e.g., murine) antibodies can be chimeric immunoglobulins, immunoglobulin chains or fragments thereof (such as Fv, Fab, Fab′, F(ab′)₂ or other target-binding subdomains of antibodies), which may contain minimal sequences derived from non-human immunoglobulin. In general, the humanized antibody may comprise substantially all of at least one, and typically two, variable domains, in which all or substantially all of the CDRs correspond to those of a non-human immunoglobulin and all or substantially all of the framework regions are those of a human immunoglobulin sequence. The humanized antibody may also comprise at least a portion of an immunoglobulin constant region (Fc), typically that of a human immunoglobulin consensus sequence.

An antibody may be a human antibody. As used herein, “human antibodies” can include antibodies having, for example, the amino acid sequence of a human immunoglobulin and may include antibodies isolated from human immunoglobulin libraries or from animals transgenic for one or more human immunoglobulins that do not express endogenous immunoglobulins. Human antibodies may be produced using transgenic mice which are incapable of expressing functional endogenous immunoglobulins, but which may express human immunoglobulin genes. Completely human antibodies that recognize a selected epitope may be generated using guided selection. In this approach, a selected non-human monoclonal antibody, e.g., a mouse antibody, may be used to guide the selection of a completely human antibody recognizing the same epitope.

An antibody may be a bispecific antibody or a dual variable domain antibody (DVD). Bispecific and DVD antibodies may be monoclonal, often human or humanized, antibodies that can have binding specificities for at least two different antigens.

An antibody may be a derivatized antibody. For example, derivatized antibodies may be modified by glycosylation, deglycosylation, defucosylation, acetylation, pegylation, phosphorylation, amidation, derivatization by known protecting/blocking groups, proteolytic cleavage, linkage to a cellular ligand or other protein.

An antibody may have a sequence that has been modified to alter at least one constant region-mediated biological effector function relative to the corresponding wild type sequence. For example, in some embodiments, the antibody can be modified to reduce at least one constant region-mediated biological effector function relative to an unmodified antibody, e.g., reduced binding to the Fc receptor (FcR). FcR binding may be reduced by, for example, mutating the immunoglobulin constant region segment of the antibody at particular regions necessary for FcR interactions.

In certain embodiments, a targeting moiety comprises an Fc domain or an Fc region; The Fc domain or Fc region may interact with one or more Fc receptors (FcRs). An Fc domain of an antibody construct may interact with Fc receptors. An Fc domain or Fc region may interact with Fc receptors found on immune cells. An Fc domain or Fc region may also mediate the interaction between effector molecules and cells, which can lead to activation of the immune system. An Fc domain or Fc region may be derived from IgG, IgA, or IgD antibody isotypes, and may comprise two identical protein fragments, which are derived from the second and third constant domains of the antibody's heavy chains. In an Fc domain or region derived from an IgG antibody isotype, the Fc domain or region may comprise a highly-conserved N-glycosylation site, which may be essential for FcR-mediated downstream effects. The Fc domain or region may be derived from IgM or IgE antibody isotypes, in which the Fc domain or region may comprise three heavy chain constant domains.

An Fc domain may interact with different types of FcRs. The different types of FcRs may include, for example, FcγRI, FcγRIIA, FcγRIIB, FcγRIIIA, FcγRIIIB, FcαRI, FcμR, FcεRI, FcεRII, and FcRn. FcRs may be located on the membrane of certain immune cells including, for example, B lymphocytes, natural killer cells, macrophages, neutrophils, follicular dendritic cells, eosinophils, basophils, platelets, and mast cells. Once the FcR is engaged by the Fc domain, the FcR may initiate functions including, for example, clearance of an antigen-antibody complex via receptor-mediated endocytosis, antibody-dependent cell-mediated cytotoxicity (ADCC), antibody dependent cell-mediated phagocytosis (ADCP), and ligand-triggered transmission of signals across the plasma membrane that can result in alterations in secretion, exocytosis, and cellular metabolism. FcRs may deliver signals when FcRs are aggregated by antibodies and multivalent antigens at the cell surface. The aggregation of FcRs with immunoreceptor tyrosine-based activation motifs (ITAMs) may sequentially activate SRC family tyrosine kinases and SYK family tyrosine kinases. ITAM comprises a twice-repeated YxxL sequence flanking seven variable residues. The SRC and SYK kinases may connect the transduced signals with common activation pathways.

In some embodiments, an Fc domain or region of the antibody construct portion of a conjugate can exhibit increased binding affinity to one or more Fc receptors. In some embodiments, an Fc domain or region can exhibit increased binding affinity to one or more Fcgamma receptors. In some embodiments, an Fc domain or region can exhibit increased binding affinity to FcRn receptors. In some embodiments, an Fc domain or region can exhibit increased binding affinity to Fcgamma and FcRn receptors.

In some embodiments, an Fc domain or region of the antibody construct portion of a conjugate can exhibit reduced binding affinity to one or more Fc receptors. In some embodiments, an Fc domain or region can exhibit reduced binding affinity to one or more Fcgamma receptors. In some embodiments, an Fc domain or region can exhibit reduced binding affinity to FcRn receptors. In some embodiments, an Fc domain or region can exhibit reduced binding affinity to Fcgamma and FcRn receptors. In some embodiments, an Fc domain is an Fc null domain or region. In some embodiments, an Fc domain or region can exhibit reduced binding affinity to FcRn receptors, but have the same or increased binding affinity to one or more Fcgamma receptors as compared to a wildtype IgG. In some embodiments, an Fc domain or region can exhibit increased binding affinity to FcRn receptors, but have the same or decreased binding affinity to one or more Fcgamma receptors.

The Fc domain may have one or more, two or more, three or more, or four or more amino acid substitutions that decrease binding of the Fc domain to an Fc receptor. In certain embodiments, an Fc domain has decreased binding affinity for one or more of FcγRI (CD64), FcγRIIA (CD32), FcγRIIIA (CD16a), FcγRIIIB (CD16b), or any combination thereof. In order to decrease binding affinity of an Fc domain or region to an Fc receptor, the Fc domain or region may comprise one or more amino acid substitutions that reduces the binding affinity of the Fc domain or region to an Fc receptor.

In certain embodiments, the one or more substitutions comprise any one or more of IgG1 heavy chain mutations corresponding to E233P, L234V, L234A, L235A, L235E, ΔG236, G237A, E318A, K320A, K322A, A327G, A330S, or P331S according to the EU index of Kabat numbering.

In some embodiments, the Fc domain or region can comprise a sequence of an IgG isoform that has been modified from the wild-type IgG sequence. In some embodiments, the Fc domain or region can comprise a sequence of the IgG1 isoform that has been modified from the wild-type IgG1 sequence. In some embodiments, the modification comprises substitution of one or more amino acids that reduce binding affinity of an IgG Fc domain or region to all Fcγ receptors. A modification can be substitution of E233, L234 and L235, such as E233P/L234V/L235A or E233P/L234V/L235A/ΔG236, according to the EU index of Kabat. A modification can be a substitution of P238, such as P238A, according to the EU index of Kabat. A modification can be a substitution of D265, such as D265A, according to the EU index of Kabat. A modification can be a substitution of N297, such as N297A, according to the EU index of Kabat. A modification can be a substitution of A327, such as A327Q, according to the EU index of Kabat. A modification can be a substitution of P329, such as P239A, according to the EU index of Kabat.

In some embodiments, an IgG Fc domain or region comprises at least one amino acid substitution that reduces its binding affinity to FcγR1, as compared to a wild-type or reference IgG Fc domain. A modification can comprise a substitution at F241, such as F241A, according to the EU index of Kabat. A modification can comprise a substitution at F243, such as F243A, according to the EU index of Kabat. A modification can comprise a substitution at V264, such as V264A, according to the EU index of Kabat. A modification can comprise a substitution at D265, such as D265A according to the EU index of Kabat.

In some embodiments, an IgG Fc domain or region comprises at least one amino acid substitution that increases its binding affinity to FcγR1, as compared to a wild-type or reference IgG Fc domain. A modification can comprise a substitution at A327 and P329, such as A327Q/P329A, according to the EU index of Kabat.

In some embodiments, the modification comprises substitution of one or more amino acids that reduce binding affinity of an IgG Fc domain or region to FcγRII and FcγRIIIA receptors. A modification can be a substitution of D270, such as D270A, according to the EU index of Kabat. A modification can be a substitution of Q295, such as Q295A, according to the EU index of Kabat. A modification can be a substitution of A327, such as A237S, according to the EU index of Kabat.

In some embodiments, the modification comprises substitution of one or more amino acids that increases binding affinity of an IgG Fc domain or region to FcγRII and FcγRIIIA receptors. A modification can be a substitution of T256, such as T256A, according to the EU index of Kabat. A modification can be a substitution of K290, such as K290A, according to the EU index of Kabat.

In some embodiments, the modification comprises substitution of one or more amino acids that increases binding affinity of an IgG Fc domain or region to FcγRII receptor. A modification can be a substitution of R255, such as R255A, according to the EU index of Kabat. A modification can be a substitution of E258, such as E258A, according to the EU index of Kabat. A modification can be a substitution of 5267, such as S267A, according to the EU index of Kabat. A modification can be a substitution of E272, such as E272A, according to the EU index of Kabat. A modification can be a substitution of N276, such as N276A, according to the EU index of Kabat. A modification can be a substitution of D280, such as D280A, according to the EU index of Kabat. A modification can be a substitution of H285, such as H285A, according to the EU index of Kabat. A modification can be a substitution of N286, such as N286A, according to the EU index of Kabat. A modification can be a substitution of T307, such as T307A, according to the EU index of Kabat. A modification can be a substitution of L309, such as L309A, according to the EU index of Kabat. A modification can be a substitution of N315, such as N315A, according to the EU index of Kabat. A modification can be a substitution of K326, such as K326A, according to the EU index of Kabat. A modification can be a substitution of P331, such as P331A, according to the EU index of Kabat. A modification can be a substitution of S337, such as S337A, according to the EU index of Kabat. A modification can be a substitution of A378, such as A378A, according to the EU index of Kabat. A modification can be a substitution of E430, such as E430A, according to the EU index of Kabat.

In some embodiments, the modification comprises substitution of one or more amino acids that increases binding affinity of an IgG Fc domain or region to FcγRII receptor and reduces the binding affinity to FcγRIIIA receptor. A modification can be a substitution of H268, such as H268A, according to the EU index of Kabat. A modification can be a substitution of R301, such as R301A, according to the EU index of Kabat. A modification can be a substitution of K322, such as K322A, according to the EU index of Kabat.

In some embodiments, the modification comprises substitution of one or more amino acids that decreases binding affinity of an IgG Fc domain or region to FcγRII receptor but does not affect the binding affinity to FcγRIIIA receptor. A modification can be a substitution of R292, such as R292A, according to the EU index of Kabat. A modification can be a substitution of K414, such as K414A, according to the EU index of Kabat.

In some embodiments, the modification comprises substitution of one or more amino acids that decreases binding affinity of an IgG Fc domain or region to FcγRII receptor and increases the binding affinity to FcγRIIIA receptor. A modification can be a substitution of S298, such as S298A, according to the EU index of Kabat. A modification can be substitution of S239, I332 and A330, such as S239D/I332E/A330L. A modification can be substitution of S239 and I332, such as S239D/I332E.

In some embodiments, the modification comprises substitution of one or more amino acids that decreases binding affinity of an IgG Fc domain or region to FcγRIIIA receptor. A modification can be substitution of F241 and F243, such as F241S/F243S or F241I/F2431, according to the EU index of Kabat.

In some embodiments, the modification comprises substitution of one or more amino acids that decreases binding affinity of an IgG Fc domain or region to FcγRIIIA receptor and does not affect the binding affinity to FcγRII receptor. A modification can be a substitution of S239, such as S239A, according to the EU index of Kabat. A modification can be a substitution of E269, such as E269A, according to the EU index of Kabat. A modification can be a substitution of E293, such as E293A, according to the EU index of Kabat. A modification can be a substitution of Y296, such as Y296F, according to the EU index of Kabat. A modification can be a substitution of V303, such as V303A, according to the EU index of Kabat. A modification can be a substitution of A327, such as A327G, according to the EU index of Kabat. A modification can be a substitution of K338, such as K338A, according to the EU index of Kabat. A modification can be a substitution of D376, such as D376A, according to the EU index of Kabat.

In some embodiments, the modification comprises substitution of one or more amino acids that increases binding affinity of an IgG Fc domain or region to FcγRIIIA receptor and does not affect the binding affinity to FcγRII receptor. A modification can be a substitution of E333, such as E333A, according to the EU index of Kabat. A modification can be a substitution of K334, such as K334A, according to the EU index of Kabat. A modification can be a substitution of A339, such as A339T, according to the EU index of Kabat. A modification can be substitution of S239 and I332, such as S239D/I332E.

In some embodiments, the modification comprises substitution of one or more amino acids that increases binding affinity of an IgG Fc domain or region to FcγRIIIA receptor. A modification can be substitution of L235, F243, R292, Y300 and P396, such as L235V/F243L/R292P/Y300L/P396L (IgG1VLPLL) according to the EU index of Kabat. A modification can be substitution of S298, E333 and K334, such as S298A/E333A/K334A, according to the EU index of Kabat. A modification can be substitution of K246, such as K246F, according to the EU index of Kabat.

Other substitutions in an IgG Fc domain that affect its interaction with one or more Fcy receptors are disclosed in U.S. Pat. Nos. 7,317,091 and 8,969,526 (the disclosures of which are incorporated by reference herein).

In some embodiments, an IgG Fc domain or region comprises at least one amino acid substitution that reduces the binding affinity to FcRn, as compared to a wild-type or reference IgG Fc domain. A modification can comprise a substitution at H435, such as H435A according to the EU index of Kabat. A modification can comprise a substitution at I253, such as I253A according to the EU index of Kabat. A modification can comprise a substitution at H310, such as H310A according to the EU index of Kabat. A modification can comprise substitutions at I253, H310 and H435, such as I253A/H310A/H435A according to the EU index of Kabat.

A modification can comprise a substitution of one amino acid residue that increases the binding affinity of an IgG Fc domain for FcRn, relative to a wildtype or reference IgG Fc domain. A modification can comprise a substitution at V308, such as V308P according to the EU index of Kabat. A modification can comprise a substitution at M428, such as M428L according to the EU index of Kabat. A modification can comprise a substitution at N434, such as N434A according to the EU index of Kabat or N434H according to the EU index of Kabat. A modification can comprise substitutions at T250 and M428, such as T250Q and M428L according to the EU index of Kabat. A modification can comprise substitutions at M428 and N434, such as M428L and N434S, N434A or N434H according to the EU index of Kabat. A modification can comprise substitutions at M252, S254 and T256, such as M252Y/S254T/T256E according to the EU index of Kabat. A modification can be a substitution of one or more amino acids selected from P257L, P257N, P257I, V279E, V279Q, V279Y, A281S, E283F, V284E, L306Y, T307V, V308F, Q311V, D376V, and N434H. Other substitutions in an IgG Fc domain that affect its interaction with FcRn are disclosed in U.S. Pat. No. 9,803,023 (the disclosure of which is incorporated by reference herein).

In certain embodiments, the antibody construct comprises an antigen binding domain and an IgG Fc domain, wherein a K_{d} for binding of the antigen binding domain to a first antigen in a presence of the ALK5 inhibitor is less than about 100 nM and no greater than about 100 times a K_{d} for binding of the antigen binding domain to the first antigen in the absence of the ALK5 inhibitor. In certain embodiments, the antibody construct comprises a K_{d} for binding of the IgG Fc domain to an Fc receptor in the presence of the ALK5 inhibitor is no greater than about 100 times a K_{d} for binding the IgG Fc domain to the Fc receptor in the absence of the ALK5 inhibitor. In certain embodiments, the first antigen is selected from MUC16, UPK1B, VTCN1, TMPRSS3, TMEM238, C1orf186, TMPRSS4, CLDN6, CLDN8 and STRA6. In certain embodiments, the first antigen is selected from MUC16, UPK1B, VTCN1, TMPRSS3, TMEM238, C1orf186 and TMPRSS4. In certain embodiments, the first antigen is MUC16. In certain embodiments, the first antigen is UPK1B. In certain embodiments, the first antigen is VTCN1. In certain embodiments, the first antigen is TMPRSS3. In certain embodiments, the first antigen is TMEM238. In certain embodiments, the first antigen is C1orf186. In certain embodiments, the first antigen is TMPRSS4. In certain embodiments, the first antigen is CLDN6. In certain embodiments, the first antigen is CLDN8. In certain embodiments, the first antigen is STRA6.

In certain embodiments, the first antigen is selected from ACSLS, AP1M2, AREG, CDH1, CDH17, CEACAM5, CEACAM6, CEACAM7, CLCA1, CLDN3, DPEP1, ERBB3, GPA33, GPRC5A, ITGA6, KRTCAP3, LSR, MUC13, NOX1, PLOD3, PLPP2, SLC12A2, SLC44A4, SLC52A2, SMIM22, ST14, TFRC, TMPRSS4, sLE(x) and TSPAN6. In certain embodiments, the first antigen is selected from CLDN4, CLDN7, EPCAM, PIGR, TMEM141, TMEM54, TSPAN1 LRG5, and TSPAN8. In certain embodiments, the first antigen is selected from ACSL5, AP1M2, AREG, CDH1, CDH17, CEACAM5, CEACAM6, CEACAM7, CLCA1, CLDN3, DPEP1, ERBB3, GPA33, GPRC5A, ITGA6, KRTCAP3, LSR, MUC13, NOX1, PLOD3, PLPP2, SLC12A2, SLC44A4, SLC52A2, SMIM22, ST14, TFRC, TMPRSS4, sLE(x), TSPAN6, CLDN4, CLDN7, EPCAM, PIGR, TMEM141, TMEM54, TSPAN1, LRG5 and TSPAN8. In certain embodiments, the first antigen is any one of ACSL5, AP1M2, AREG, CDH1, CDH17, CEACAM5, CEACAM6, CEACAM7, CLCA1, CLDN3, DPEP1, ERBB3, GPA33, GPRC5A, ITGA6, KRTCAP3, LSR, MUC13, NOX1, PLOD3, PLPP2, SLC12A2, SLC44A4, SLC52A2, SMIM22, ST14, TFRC, TMPRSS4, sLE(x), TSPAN6, CLDN4, CLDN7, EPCAM, PIGR, TMEM141, TMEM54, TSPAN1, LRG5 and TSPAN8.

In certain embodiment the first antigen is selected from LRRC15, ADAM12, MMP14, GPX8, PDPN, CDH11 and F2RL2.

In certain embodiments, the first antigen is selected from an antigen on myofibroblasts, such as LRRC15, ADAM12, FAP, and CDH11.

An antibody construct may comprise an antibody with modifications of at least one amino acid residue. Modifications may be substitutions, additions, mutations, deletions, or the like. An antibody modification can be an insertion of an unnatural amino acid.

In certain embodiments, the antibody construct comprises a murine, chimeric, humanized or human antibody. The antibody can be an anti-tumor antigen antibody such as for example, an anti-LRRC15, anti-ADAM12, anti-MMP14, anti-GPX8, anti-PDPN, anti-CDH11 and anti-F2RL2 antibody. In some aspects, the antibody is an internalizing antibody.

The antibody can be an anti-tumor antigen antibody wherein the tumor antigen is selected from an antigen on myofibroblasts, such as LRRC15, ADAM12, FAP, and CDH11.

Methods of making antibodies and antigen binding domains are well known in the art. In some aspects, antibodies known in the art are used in the present methods. See, for example, the monoclonal antibody database of IMGT®, the International Immunogenetics information system that is the global reference in immunogenetics and immunoinformatics for antibodies known in the art. For exemplary anti-LRRC15 antibodies, see, for example, WO2017/095805 and WO2017/095808 (the disclosures of which are incorporated by reference herein).

The present invention, inter alia, provides conjugates comprising antibodies and antigen fragments thereof that specifically bind to the antigens described here. The conjugates can be used to treat disease.

### Target Binding Domain

An antibody construct may further comprise a target binding domain. A target binding domain comprises a domain that specifically binds to a target. A target binding domain may comprise an antigen binding domain. A target binding domain may be a domain that can specifically bind to an antigen. A target binding domain may be an antigen-binding portion of an antibody or an antibody fragment. A target binding domain may be one or more fragments of an antibody that can retain the ability to specifically bind to an antigen. A target binding domain may be any antigen binding fragment. A target binding domain may be in a scaffold, in which a scaffold is a supporting framework for the antigen binding domain. A target binding domain may comprise an antigen binding domain in a scaffold.

A target binding domain may comprise an antigen binding domain which can refer to a portion of an antibody comprising the antigen recognition portion, i.e., an antigenic determining variable region of an antibody sufficient to confer recognition and specific binding of the antigen recognition portion to a target, such as an antigen, i.e., the epitope.

An Fv can be the minimum antibody fragment which contains a complete antigen-recognition and antigen-binding site. This region may consist of a dimer of one heavy chain and one light chain variable domain in tight, non-covalent association. In this configuration, the three CDRs of each variable domain may interact to define an antigen-binding site on the surface of the V_{H}-V_{L} dimer. A single variable domain (or half of an Fv comprising only three CDRs specific for an antigen) can recognize and bind antigen, although at a lower affinity than the entire binding site.

A target binding domain may be at least 80% homologous to a specific antigen binding domain selected from, but not limited to, a monoclonal antibody, a polyclonal antibody, a recombinant antibody, or a functional fragment thereof, for example, a heavy chain variable domain (V_{H}) and a light chain variable domain (V_{L}), or a single chain variable fragment (scFv), or a DARPin, an affimer, an avimer, a knottin, a monobody, an affinity clamp, an ectodomain, a receptor ectodomain, a receptor, a cytokine, a ligand, an immunocytokine, a T cell receptor, or a recombinant T cell receptor.

A target binding domain may be attached to an antibody construct. For example, an antibody construct may be fused with a target binding domain to create an antibody construct target binding domain fusion. The antibody construct-target binding domain fusion may be the result of the nucleic acid sequence of the target binding domain being expressed in frame with the nucleic acid sequence of the antibody construct. The antibody construct-target binding domain fusion may be the result of an in-frame genetic nucleotide sequence encoding the antibody construct, or a contiguous peptide sequence of the antibody construct, with the target binding domain. As another example, a target binding domain may be linked to an antibody construct. A target binding domain may be linked to an antibody construct by a chemical conjugation. A target binding domain may be attached to a terminus of an Fc domain or Fc region. A target binding domain may be attached to a terminus of an Fc domain or Fc region. A target binding domain may be attached to a terminus of an antibody construct. A target binding domain may be attached to a terminus of an antibody. A target binding domain may be attached to a light chain of an antibody. A target binding domain may be attached to a terminus of a light chain of an antibody. A target binding domain may be attached to a heavy chain of an antibody. A target binding domain may be attached to a terminus of a heavy chain of an antibody. The terminus may be a C-terminus. An antibody construct may be attached to 1, 2, 3, and/or 4 target binding domains. The target binding domain may direct the antibody construct to, for example, a particular cell or cell type. A target binding domain of an antibody construct may be selected in order to recognize an antigen, e.g., an antigen expressed on an immune cell. An antigen can be a peptide or fragment thereof. An antigen may be expressed on an immune cell, such as an antigen-presenting cell. An antigen may be expressed on a dendritic cell, a macrophage, or a B cell. As another example, an antigen may be a tumor antigen. The tumor antigen may be any tumor antigen. When multiple target binding domains are attached to an antibody construct, the target binding domains may bind to the same antigen. When multiple target binding domains are attached to an antibody construct, the target binding domains may bind different antigens.

In certain embodiments, an antibody construct specifically binds a second antigen. In certain embodiments, the target binding domain is linked, e.g., covalently bound, to the antibody construct at a C-terminal end of the Fc domain.

### Compounds

The present disclosure provides, inter alia, compounds and salts thereof. The presently disclosed compounds and salts thereof may be free drugs or may be components of, for example, a conjugate (e.g., as described herein). For example, the compounds and salts may be covalently bound, to linkers, L³, which may further be covalently bound to antibody constructs.

In some aspects, the present disclosure provides a compound represented by the structure of Formulas (I):

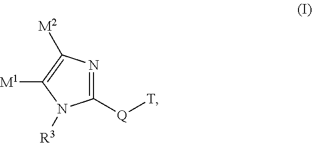

or a salt thereof, wherein:

M¹ and M² are independently selected from

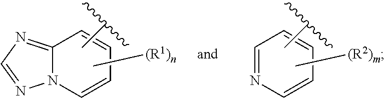

R¹ and R² are independently selected at each occurrence from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, and —CN; —C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle; and

a C₃-C₁₀ carbocycle and a 3- to 10-membered heterocycle, each of which is optionally substituted with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, —C₁-C₆ alkyl, —C₂-C₆ alkenyl, and —C₂-C₆ alkynyl;

R³ is selected from hydrogen and —C₁-C₁₀ alkyl optionally substituted with one or more substituents independently selected from a halogen, —NO₂, ═O, ═S, ═N(R¹⁰), —CN, —OR¹⁰, —SR¹⁰, —N(R¹⁰)₂, —C(O)R¹⁰, —C(O)N(R¹⁰)₂, —N(R¹⁰)C(O)₁₀, —C(O)OR¹⁰, and —OC(O)R¹⁰;

n and m are independently selected from 0, 1, 2, 3, and 4;

Q is selected from a bond, —(CR¹⁰₂)ₚ—, —(CR¹⁰₂)_{q}C(═O)(CR¹⁰₂)_{q}—, —(CR¹⁰₂)_{q}C(═S)(CR¹⁰₂)_{q}—, —(CR¹⁰₂)_{q}C(═NR¹⁰)(CR¹⁰₂)_{q}, —(CR¹⁰₂)_{q}O(CR¹⁰₂)_{q}—, —(CR¹⁰₂)_{q}S(CR¹⁰₂)_{q}—, —(CR¹⁰₂)_{q}N(R¹⁰)(CR¹⁰₂)_{q}—, —(CR¹⁰₂)_{q}OC(═O)O(CR¹⁰₂)_{q}—, —(CR¹⁰₂)_{q}C(═O)N(R¹⁰)(CR¹⁰₂)_{q}—, —(CR¹⁰₂)_{q}N(R¹⁰)C(═O)(CR¹⁰₂)_{q}—, and —(CR¹⁰₂)_{q}N(R¹⁰)SO₂(CR¹⁰₂)_{q}—;

p is selected from 1, 2, 3, 4, and 5;

q is independently selected at each occurrence from 0, 1, 2, 3, 4, and 5;

T is selected from an optionally substituted saturated C₃-C₇ carbocycle, an optionally substituted C₅₋₁₂ bicyclic carbocycle, and an optionally substituted 4- to 12-membered heterocycle, wherein T is optionally substituted with one or more substituents independently selected at each occurrence from R¹³;

R¹³ is independently selected at each occurrence from: a halogen, —OR¹⁰, —SR¹⁰, —N(R¹⁰)₂, —C(O)R¹⁰, —C(O)N(R¹⁰)₂, —N(R¹⁰)C(R¹⁰), —C(O)OR¹⁰, —OC(O)R¹⁰, —S(O)R¹⁰, —S(O)₂R¹⁰, —S(O)₂N(R¹⁰₂, —P(O)(OR¹⁰)₂, —OP(O)(OR¹⁰)₂, —NO₂, ═O, ═S, ═N(R¹⁰), and —CN; —C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle; and

a C₃-C₁₀ carbocycle and a 3- to 10-membered heterocycle, each of which is optionally substituted with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, —C₁-C₆ alkyl, —C₂-C₆ alkenyl, and —C₂-C₆ alkynyl; and

R¹⁰ is independently selected at each occurrence from: hydrogen; —C₁-C₁₀ alkyl, —C₂-C₁₀ alkenyl, and —C₂-C₁₀ alkynyl, each of which is optionally substituted at each occurrence with one or more substituents independently selected from a halogen, —OH, —CN, —NO₂, —NH₂, ═O, ═S, —O—C₁-C₁₀ alkyl, C₃-C₁₂ carbocycle, and a 3- to 12-membered heterocycle; and a C₃-C₁₂ carbocycle and a 3- to 12-membered heterocycle, each of which is optionally substituted at each occurrence with one or more substituents independently selected from a halogen, —OH, —CN, —NO₂, —NH₂, ═O, ═S, —C₁-C₁₀ alkyl, —O—C₁-C₁₀ alkyl, and —C₁-C₁₀ haloalkyl.

In some embodiments, one of M¹ and M² is

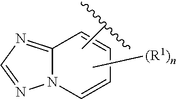

and the other of M¹ and M^{2 is}

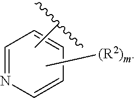

For example, M¹ may be

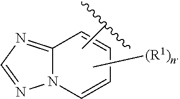

In some embodiments, the compound or salt is represented by Formula (Ia):

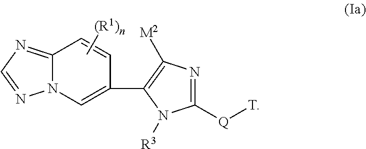

In some embodiments, M² is

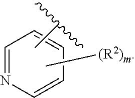

In some embodiments, the compound or salt is represented by Formula (Ib):

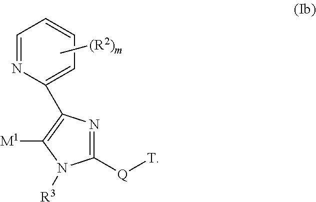

In some embodiments, M¹ is

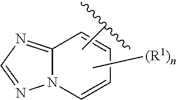

### and M² is

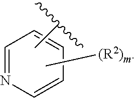

In other embodiments, M¹ is

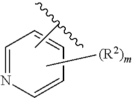

### and M² is

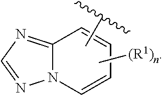

In some embodiments, the compound or salt is represented by Formula (Ic) or Formula (Id):

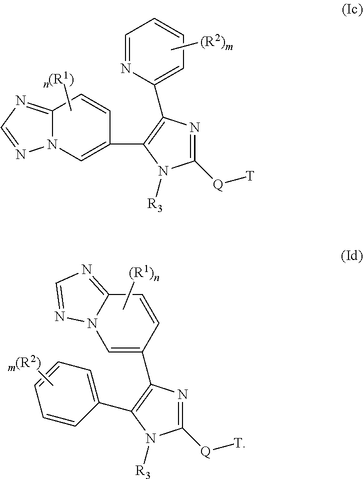

In some embodiments, a compound of Formula (I) is represented by Formula (Ie):

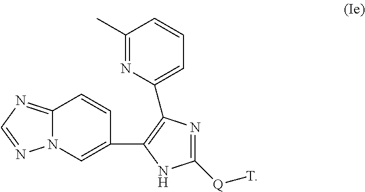

In some embodiments, R³ for a compound or salt of any of Formulas (I), (Ia), (Ib), (Ic), and (Id) is selected from hydrogen and —C₁-C₁₀ alkyl optionally substituted with one or more substituents independently selected from a halogen, —NO₂, —CN, —OR¹⁰, —N(R¹⁰)₂, —C(O)R¹⁰, —C(O)N(R¹⁰)₂, —N(R¹⁰)C(O)R¹⁰, —C(O)OR¹⁰, and —OC(O)R¹⁰. For example, for a compound or salt of any of Formulas (I), (Ia), (Ib), (Ic), and (Id), R³ may be selected from hydrogen and —C₁-C₁₀ alkyl optionally substituted with one or more substituents independently selected from a halogen, —NO₂, —CN, —OR¹⁰, —SR¹⁰, and —N(R¹⁰)₂. In some embodiments, for a compound or salt of any of Formulas (I), (Ia), (Ib), (Ic), and (Id), R³ is hydrogen.

In some embodiments, for a compound or salt of any of Formulas (I), (Ia), (Ib), (Ic), and (Id), n is 0. In other embodiments, for a compound or salt of any of Formulas (I), (Ia), (Ib), (Ic), and (Id), n is selected from 1, 2, 3, and 4. In some embodiments, for a compound or salt of any of Formulas (I), (Ia), (Ib), (Ic), and (Id), each le is independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)², —P(O)(OR¹⁰)₂, —OP(O)(OR¹⁰)₂, —NO₂, and —CN; and —C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)²R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle. In certain embodiments, for a compound or salt of any of Formulas (I), (Ia), (Ib), (Ic), and (Id), each R¹ is selected from a halogen, —OR¹⁰, —SR¹⁰, —N(R¹⁰)₂, —NO₂, and —CN; and —C₁-C₁₀ alkyl optionally substituted at each occurrence with one or more substituents independently selected from a halogen OR¹⁰, —SR¹⁰, —N(R¹⁰)₂, —C(O)R¹⁰, —NO₂, and —CN. In some embodiments, for a compound or salt of any of Formulas (I), (Ia), (Ib), (Ic), and (Id), each R¹ is independently selected from a —C₁-C₁₀ alkyl optionally substituted at each occurrence with one or more substituents independently selected from a halogen, —OR¹⁰, —SR¹⁰, —N(R¹⁰)₂, —C(O)R¹⁰, —NO₂, and —CN.

In some embodiments, for a compound or salt of any of Formulas (I), (Ia), (Ib), (Ic), and (Id), m is 0. In other embodiments, for a compound or salt of any of Formulas (I), (Ia), (Ib), (Ic), and (Id), m is selected from 1, 2, 3, and 4. In some embodiments, for a compound or salt of any of Formulas (I), (Ia), (Ib), (Ic), and (Id), each R² is independently selected from a halogen, —OR¹⁰, SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)², —P(O)(OR¹⁰)₂, —OP(O)(OR¹⁰)₂, —NO₂, and —CN; and —C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle. In certain embodiments, for a compound or salt of any of Formulas (I), (Ia), (Ib), (Ic), and (Id), each R² is independently selected from a halogen, —OR¹⁰, —SR¹⁰, —N(R¹⁰)₂, —NO₂, and —CN; and —C₁-C₁₀ alkyl optionally substituted at each occurrence with one or more substituents independently selected from a halogen, —OR¹⁰, —SR¹⁰, —N(R¹⁰)₂, —C(O)R¹⁰, —NO₂, and —CN. In some embodiments, for a compound or salt of any of Formulas (I), (Ia), (Ib), (Ic), and (Id), each R² is independently selected from —OR¹⁰ and —C₁-C₁₀ alkyl optionally substituted with one or more substituents independently selected from a halogen, —OR¹⁰, —SR¹⁰, —N(R¹⁰)₂, —C(O)R¹⁰, —NO₂, and —CN. In some embodiments, for a compound or salt of any of Formulas (I), (Ia), (Ib), (Ic), and (Id), m is 1 and R² is —CH₃. For example, for a compound or salt of any of Formulas (I), (Ia), (Ib), (Ic), and (Id), M² may be

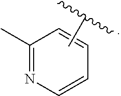

In a particular example, for a compound or salt of any of Formulas (I), (Ia), (Ib), (Ic), and (Id), M² is

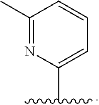 [SMILES: *c1cccc(C)n1]

In some embodiments, the compound of Formula (I) is represented by Formula (Ie)

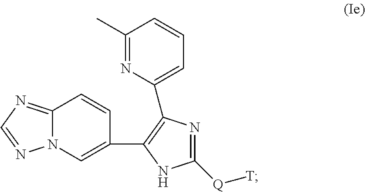

or a salt thereof.

In some embodiments, for a compound or salt of any of Formulas (I), (Ia), (Ib), (Ic), (Id), and (Ie), Q is selected from —(CR¹⁰₂)ₚ—, —(CR¹⁰₂)_{q}O(CR¹⁰₂)_{q}—, —(CR¹⁰₂)_{q}SS(CR¹⁰₂)_{q}—, and —(CR¹⁰₂)_{q}NR¹⁰(CR¹⁰₂)_{q}—, where p is selected from 1, 2, 3, 4, and 5 and q is independently selected at each occurrence from 0, 1, 2, 3, 4, and 5. In some embodiments, for a compound or salt of any of Formulas (I), (Ia), (Ib), (Ic), (Id), and (Ie), Q is selected from —(CR¹⁰₂)ₚ—. For example, for a compound or salt of any of Formulas (I), (Ia), (Ib), (Ic), (Id), and (Ie), p may be 1 such that Q is) —C(R¹⁰)₂—. Alternatively, for a compound or salt of any of Formulas (I), (Ia), (Ib), (Ic), (Id), and (Ie), Q may be selected from —(CR¹⁰₂)_{q}O(CR¹⁰₂)_{q}—, —(CR¹⁰₂)_{q}S(CR¹⁰₂)_{q}—, and —(CR¹⁰₂)_{q}NR¹⁰(CR¹⁰₂)_{q}—. In some embodiments, for a compound or salt of any of Formulas (I), (Ia), (Ib), (Ic), (Id), and (Ie), Q is —CH₂NH—. In some embodiments, for a compound or salt of any of Formulas (I), (Ia), (Ib), (Ic), (Id), and (Ie), Q is selected from —(CR¹⁰₂)_{q}NR¹⁰(CR¹⁰₂)_{q}—. In some embodiments, for a compound or salt of any of Formulas (I), (Ia), (Ib), (Ic), (Id), and (Ie), Q is selected from —(CR¹⁰₂)_{q}NR¹⁰—. In some embodiments, for a compound or salt of any of Formulas (I), (Ia), (Ib), (Ic), (Id), and (Ie), Q is selected from —(CR¹⁰₂)_{q}NR¹⁰—. In some embodiments, for a compound or salt of any of Formulas (I), (Ia), (Ib), (Ic), (Id), and (Ie), Q is —CR¹⁰₂)_{q}NR¹⁰(CR¹⁰₂)₁₋₂—. In some embodiments, for a compound or salt of any of Formulas (I), (Ia), (Ib), (Ic), (Id), and (Ie), Q is —CH₂NHCH₂— or —CH₂NHCH₂CH₂—. In another example, for a compound or salt of any of Formulas (I), (Ia), (Ib), (Ic), (Id), and (Ie), Q is —CH₂NHCH₂—.

In some embodiments, for a compound or salt of any of Formulas (I), (Ia), (Ib), (Ic), (Id), and (Ie), each R¹⁰ is independently selected from hydrogen; and —C₁-C₁₀ alkyl, —C₂-C₁₀ alkenyl, and —C₂-C₁₀ alkynyl, each of which is optionally substituted at each occurrence with one or more substituents independently selected from a halogen, —OH, —CN, —NO₂, and —NH₂. In certain embodiments, for a compound or salt of any of Formulas (I), (Ia), (Ib), (Ic), (Id), and (Ie), R¹⁰ is hydrogen at each occurrence.

In some embodiments, for a compound or salt of any one of Formulas (I), (Ia), (Ib), (Ic), (Id) and (Ie), each R¹⁰ of Q is hydrogen at each occurrence.

In some embodiments, for a compound or salt of any of Formulas (I), (Ia), (Ib), (Ic), (Id), and (Ie), T is selected from a saturated C₃-C₇ carbocycle optionally substituted with one or more substituents independently selected at each occurrence from R¹³. For example, for a compound or salt of any of Formulas (I), (Ia), (Ib), (Ic), (Id), and (Ie), T may be a saturated C₃ carbocycle optionally substituted with one or more substituents independently selected at each occurrence from R¹³. In some embodiments, for a compound or salt of any of Formulas (I), (Ia), (Ib), (Ic), (Id), and (Ie), T may be selected from:

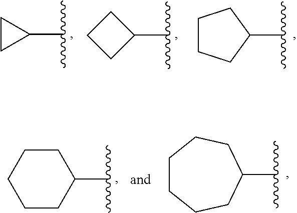

each of which is optionally substituted with one or more substituents independently selected at each occurrence from R¹³. In some embodiments, for a compound or salt of any of Formulas (I), (Ia), (Ib), (Ic), (Id), and (Ie), T is selected from

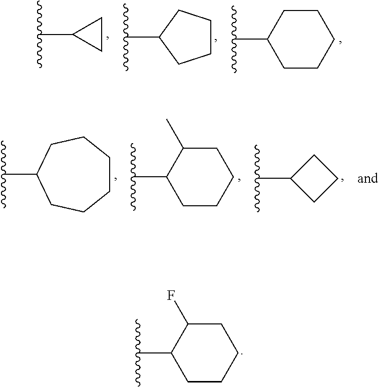

For example, for a compound or salt of any of Formulas (I), (Ia), (Ib), (Ic), (Id), and (Ie), T may be:

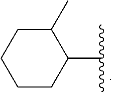 [SMILES: *C1CCCCC1C]

In some embodiments, for a compound or salt of any of Formulas (I), (Ia), (Ib), (Ic), (Id), and (Ie), T is selected from a C₅₋₁₂ bicyclic carbocycle optionally substituted with one or more substituents independently selected at each occurrence from R¹³. In certain embodiments, for a compound or salt of any of Formulas (I), (Ia), (Ib), (Ic), (Id), and (Ie), T is selected from a saturated C₅₋₁₂ bridged carbocycle optionally substituted with one or more substituents independently selected at each occurrence from R¹³, such as an optionally substituted C₅ bridged carbocycle. For example, for a compound or salt of any of Formulas (I), (Ia), (Ib), (Ic), (Id), and (Ie), T may be selected from:

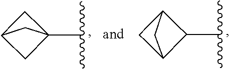 [SMILES: *C12CC(C1)C2 ; *C1C2CC1C2]

each of which is optionally substituted with one or more substituents independently selected at each occurrence from R¹³.

In other embodiments, for a compound or salt of any of Formulas (I), (Ia), (Ib), (Ic), (Id), and (Ie), T is selected from C₈₋₁₁ bicyclic carbocycle optionally substituted with one or more substituents independently selected at each occurrence from R¹³. In some embodiments, for a compound or salt of any of Formulas (I), (Ia), (Ib), (Ic), (Id), and (Ie), T is a fused bicyclic ring. In certain embodiments, for a compound or salt of any of Formulas (I), (Ia), (Ib), (Ic), (Id), and (Ie), T is selected from a C₈₋₁₁ fused bicyclic carbocycle. In certain embodiments, for a compound or salt of any of Formulas (I), (Ia), (Ib), (Ic), (Id), and (Ie), T is selected from:

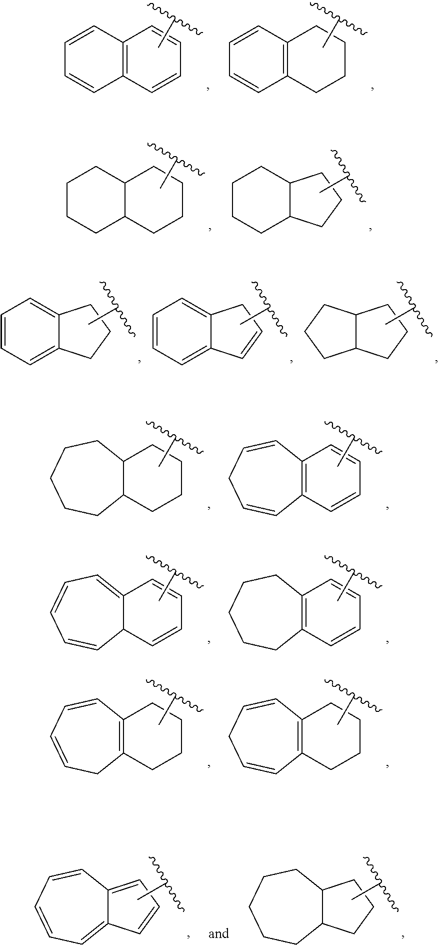

and wherein T is optionally substituted with one or more substituents independently selected at each occurrence from R¹³. For example, for a compound or salt of any of Formulas (I), (Ia), (Ib), (Ic), (Id), and (Ie), T may be selected from:

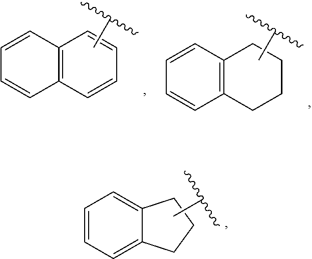

wherein T is optionally substituted with one or more substituents independently selected at each occurrence from R¹³. In certain embodiments, for a compound or salt of any of Formulas (I), (Ia), (Ib), (Ic), (Id), and (Ie), T is

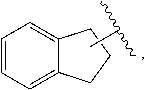

wherein T is optionally substituted with one or more substituents independently selected at each occurrence from R¹³. In some embodiments, for a compound or salt of any of Formulas (I), (Ia), (Ib), (Ic), (Id), and (Ie), T may be selected from:

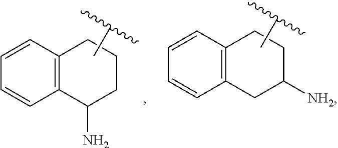

In some embodiments, for a compound or salt of any of Formulas (I), (Ia), (Ib), (Ic), (Id), and (Ie), T may be selected from:

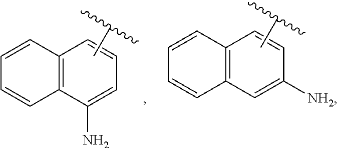

In certain embodiments, for a compound or salt of any of Formulas (I), (Ia), (Ib), (Ic), (Id), and (Ie), T is selected from

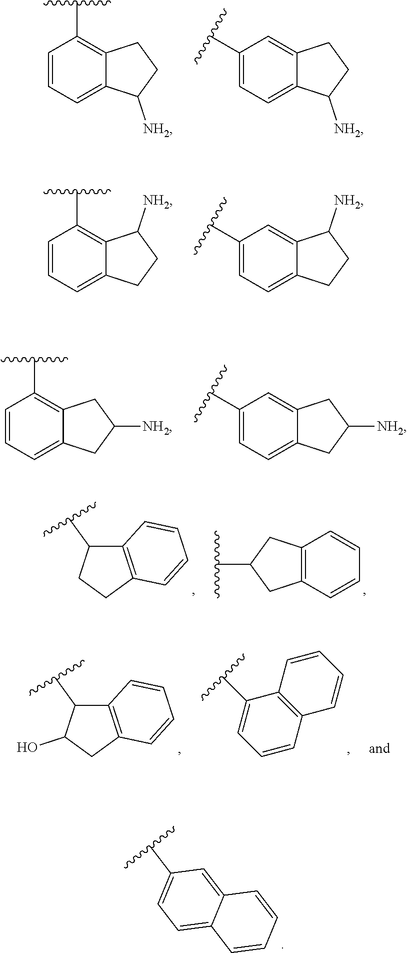

In some embodiments, for a compound or salt of any of Formulas (I), (Ia), (Ib), (Ic), (Id), and (Ie), T is selected from naphthalene, 1,2,3,4-tetrahydronaphthalene, decahydronaphthalene, octahydro-1H-indene, 2,3-dihydro-1H-indene, 1H-indene, octahydropentalene, decahydro-1H-benzo[7]annulene, 7H-benzo[7]annulene, 4aH-benzo[7]annulene, 6,7,8,9-tetrahydro-5H-benzo[7]annulene, 2,3,4,5-tetrahydro-1H-benzo[7]annulene, 2,3,4,7-tetrahydro-1H-benzo[7]annulene, azulene, and decahydroazulene, and wherein T is optionally substituted with one or more substituents independently selected at each occurrence from R¹³.

In some embodiments, for a compound or salt of any of Formulas (I), (Ia), (Ib), (Ic), (Id), and (Ie), T is selected from a 4- to 12-membered heterocycle optionally substituted with one or more substituents independently selected at each occurrence from R¹³. In certain embodiments, for a compound or salt of any of Formulas (I), (Ia), (Ib), (Ic), (Id), and (Ie), T is selected from

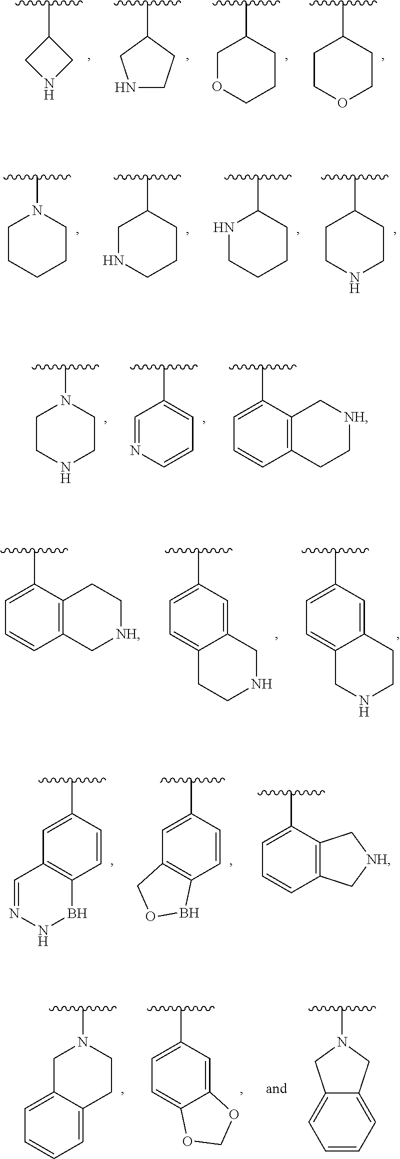

only one of which is optionally substituted with one or more substituents independently selected at each occurrence from R¹³. In certain embodiments, for a compound or salt of any of Formulas (I), (Ia), (Ib), (Ic), (Id), and (Ie), T is selected from

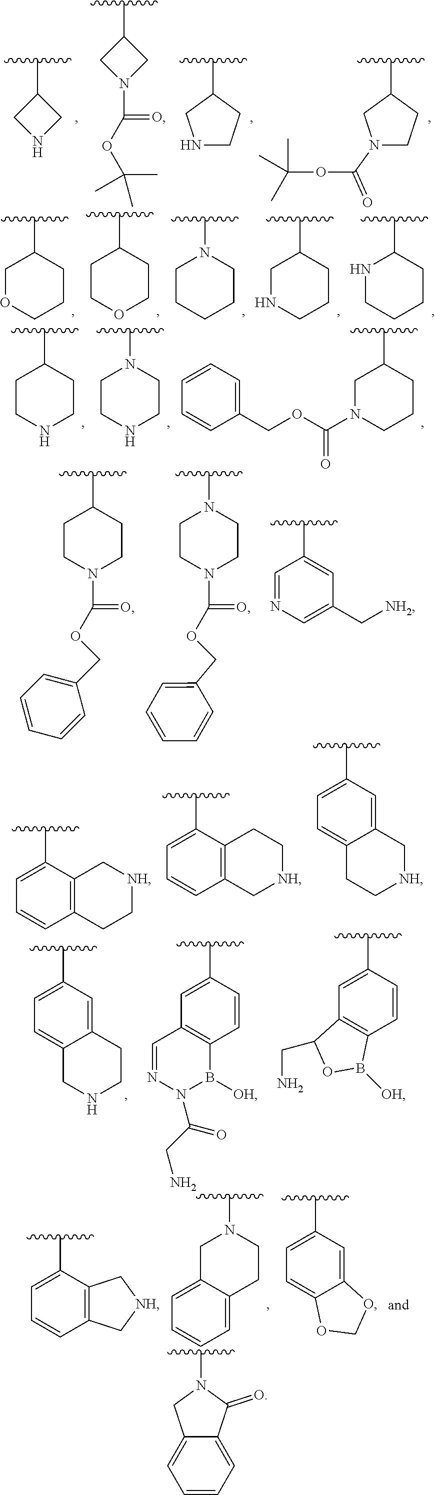

In certain embodiments, for a compound or salt of any of Formulas (I), (Ia), (Ib), (Ic), (Id), and (Ie), T is selected from a 7- to 12-membered bicyclic heterocycle optionally substituted with one or more substituents independently selected at each occurrence from R¹³. In some embodiments, for a compound or salt of any of Formulas (I), (Ia), (Ib), (Ic), (Id), and (Ie), T is selected from an 8- to 11-membered bicyclic heterocycle optionally substituted with one or more substituents independently selected at each occurrence from R¹³, such as an 8- to 11-membered bicyclic heteroaryl group.

In some embodiments, for a compound or salt of any of Formulas (I), (Ia), (Ib), (Ic), (Id), and (Ie), T is selected from:

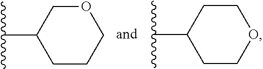

wherein T is optionally substituted with one or more substituents independently selected at each occurrence from R¹³.

In some embodiments, for a compound or salt of any one of Formulas (I), (Ia), (Ib), (Ic), (Id) and (Ie), -Q-T is selected from

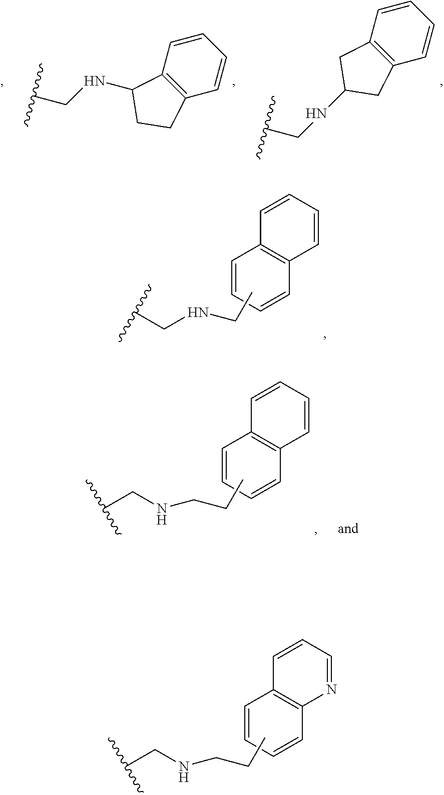

wherein T is optionally substituted with one or more substituents independently selected at each occurrence from R¹³.

In some embodiments, -Q-T is selected from

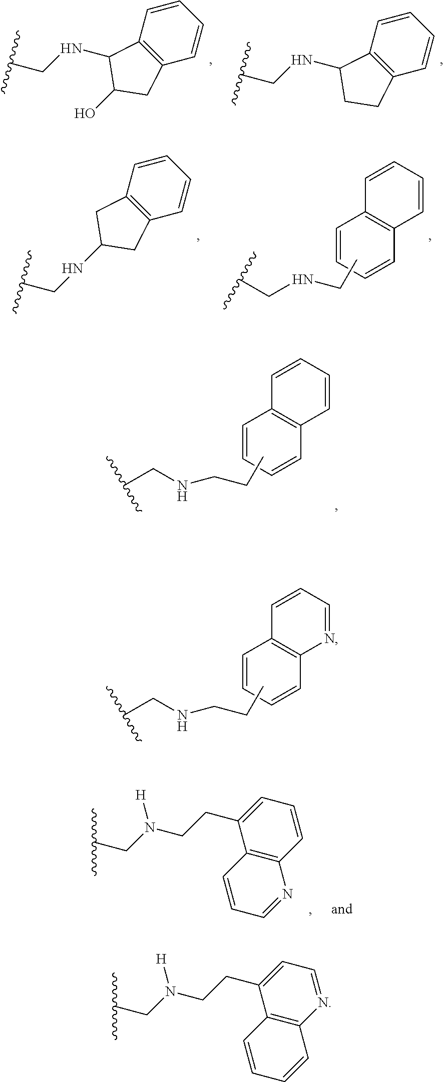

In some embodiments, for a compound or salt of any of Formulas (I), (Ia), (Ib), (Ic), (Id), and (Ie), T has one to four ring heteroatoms independently selected from N, O, S, and B. In certain embodiments, for a compound or salt of any of Formulas (I), (Ia), (Ib), (Ic), (Id), and (Ie), T has at least one ring heteroatom that is boron. For example, for a compound or salt of any of Formulas (I), (Ia), (Ib), (Ic), (Id), and (Ie), T may be selected from:

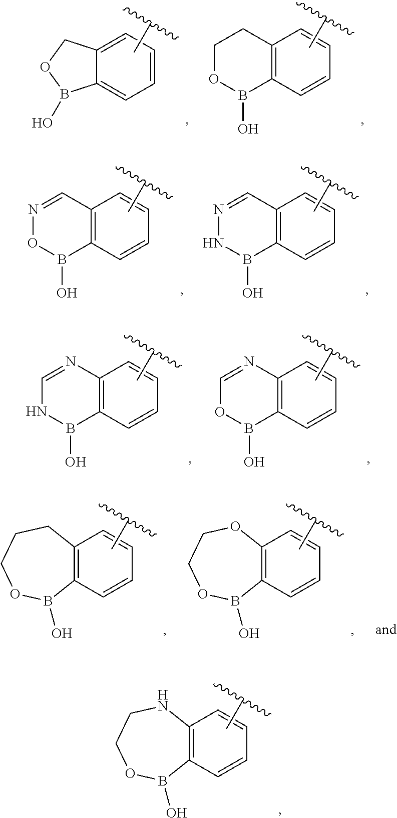

wherein T is optionally substituted with one or more substituents independently selected at each occurrence from R¹³. In certain embodiments, for a compound or salt of any of Formulas (I), (Ia), (Ib), (Ic), (Id), and (Ie), T may be selected from

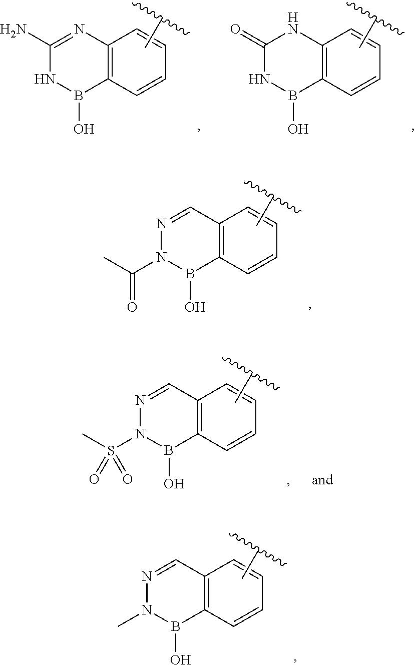

wherein T is optionally substituted with one or more substituents independently selected at each occurrence from R¹³.

In some embodiments, for a compound or salt of any of Formulas (I), (Ia), (Ib), (Ic), (Id), and (Ie), T is represented by:

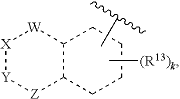

wherein dashed lines represent single or double bonds, valence permitting; k is selected from 0, 1, 2, and 3; and W, X, Y, and Z are independently selected from N(R¹⁰)_{g} and C(R¹⁰)ₕ, wherein g is selected from 0 and 1 and h is selected from 1 and 2, and wherein a straight line linked to a wavy line indicates connectivity to Q from any position, valence permitting, of the bicyclic heterocycle. In some embodiments, for a compound or salt of any of Formulas (I), (Ia), (Ib), (Ic), (Id), and (Ie), T is represented by:

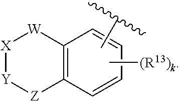

In other embodiments, for a compound or salt of any of Formulas (I), (Ia), (Ib), (Ic), (Id), and (Ie), T is represented by:

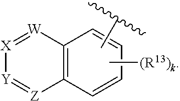

In some embodiments, for a compound or salt of any of Formulas (I), (Ia), (Ib), (Ic), (Id), and (Ie), T is selected from:

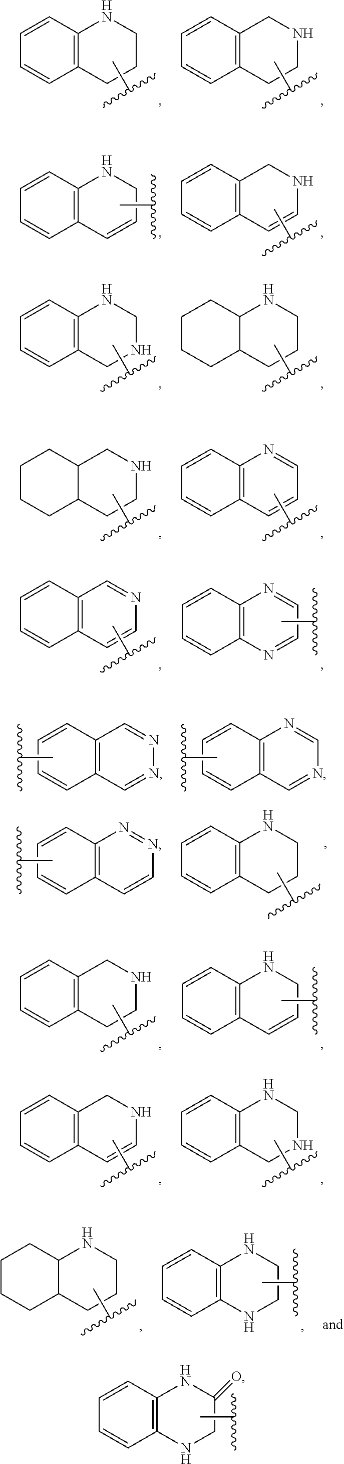

wherein T is optionally substituted with one or more substituents independently selected at each occurrence from R¹³. For example, for a compound or salt of any of Formulas (I), (Ia), (Ib), (Ic), (Id), and (Ie), T may be selected from:

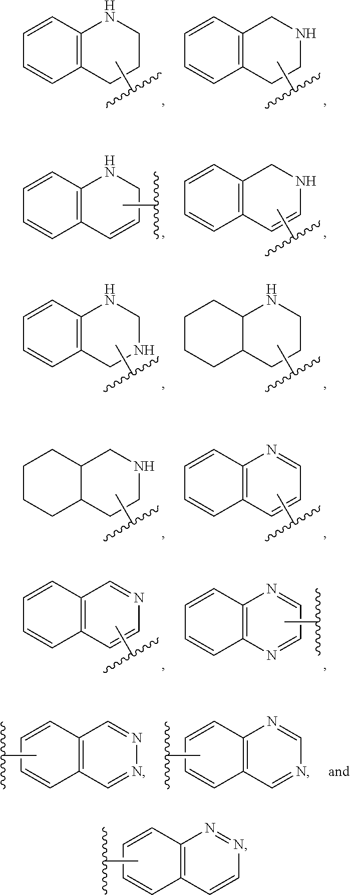

wherein T is optionally substituted with one or more substituents independently selected at each occurrence from R¹³. In certain embodiments, for a compound or salt of any of Formulas (I), (Ia), (Ib), (Ic), (Id), and (Ie), T is selected from

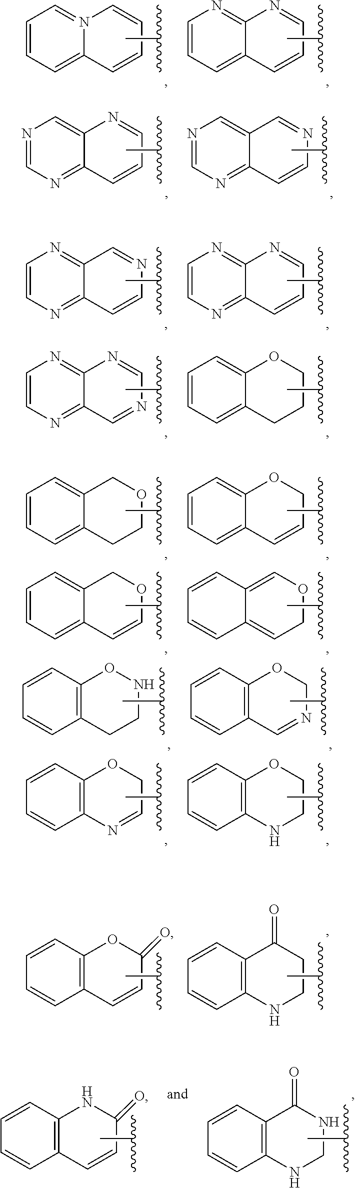

wherein T is optionally substituted with one or more substituents independently selected at each occurrence from R¹³. In some embodiments, for a compound or salt of any of Formulas (I), (Ia), (Ib), (Ic), (Id), and (Ie), T may be selected from:

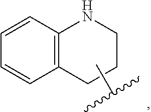

wherein T is optionally substituted with one or more substituents independently selected at each occurrence from R¹³. For example, for a compound or salt of any of Formulas (I), (Ia), (Ib), (Ic), (Id), and (Ie), T may be selected from:

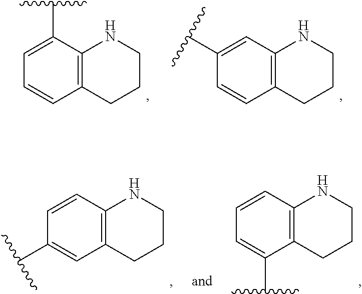

wherein T is optionally substituted with one or more substituents independently selected at each occurrence from R¹³. In some embodiments, for a compound or salt of any of Formulas (I), (Ia), (Ib), (Ic), (Id), and (Ie), T may be selected from:

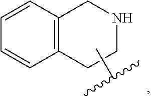

wherein T is optionally substituted with one or more substituents independently selected at each occurrence from R¹³. For example, for a compound or salt of any of Formulas (I), (Ia), (Ib), (Ic), (Id), and (Ie), T may be selected from:

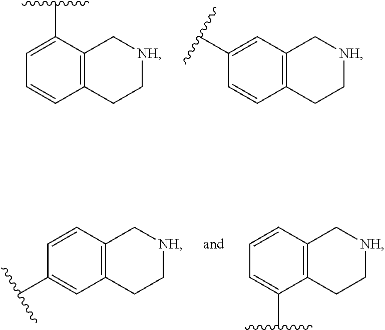

wherein T is optionally substituted with one or more substituents independently selected at each occurrence from R¹³. In some embodiments, for a compound or salt of any of Formulas (I), (Ia), (Ib), (Ic), (Id), and (Ie), T may be selected from:

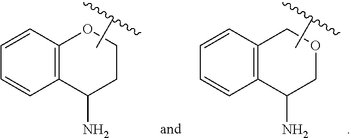

In some embodiments, for a compound or salt of any of Formulas (I), (Ia), (Ib), (Ic), (Id), and (Ie), T may be selected from:

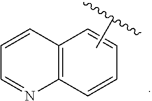

For example, for a compound or salt of any of Formulas (I), (Ia), (Ib), (Ic), (Id), and (Ie), T may be selected from:

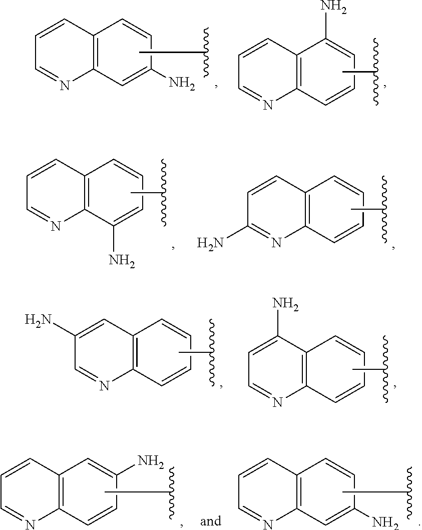

In some embodiments, for a compound or salt of any of Formulas (I), (Ia), (Ib), (Ic), (Id), and (Ie), T is selected from:

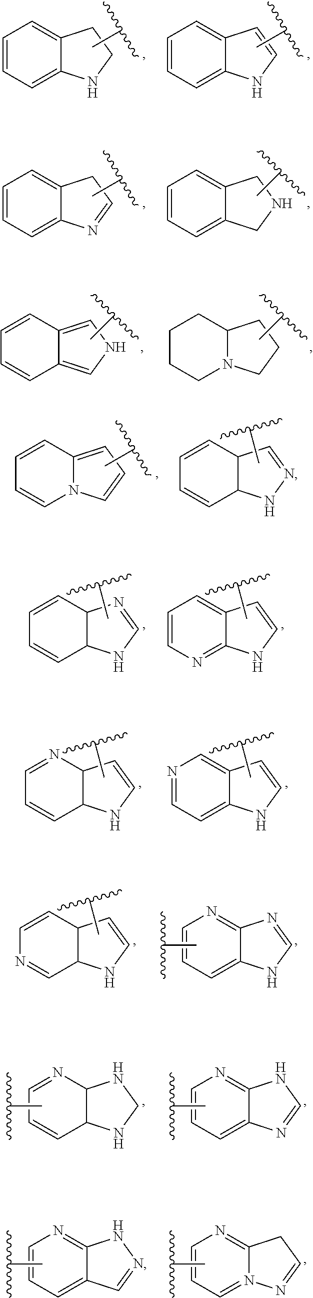

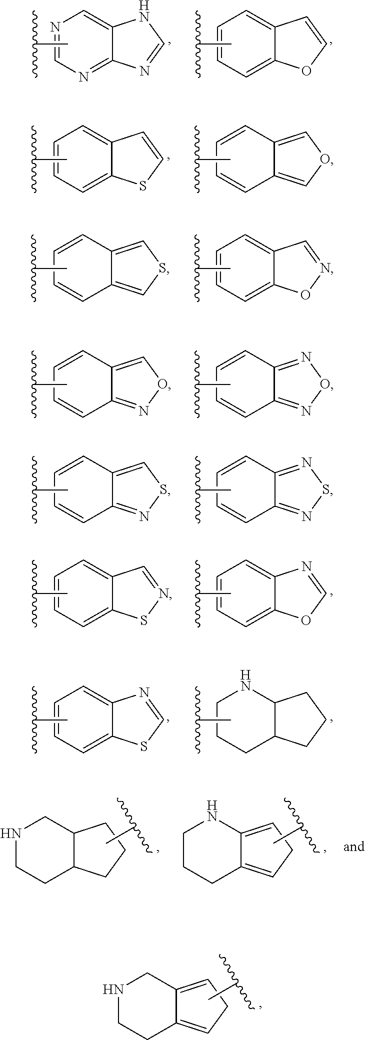

wherein T is optionally substituted with one or more substituents independently selected at each occurrence from R¹³.

In some embodiments, for a compound or salt of any of Formulas (I), (Ia), (Ib), (Ic), (Id), and (Ie), T is selected from:

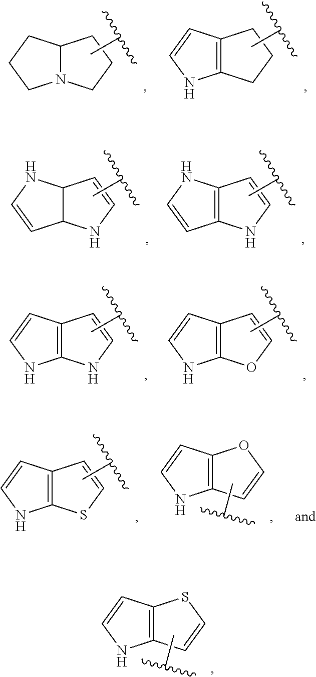

wherein T is optionally substituted with one or more substituents independently selected at each occurrence from R¹³.

In some embodiments, for a compound or salt of any of Formulas (I), (Ia), (Ib), (Ic), (Id), and (Ie), T is selected from:

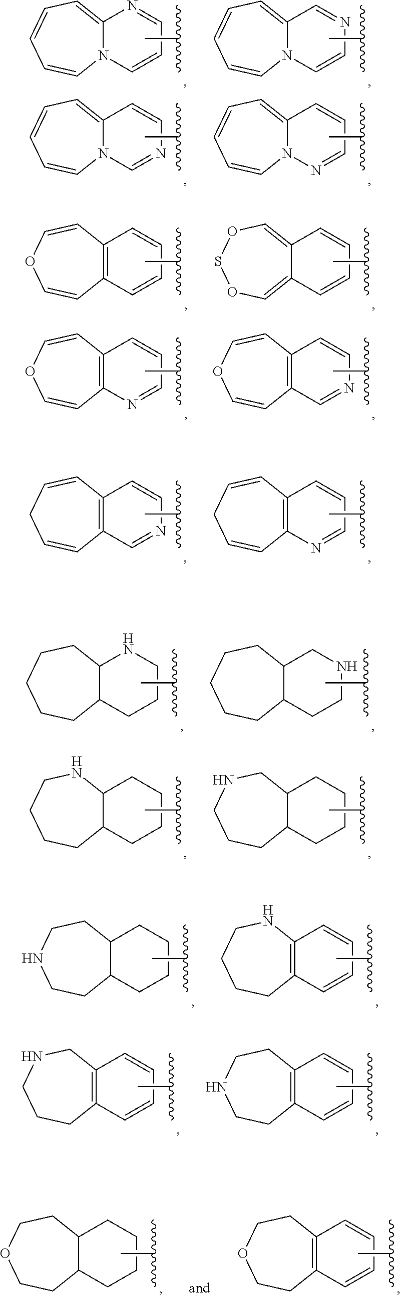

wherein T is optionally substituted with one or more substituents independently selected at each occurrence from R¹³.

In some embodiments, for a compound or salt of any of Formulas (I), (Ia), (Ib), (Ic), (Id), and (Ie), T is selected from:

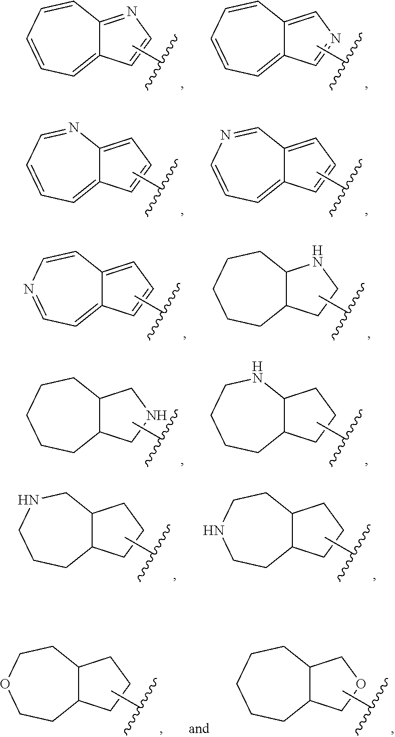

wherein T is optionally substituted with one or more substituents independently selected at each occurrence from R¹³.

In some embodiments, for a compound or salt of any of Formulas (I), (Ia), (Ib), (Ic), (Id), and (Ie), T is selected from 1,2,3,4-tetrahydroquinoline, 1,2,3,4-tetrahydroisoquinoline, 1,2-dihydroquinoline, 1,2-dihydroisoquinoline, 1,2,3,4-tetrahydroquinazoline, decahydroquinoline, decahydroisoquinoline, quinoline, isoquinoline, quinoxaline, phthalazine, quinazoline, and cinnoline, wherein T is optionally substituted with one or more substituents independently selected at each occurrence from R¹³.

In some embodiments, for a compound or salt of any of Formulas (I), (Ia), (Ib), (Ic), (Id), and (Ie), each R¹³ may be independently selected from: a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰₂, —P(O)(OR¹⁰)₂, —OP(O)(OR¹⁰)₂, —NO₂, ═O, ═S, ═N(R¹⁰), and —CN; —C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰₂), —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle. In certain embodiments, for a compound or salt of any of Formulas (I), (Ia), (Ib), (Ic), (Id), and (Ie), each R¹³ may be independently selected from a halogen, —OR¹⁰, —SR¹⁰ , —N(R¹⁰)₂, —NO₂, and —CN; and —C₁-C₁₀ alkyl optionally substituted at each occurrence with one or more substituents independently selected from a halogen, —OR¹⁰, —SR¹⁰, —N(R¹⁰)₂, —C(O)R¹⁰, —NO₂, and —CN.

In certain embodiments, for a compound or salt of Formula (Ia), (Ib), (Ic), (Id), or (Ie):

Q is selected from a bond, —(CR¹⁰₂)ₚ—, and —(CR¹⁰₂)_{q}NR¹⁰(CR¹⁰₂)_{q}—;

T is selected from an optionally substituted saturated C₃-C₇ carbocycle, an optionally substituted C₅₋₁₂ bicyclic carbocycle, and an optionally substituted 4- to 12-membered heterocycle, wherein T is optionally substituted with one or more substituents independently selected at each occurrence from R¹³ wherein R¹³ is independently selected at each occurrence from halogen, —OR¹⁰, —N(R¹⁰)₂, —C(O)R¹⁰, —C(O)OR¹⁰, —N(R¹⁰)C(O)R¹⁰, and —C₁-C₃alkyl optionally substituted with one or more substituents independently selected from a halogen, —OR¹⁰ and —N(R¹⁰)₂; and

R¹⁰ is as set forth herein (preferably H or C₁-C₃ alkyl).

In certain embodiments, for a compound or salt of Formula (Ia), (Ib), (Ic), (Id), or (Ie):

Q is selected from a bond, —(CR¹⁰₂)ₚ—, and —(CR¹⁰₂)_{q}NR¹⁰(CR¹⁰₂)_{q}—;

T is selected from an optionally substituted saturated C₃-C₇ carbocycle, an optionally substituted C₅₋₁₂ bicyclic carbocycle, and an optionally substituted 4- to 12-membered heterocycle, wherein T is optionally substituted with one or more substituents independently selected at each occurrence from R¹³ wherein R¹³ is independently selected at each occurrence from halogen, —OR¹⁰, —N(R¹⁰)₂, and —C₁-C₃ alkyl optionally substituted with one or more substituents independently selected from a halogen, —OR¹⁰ and —N(R¹⁰)₂; and

R¹⁰ is as set forth herein (preferably H or C₁-C₃ alkyl).

In certain embodiments, for a compound or salt of Formula (Ia), (Ib), (Ic), (Id), or (Ie):

Q is selected from a bond, —CH₂—, —CH₂NH—, —CH₂NHCH₂—, and —CH₂NHCH₂CH₂—; and T is selected from:

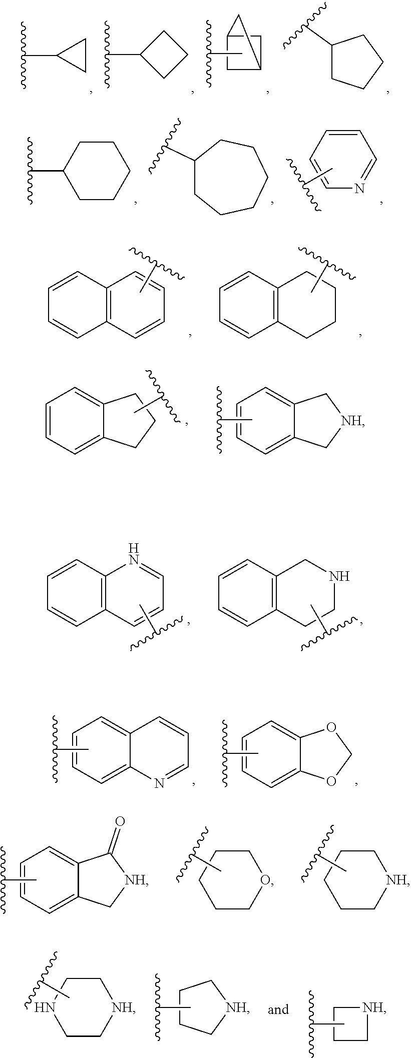

wherein T is optionally substituted with one or more substituents independently selected at each occurrence from R¹³ wherein R¹³ is independently selected at each occurrence from halogen, —OR¹⁰, —N(R¹⁰)₂, —C(O)R¹⁰, —C(O)OR¹⁰, —N(R¹⁰)C(O)R¹⁰, and —C₁-C₃ alkyl optionally substituted with one or more substituents independently selected from a halogen, —OR¹⁰ and —N(R¹⁰)₂; and

R¹⁰ is as set forth herein (preferably H or C₁-C₃ alkyl).

In certain embodiments, for a compound or salt of Formula (Ia), (Ib), (Ic), (Id), or (Ie):

Q is selected from —CH₂—, —CH₂NH—, —CH₂NHCH₂—, and —CH₂NHCH₂CH₂—; and

T is selected from:

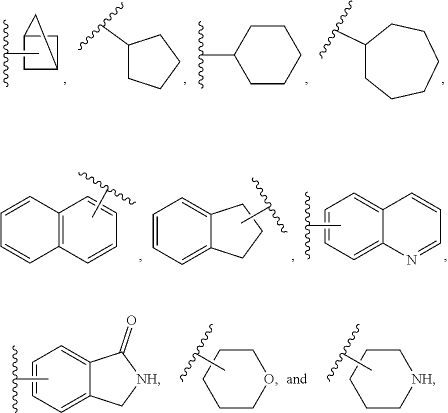

and wherein T is optionally substituted with one or more substituents independently selected at each occurrence from R¹³ wherein R¹³ is independently selected at each occurrence from halogen, —OH, —NH₂, and —C₁-C₃ alkyl.

In some embodiments, the compound is selected from:

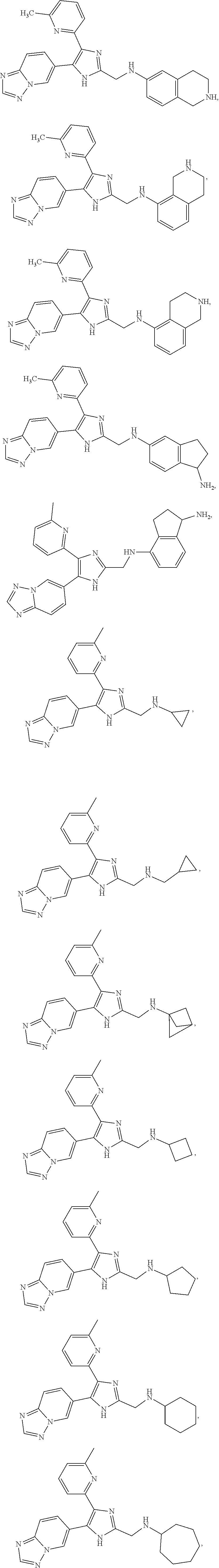

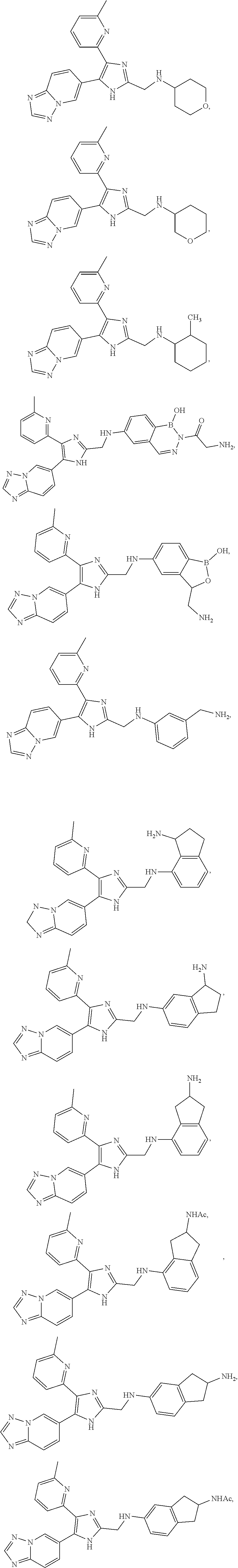

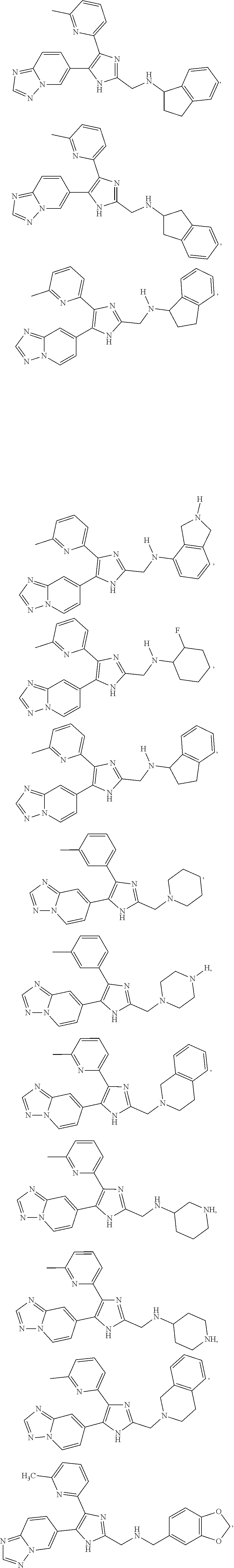

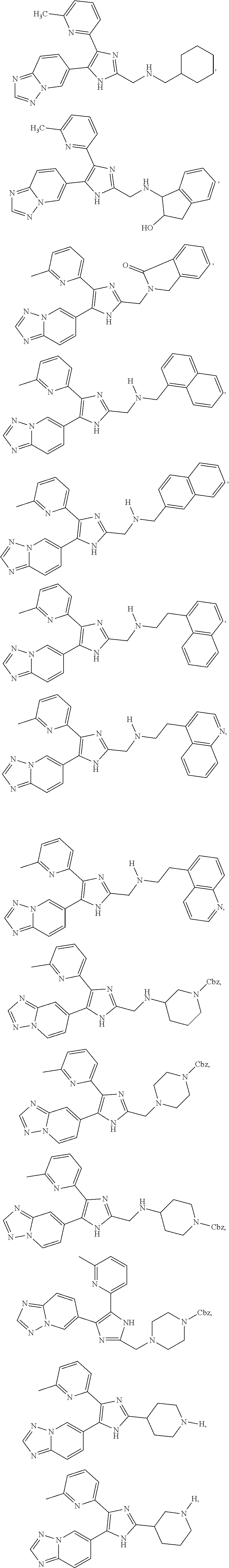

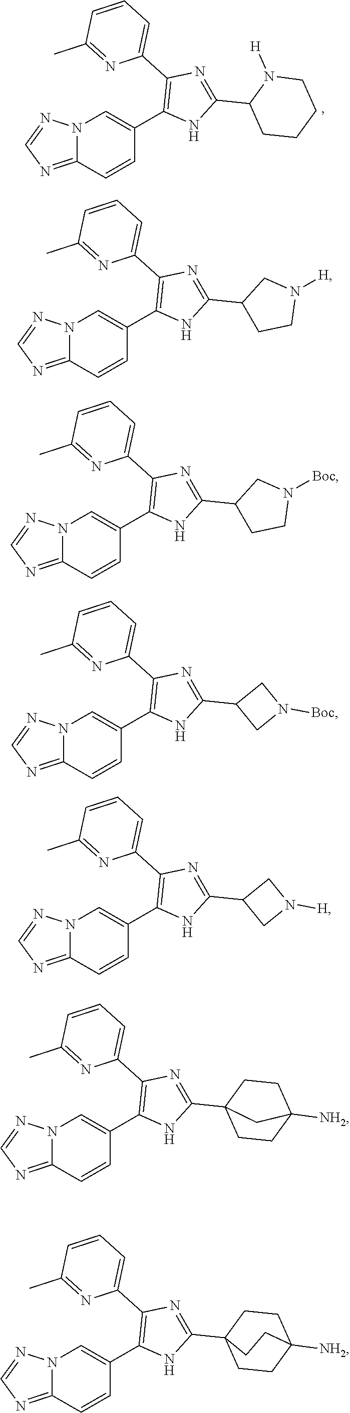

and a salt of any one thereof.

In some embodiments, the compound is selected from:

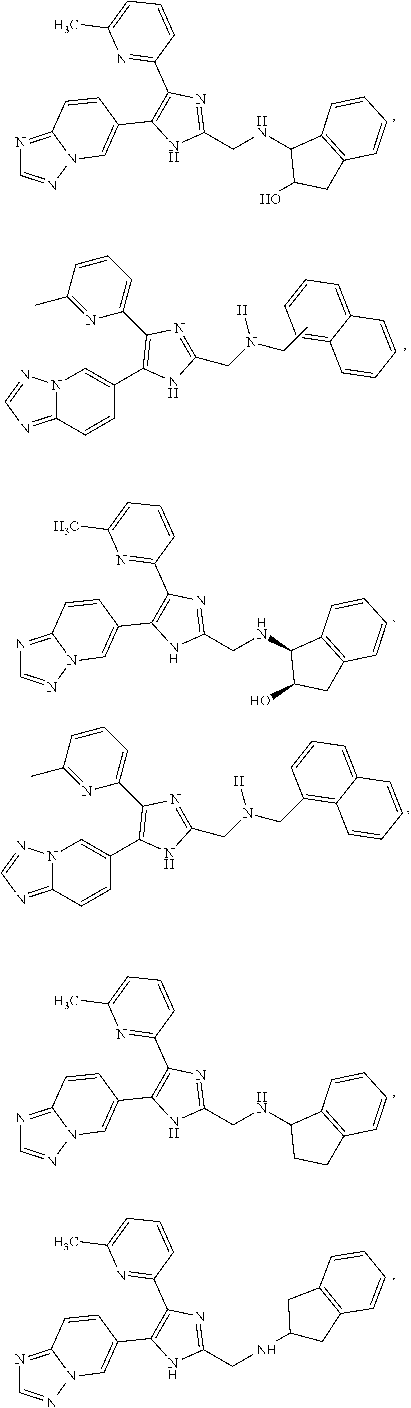

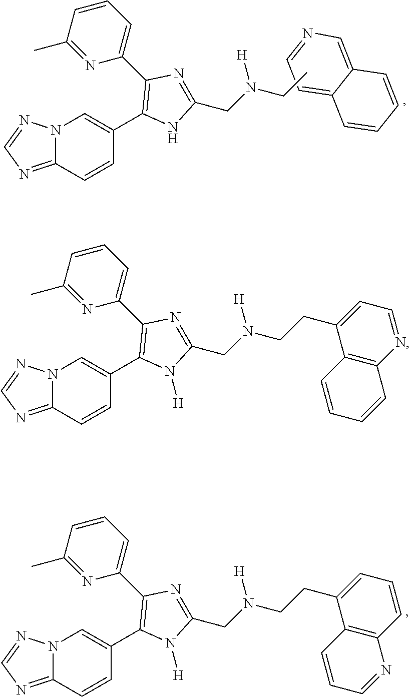

and a salt of any one thereof.

In some aspects, the present disclosure provides a method for treating cancer. In some embodiments, the present disclosure provides a method comprising administering a conjugate, compound or salt of Formulas (I), (Ia), (Ib), (Ic), (Id) and (Ie) to a subject in need thereof.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds of any one of Formulas (I), (Ia), (Ib), (Ic), (Id) and (Ie) may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

With respect to the small molecules described herein, e.g., compounds of Formula (I), (Ia), (Ib), (Ic), (Id) and (Ie), the terms administration of and administering a compound should be understood to mean providing a compound of the invention or a prodrug of the compound of the invention to the individual in need.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In certain embodiments, the prodrug may be converted, e.g., enzymatically or chemically, to the parent compound under the conditions within a cell. In certain embodiments, the parent compound includes an acidic moiety, e.g., resulting from the hydrolysis of the prodrug, which may be charged under the conditions within the cell. In some embodiments, the prodrug is converted to the parent compound once it has passed through the cell membrane into a cell. In certain embodiments, the parent compound has diminished cell membrane permeability properties relative to the prodrug, such as decreased lipophilicity and increased hydrophilicity.

In certain embodiments, the parent compound with the acidic moiety is retained within a cell for a longer duration than the same compound without the acidic moiety.

The parent compound with an acidic moiety may be retained within the cell, i.e., drug residence, for 10% or longer, such as 15% or longer, such as 20% or longer, such as 25% or longer, such as 30% or longer, such as 35% or longer, such as 40% or longer, such as 45% or longer, such as 50% or longer, such as 55% or longer, such as 60% or longer, such as 65% or longer, such as 70% or longer, such as 75% or longer, such as 80% or longer, such as 85% or longer, or even 90% or longer relative to the same compound without an acidic moiety.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Linkers

A compound or salt of the present disclosure (e.g., a compound or salt of Formulas (I), (Ia), (Ib), (Ic), (Id) and (Ie)) may be linked to a linker, L³. It will be understood by the skilled practitioner that not all compounds of the present invention are meant to be attached to linkers, only those that have suitable attachment sites.

A linker may be linked to a compound or salt of the present disclosure at any available position. For example, a compound of Formula (I) may comprise a linker L³ in place of R³, as shown below:

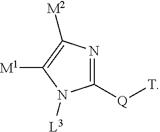

Alternatively, a linker L³ may be linked to a compound of Formula I through a substituent R³ other than hydrogen, as represented by:

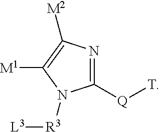

In some embodiments, a linker L³ may be linked to a moiety M¹ or M² of a compound of Formula (I). In some embodiments, the compound or salt attached to a linker L³ is represented by:

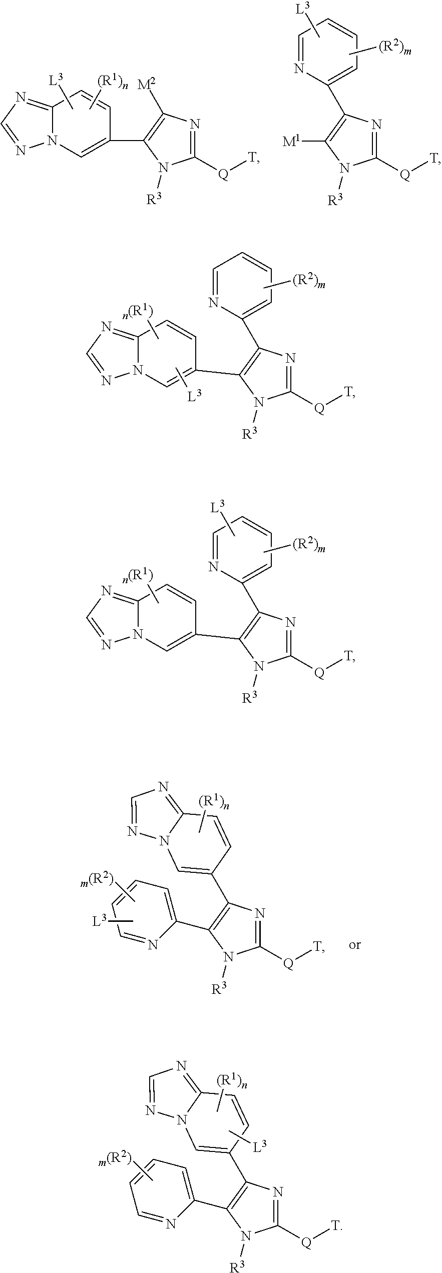

In other embodiments, a linker L³ may be linked to a subsituent of a moiety M¹ or M² of a compound of Formula (I). In some embodiments, the compound or salt attached to a linker L³ is represented by:

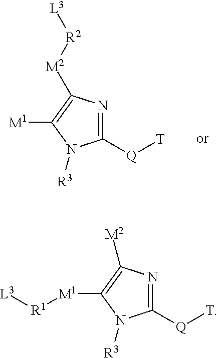

In the species above, M¹ or M² may optionally be further substituted with one or more moieties R¹ or R², respectively.

In some embodiments, a linker L³ may be linked to a moiety T of a compound of Formula (I). In some embodiments, the compound or salt attached to a linker L³ is represented by:

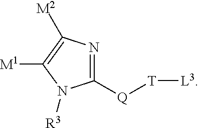

In some embodiments, the compound or salt attached to a linker L³ may be represented by:

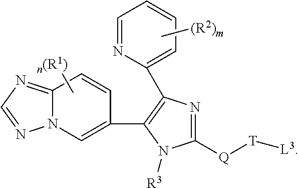

In the structures above, the linker L³ may be attached to an endocyclic atom of the ring system of T. For example, L³ may be attached to a nitrogen atom of the ring system of T (e.g., an endocyclic nitrogen).

In some embodiments, a linker L³ may be linked to a moiety Q of a compound of Formula (I). In some embodiments, the compound or salt attached to a linker L³ is represented by:

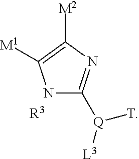

In some embodiments, the compound or salt attached to a linker L³ may be represented by:

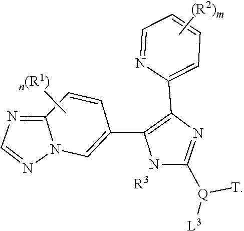

In the structures above, the linker L³ may be attached to an atom of Q. For example, L³ may be attached to a nitrogen atom of the group Q.

In some embodiments, a linker L³ may be linked to a subsituent of a moiety T of a compound of Formula (I). In some embodiments, the compound or salt attached to a linker L³ is represented by:

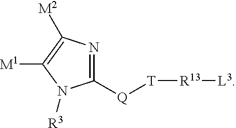

In some embodiments, the compound or salt attached to a linker L³ may be represented by:

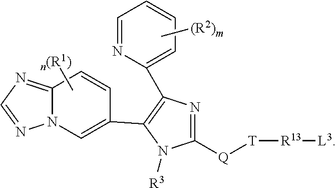

In the structures above, the linker L³ may be attached to an exocyclic atom of the ring system of T. For example, L³ may be attached to an exocyclic nitrogen atom (e.g., of an amine or amine-containing substituent) or oxygen atom (e.g., of a hydroxyl or hydroxyl-containing sub stituent).

Compounds of the present invention are provided in their unconjugated and conjugated forms. For example, it will be understood by the skilled artisan that compounds that can be be attached to a linker contain a suitable atom for attachment to the linker. In some aspects, linker L³ is covalently bound to a substitutable nitrogen or substitutable oxygen of the compound. As an example, it will be understood that the following compound, if desired,

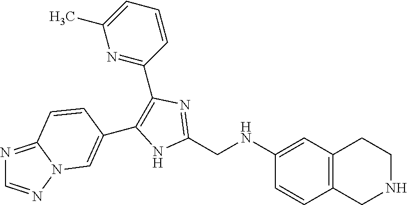 [SMILES: Cc1cccc(-c2nc(CNc3ccc4c(c3)CCNC4)[nH]c2-c2ccc3ncnn3c2)n1]

can be attached to a linker L³, for example, at the nitrogen of the tetrahydroisoquinoline ring. When attached to a linker L³, such a compound can be represented as follows wherein

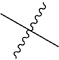

represents the point of attachment to the linker:

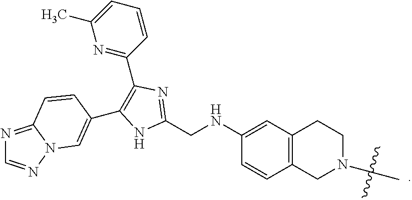 [SMILES: *N1CCc2cc(NCc3nc(-c4cccc(C)n4)c(-c4ccc5ncnn5c4)[nH]3)ccc2C1]

Accordingly, included in the present invention, are, for example, the following compounds wherein

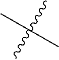

represents the point of attachment to linker L³:

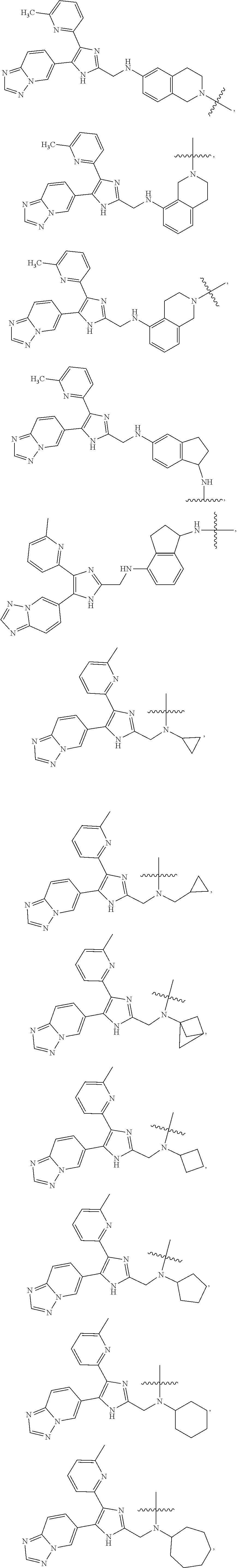

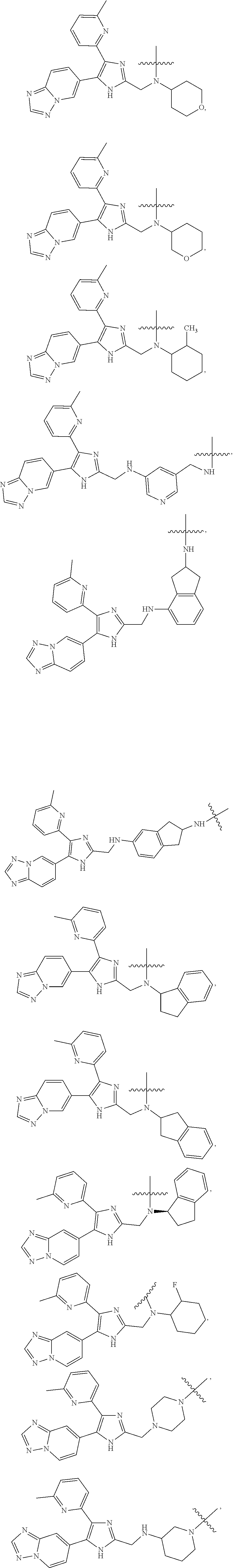

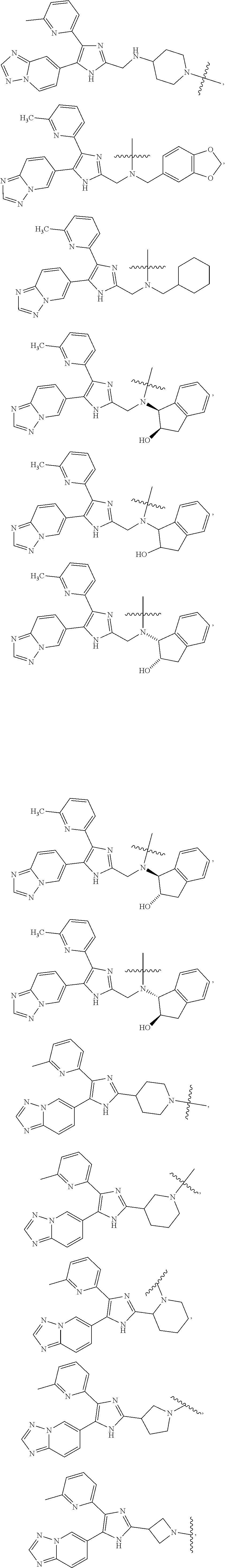

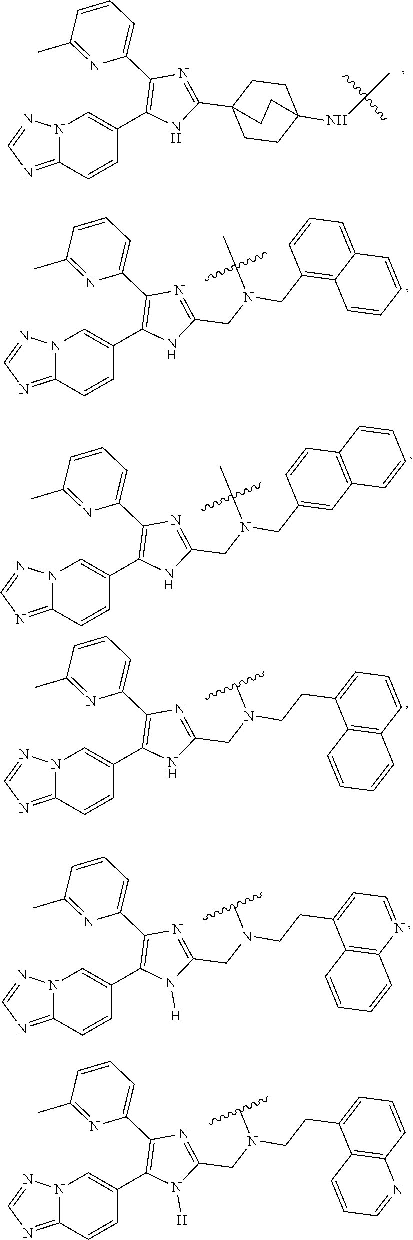

and a salt of any one thereof.

In some aspects, the present disclosure provides a compound selected from:

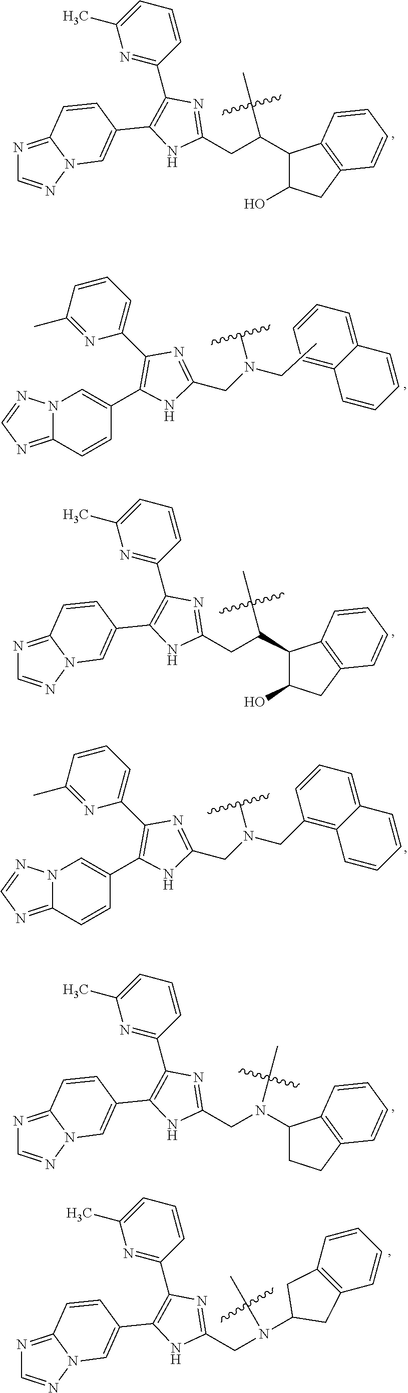

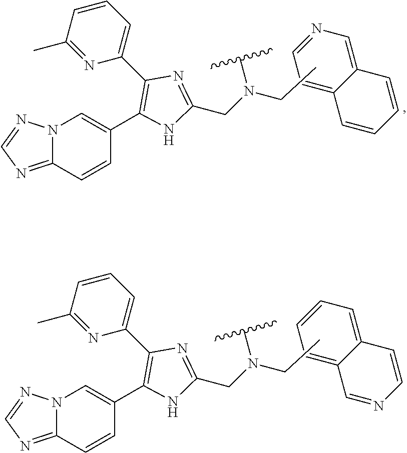

and a salt of any one thereof where

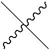

represents the point of attachment to linker L³.

L³ may be a cleavable linker or a non-cleavable linker. In certain embodiments, the linker is further bound to an antibody construct and referred to as a conjugate or an antibody construct conjugate. Linkers of the conjugates described herein may not affect the binding of active portions of a conjugate, e.g., the antigen binding domains, Fc domains, target binding domains, antibodies, compounds, or the like, to a target, which can be a cognate binding partner such as an antigen. A conjugate may comprise multiple linkers, each having one or more compounds (e.g., the same compounds or different compounds) attached. These linkers can be the same linkers or different linkers.

A linker can be short, flexible, rigid, cleavable (e.g., cleavable by a lysosomal enzyme), non-cleavable, hydrophilic, or hydrophobic. A linker can contain segments that have different characteristics, such as segments of flexibility or segments of rigidity. The linker can be chemically stable to extracellular environments, for example, chemically stable in the blood stream, or may include linkages that are not stable or selectively stable. The linker can include linkages that are designed to cleave and/or immolate or otherwise breakdown specifically or non-specifically inside cells. A cleavable linker can be sensitive to enzymes. A cleavable linker can be cleaved by enzymes such as proteases. A cleavable linker may comprise a valine-citrulline (Val-Cit) dipeptide or a valine-alanine (Val-Ala) dipeptide. A valine-citrulline- or valine-alanine-containing linker can contain a pentafluorophenyl group. A valine-citrulline- or valine-alanine-containing linker can contain a maleimide or succinimide group. A valine-citrulline- or valine-alanine-containing linker can contain a para aminobenzyl alcohol (PABA) group. A valine-citrulline- or valine-alanine-containing linker can contain a PABA group and a pentafluorophenyl group. A valine-citrulline- or valine-alanine-containing linker can contain a PABA group and a maleimide or succinimide group.

A cleavable linker can include a maleimido group, such as maleimdocaproyl, attached to a peptide. The peptide can be, for example, valine-citrulline, valine-lysine, valine-alanine, or the like.

A non-cleavable linker can be protease insensitive. A non-cleavable linker can be maleimidocaproyl linker. A maleimidocaproyl linker can comprise N-maleimidomethylcyclohexane-l-carboxylate. A maleimidocaproyl linker can contain a succinimide group. A maleimidocaproyl linker can contain pentafluorophenyl group. A linker can be a combination of a maleimidocaproyl group and one or more polyethylene glycol molecules. A linker can be a maleimide-PEG4 linker. A linker can be a combination of a maleimidocaproyl linker containing a succinimide group and one or more polyethylene glycol molecules. A linker can be a combination of a maleimidocaproyl linker containing a pentafluorophenyl group and one or more polyethylene glycol molecules. A linker can contain maleimides linked to polyethylene glycol molecules in which the polyethylene glycol can allow for more linker flexibility or can be used lengthen the linker. A linker can be a (maleimidocaproyl)-(valine-citrulline)-(para-aminobenzyloxycarbonyl) linker. A linker can be a THIOMAB linker. A THIOMAB linker can be a (maleimidocaproyl)-(valine-citrulline)-(para-aminobenzyloxycarbonyl)- linker.

A linker can also comprise alkylene, alkenylene, alkynylene, polyether, polyester, polyamide group(s) and also, polyamino acids, polypeptides, cleavable peptides, or aminobenzylcarbamates. A linker can contain a maleimide at one end and an N-hydroxysuccinimidyl ester at the other end. A linker can contain a lysine with an N-terminal amine acetylated, and a valine-citrulline cleavage site. A linker can be a link created by a microbial transglutaminase, wherein the link can be created between an amine-containing moiety and a moiety engineered to contain glutamine as a result of the enzyme catalyzing a bond formation between the acyl group of a glutamine side chain and the primary amine of a lysine chain. A linker can contain a reactive primary amine. A linker can be a Sortase A linker, as known in the art.

In the conjugates described herein, any suitable compound of the present invention (e.g., compound or salt of any one of Formulas (I), (Ia), (Ib), (Ic), (Id) and (Ie)) is linked to an antibody construct by way of a linker(s), L³. L³ may be selected from any of the linkers discussed herein. The linker linking the compound or salt to the antibody construct of a conjugate may be short, long, hydrophobic, hydrophilic, flexible or rigid, or may be composed of segments that each independently have one or more of the above-mentioned properties such that the linker may include segments having different properties. The linkers may be polyvalent such that they covalently link more than one compound or salt to a residue of an antibody construct, or monovalent such that covalently they link a single compound or salt to a residue of an antibody construct.

Linkers (referred to as L³ herein) may have from about 10 to about 500 atoms in a linker, such as from about 10 to about 400 atoms, such as from about 10 to about 300 atoms in a linker. In certain embodiments, linkers have from about 30 to about 400 atoms, such as from about 30 to about 300 atoms in the linker.

As will be appreciated by skilled artisans, the linkers may link a compound or salt of the present invention (e.g., a compound of any one of Formulas (I), (Ia), (Ib), (Ic), (Id) and (Ie)) to an antibody construct by covalent linkages between the linker and the antibody construct and compound. As used herein, the term linker may be used to refer to (i) unconjugated forms of the linker that include a functional group capable of covalently linking the linker to a compound(s) or salt(s) of any one of Formulas (I), (Ia), (Ib), (Ic), (Id) and (Ie)) and a functional group capable of covalently linking the linker to an antibody construct; (ii) partially conjugated forms of the linker that include a functional group capable of covalently linking the linker to an antibody construct and that is covalently linked to a compound(s) or salt(s) of any one of Formulas (I), (Ia), (Ib), (Ic), (Id) and (Ie), or vice versa; and (iii) fully conjugated forms of the linker that is covalently linked to both a compound(s) or salt(s) of any one of Formulas (I), (Ia), (Ib), (Ic), (Id) and (Ie) and an antibody construct. One embodiment pertains to a conjugate formed by contacting an antibody construct that binds a cell surface receptor or tumor-associated antigen expressed on a tumor cell with a linker-compound described herein under conditions in which the linker-compound covalently links to the antibody construct. One embodiment pertains to a method of making a conjugate formed by contacting a linker-compound described herein under conditions in which the linker-compound covalently links to the antibody construct. One embodiment pertains to a method of stimulating immune activity in a cell that expresses a target antigen, comprising contacting the cell with a conjugate described herein that is capable of binding the cell, under conditions in which the conjugate binds the cell.

In some embodiments, L³ is a non-cleavable linker. In other embodiments, L³ is cleavable linker such as a linker cleavable by a lysosomal enzyme. In some embodiments, L³ is covalently bound to a substitutable nitrogen or substitutable oxygen of the compound. In some embodiments, L³ is covalently bound to a substitutable nitrogen of the compound.

In some embodiments, L³ is represented by the formula:

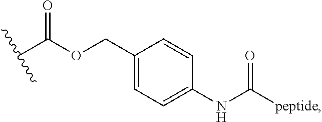

wherein peptide is a group comprising the carbonyl group shown and from one to ten amino acids.

In some embodiments, L³ is represented by the formula:

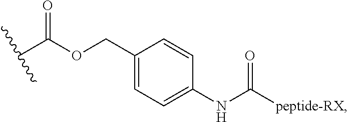

wherein peptide is a group comprising the carbonyl group shown and from one to ten amino acids and RX is a reactive moiety, and [symbol] represents the point of attachment to the compound. In certain embodiments, L³ is represented by the formula:

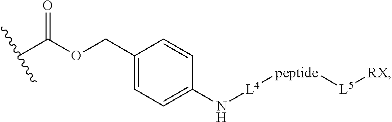

wherein L⁴ represents the C-terminus of the peptide and L⁵ is selected from a bond, alkylene and heteroalkylene, wherein L⁵ is optionally substituted with one or more groups independently selected from R³²; peptide is a group comprising from one to 10 amino acids; RX is a reactive moiety; and [symbol] represents the point of attachment to the compound, where R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, and C₂₋₁₀ alkynyl, each of which is independently optionally substituted at each occurrence with one or more substituents selected from halogen, —OH, —CN, —O-alkyl, —SH, ═O, ═S, —NH₂, —NO₂, and

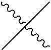

represents point of attachment to the compound or salt. The reactive moiety may be selected from an electrophile, e.g., an α, β-unsaturated carbonyl, such as a maleimide, and may include a leaving group. In some embodiments, the peptide comprises Cit-Val, Val-Cit, Ala-Val, or Val-Ala. For example, the peptide may comprise Val-Cit or Val-Ala. In some embodiments -L³ is represented by the formula:

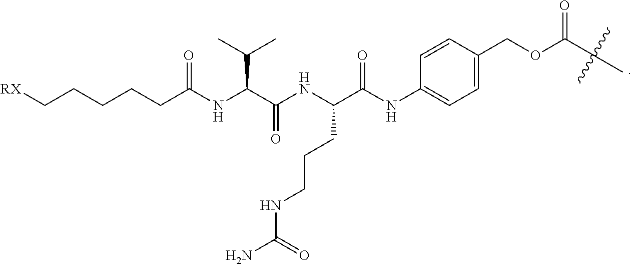

In some embodiments, L³ is represented by the formula:

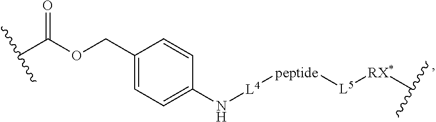

wherein L⁴ represents the C-terminus of the peptide and L⁵ is selected from a bond, alkylene and heteroalkylene, wherein L⁵ is optionally substituted with one or more groups independently selected from R³²; peptide is a group comprising from one to 10 amino acids; [symbol] at the left of the moiety represents the point of attachment to the compound; [symbol] at the right of the moiety represents the point of attachment to a residue of an antibody construct; and RX* is a reactive moiety that has reacted with a moiety on the antibody construct to form a conjugate with an ALK5 inhibitor, where R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, C₂₋₁₀ alkynyl, each of which is independently optionally substituted at each occurrence with one or more substituents selected from halogen, —OH, —CN, —O-alkyl, —SH, ═O, ═S, —NH₂, and —NO₂. In some embodiments, RX* is a bond, a succinimide moiety, or a hydrolyzed succinimide moiety. In some embodiments, the peptide comprises Cit-Val, Val-Cit, Ala-Val, or Val-Ala. For example, the peptide may comprise Val-Cit or Val-Ala. In some embodiments, a peptide is Cit-Val. For example, the linker may be represented by:

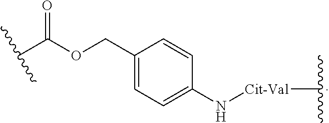

In some embodiments, L³ is selected from:

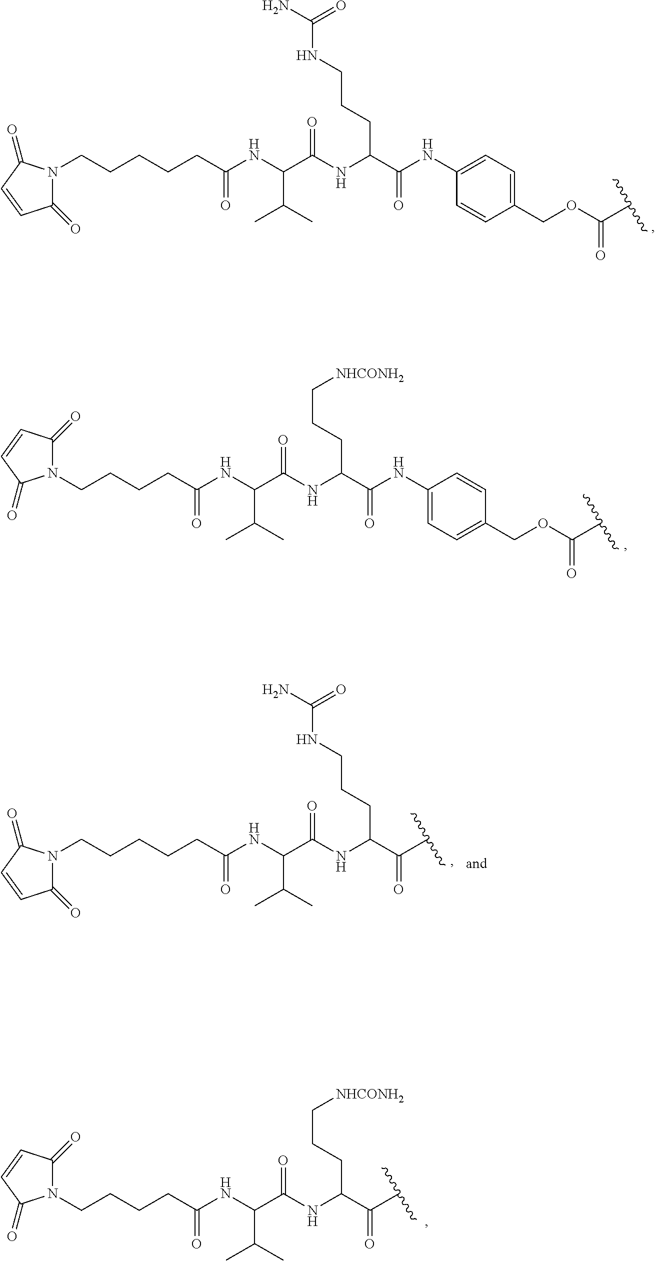

in which the wavy line indicates the point of attachment to the compound.

In some embodiments, for a compound or salt of any one of Formulas (I), (Ia), (Ib), (Ic), (Id) and (Ie) that is covalently bound to a linker -L³, the compound bound to a linker is represented by:

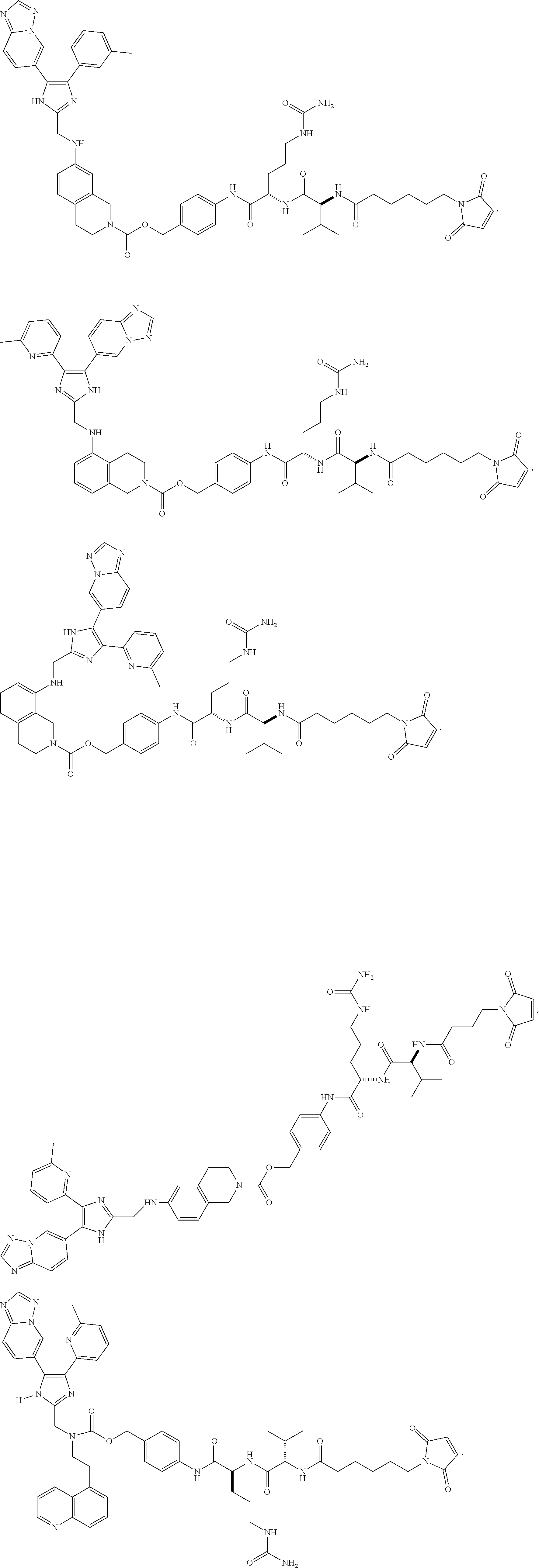

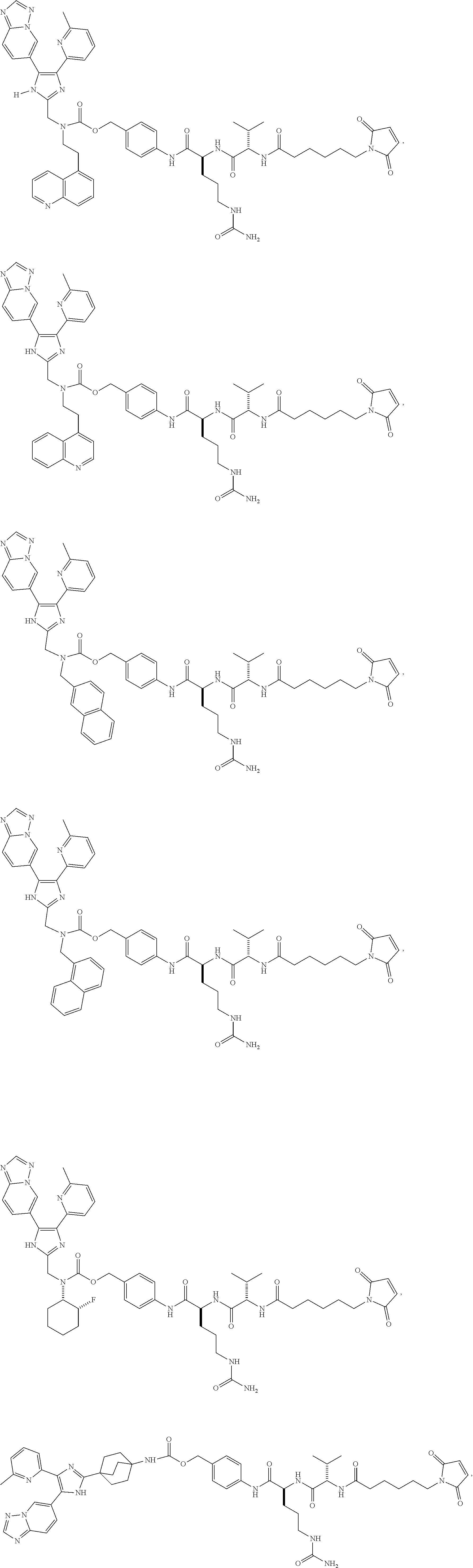

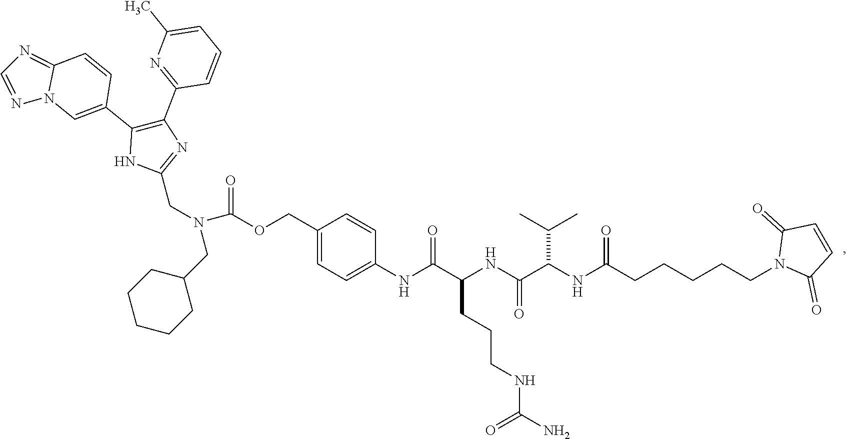

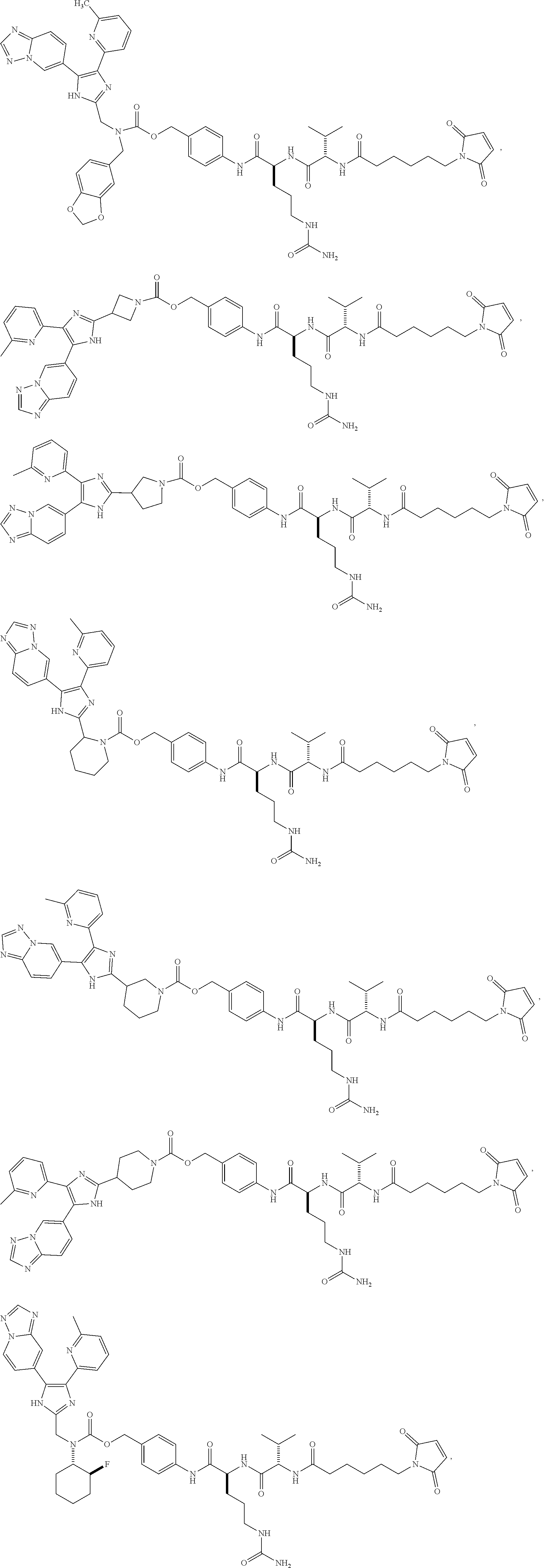

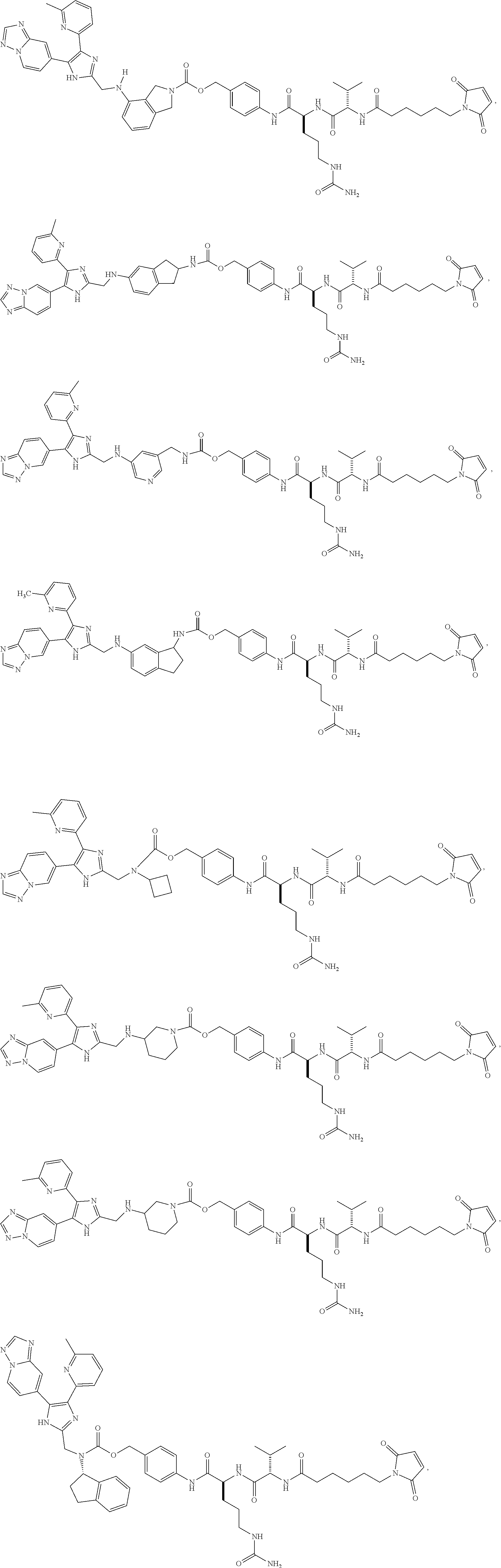

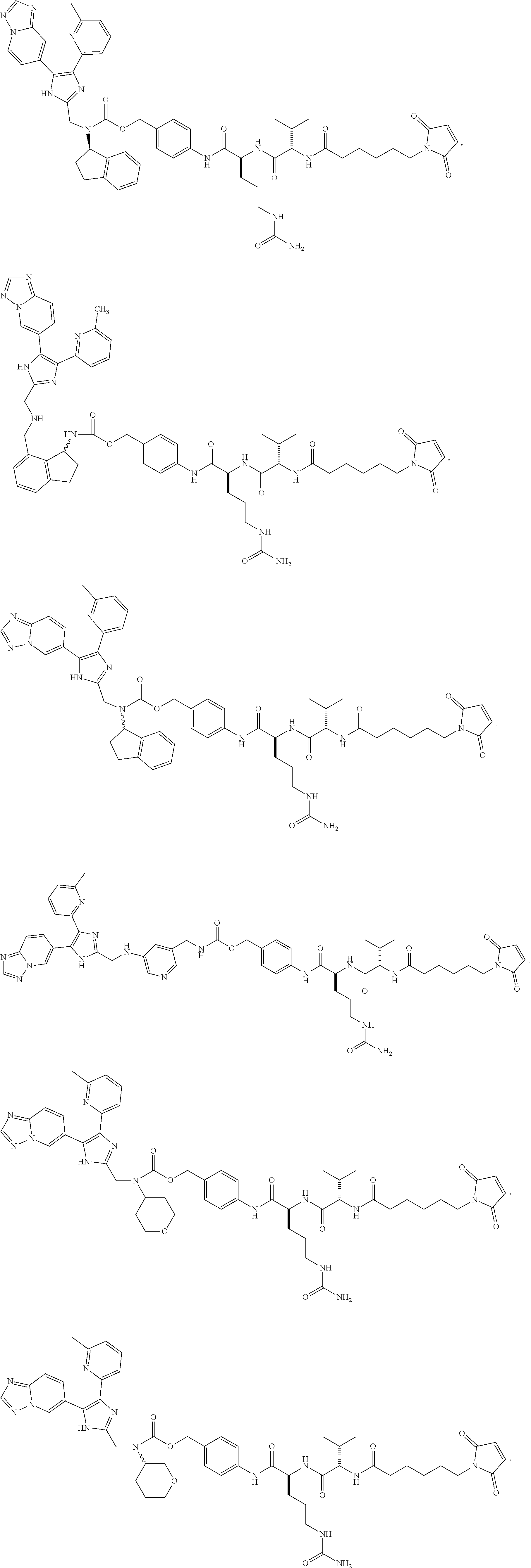

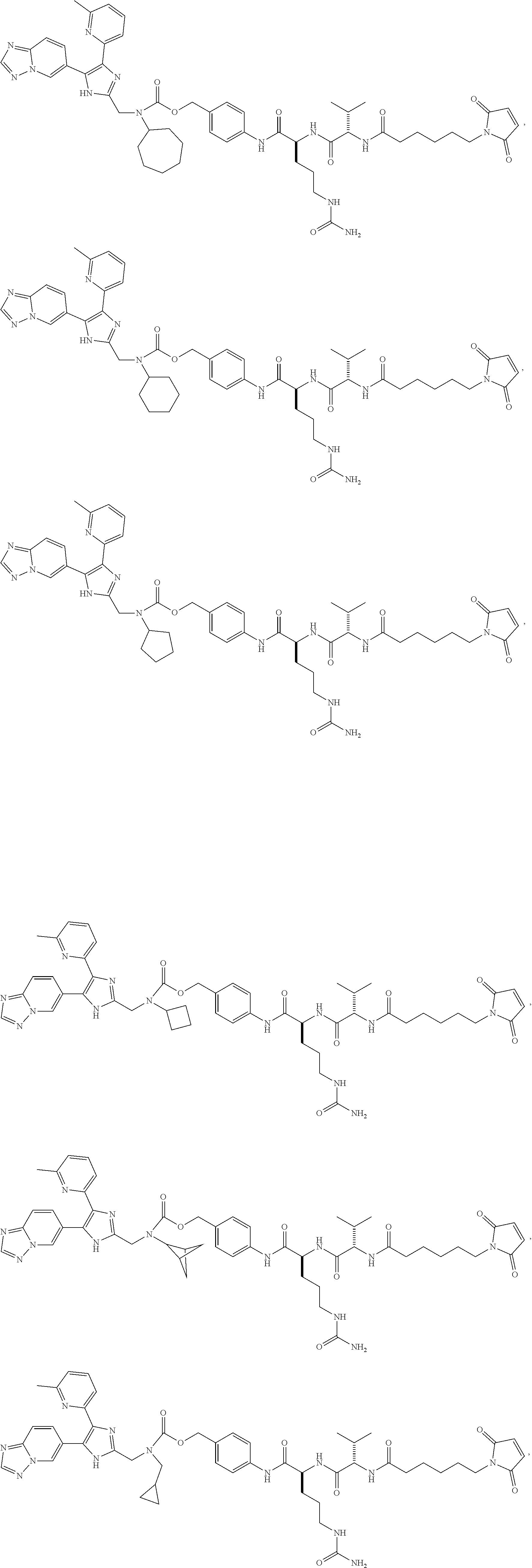

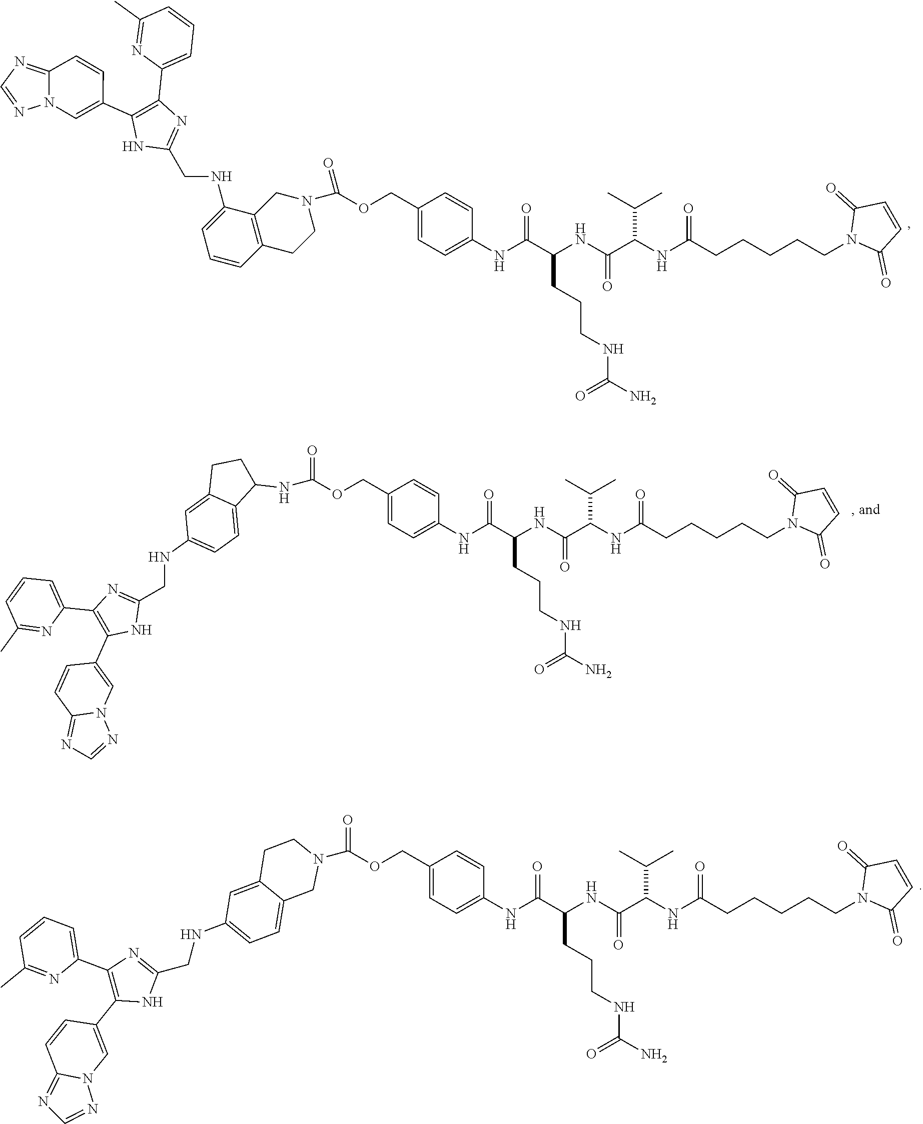

In some embodiments, L³ is represented by the formula:

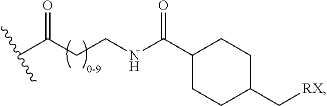

wherein RX comprises a reactive moiety such as a maleimide or a leaving group and [symbol]
represents the point of attachment to the compound. In some embodiments, L³ is represented by the formula:

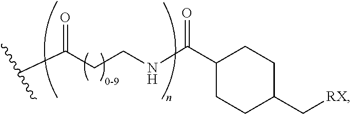

wherein RX comprises a reactive moiety such as a maleimide or a leaving group, n=0-9, and [symbol] represents the point of attachment to the compound. For example, L³ may be represented by the formula:

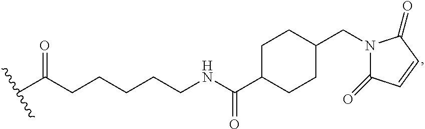 [SMILES: *C(=O)CCCCCNC(=O)C1CCC(CN2C(=O)C=CC2=O)CC1]

in which the wavy line indicates the point of attachment to the compound.

In some embodiments, L³ is represented by the formula:

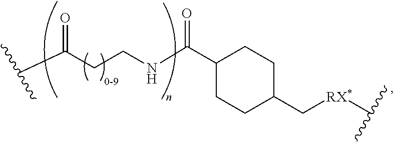

wherein RX* is a bond, a succinimide moiety, or a hydrolyzed succinimide moiety; [symbol] at the right of the moiety represents the point of attachment to a residue of an antibody construct; n=0-9; and [symbol] at the left of the moiety represents the point of attachment to the compound.

A linker may be a polyvalent linker that may be used to link one or more compounds or salts of any one of Formulas (I), (Ia), (Ib), (Ic), (Id) and (Ie) to a residue of an antibody construct are described. For example, Fleximer® linker technology has the potential to enable high-DAR (drug-to-antibody ratio) conjugates with useful physicochemical properties. As shown below, the Fleximer® linker technology is based on incorporating drug molecules into a solubilizing poly-acetal backbone via a sequence of ester bonds:

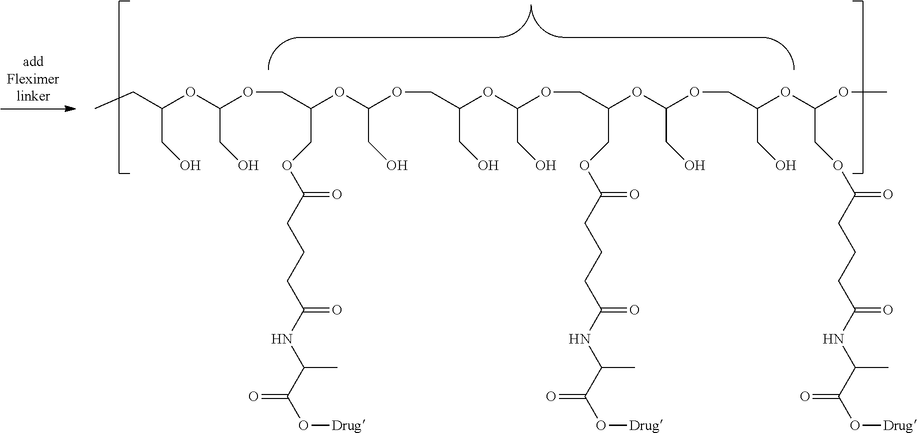

The methodology renders highly-loaded conjugates (DAR up to 20) whilst maintaining good physicochemical properties. This methodology could be utilized with a compound or salt of any one of Formulas (I), (Ia), (Ib), (Ic), (Id) and (Ie).

In certain embodiments, to utilize the Fleximer® linker technology depicted above, an aliphatic alcohol can be present or introduced into a compound or salt thereof (e.g., an ALK5 inhibitor, as described herein). The alcohol moiety is then conjugated to an alanine moiety, which is then synthetically incorporated into the Fleximer® linker. Liposomal processing of the conjugate in vitro releases the parent alcohol-containing drug.

By way of example and not limitation, some cleavable and noncleavable linkers that may be included in the conjugates described herein are described below, in addition to those previously described.

Cleavable linkers can be cleavable in vitro and in vivo. Cleavable linkers can include chemically or enzymatically unstable or degradable linkages. Cleavable linkers can rely on processes inside the cell to liberate a compound or salt thereof (e.g., an ALK5 inhibitor), such as reduction in the cytoplasm, exposure to acidic conditions in the lysosome, or cleavage by specific proteases or other enzymes within the cell. Cleavable linkers can incorporate one or more chemical bonds that are either chemically or enzymatically cleavable while the remainder of the linker can be non-cleavable.

A linker can contain a chemically labile group such as hydrazone and/or disulfide groups. Linkers comprising chemically labile groups can exploit differential properties between the plasma and some cytoplasmic compartments. The intracellular conditions that can facilitate release of a compound or salt thereof (e.g., an ALK5 inhibitor) for hydrazone containing linkers can be the acidic environment of endosomes and lysosomes, while the disulfide containing linkers can be reduced in the cytosol, which can contain high thiol concentrations, e.g., glutathione. The plasma stability of a linker containing a chemically labile group can be increased by introducing steric hindrance using substituents near the chemically labile group.

Acid-labile groups, such as hydrazone, can remain intact during systemic circulation in the blood's neutral pH environment (pH 7.3-7.5) and can undergo hydrolysis and can release a compound or salt thereof (e.g., an ALK5 inhibitor) once the conjugate comprising the antibody construct, the compound, and the linker is internalized into mildly acidic endosomal (pH 5.0-6.5) and lysosomal (pH 4.5-5.0) compartments of the cell. This pH dependent release mechanism can be associated with nonspecific release of the drug. To increase the stability of the hydrazone group of the linker, the linker can be varied by chemical modification, e.g., substitution, allowing tuning to achieve more efficient release in the lysosome with a minimized loss in circulation.

Hydrazone-containing linkers can contain additional cleavage sites, such as additional acid-labile cleavage sites and/or enzymatically labile cleavage sites. Exemplary hydrazone-containing linkers can include, for example, the following structures:

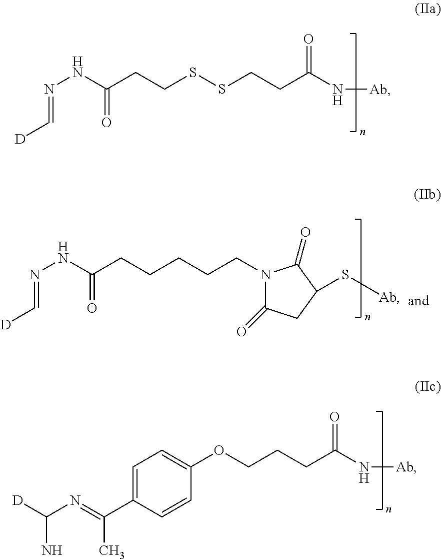

wherein D is a compound or salt of any one of Formulas (I), (Ia), (Ib), (Ic), (Id) and (Ie) and Ab is an antibody construct, respectively, and n represents the number of compound-bound linkers bound to the antibody construct. In certain linkers, such as linker (IIa), the linker can comprise two cleavable groups, a disulfide and a hydrazone moiety. For such linkers, effective release of the unmodified free compound can require acidic pH or disulfide reduction and acidic pH. Linkers such as (IIb) and (IIc) can be effective with a single hydrazone cleavage site. Acid-labile linkers may also include silyl ethers. For example, a linker for use in a conjugate described herein may be represented by:

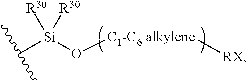

in which each R³° is independently selected from optionally substituted C₁-₆ alkyl and phenyl; the wavy line indicates an attachment to a compound or salt of any one of Formulas (I), (Ia), (Ib), (Ic), (Id) and (Ie);and RX includes a reactive moiety such as a leaving group or a maleimide. In certain embodiments, L³ is represented by the formula:

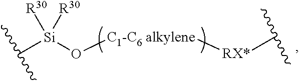

in which each R³⁰ is independently selected from optioinally substituted C₁₋₆ alkyl and phenyl; the wavy line on the left indicates an attachment to a compound or salt of any one of Formulas (I), (Ia), (Ib), (Ic), (Id) and (Ie); the wavy line on the right indicates attachment to a residue of an antibody construct; and RX* is a bond, a succinimide moiety, or a hydrolyzed succinimide moiety.

Other acid-labile groups that can be included in linkers include cis-aconityl-containing linkers. cis-Aconityl chemistry can use a carboxylic acid juxtaposed to an amide bond to accelerate amide hydrolysis under acidic conditions.

Cleavable linkers can also include a disulfide group. Disulfides can be thermodynamically stable at physiological pH and can be designed to release the compound (e.g., ALK5 inhibitor) upon internalization inside cells, wherein the cytosol can provide a significantly more reducing environment compared to the extracellular environment. Scission of disulfide bonds can require the presence of a cytoplasmic thiol cofactor, such as (reduced) glutathione (GSH), such that disulfide-containing linkers can be reasonably stable in circulation, selectively releasing the compound (e.g., ALK5 inhibitor) in the cytosol. The intracellular enzyme protein disulfide isomerase, or similar enzymes capable of cleaving disulfide bonds, can also contribute to the preferential cleavage of disulfide bonds inside cells. GSH can be present in cells in the concentration range of 0.5-10 mM compared with a significantly lower concentration of GSH or cysteine, the most abundant low-molecular weight thiol, in circulation at approximately 5 μM. Tumor cells, where irregular blood flow can lead to a hypoxic state, can result in enhanced activity of reductive enzymes and therefore even higher glutathione concentrations. The in vivo stability of a disulfide-containing linker can be enhanced by chemical modification of the linker, e.g., use of steric hindrance adjacent to the disulfide bond.

Disulfide-containing linkers can include the following structures:

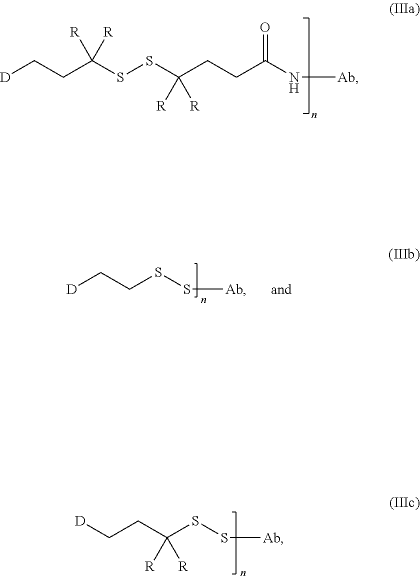

wherein D is a compound or salt of any one of Formulas (I), (Ia), (Ib), (Ic), (Id) and (Ie)and Ab is an antibody construct, respectively, n represents the number of compounds bound to linkers (L³) bound to the antibody construct and R is independently selected at each occurrence from hydrogen or alkyl, for example. Increasing steric hindrance adjacent to the disulfide bond can increase the stability of the linker. Structures such as (IIIa) and (IIIc) can show increased in vivo stability when one or more R groups is selected from a lower alkyl group such as methyl.

Another type of linker that can be used is a linker that is specifically cleaved by an enzyme. For example, the linker can be cleaved by a lysosomal enzyme. Such linkers can be peptide-based or can include peptidic regions that can act as substrates for enzymes. Peptide based linkers can be more stable in plasma and extracellular milieu than chemically labile linkers.

Peptide bonds can have good serum stability, as lysosomal proteolytic enzymes can have very low activity in blood due to endogenous inhibitors and the unfavorably high pH value of blood compared to lysosomes. Release of a compound (e.g., an ALK5 inhibitor) from a residue of an antibody construct can occur due to the action of lysosomal proteases, e.g., cathepsin and plasmin. These proteases can be present at elevated levels in certain tumor tissues. The linker can be cleavable by a lysosomal enzyme. The lysosomal enzyme can be, for example, cathepsin B, β-glucuronidase, or β-galactosidase.

The cleavable peptide can be selected from tetrapeptides or dipeptides. Exemplary dipeptides are Val-Cit, Val-Ala, and Phe-Lys. Dipeptides can have lower hydrophobicity compared to longer peptides.

A variety of dipeptide-based cleavable linkers can be used in conjugates described herein. Enzymatically cleavable linkers can include a self-immolative component to spatially separate a compound (e.g., an ALK5 inhibitor) from the site of enzymatic cleavage. The direct attachment of a compound to a peptide linker can result in proteolytic release of the compound or of an amino acid adduct of the compound, thereby impairing its activity. The use of a self-immolative spacer can allow for the elimination of the fully active, chemically unmodified compound upon amide bond hydrolysis.

One self-immolative component (e.g., spacer) can be a bifunctional para-aminobenzyl alcohol group, which can link to the peptide through the amino group, forming an amide bond, while amine-containing compounds (e.g., ALK5 inhibitors) can be attached through carbamate functionalities to the benzylic hydroxyl group of the linker (to give a p-amidobenzylcarbamate, PABC). The resulting pro-compound can be activated upon protease-mediated cleavage, leading to a 1,6-elimination reaction releasing the unmodified compound, carbon dioxide, and remnants of the linker group. The following scheme depicts the fragmentation of p-amidobenzyl carbamate and release of the compound:

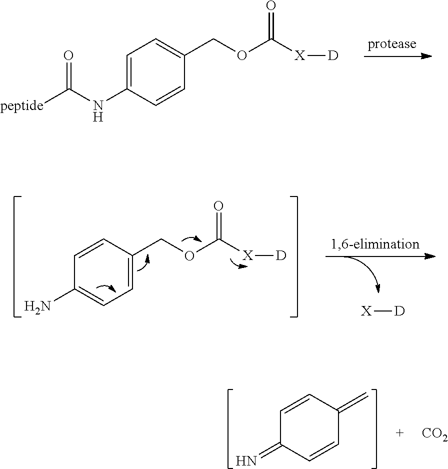

wherein X-D represents the unmodified compound. Heterocyclic variants of this self-immolative group have also been described.

Another example of a self-immolative component includes a methylene carbamate unit. A self-immolative component may have the form:

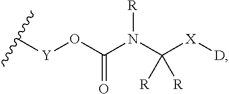

in which the wavy line represents the point of attachment to additional linker components or a residue of an antibody construct; Y is an activatable self-immolative moiety; X-D is a compound or salt thereof as described herein, where X is, for example, a heteroatom such as oxygen; and R is independently selected at each occurrence from hydrogen, optionally substituted C₁₋₆ alkylene, an optionally substituted C₃₋₁₂ carbocycle, and an optionally substituted 3- to 12-membered heterocycle, and a carbocycle or heterocycle interspersed with one or more alkylene groups.

The enzymatically cleavable linker can be a β-glucuronic acid-based linker. Facile release of the compound (e.g., ALK5 inhibitor) can be realized through cleavage of the β-glucuronide glycosidic bond by the lysosomal enzyme β-glucuronidase. This enzyme can be abundantly present within lysosomes and can be overexpressed in some tumor types, while the enzyme activity outside cells can be low. β-Glucuronic acid-based linkers can be used to circumvent the tendency of a conjugate to undergo aggregation due to the hydrophilic nature of β-glucuronides. In certain embodiments, β-glucuronic acid-based linkers can link an antibody construct to a hydrophobic compound. The following scheme depicts the release of a compound (D) from an antibody construct compound conjugate containing a β-glucuronic acid-based linker:

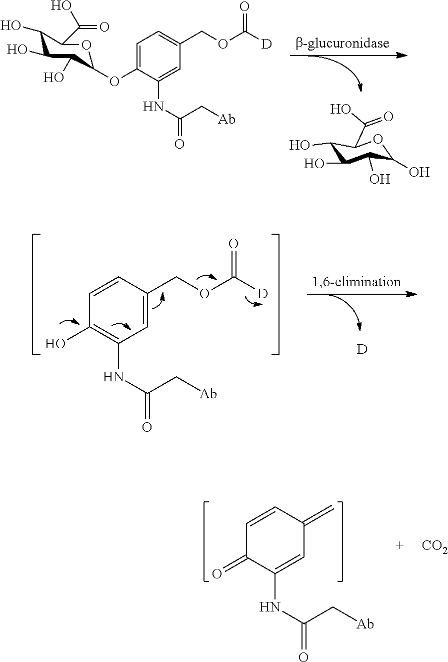

in which Ab indicates the antibody construct.

A variety of cleavable β-glucuronic acid-based linkers useful for linking drugs such as auristatins, camptothecin and doxorubicin analogues, CBI minor-groove binders, and psymberin to antibodies have been described. These β-glucuronic acid-based linkers may be used in the conjugates described herein. In certain embodiments, the enzymatically cleavable linker is a β-galactoside-based linker. β-Galactoside is present abundantly within lysosomes, while the enzyme activity outside cells is low.

Additionally, compounds (e.g., ALK5 inhibitors) containing a phenol group can be covalently bonded to a linker through the phenolic oxygen. One such linker relies on a methodology in which a diamino-ethane “Space Link” is used in conjunction with traditional “PABO”-based self-immolative groups to deliver phenols.

Cleavable linkers can include non-cleavable portions or segments, and/or cleavable segments or portions can be included in an otherwise non-cleavable linker to render it cleavable. By way of example only, polyethylene glycol (PEG) and related polymers can include cleavable groups in the polymer backbone. For example, a polyethylene glycol or polymer linker can include one or more cleavable groups such as a disulfide, a hydrazone or a dipeptide.

Other degradable linkages that can be included in linkers can include ester linkages formed by the reaction of PEG carboxylic acids or activated PEG carboxylic acids with alcohol groups on a compound (e.g., ALK5 inhibitor), wherein such ester groups can hydrolyze under physiological conditions to release the compound. Hydrolytically degradable linkages can include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol; and oligonucleotide linkages formed by a phosphoramidite group, including but not limited to, at the end of a polymer, and a 5′ hydroxyl group of an oligonucleotide.

A linker can contain an enzymatically cleavable peptide moiety, for example, a linker comprising structural formula (IVa), (IVb), (IVc), or (IVd):

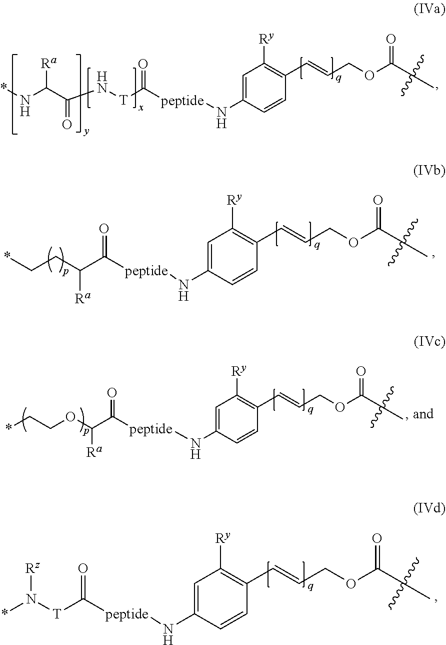

or a salt thereof, in which: “peptide” represents a peptide (illustrated in N→C orientation, in which peptide includes the amino and carboxy “termini”) that is cleavable by a lysosomal enzyme; T represents a polymer comprising one or more ethylene glycol units or an alkylene chain, or combinations thereof R^{a} is selected from hydrogen, alkyl, sulfonate and methyl sulfonate; R^{y} is hydrogen or C₁₋₄ alkyl-(O)ᵣ—(C₁₋₄ alkylene)ₛ-G¹ or C₁₋₄ alkyl-(N)-[(C₁₋₄ alkylene)-G¹]₂; R^{z} is C₁₋₄ alkyl-(O)ᵣ—(C₁₋₄ alkylene)ₛ-G²; G¹ is SO₃H, CO₂H, PEG 4-32, or a sugar moiety; G² is SO₃H, CO₂H, or a PEG 4-32 moiety; r is 0 or 1; s is 0 or 1; p is an integer ranging from 0 to 5; q is 0 or 1; xis 0 or 1; y is 0 or 1; [symbol] represents the point of attachment of the linker to a compound or salt of any one of Formulas (I), (Ia), (Ib), (Ic), (Id) and (Ie); and * represents the point of attachment to another portion of the linker or to a residue of an antibody construct.

In certain embodiments, the peptide can be selected from natural amino acids, unnatural amino acids or combinations thereof. In certain embodiments, the peptide can be selected from a tripeptide or a dipeptide. In particular embodiments, the dipeptide can comprise L-amino acids and be selected from: Val-Cit; Cit-Val; Ala-Ala; Ala-Cit; Cit-Ala; Asn-Cit; Cit-Asn; Cit-Cit; Val-Glu; Glu-Val; Ser-Cit; Cit—Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit, or salts thereof.

Exemplary embodiments of linkers according to structural formula (IVa) are illustrated below (as illustrated, the linkers include a reactive group suitable for covalently linking the linker to an antibody construct):

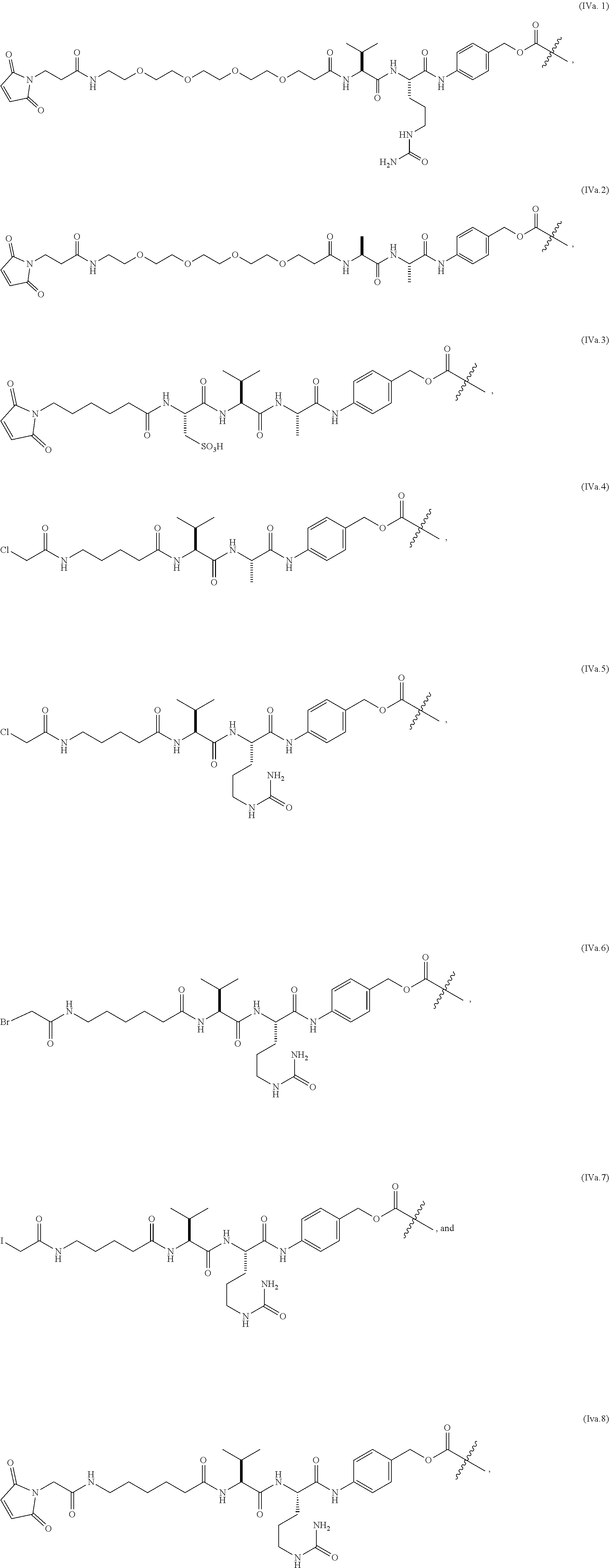

wherein [symbol] indicates an attachment site of a linker (L³) to a compound or salt of any one of Formulas (I), (Ia), (Ib), (Ic), (Id) and (Ie).

Exemplary embodiments of linkers according to structural formula (IVb), (IVc), or (IVd) that can be included in the conjugates described herein can include the linkers illustrated below (as illustrated, the linkers include a reactive group suitable for covalently linking the linker to an antibody construct):

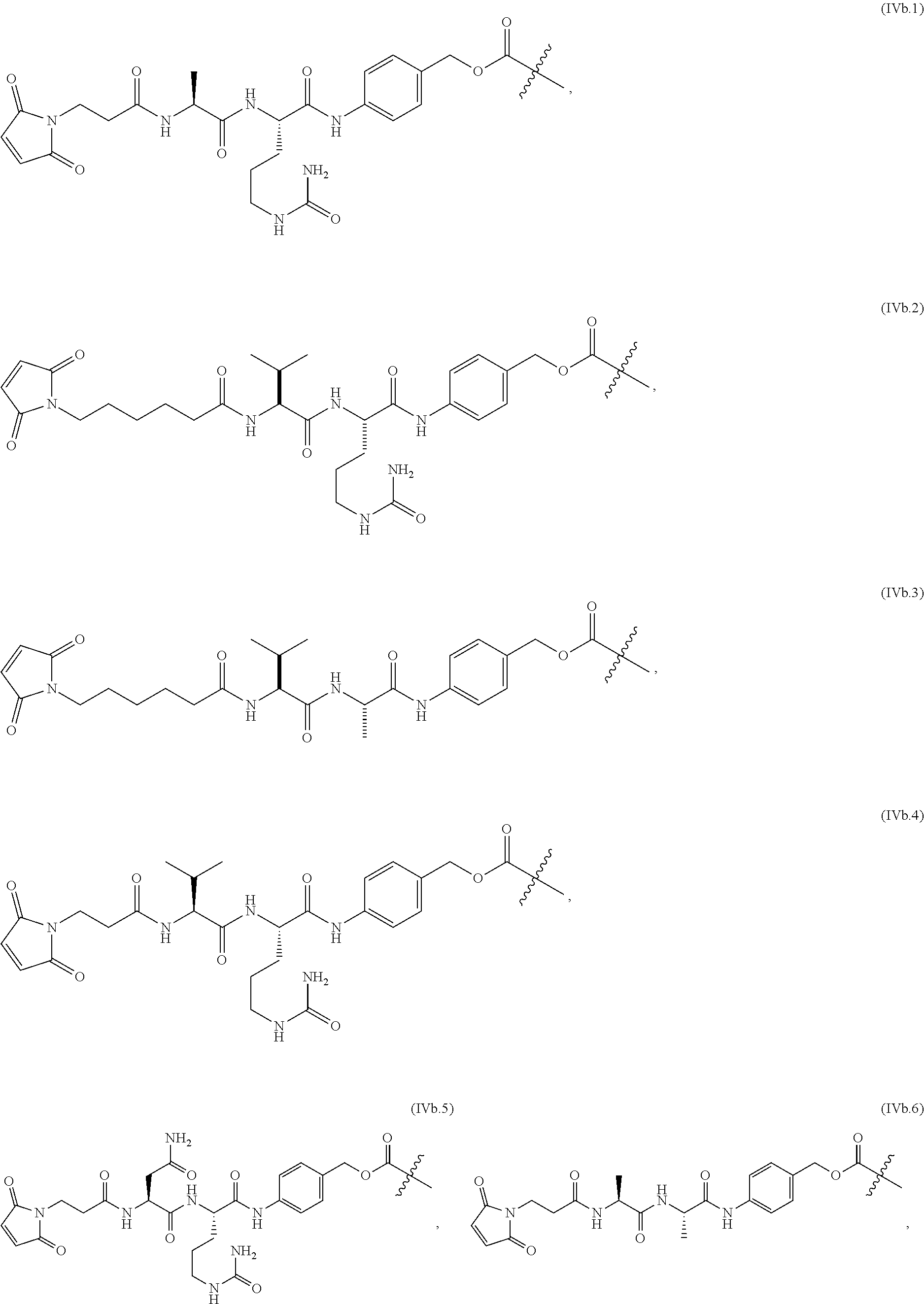 [SMILES: *C(=O)OCc1ccc(NC(=O)[C@H](CCCNC(N)=O)NC(=O)[C@H](C)NC(=O)CCN2C(=O)C=CC2=O)cc1 ; *C(=O)OCc1ccc(NC(=O)[C@H](CCCNC(N)=O)NC(=O)[C@@H](NC(=O)CCCCCN2C(=O)C=CC2=O)C(C)C)cc1 ; *C(=O)OCc1ccc(NC(=O)[C@H](C)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@@H](NC(=O)CCN2C(=O)C=CC2=O)C(C)C)cc1 ; *C(=O)OCc1ccc(NC(=O)[C@H](CCCNC(N)=O)NC(=O)[C@H](CC(N)=O)NC(=O)CCN2C(=O)C=CC2=O)cc1 ; *C(=O)OCc1ccc(NC(=O)[C@H](C)NC(=O)[C@H](C)NC(=O)CCN2C(=O)C=CC2=O)cc1]

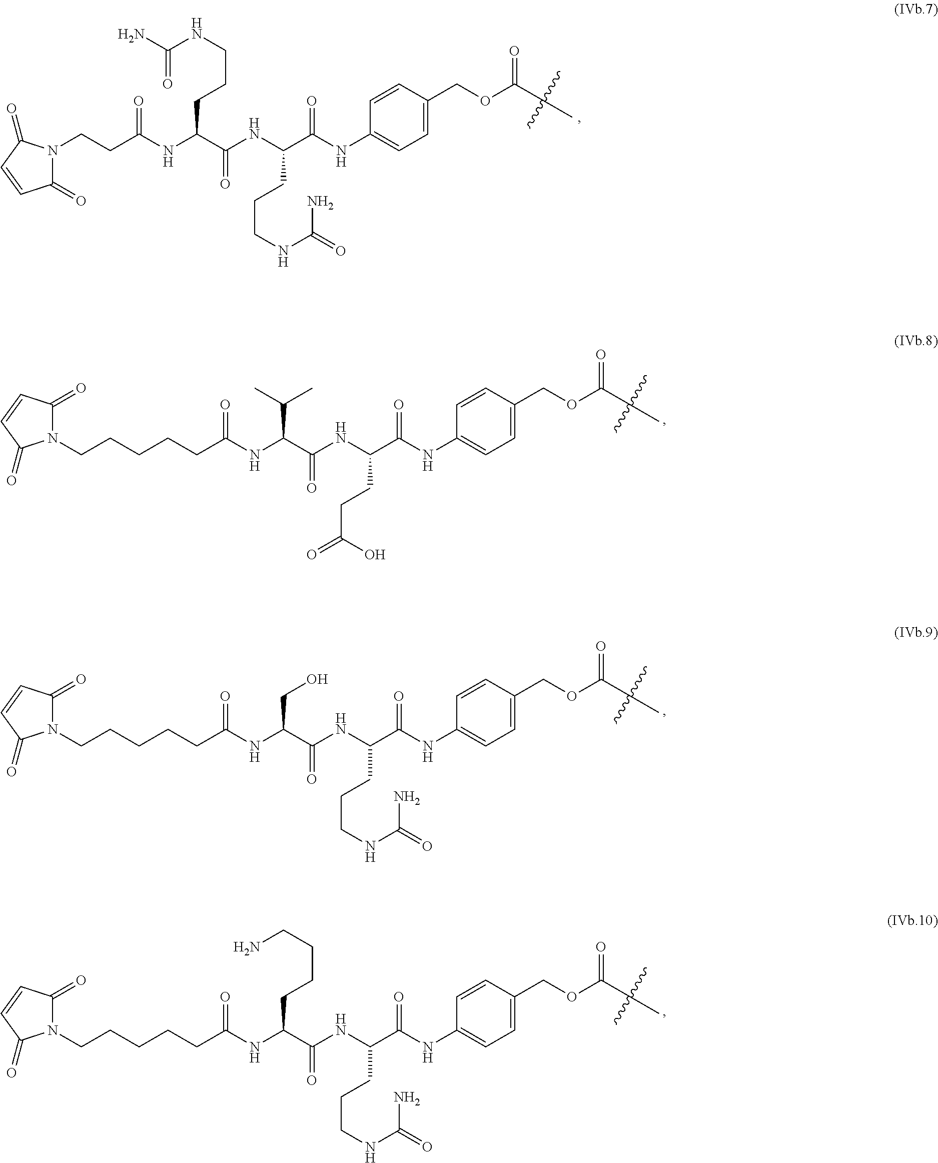 [SMILES: *C(=O)OCc1ccc(NC(=O)[C@H](CCCNC(N)=O)NC(=O)[C@H](CCCNC(N)=O)NC(=O)CCN2C(=O)C=CC2=O)cc1 ; *C(=O)OCc1ccc(NC(=O)[C@H](CCC(=O)O)NC(=O)[C@@H](NC(=O)CCCCCN2C(=O)C=CC2=O)C(C)C)cc1 ; *C(=O)OCc1ccc(NC(=O)[C@H](CCCNC(N)=O)NC(=O)[C@H](CO)NC(=O)CCCCCN2C(=O)C=CC2=O)cc1 ; *C(=O)OCc1ccc(NC(=O)[C@H](CCCNC(N)=O)NC(=O)[C@H](CCCCN)NC(=O)CCCCCN2C(=O)C=CC2=O)cc1]

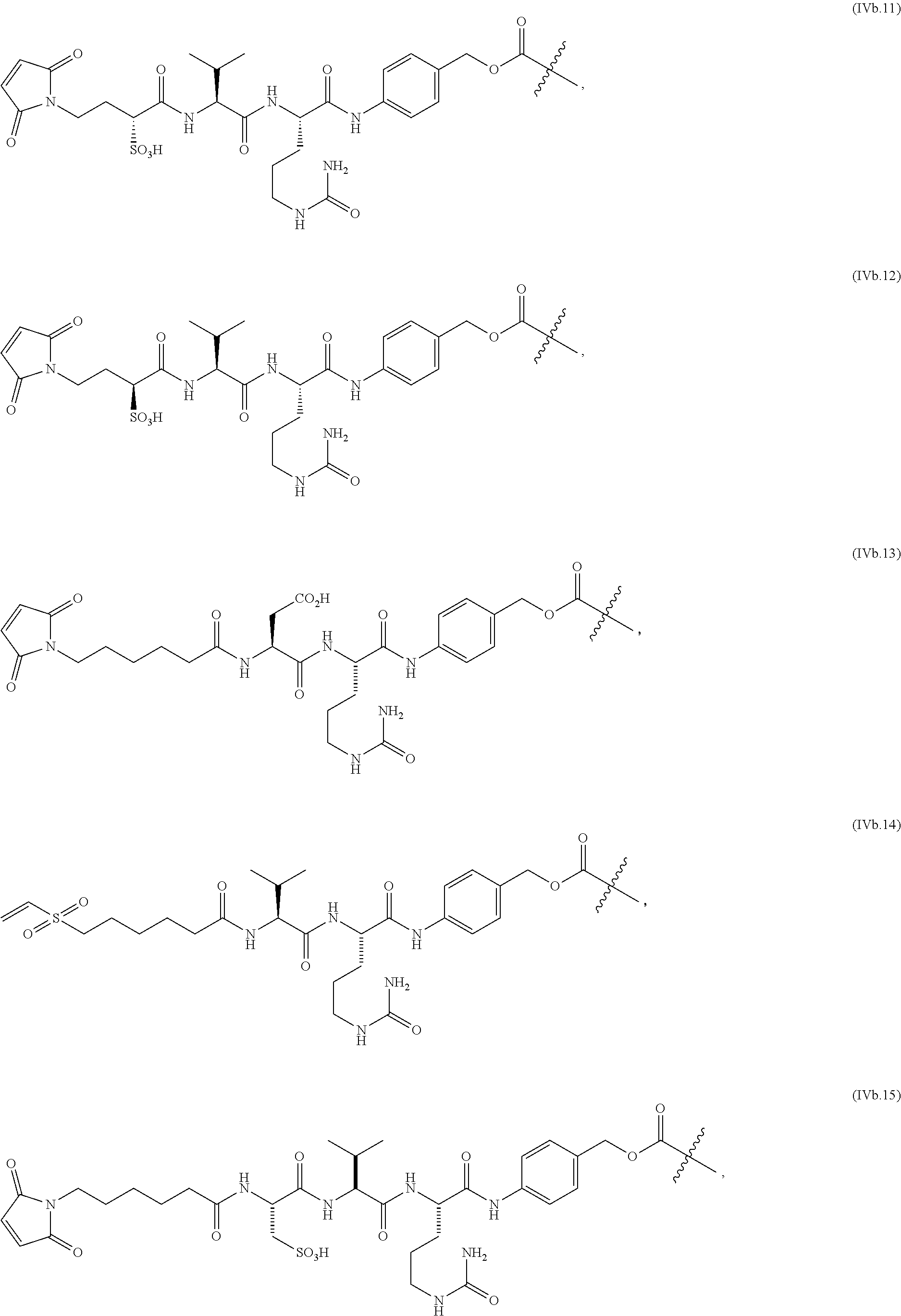 [SMILES: *C(=O)OCc1ccc(NC(=O)[C@H](CCCNC(N)=O)NC(=O)[C@@H](NC(=O)[C@@H](CCN2C(=O)C=CC2=O)S(=O)(=O)O)C(C)C)cc1 ; *C(=O)OCc1ccc(NC(=O)[C@H](CCCNC(N)=O)NC(=O)[C@@H](NC(=O)[C@H](CCN2C(=O)C=CC2=O)S(=O)(=O)O)C(C)C)cc1 ; *C(=O)OCc1ccc(NC(=O)[C@H](CCCNC(N)=O)NC(=O)[C@H](CC(=O)O)NC(=O)CCCCCN2C(=O)C=CC2=O)cc1 ; *C(=O)OCc1ccc(NC(=O)[C@H](CCCNC(N)=O)NC(=O)[C@@H](NC(=O)CCCCCS(=O)(=O)C=C)C(C)C)cc1 ; *C(=O)OCc1ccc(NC(=O)[C@H](CCCNC(N)=O)NC(=O)[C@@H](NC(=O)[C@H](CS(=O)(=O)O)NC(=O)CCCCCN2C(=O)C=CC2=O)C(C)C)cc1]

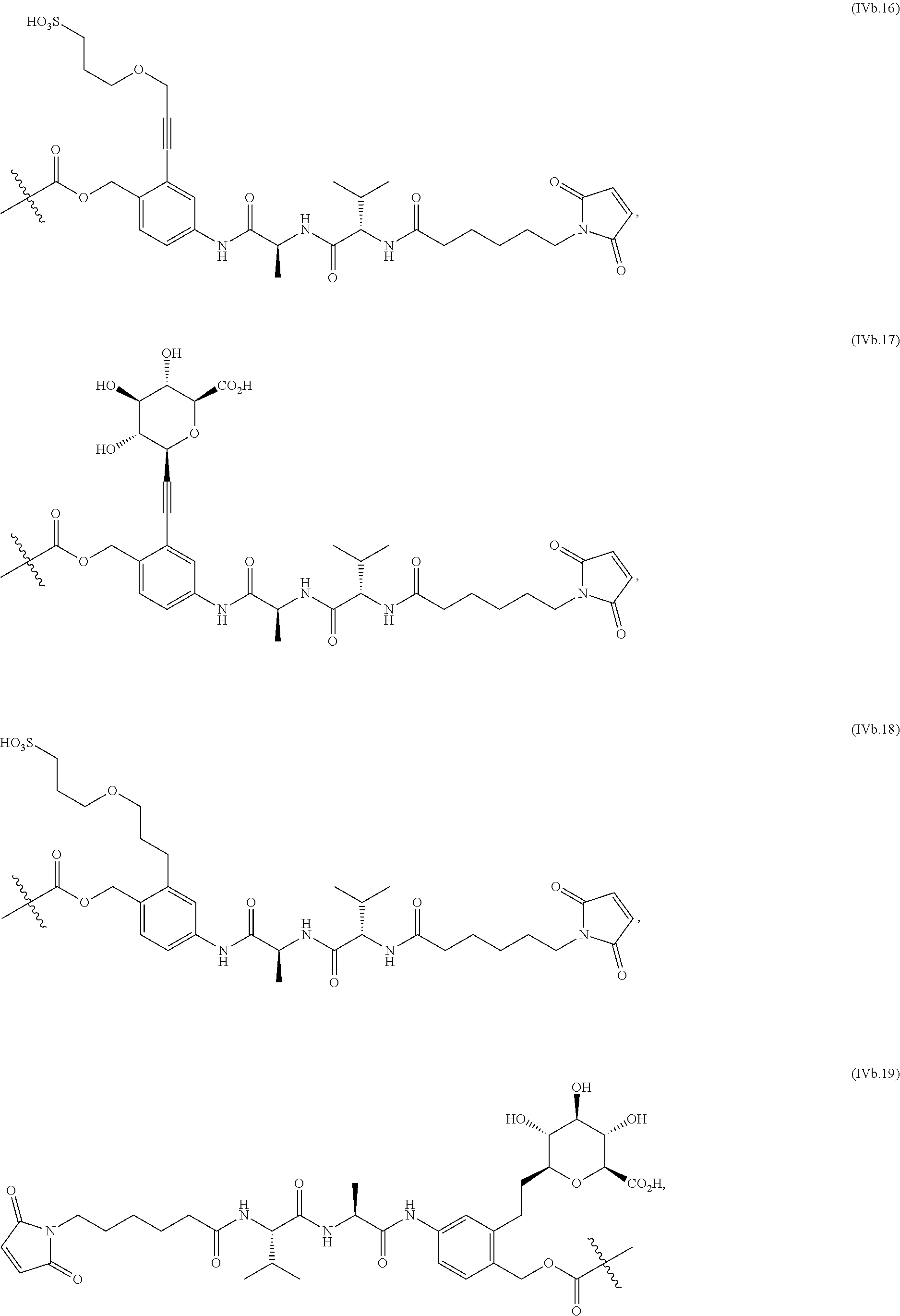 [SMILES: *C(=O)OCc1ccc(NC(=O)[C@H](C)NC(=O)[C@@H](NC(=O)CCCCCN2C(=O)C=CC2=O)C(C)C)cc1C#CCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1C#C[C@@H]1O[C@H](C(=O)O)[C@@H](O)[C@H](O)[C@H]1O ; *C(=O)OCc1ccc(NC(=O)[C@H](C)NC(=O)[C@@H](NC(=O)CCCCCN2C(=O)C=CC2=O)C(C)C)cc1CCCOCCCS(=O)(=O)O ; *C(=O)OCc1ccc(NC(=O)[C@H](C)NC(=O)[C@@H](NC(=O)CCCCCN2C(=O)C=CC2=O)C(C)C)cc1CC[C@@H]1O[C@H](C(=O)O)[C@@H](O)[C@H](O)[C@H]1O]

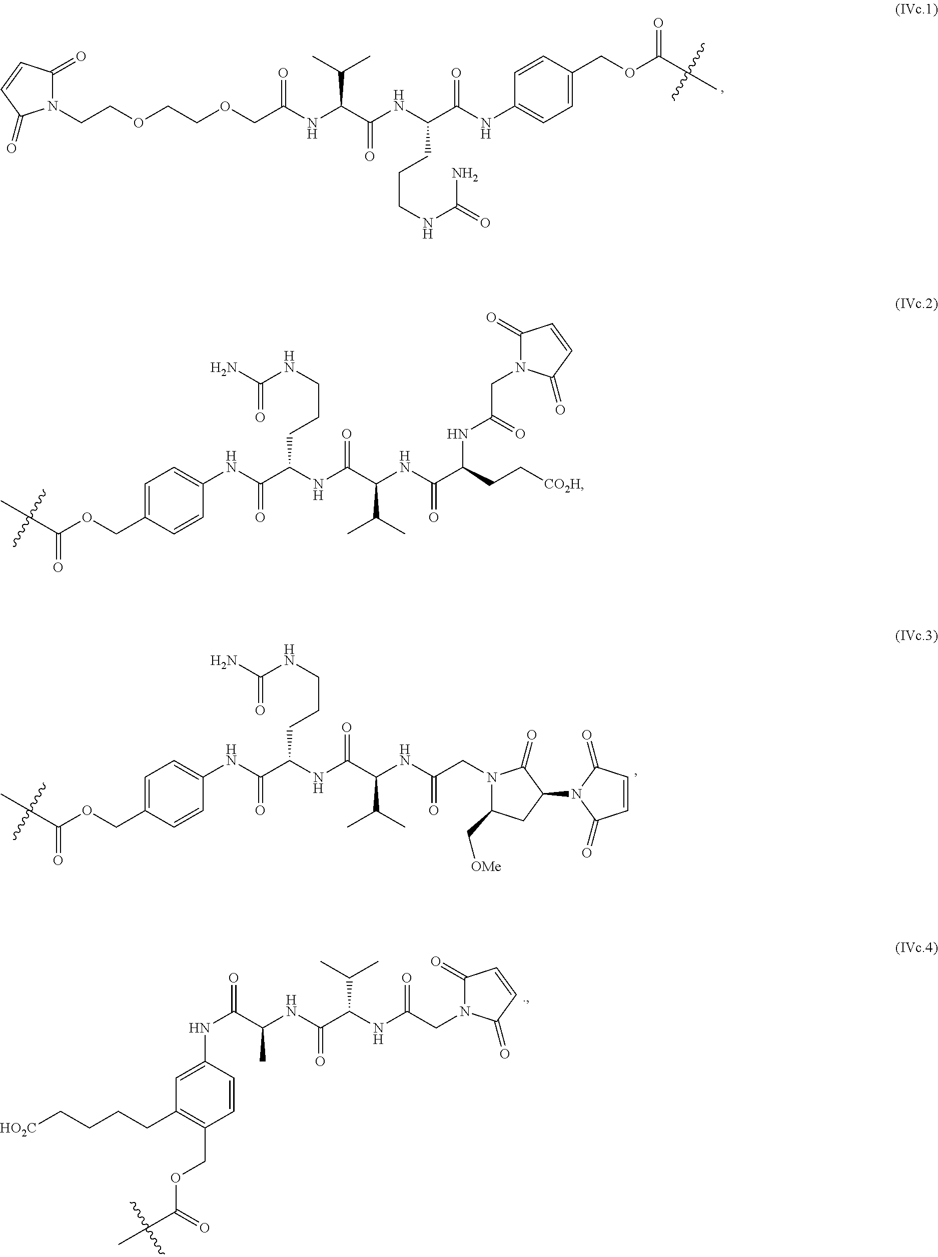 [SMILES: *C(=O)OCc1ccc(NC(=O)[C@H](CCCNC(N)=O)NC(=O)[C@@H](NC(=O)COCCOCCN2C(=O)C=CC2=O)C(C)C)cc1 ; *C(=O)OCc1ccc(NC(=O)[C@H](CCCNC(N)=O)NC(=O)[C@@H](NC(=O)[C@H](CCC(=O)O)NC(=O)CN2C(=O)C=CC2=O)C(C)C)cc1 ; *C(=O)OCc1ccc(NC(=O)[C@H](CCCNC(N)=O)NC(=O)[C@@H](NC(=O)CN2C(=O)[C@@H](N3C(=O)C=CC3=O)C[C@H]2COC)C(C)C)cc1 ; *C(=O)OCc1ccc(NC(=O)[C@H](C)NC(=O)[C@@H](NC(=O)CN2C(=O)C=CC2=O)C(C)C)cc1CCCCC(=O)O]

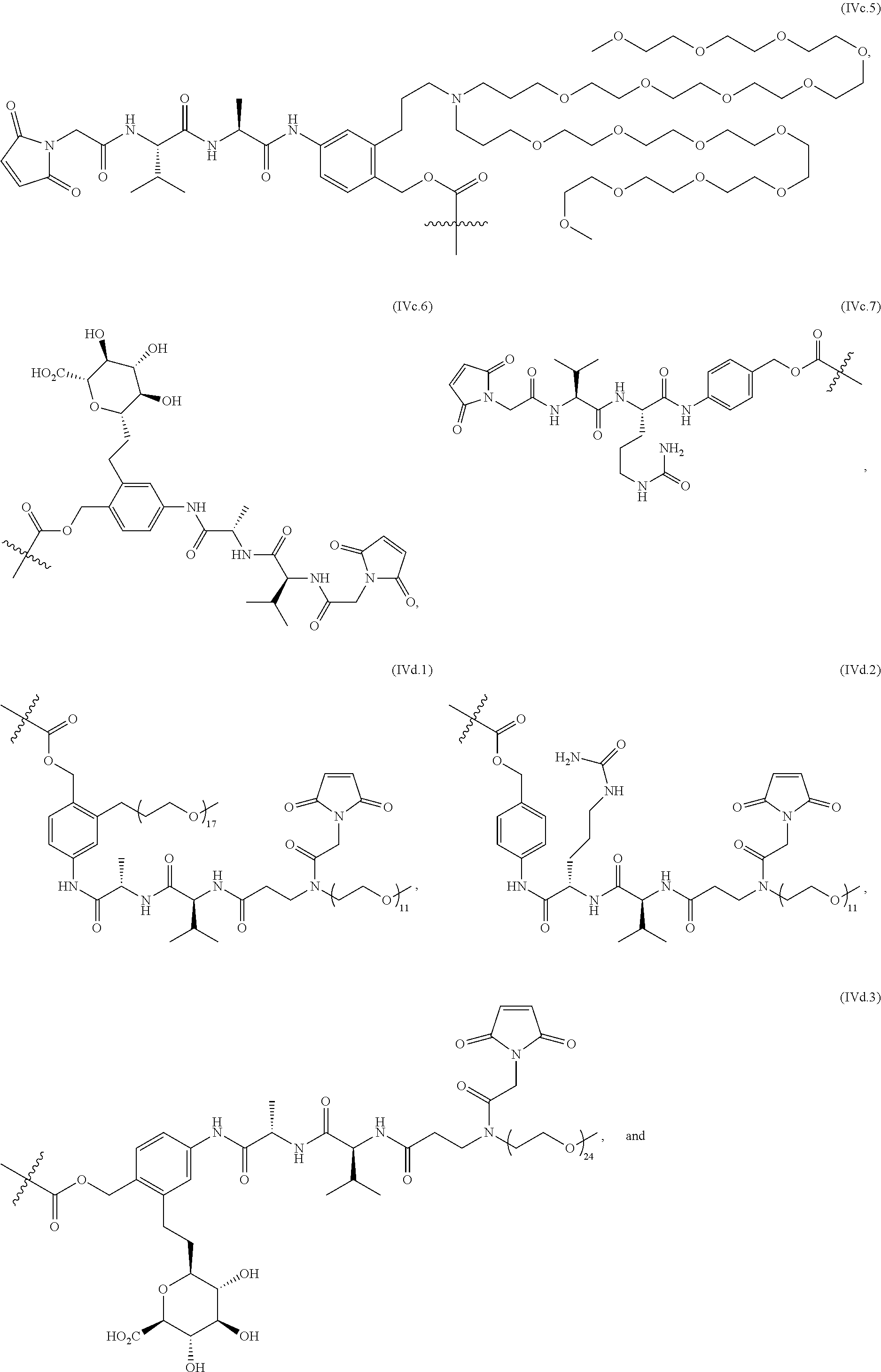

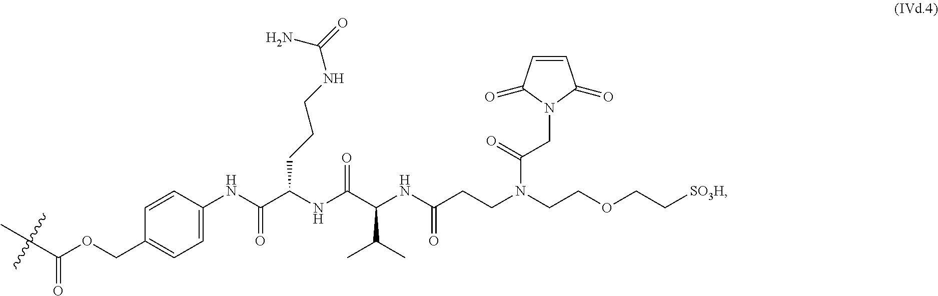 [SMILES: *C(=O)OCc1ccc(NC(=O)[C@H](CCCNC(N)=O)NC(=O)[C@@H](NC(=O)CCN(CCOCCS(=O)(=O)O)C(=O)CN2C(=O)C=CC2=O)C(C)C)cc1]

wherein [symbol] indicates an attachment site to a compound or salt of any one of Formulas (I), (Ia), (Ib), (Ic), (Id) and (Ie).

The linker can contain an enzymatically cleavable sugar moiety, for example, a linker comprising structural formula (Va), (Vb), (Vc), (Vd), or (Ve):

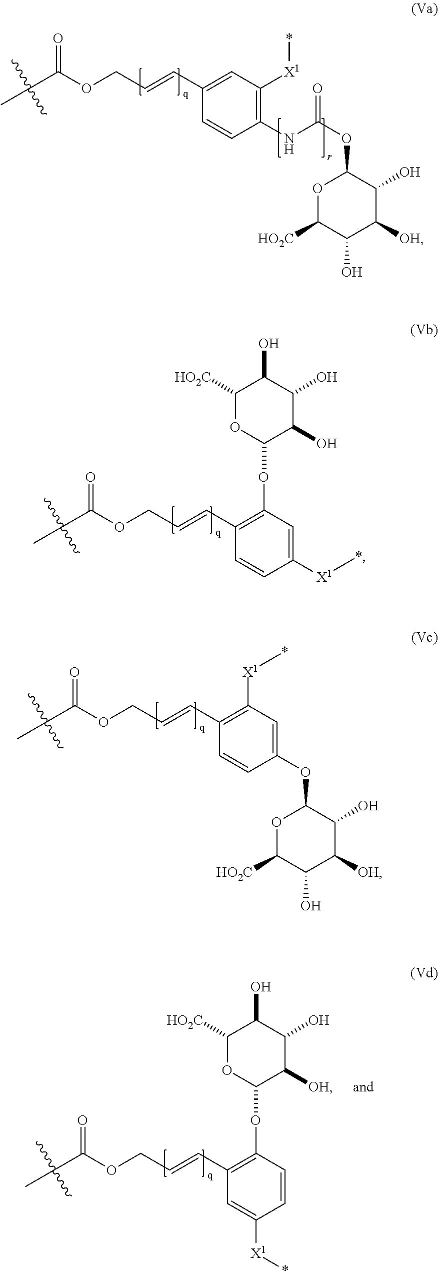

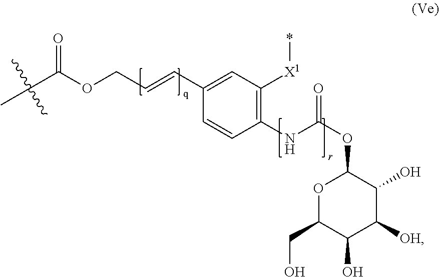

or a salt thereof, wherein: q is 0 or 1; r is 0 or 1; X¹ is CH₂, O or NH; [symbol] represents the point of attachment of the linker (L³) to the compound or salt of any one of Formulas (I), (Ia), (Ib), (Ic), (Id) and (Ie); and * represents the point of attachment to another portion of the linker or to a residue of an antibody construct.

Exemplary embodiments of linkers according to structural formula (Va) that may be included in the conjugates described herein include the linkers illustrated below (as illustrated, the linkers include a group suitable for covalently linking the linker to an antibody construct):

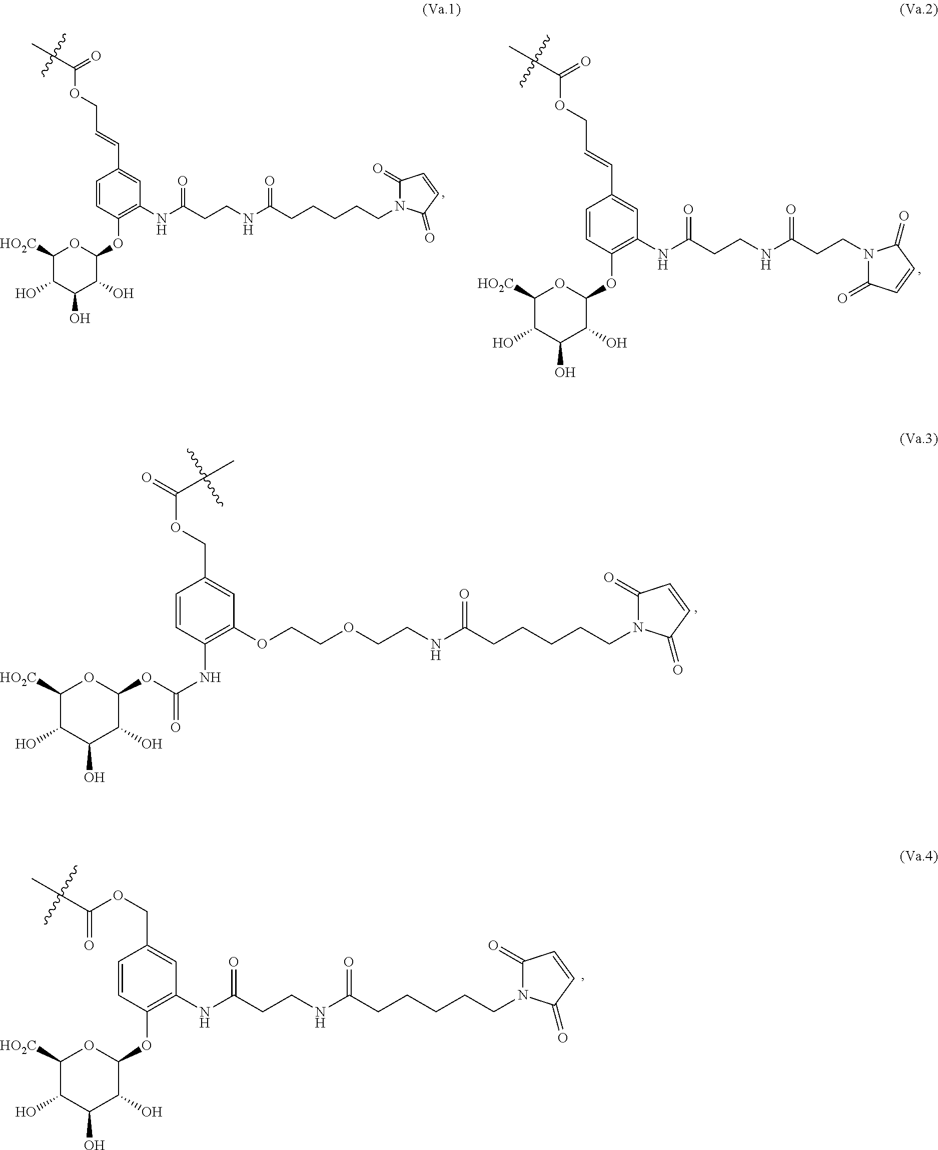

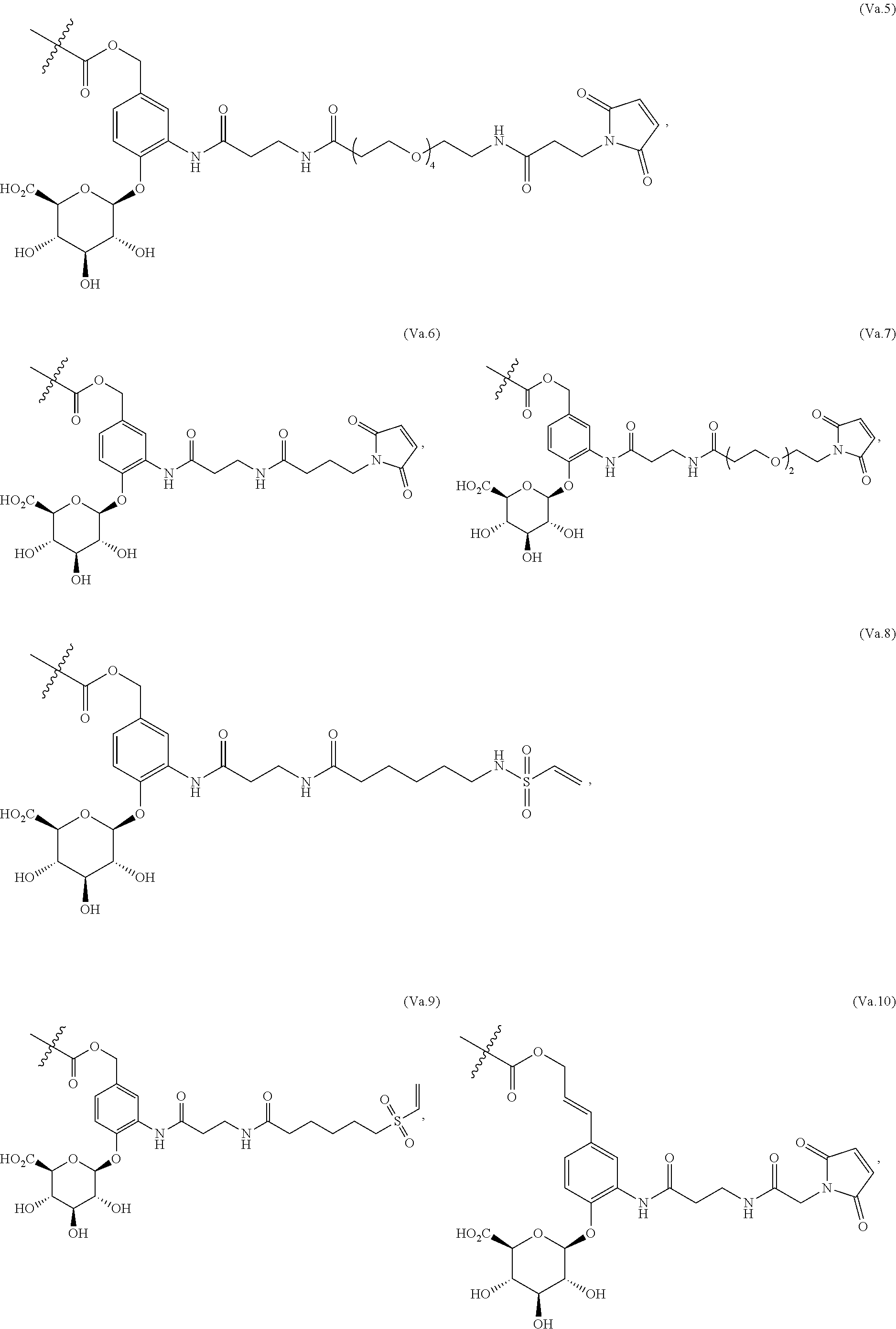

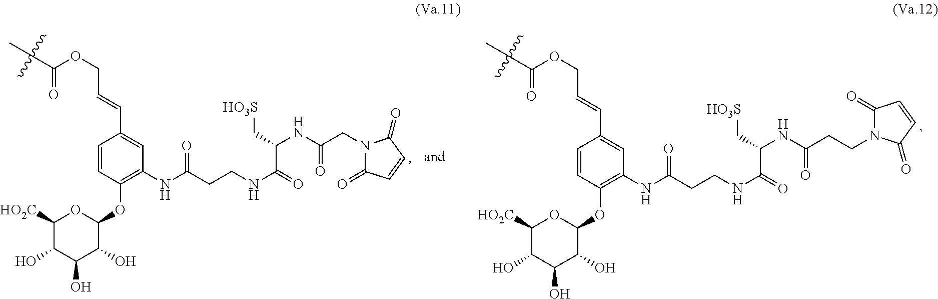

wherein [symbol] represents the point of attachment of the linker (L³) to the compound or salt of any one of Formulas (I), (Ia), (Ib), (Ic), (Id) and (Ie).

Exemplary embodiments of linkers according to structural formula (Vb) that may be included in the conjugates described herein include the linkers illustrated below (as illustrated, the linkers include a group suitable for covalently linking the linker to an antibody construct):

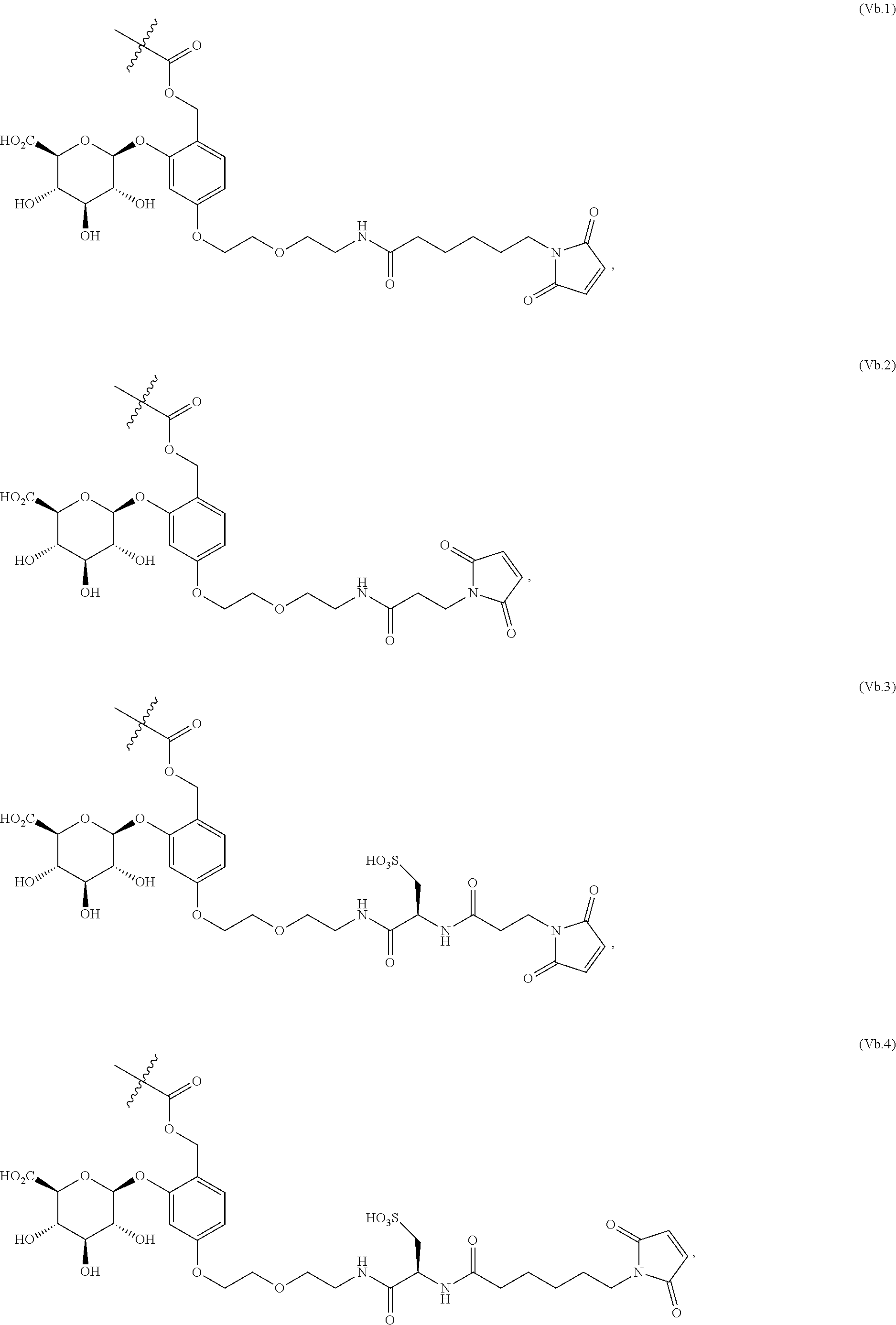

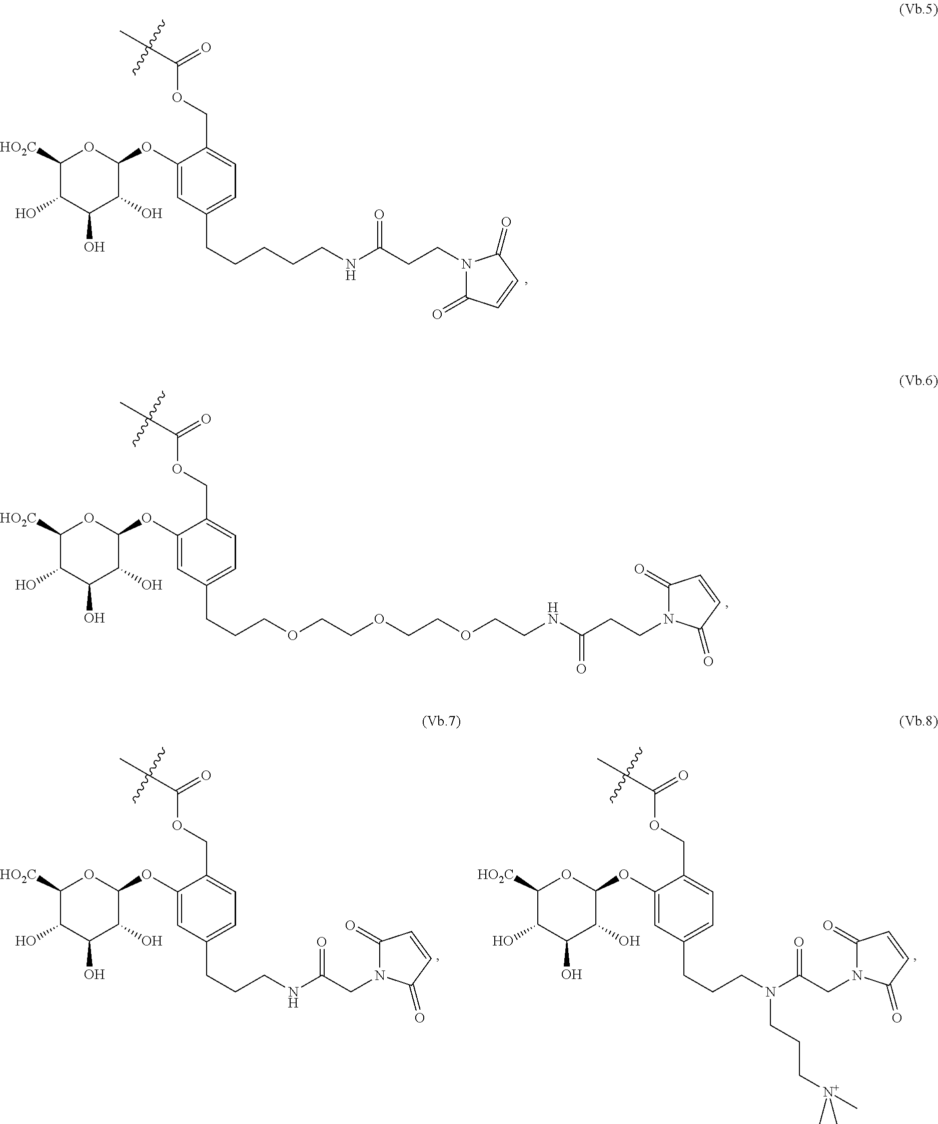

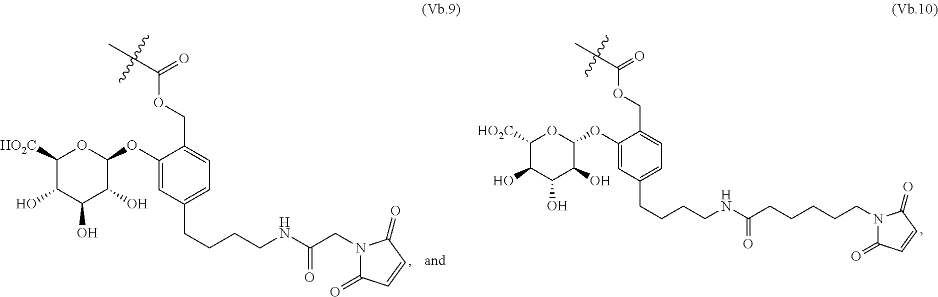

wherein [symbol] represents the point of attachment of the linker (L³) to the compound or salt of any one of Formulas (I), (Ia), (Ib), (Ic), (Id) and (Ie).

Exemplary embodiments of linkers according to structural formula (Vc) that may be included in the conjugates described herein include the linkers illustrated below (as illustrated, the linkers include a group suitable for covalently linking the linker to an antibody construct):

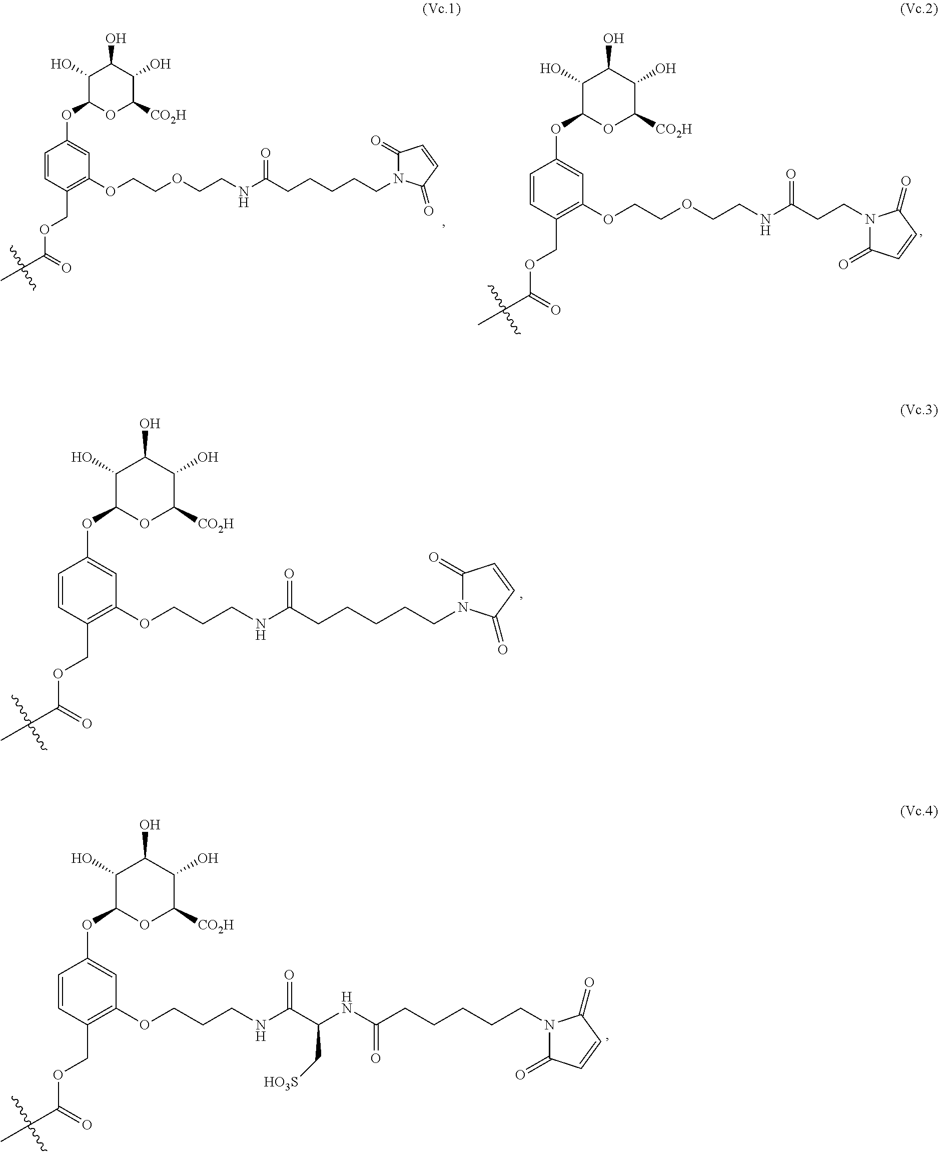

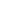

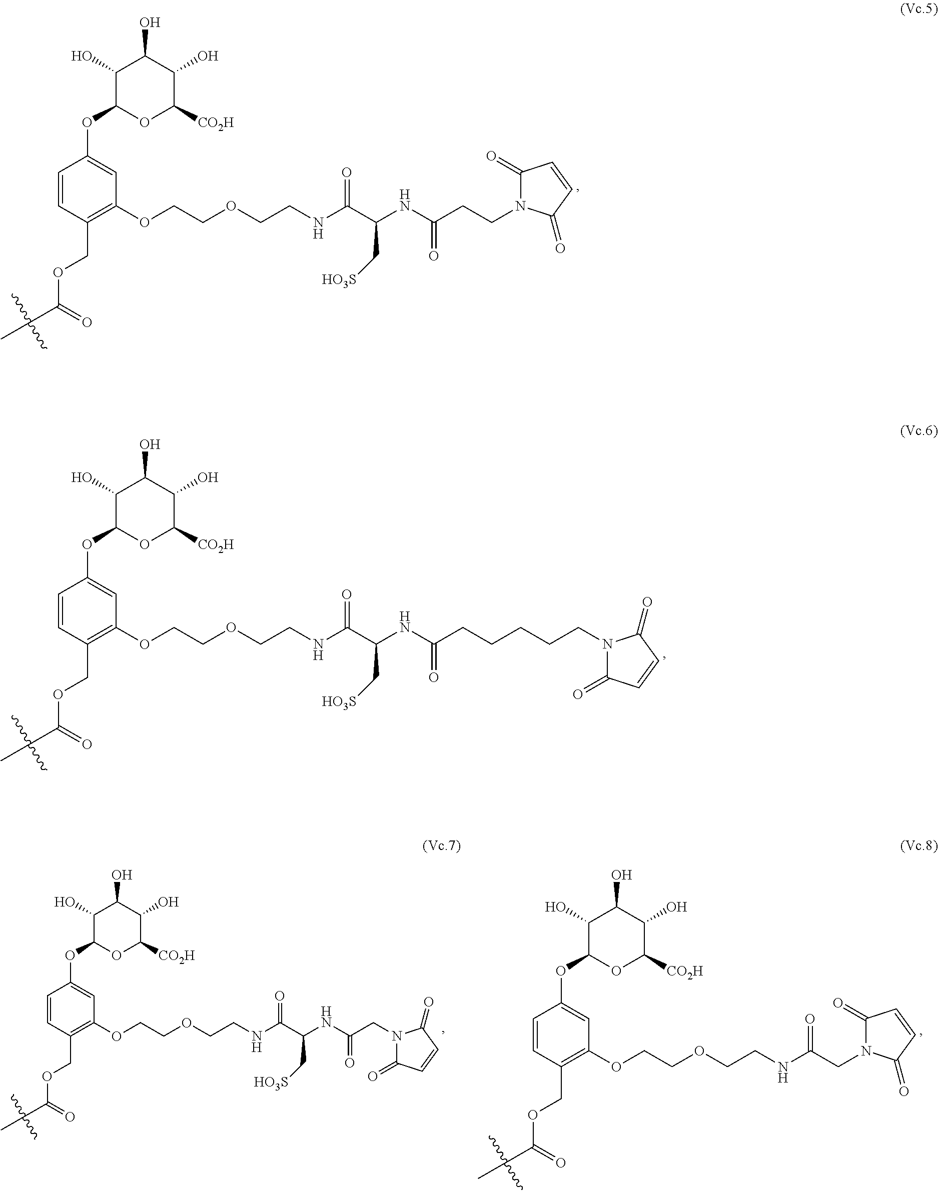

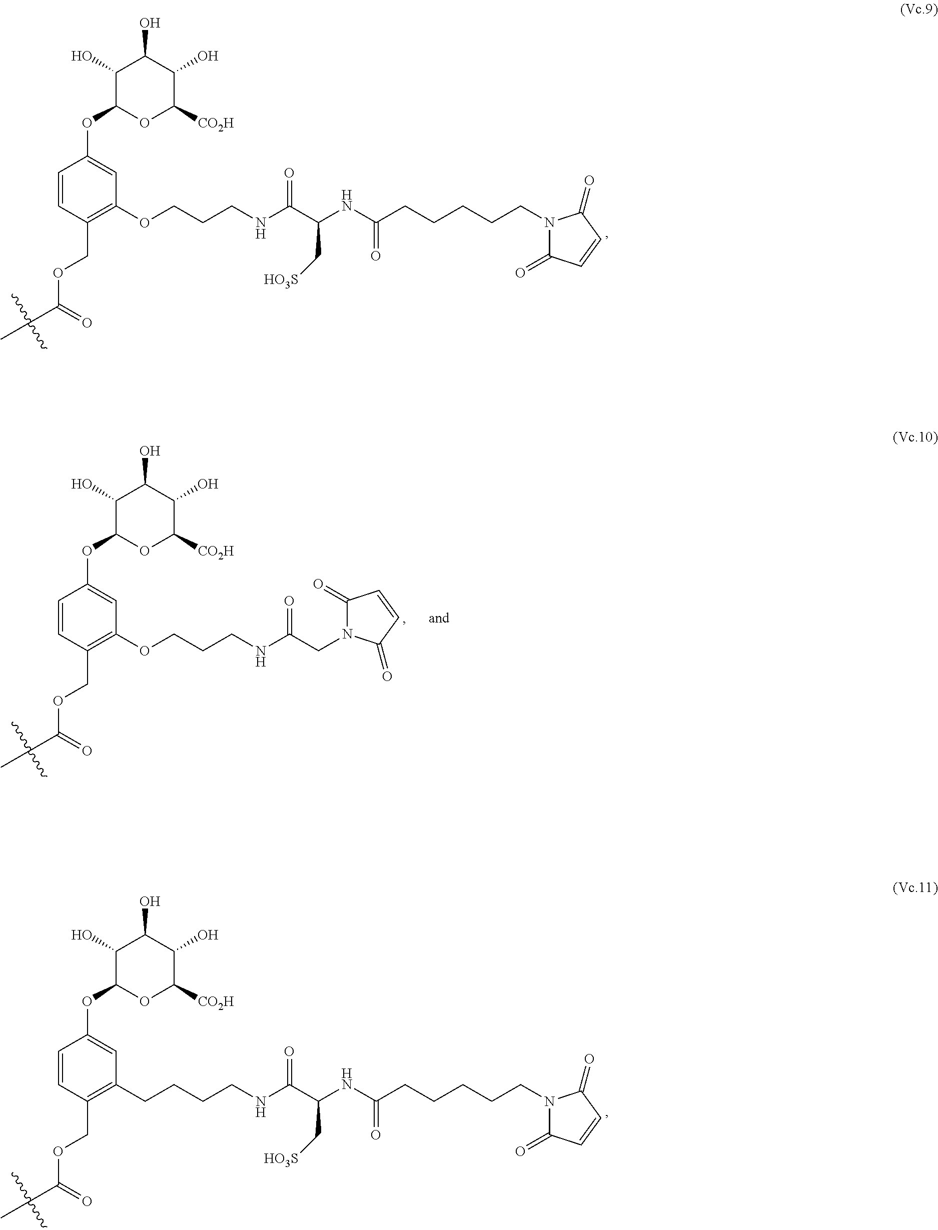

wherein [symbol] represents the point of attachment of the linker (L³) to the compound or salt of any one of Formulas (I), (Ia), (Ib), (Ic), (Id) and (Ie).

Exemplary embodiments of linkers according to structural formula (Vd) that may be included in the conjugates described herein include the linkers illustrated below (as illustrated, the linkers include a group suitable for covalently linking the linker to an antibody construct):

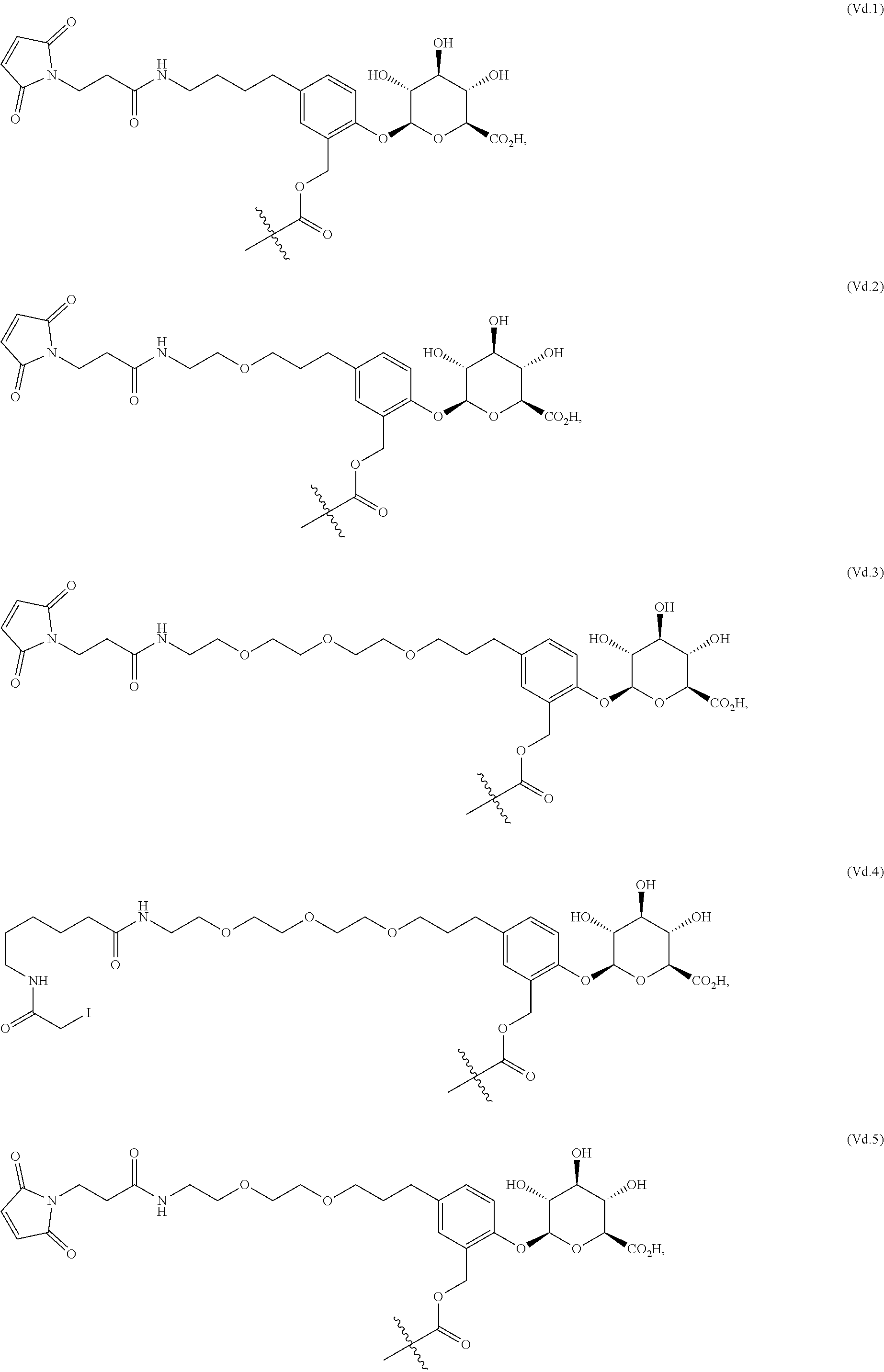

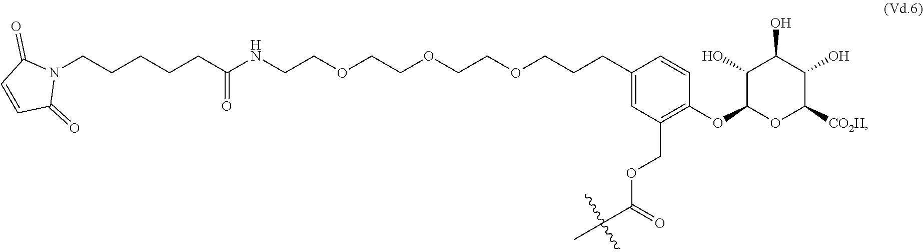

wherein [symbol] represents the point of attachment of the linker (L³) to the compound or salt of any one of Formulas (I), (Ia), (Ib), (Ic), (Id) and (Ie).

Exemplary embodiments of linkers according to structural formula (Ve) that may be included in the conjugates described herein include the linkers illustrated below (as illustrated, the linkers include a group suitable for covalently linking the linker to an antibody construct):

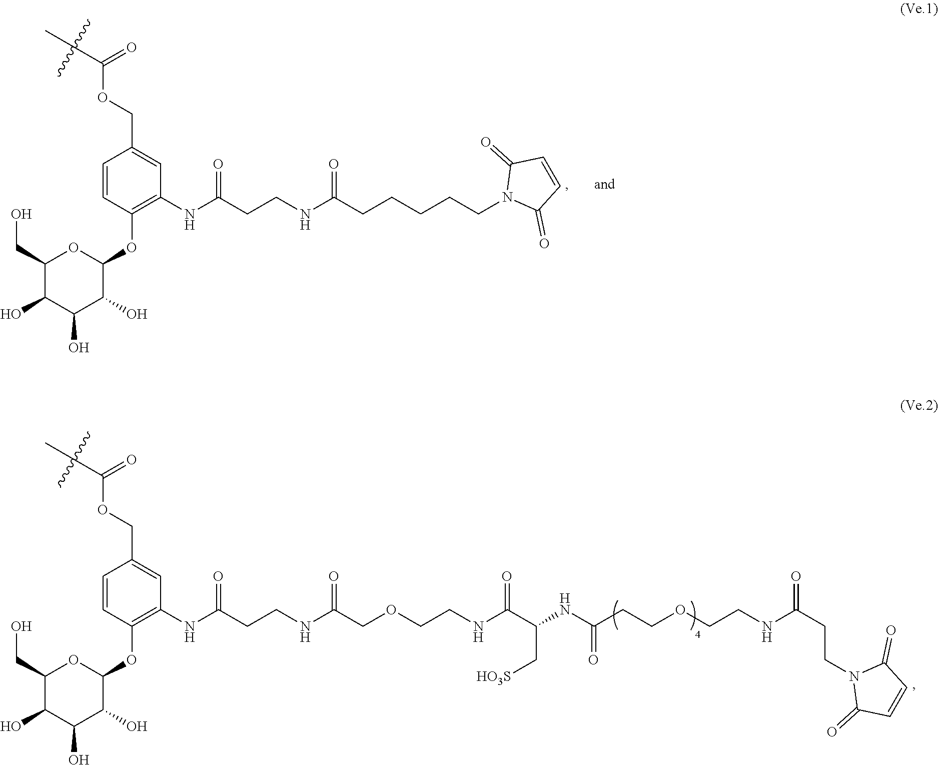

wherein [symbol] represents the point of attachment of the linker (L³) to the compound or salt of any one of Formulas (I), (Ia), (Ib), (Ic), (Id) and (Ie).

Although cleavable linkers can provide certain advantages, the linkers comprising the conjugate described herein need not be cleavable. For non-cleavable linkers, the compound (e.g., ALK5 inhibitor) release may not depend on the differential properties between the plasma and some cytoplasmic compartments. The release of the compound can occur after internalization of the conjugate via antigen-mediated endocytosis and delivery to lysosomal compartment, where the antibody construct can be degraded to the level of amino acids through intracellular proteolytic degradation. This process can release a compound derivative (a metabolite of the conjugate containing a non-cleavable linker-compound), which is formed by the compound (e.g., ALK5 inhibitor), the linker, and the amino acid residue or residues to which the linker was covalently attached. The compound derivative from conjugates with non-cleavable linkers can be more hydrophilic and less membrane permeable, which can lead to less bystander effects and less nonspecific toxicities compared to conjugates with a cleavable linker. Conjugates with non-cleavable linkers can have greater stability in circulation than conjugates with cleavable linkers. Non-cleavable linkers can include alkylene chains, or can be polymeric, such as, for example, based upon polyalkylene glycol polymers, amide polymers, or can include segments of alkylene chains, polyalkylene glycols and/or amide polymers. The linker can contain a polyethylene glycol segment having from 1 to 6 ethylene glycol units.

The linker can be non-cleavable in vivo, for example, a linker according to the formulations below:

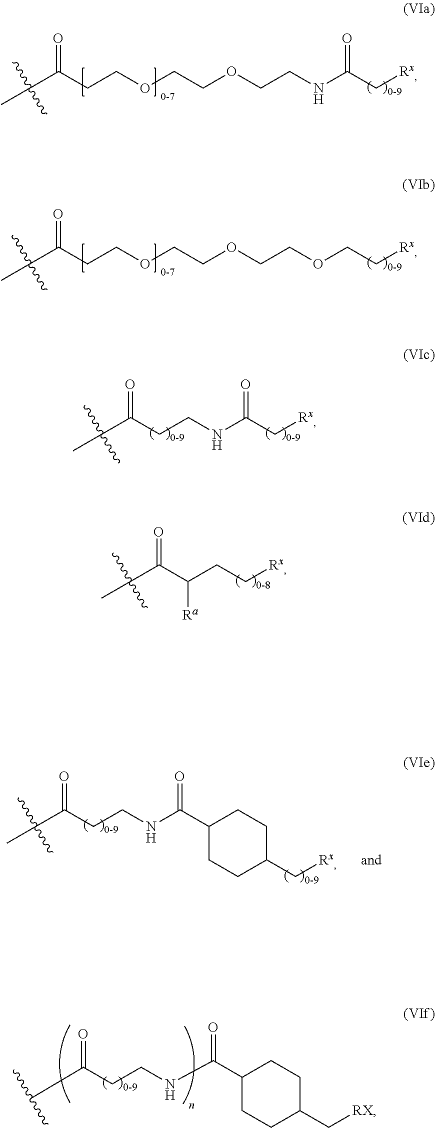

or salts thereof, in which: R^{a} is selected from hydrogen, alkyl, sulfonate and methyl sulfonate; R^{x} is a reactive moiety including a functional group capable of covalently linking the linker to an antibody construct; and [symbol] represents the point of attachment of the linker (L³) to the compound or salt of any one of Formulas (I), (Ia), (Ib), (Ic), (Id) and (Ie).

Exemplary embodiments of linkers according to structural formula (VIa)-(VIf) that may be included in the conjugates described herein include the linkers illustrated below (as illustrated, the linkers include a group suitable for covalently linking the linker to an antibody construct, and [symbol] represents the point of attachment of the linker (L³) to the compound or salt of any one of Formulas (I), (Ia), (Ib), (Ic), (Id) and (Ie):

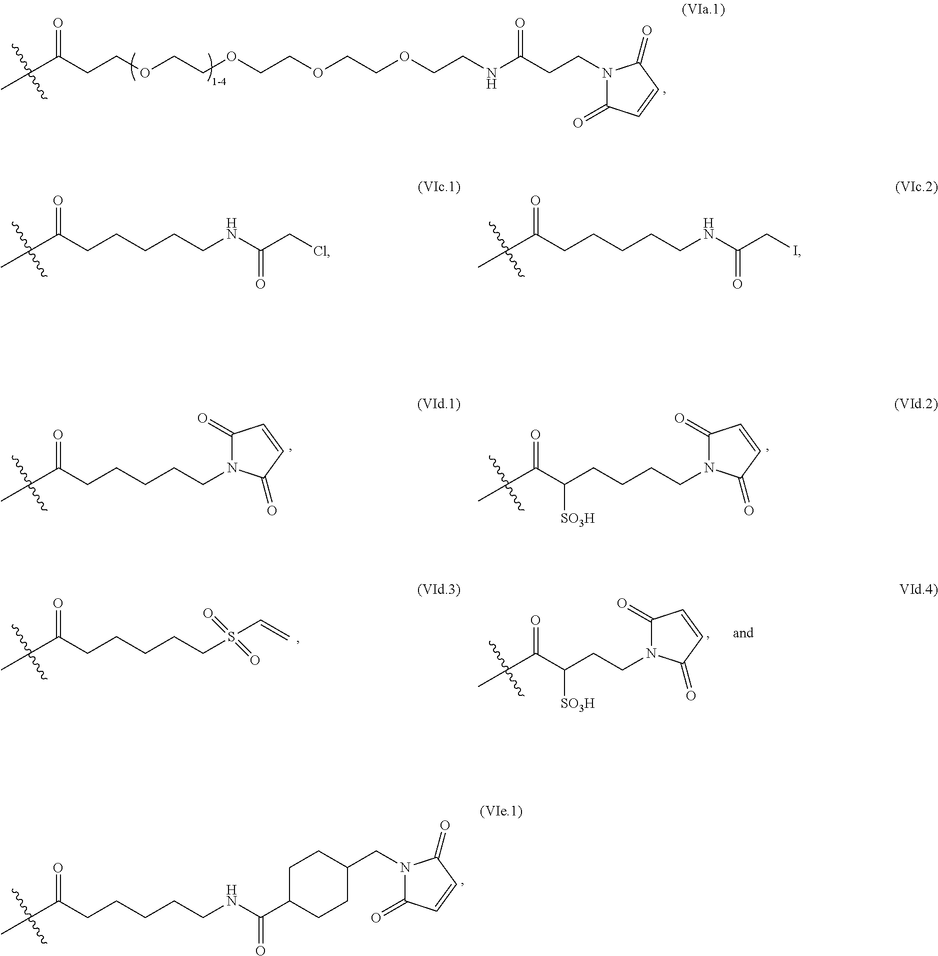

wherein [symbol] represents the point of attachment of the linker (L³) to the compound or salt of any one of Formulas (I), (Ia), (Ib), (Ic), (Id) and (Ie).

Attachment groups that are used to attach the linkers to an antibody construct can be electrophilic in nature and include, for example, maleimide groups, alkynes, alkynoates, allenes and allenoates, activated disulfides, active esters such as NHS esters and HOBt esters, haloformates, acid halides, alkyl, and benzyl halides such as haloacetamides. There are also emerging technologies related to “self-stabilizing” maleimides and “bridging disulfides” that can be used in accordance with the disclosure.

Maleimide groups are frequently used in the preparation of conjugates because of their specificity for reacting with thiol groups of, for example, cysteine groups of the antibody residue of a conjugate. The reaction between a thiol group of an antibody residue and a drug with a linker including a maleimide group proceeds according to the following scheme:

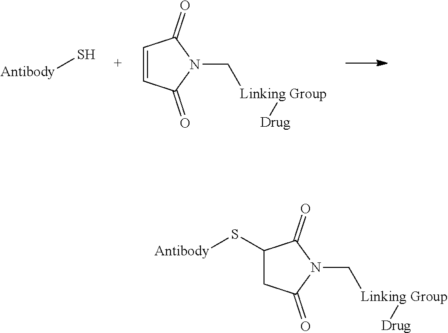

The reverse reaction leading to maleimide elimination from a thio-substituted succinimide may also take place. This reverse reaction is undesirable as the maleimide group may subsequently react with another available thiol group such as other proteins in the body having available cysteines. Accordingly, the reverse reaction can undermine the specificity of a conjugate. One method of preventing the reverse reaction is to incorporate a basic group into the linker shown in the scheme above. Without wishing to be bound by theory, the presence of the basic group may increase the nucleophilicity of nearby water molecules to promote ring-opening hydrolysis of the succinimide group. The hydrolyzed form of the attachment group is resistant to deconjugation in the presence of plasma proteins. So-called “self-stabilizing” linkers provide conjugates with improved stability. A representative schematic is shown below:

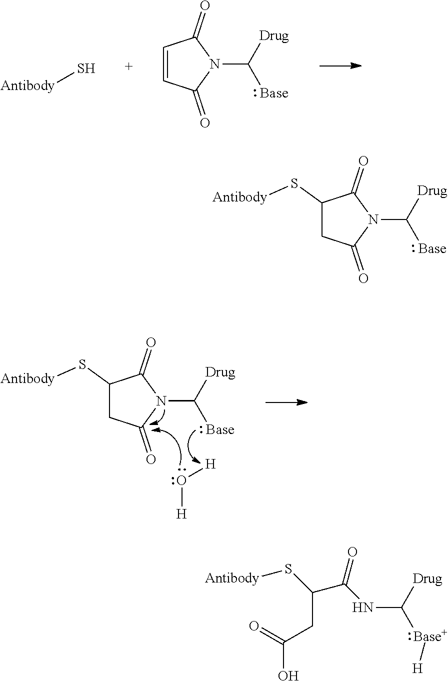

The hydrolysis reaction schematically represented above may occur at either carbonyl group of the succinimide group. Accordingly, two possible isomers may result, as shown below:

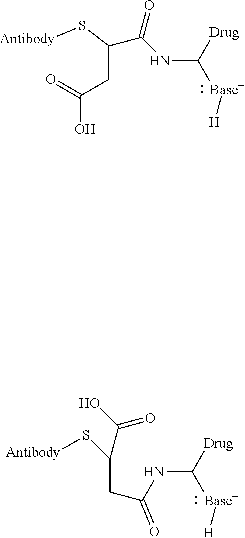

The identity of the base as well as the distance between the base and the maleimide group can be modified to tune the rate of hydrolysis of the thio-substituted succinimide group and optimize the delivery of a conjugate to a target by, for example, improving the specificity and stability of the conjugate.

Bases suitable for inclusion in a linker L³ described herein with a maleimide group prior to conjugation to an antibody construct may facilitate hydrolysis of a nearby succinimide group formed after conjugation of the antibody construct to the linker. Bases may include, for example, amines (e.g., —N(R²⁶)(R²⁷), where R²⁶ and R²⁷ are independently selected from H and C₁₋₆ alkyl), nitrogen-containing heterocycles (e.g., a 3- to 12-membered heterocycle including one or more nitrogen atoms and optionally one or more double bonds), amidines, guanidines, and carbocycles or heterocycles substituted with one or more amine groups (e.g., a 3- to 12-membered aromatic or non-aromatic cycle optionally including a heteroatom such as a nitrogen atom and substituted with one or more amines of the type —N(R²⁶)(R²⁷), where R²⁶ and R²⁷ are independently selected from H or C₁₋₆ alkyl). A basic unit may be separated from a maleimide group by, for example, an alkylene chain of the form —(CH₂)ₛ—, where s is an integer from 0 to 10. An alkylene chain may be optionally substituted with other functional groups as described herein.

A linker L³ described herein with a maleimide group may include an electron withdrawing group such as, but not limited to, —C(O)R, ═O, —CN, —NO₂, —CX₃, —X, —COOR, —CONR₂, —COR, —COX, —SO₂R, —SO₂OR, —SO₂NHR, —SO₂NR₂, —PO₃R₂, —P(O)(CH₃)NHR, —NO , —NR₃⁺, —CR═CR₂, and —C≡CR, where each R is independently selected from H and C₁₋₆ alkyl and each X is independently selected from F, Br, Cl, and I. Self-stabilizing linkers may also include aryl, e.g., phenyl, or heteroaryl, e.g., pyridine, groups optionally substituted with electron withdrawing groups such as those described herein.

Examples of self-stabilizing linkers are provided in, e.g., U.S. Patent Publication Number 2013/0309256, the linkers of which are incorporated by reference herein. It will be understood that a self-stabilizing linker useful in conjunction with the compounds of the present invention may be equivalently described as unsubstituted maleimide-including linkers, thio-substituted succinimide-including linkers, or hydrolyzed, ring-opened thio-substituted succinimide-including linkers.

In certain embodiments, L³ comprises a stabilizing linker moiety selected from:

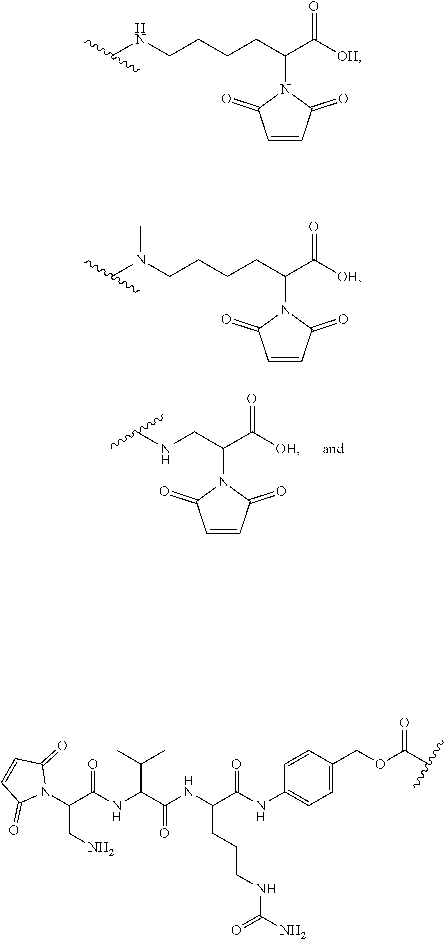

In the scheme provided above, the bottom structure may be referred to as (maleimido)-DPR-Val-Cit-PAB, where DPR refers to diaminopropinoic acid, Val refers to valine, Cit refers to citrulline, and PAB refers to para-aminobenzylcarbonyl. [symbol] represents the point of attachment to compound or salt of any one of Formulas (I), (Ia), (Ib), (Ic), (Id) and (Ie).

A method for bridging a pair of sulfhydryl groups derived from reduction of a native hinge disulfide bond has been disclosed and is depicted in the schematic below. An advantage of this methodology is the ability to synthesize homogenous DAR4 conjugates by full reduction of IgGs (to give 4 pairs of sulfhydryls from interchain disulfides) followed by reaction with 4 equivalents of the alkylating agent. Conjugates containing “bridged disulfides” are also claimed to have increased stability.

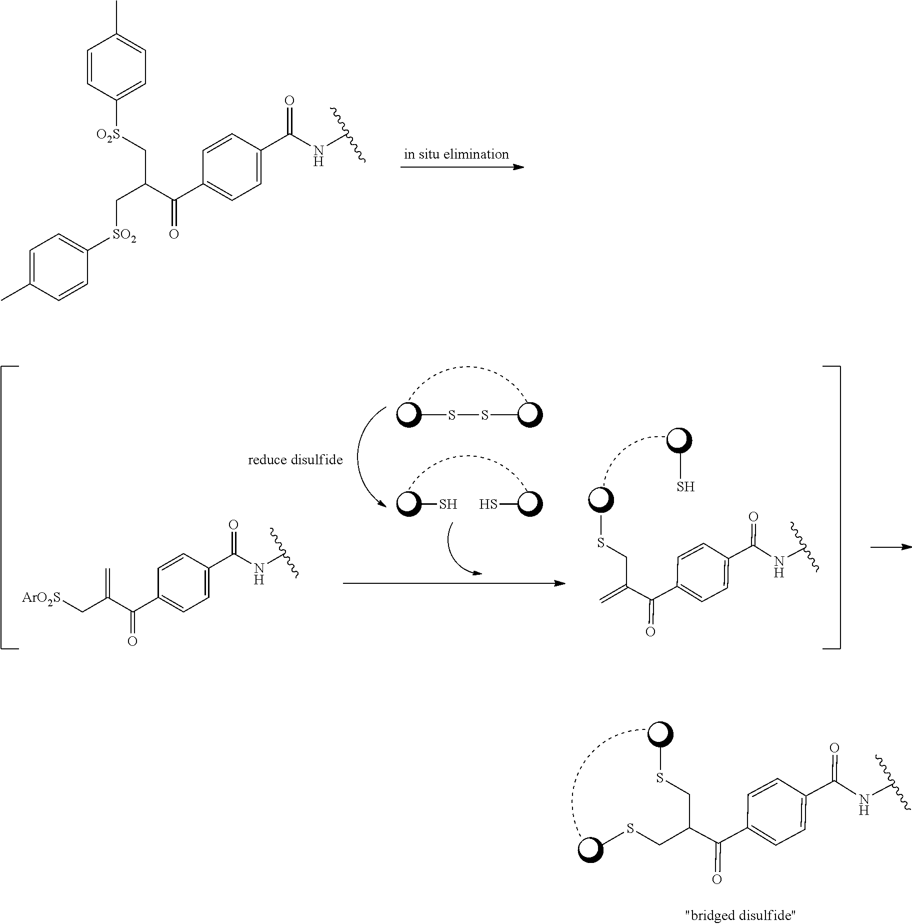

Similarly, as depicted below, a maleimide derivative that is capable of bridging a pair of sulfhydryl groups has been developed.

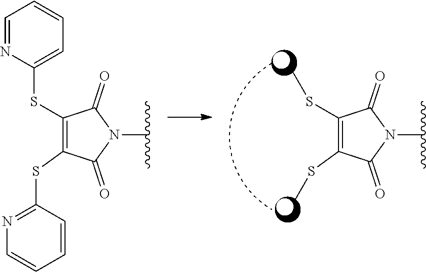

A linker L³ can contain the following structural formulas (VIIa), (VIIb), or (VIIc):

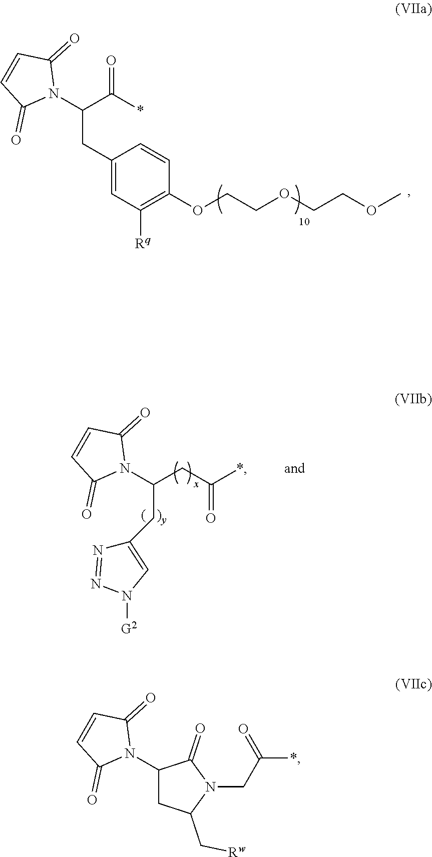

or salts thereof, wherein: R^{q} is H or —O—(CH₂CH₂O)₁₁—CH₃; x is 0 or 1; y is 0 or 1; G² is —CH₂CH₂CH₂SO₃H or —CH₂CH₂O—(CH₂CH₂O)₁₁—CH₃; R^{w} is —O—CH₂CH₂SO₃H or —NH(CO)—CH₂CH₂O—(CH₂CH₂O)₁₂—CH₃; and * represents the point of attachment to another portion of the linker or a site of attachment to a compound or salt of any one of Formulas (I), (Ia), (Ib), (Ic), (Id) and (Ie). Exemplary embodiments of linkers according to structural formula (VIIa) and (VIIb) that can be included in the conjugates described herein can include the linkers illustrated below (as illustrated, the linkers can include a group suitable for covalently linking the linker to a residue of an antibody construct):

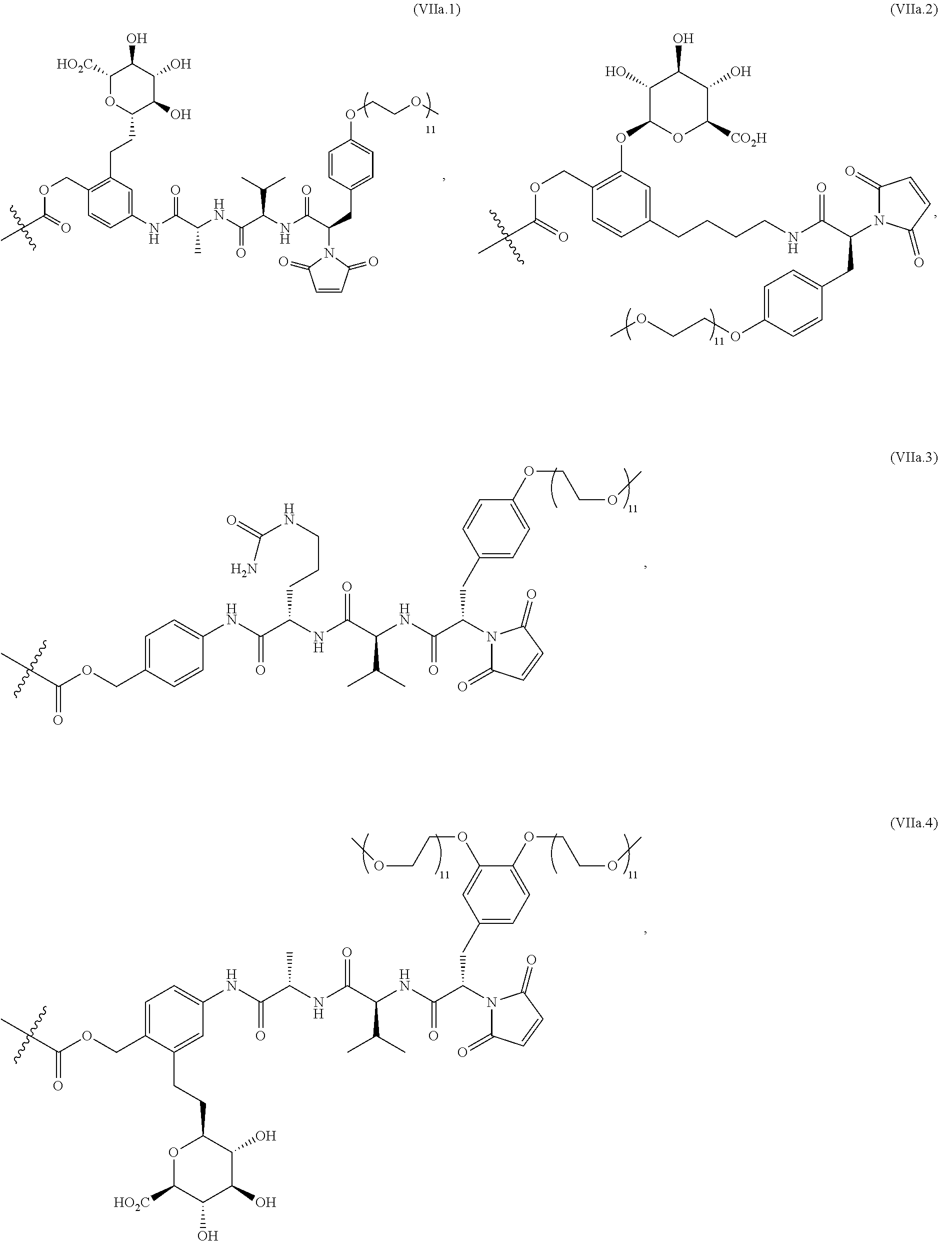

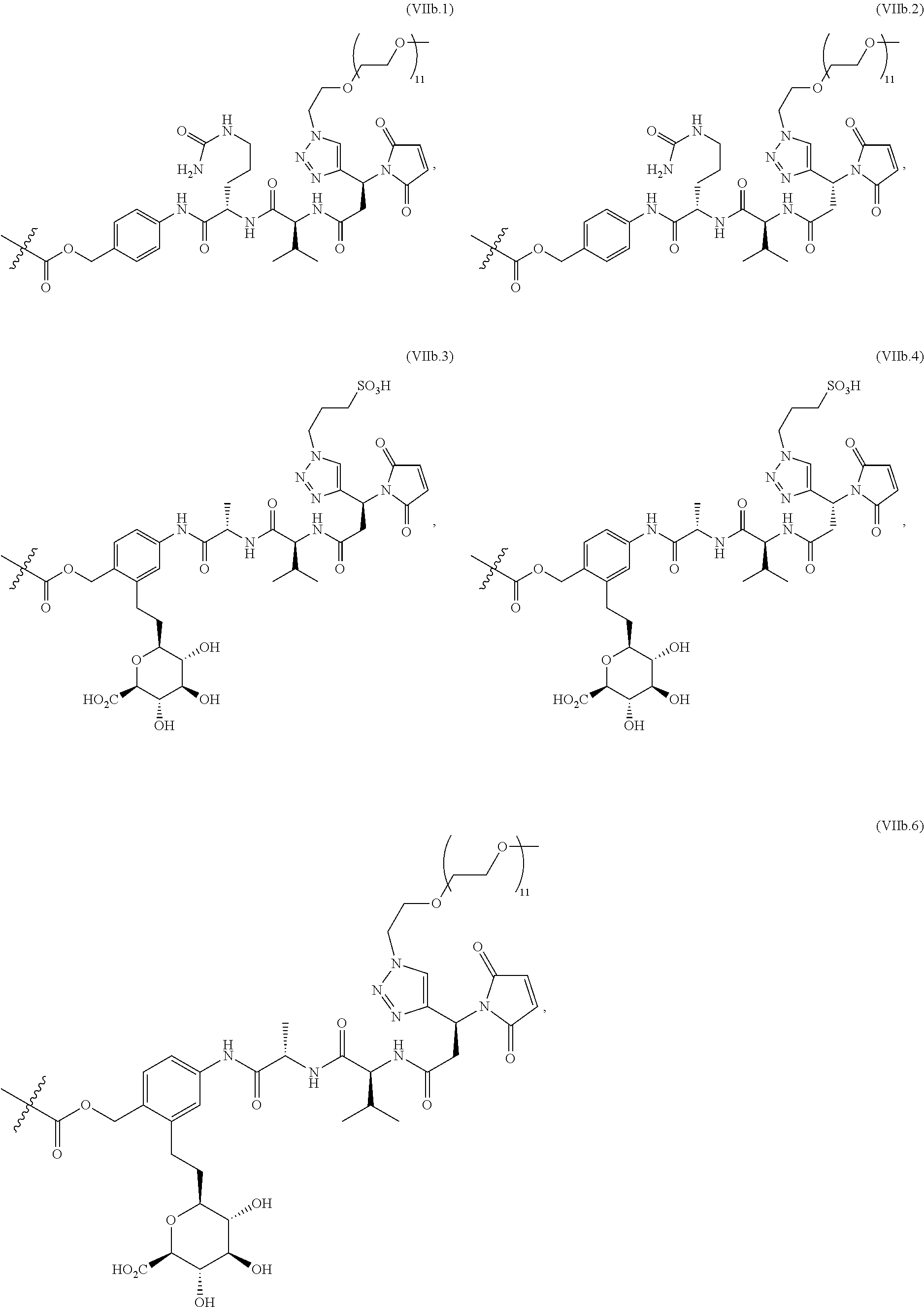

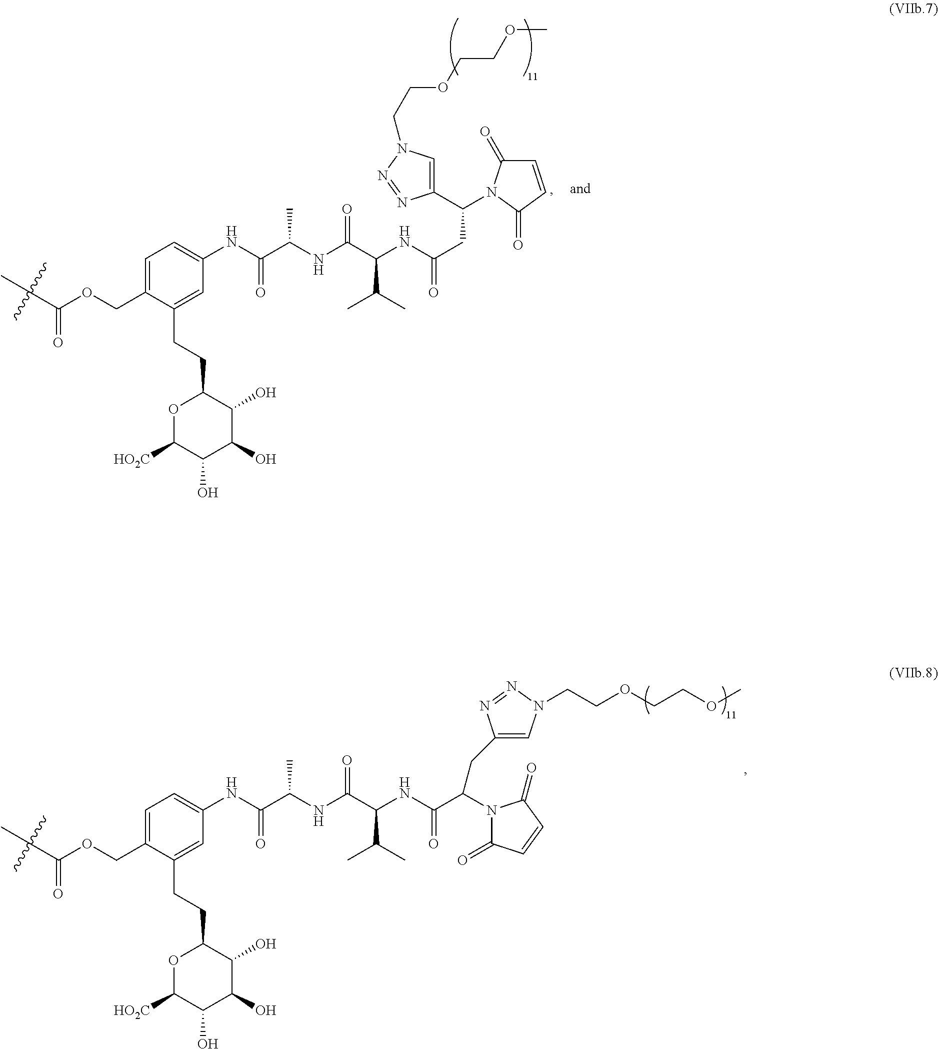

wherein [symbol] represents the point of attachment of the linker (L³) to the compound or salt of any one of Formulas (I), (Ia), (Ib), (Ic), (Id) and (Ie).

Exemplary embodiments of linkers according to structural formula (VIIc) that can be included in the conjugates described herein can include the linkers illustrated below (as illustrated, the linkers include a group suitable for covalently linking the linker to an antibody construct):

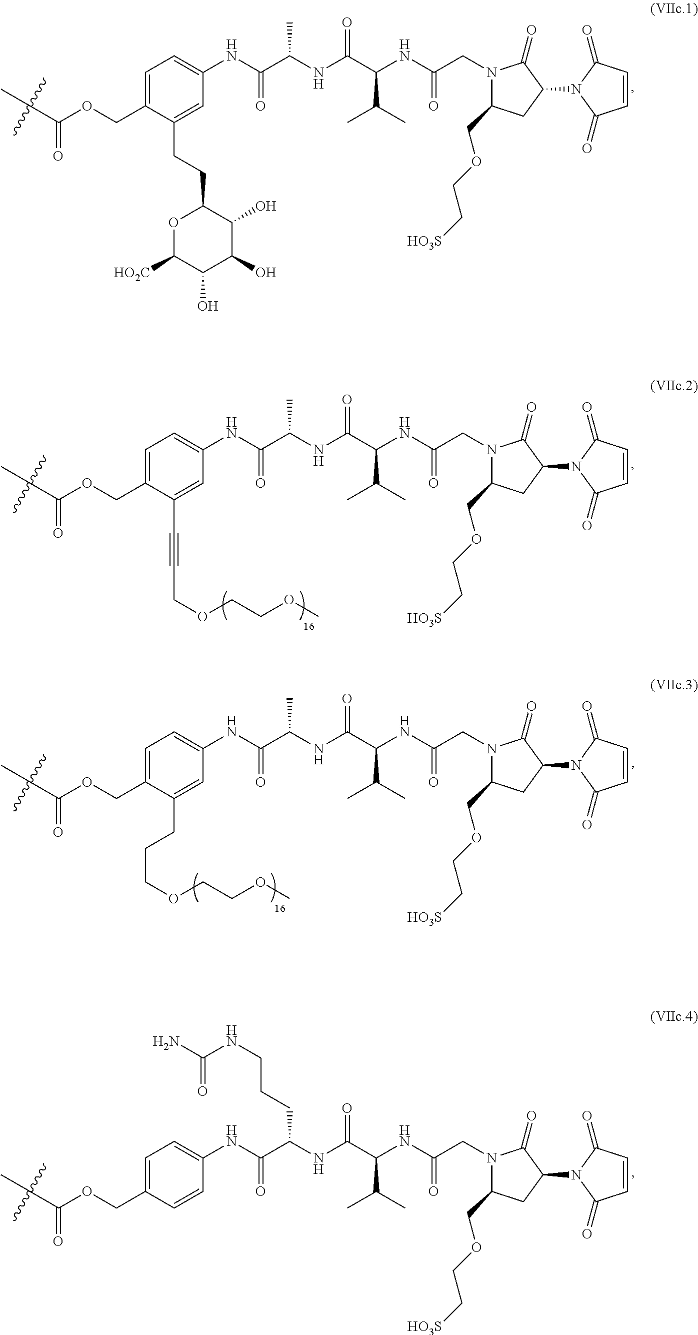

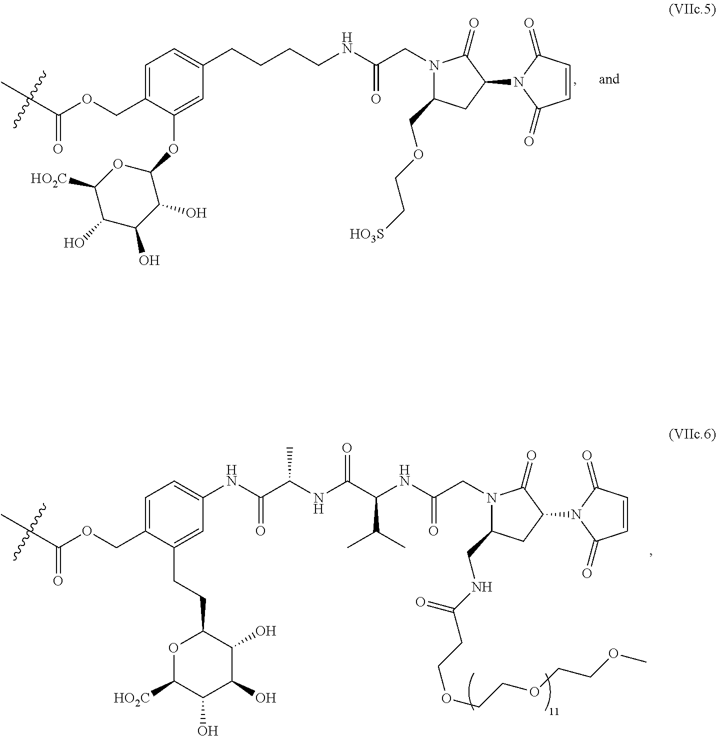

wherein [symbol] represents the point of attachment of the linker (L³) to the compound or salt of any one of Formulas (I), (Ia), (Ib), (Ic), (Id) and (Ie).

In some embodiments, a compound bound to a linker L³ is represented by:

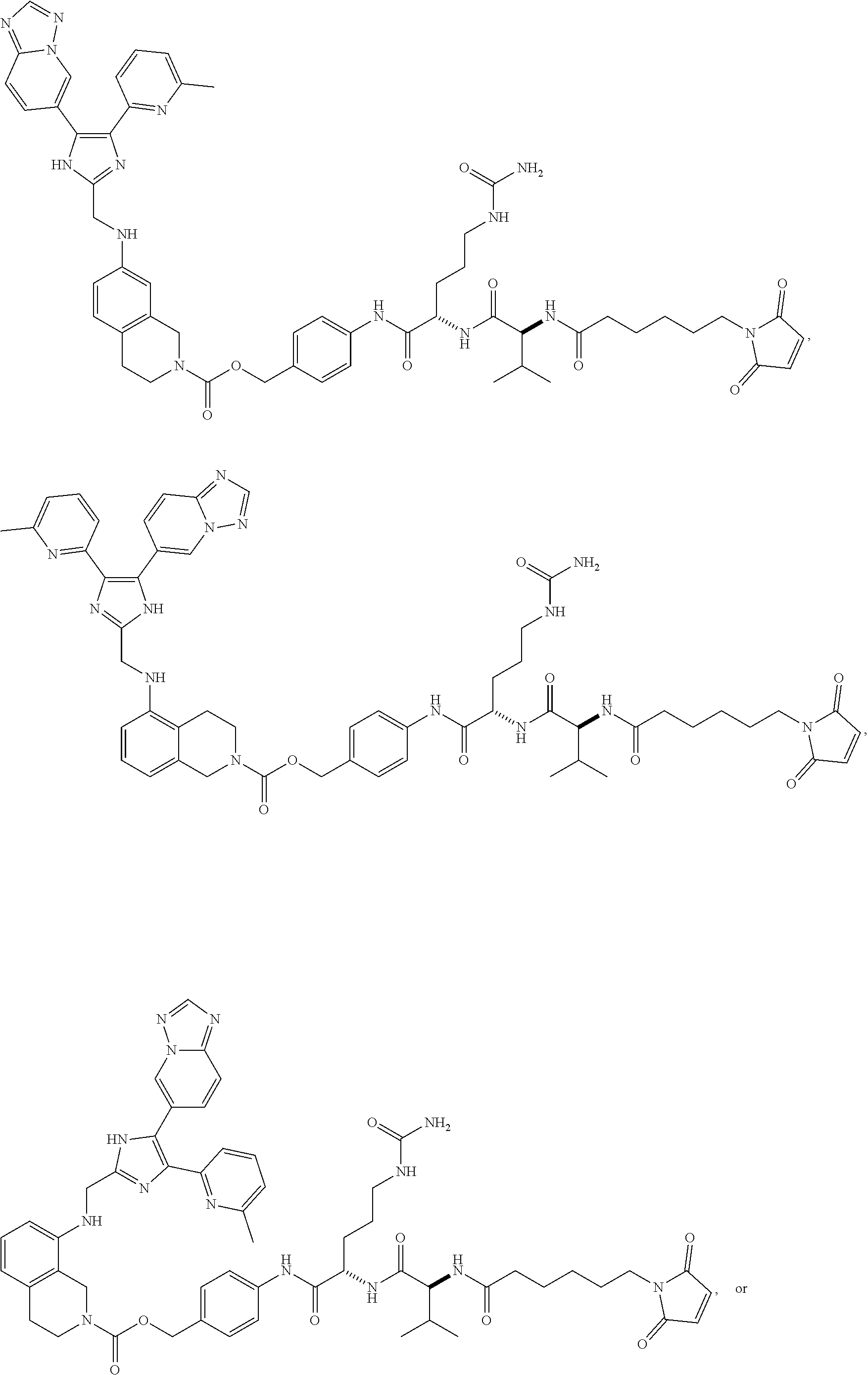 [SMILES: Cc1cccc(-c2nc(CNc3ccc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)[nH]c2-c2ccc3ncnn3c2)n1 ; Cc1cccc(-c2nc(CNc3cccc4c3CCN(C(=O)OCc3ccc(NC(=O)[C@H](CCCNC(N)=O)NC(=O)[C@@H](NC(=O)CCCCCN5C(=O)C=CC5=O)C(C)C)cc3)C4)[nH]c2-c2ccc3ncnn3c2)n1 ; Cc1cccc(-c2nc(CNc3cccc4c3CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)[nH]c2-c2ccc3ncnn3c2)n1]

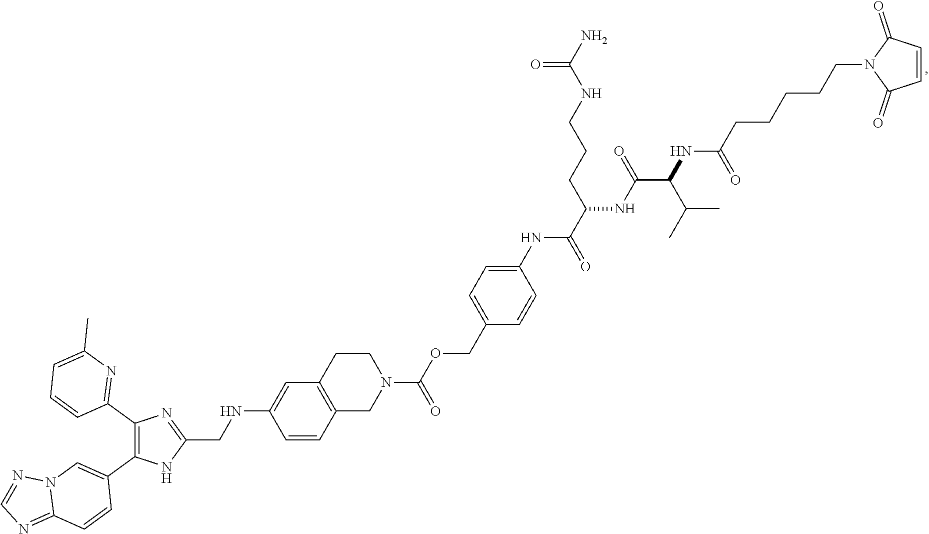 [SMILES: Cc1cccc(-c2nc(CNc3ccc4c(c3)CCN(C(=O)OCc3ccc(NC(=O)[C@H](CCCNC(N)=O)NC(=O)[C@@H](NC(=O)CCCCCN5C(=O)C=CC5=O)C(C)C)cc3)C4)[nH]c2-c2ccc3ncnn3c2)n1]

As is known by skilled artisans, the linker selected for a particular conjugate may be influenced by a variety of factors including, but not limited to, the site of attachment to the residue of an antibody construct (e.g., Lys, Cys or other amino acid residues), structural constraints of the drug pharmacophore (e.g., compound that may be an ALK5 inhibitor) and the lipophilicity of the drug. The specific linker selected for a conjugate should seek to balance these different factors for the specific antibody construct-ALK5 inhibitor combination.

For example, ALK5 inhibitors and conjugates including the same may effect bystander action on antigen-negative cells present in the vicinity of the antigen-positive target cells. The mechanism of bystander activity by conjugates has indicated that metabolic products formed during intracellular processing of the conjugates may play a role. Neutral and hydrophobic metabolites generated by metabolism of the conjugates in antigen-positive cells appear to play a role in bystander activity while charged and hydrophilic metabolites may be prevented from diffusing across the membrane into the medium, or from the medium across the membrane, and therefore cannot affect bystander activity. In certain embodiments, the linker is selected to attenuate the bystander activity caused by cellular metabolites of the conjugate. In certain embodiments, the linker is selected to increase the bystander effect.

The properties of the linker, or linker-compound, may also impact aggregation of the conjugate under conditions of use and/or storage. Typically, conjugates reported in the literature contain no more than 3-4 drug molecules per antibody molecule. Attempts to obtain higher drug-to-antibody ratios (“DAR”) often failed, particularly if both the drug and the linker were hydrophobic, due to aggregation of the conjugate. In many instances, DARs higher than 3-4 could be beneficial as a means of increasing potency. In instances where the compound (e.g., ALK5 inhibitor) is more hydrophobic in nature, it may be desirable to select linkers that are relatively hydrophilic as a means of reducing conjugate aggregation, especially in instances where DARs greater than 3-4 are desired. Thus, in certain embodiments, the linker incorporates chemical moieties that reduce aggregation of the conjugates during storage and/or use. A linker may incorporate polar or hydrophilic groups such as charged groups or groups that become charged under physiological pH to reduce the aggregation of the conjugates. For example, a linker may incorporate charged groups such as salts or groups that deprotonate, e.g., carboxylates, or protonate, e.g., amines, at physiological pH.

In some embodiments, the aggregation of the conjugates during storage or use is less than about 40% as determined by size-exclusion chromatography (SEC). In particular embodiments, the aggregation of the conjugates during storage or use is less than 35%, such as less than about 30%, such as less than about 25%, such as less than about 20%, such as less than about 15%, such as less than about 10%, such as less than about 5%, such as less than about 4%, or even less, as determined by size-exclusion chromatography (SEC).

### Attachment of Linkers to Antibody Construct

The conjugates described herein may comprise a linker, e.g., a peptide containing linker. Linkers of the conjugates and methods described herein may not affect the binding of active portions of a conjugate (e.g., active portions include antigen binding domains, Fc domains, target binding domains, antibodies, compounds or salts disclosed herein [e.g., Formulas (I), (Ia), (Ib), (Ic), (Id) and (Ie)]), to a target, which can be a cognate binding partner such as an antigen. A linker can form a linkage between different parts of a conjugate, e.g., between an antibody construct and a compound or salt of the disclosure. In certain embodiments, a conjugate comprises multiple linkers. In certain embodiments, wherein a conjugate comprises multiple linkers, the linkers may be the same linkers or different linkers.

A linker may be bound to an antibody construct by a bond between the antibody construct and the linker. A linker may be bound to an anti-tumor antigen antibody construct by a bond between the anti-tumor antigen antibody construct and the linker. A linker may be bound to a terminus of an amino acid sequence of an antibody construct, or could be bound to a side chain modification to the antibody construct, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc region of an antibody construct, or may be bound to a side chain modification of an Fc region of an antibody construct, such as the side chain of a lysine, serine, threonine, cysteine, tyrosine, aspartic acid, glutamine, a non-natural amino acid residue, or glutamic acid residue. A linker may be bound to a terminus of an amino acid sequence of an Fc domain of an antibody construct, or may be bound to a side chain modification of an Fc domain of an antibody construct, such as the side chain of a lysine, serine, threonine, cysteine, tyrosine, aspartic acid, glutamine, a non-natural amino acid residue, or glutamic acid residue.

A linker may be bound to an antibody construct at a hinge cysteine. A linker may be bound to an antibody construct at a light chain constant domain lysine. A linker may be bound to an antibody construct at a heavy chain constant domain lysine. A linker may be bound to an antibody construct at an engineered cysteine in the light chain. A linker may be bound to an antibody construct at an Fc region lysine. A linker may be bound to an antibody construct at an Fc domain lysine. A linker may be bound to an antibody construct at an Fc region cysteine. A linker may be bound to an antibody construct at an Fc domain cysteine. A linker may be bound to an antibody construct at a light chain glutamine, such as an engineered glutamine. A linker may be bound to an antibody construct at a heavy chain glutamine, such as an engineered glutamine. A linker may be bound to an antibody construct at an unnatural amino acid engineered into the light chain. A linker may be bound to an antibody construct at an unnatural amino acid engineered into the heavy chain. Amino acids can be engineered into an amino acid sequence of an antibody construct as described herein, for example, a linker of a conjugate. Engineered amino acids may be added to a sequence of existing amino acids. Engineered amino acids may be substituted for one or more existing amino acids of a sequence of amino acids.

A linker may be conjugated to an antibody construct via a sulfhydryl group on the antibody construct. A linker may be conjugated to an antibody construct via a primary amine on the antibody construct. A linker may be conjugated to an antibody construct via a residue of an unnatural amino acid on an antibody construct, e.g., a ketone moiety.

When one or more linkers are bound to an antibody construct at the sites described herein, an Fc domain of the antibody construct can bind to Fc receptors. In certain embodiments, an antibody construct bound through a linker or an antibody construct bound to a linker bound to a compound or salt of any one of Formulas (I), (Ia), (Ib), (Ic), (Id) and (Ie), retains the ability of the Fc domain of the antibody to bind to Fc receptors. In certain embodiments, when a linker is connected to an antibody construct at the sites described herein, the antigen binding domain of an antibody construct bound to a linker or an antibody construct bound through a linker bound to a compound or salt of any one of Formulas (I), (Ia), (Ib), (Ic), (Id) and (Ie)can bind its antigen. In certain embodiments, when a linker is connected to an antibody construct at the sites described herein, a target binding domain of an antibody construct bound to a linker or an antibody construct bound to a linker bound to a compound or salt of any one of Formulas (I), (Ia), (Ib), (Ic), (Id) and (Ie) can bind its antigen.

In certain embodiments, a compound or a compound-linker of any one of Formulas (I), (Ia), (Ib), (Ic), (Id) and (Ie) disclosed herein is attached to an antibody Fc region or domain at an engineered cysteine residue. In some embodiments, the engineered cysteine residue is at one or more of positions HC S239C, LC V205C, LC A114C, HC A140C, LC K149C, LC S168C, LC S153C, LC A127C, HC T116C, and HC S115C, where HC refers to heavy chain, LC refers t light chain and the numbering of amino acid residues in the Fc region is according to the EU index as in Kabat. In certain embodiments, a compound or a compound-linker of any one of Formulas (I), (Ia), (Ib), (Ic) (Id) and (Ie)disclosed herein is attached to an amino acid residue of an IgG Fc domain disclosed herein selected from: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335 336, 396, 428, or any subset thereof wherein numbering of amino acid residues in the Fc domain is according to the EU index as in Kabat. In certain embodiments, a compound or a compound-linker of any one of Formulas (I), (Ia), (Ib), (Ic), (Id), and (Ie) disclosed herein may not be attached to an amino acid residue of an IgG Fc domain disclosed herein selected from: 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 292, 293, 294, 295, 296, 297, 298, 299, 300, 302, 305, 313, 317, 318, 320, 322, 323, 324, 325, 326, 327, 328, 329, 330, 331, 332, 333, 334, 335 336, 396, 428, or any subset thereof wherein numbering of amino acid residues in the Fc domain is according to the EU index as in Kabat.

### Pharmaceutical Formulations

The compositions, conjugates and methods described herein can be considered useful as pharmaceutical compositions for administration to a subject in need thereof. Pharmaceutical compositions can comprise at least one of the compounds, salts, or conjugates described herein and one or more pharmaceutically acceptable carriers, diluents, excipients, stabilizers, dispersing agents, suspending agents, and/or thickening agents. The composition can comprise the conjugate having an antibody construct and a compound or salt of any one of Formulas (I), (Ia), (Ib), (Ic), (Id) and (Ie)connected via a linker, as described herein. The composition can comprise the conjugate having an antibody construct, a target binding domain, and a compound or salt of any one of Formulas (I), (Ia), (Ib), (Ic), (Id) and (Ie) connected via a linker, as described herein. A composition can comprise any conjugate described herein. A pharmaceutical composition can comprise at least the compounds, salts or conjugates described herein and one or more of buffers, antibiotics, steroids, carbohydrates, drugs (e.g., chemotherapy drugs), radiation, polypeptides, chelators, adjuvants and/or preservatives.

Pharmaceutical compositions can be formulated using one or more physiologically-acceptable carriers comprising excipients and auxiliaries. A formulation can be modified depending upon the route of administration chosen. Pharmaceutical compositions comprising a compound, salt or conjugate as described herein can be manufactured, for example, by lyophilizing the compound, salt or conjugate, mixing, dissolving, emulsifying, encapsulating or entrapping the conjugate. The pharmaceutical compositions can also include the compounds, salts or conjugates described herein in a free-base form or pharmaceutically-acceptable salt form.

Methods for formulation of the conjugates described herein can include formulating any of the compounds, salts, or conjugates described herein with one or more inert, pharmaceutically-acceptable excipients or carriers to form a solid, semi-solid, or liquid composition. Solid compositions can include, for example, powders, tablets, dispersible granules, and capsules, and, in some aspects, the solid compositions further contain nontoxic, auxiliary substances such as wetting or emulsifying agents, pH buffering agents, and other pharmaceutically-acceptable additives. Alternatively, the compounds, salts, or conjugates described herein can be lyophilized or in powder form for re-constitution with a suitable vehicle, e.g., sterile pyrogen-free water, before use.

Pharmaceutical compositions described herein can comprise at least one active ingredient (e.g., a compound, salt, or conjugate). The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles, and nanocapsules) or in macroemulsions.

Pharmaceutical compositions as described herein often further can comprise more than one active compound (e.g., a compound, salt, or conjugate and other agents) as necessary for the particular indication being treated. The active compounds can have complementary activities that do not adversely affect each other. For example, the composition can also comprise a chemotherapeutic agent, a cytotoxic agent, a cytokine, a growth-inhibitory agent, an anti-hormonal agent, an anti-angiogenic agent, and/or a cardioprotectant. Such molecules can be present in combination in amounts that are effective for the purpose intended.

The compositions and formulations can be sterilized. Sterilization can be accomplished by, for example, filtration (e.g., sterile filtration).

The compositions described herein can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the compositions described herein can be lyophilized or in powder form for reconstitution with a suitable vehicle, e.g., sterile pyrogen-free water, before use.

For parenteral administration, the compounds, salts or conjugates can be formulated in a unit dosage injectable form (e.g., a solution, suspension, emulsion) in association with a pharmaceutically acceptable parenteral vehicle. Such vehicles can be inherently non-toxic, and non-therapeutic. Vehicles can be water, saline, Ringer's solution, dextrose solution, and 5% human serum albumin. Non-aqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release preparations can include semipermeable matrices of solid hydrophobic polymers that can contain the compound, salt, or conjugate, and these matrices can be in the form of shaped articles (e.g., films or microcapsules). Examples of sustained-release matrices can include polyesters, hydrogels (e.g., poly(2-hydroxyethyl-methacrylate), or poly (vinyl alcohol)), polylactides, copolymers of L-glutamic acid and y ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations described herein can be prepared for storage by mixing a compound, salt, or conjugate with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

Pharmaceutical formulations of the conjugates described herein may have an average drug-antibody contruct ratio selected from about 1 to about 10, wherein the drug is a compound or salt of any one of Formulas (I), (Ia), (Ib), (Ic), (Id) and (Ie). In certain embodiments, the average DAR of the formulation is from about 2 to about 8, such as from about 3 to about 8, such as from about 3 to about 7, such as from about 1 to 3 or such as from about 3 to 5. In certain embodiments, a pharmaceutical formulation has an average DAR of about 3, about 3.5, about 4, about 4.5, about 5, about 5.5, about 6, or about 6.6.

### Therapeutic Applications

The compositions, conjugates and methods of the present disclosure can be useful for a plurality of different subjects including, but are not limited to, a mammal, human, non-human mammal, domesticated animal (e.g., laboratory animals, household pets, or livestock), non-domesticated animal (e.g., wildlife), dog, cat, rodent, mouse, hamster, cow, bird, chicken, fish, pig, horse, goat, sheep, rabbit, and any combination thereof.

The compositions, conjugates and methods described herein can be useful as a therapeutic, for example, a treatment that can be administered to a subject in need thereof. A therapeutic effect of the present disclosure can be obtained in a subject by reduction, suppression, remission, or eradication of a disease state, including, but not limited to, a symptom thereof. A therapeutic effect in a subject having a disease or condition, or pre-disposed to have or beginning to have the disease or condition, can be obtained by a reduction, a suppression, a prevention, a remission, or an eradication of the condition or disease, or pre-condition or pre-disease state.

In practicing the methods described herein, therapeutically-effective amounts of the compositions and conjugates described herein can be administered to a subject in need thereof, often for treating and/or preventing a condition or progression thereof. A pharmaceutical composition can affect the physiology of the subject, such as the immune system, an inflammatory response, or other physiologic affect. A therapeutically-effective amount can vary depending on the severity of the disease, the age and relative health of the subject, the potency of the compounds used, and other factors.

“Treat” and/or “treating” refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. “Treat” can be used herein to refer to a method that results in some level of treatment or amelioration of the disease or condition, and can contemplate a range of results directed to that end, including but not restricted to prevention of the condition entirely.

“Prevent,” “preventing,” and the like refer to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual. Preventing can also refer to preventing re-occurrence of a disease or condition in a patient that has previously been treated for the disease or condition, e.g., by preventing relapse.

A therapeutically effective amount can be the amount of a composition or an active component thereof sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment, and can be ascertainable by one skilled in the art using known techniques.

The conjugates described herein that can be used in therapy can be formulated and dosages established in a fashion consistent with good medical practice taking into account the disease or condition to be treated, the condition of the individual patient, the site of delivery of the composition, the method of administration, and other factors known to practitioners. The compositions described herein can be prepared according to the description of preparation described herein.

Pharmaceutical compositions can be used in the methods described herein and can be administered to a subject in need thereof using a technique known to one of ordinary skill in the art which can be suitable as a therapy for the disease or condition affecting the subject. One of ordinary skill in the art would understand that the amount, duration and frequency of administration of a pharmaceutical composition described herein to a subject in need thereof depends on several factors including, for example, but not limited to, the health of the subject, the specific disease or condition of the patient, the grade or level of a specific disease or condition of the patient, the additional therapeutics the subject is being or has been administered, and the like.

The methods and compositions described herein can be for administration to a subject in need thereof. Often, administration of the compositions described herein can include routes of administration, non-limiting examples of administration routes include intravenous, intraarterial, subcutaneous, subdural, intramuscular, intracranial, intrasternal, intratumoral, or intraperitoneal. Additionally, a pharmaceutical composition can be administered to a subject by additional routes of administration, for example, by inhalation, oral, dermal, intranasal, or intrathecal administration.

Compositions of the present disclosure can be administered to a subject in need thereof in a first administration, and in one or more additional administrations. The one or more additional administrations can be administered to the subject in need thereof minutes, hours, days, weeks or months following the first administration. Any one of the additional administrations can be administered to the subject in need thereof less than 21 days, or less than 14 days, less than 10 days, less than 7 days, less than 4 days, or less than 1 day after the first administration. The one or more administrations can occur more than once per day, more than once per week, or more than once per month. The administrations can be weekly, biweekly (every two weeks), every three weeks, monthly, or bimonthly.

The compositions and methods provided herein can be useful for the treatment of a plurality of diseases, conditions, preventing a disease or a condition in a subject or other therapeutic applications for subjects in need thereof. Often the compositions and methods provided herein can be useful for treatment of hyperplastic conditions, including but not limited to, neoplasms, cancers, tumors and the like. The compositions, conjugates and methods provided herein can be useful for specifically targeting ALK5. In one embodiment, the compounds of the present disclosure serve as ALK5 agonists and activate an immune response. In another embodiment, the conjugates of the present disclosure serve as ALK5 agonists and activate an immune response. A condition, such as a cancer, can be associated with expression of an antigen on the cancer cells. Often, the antigen expressed by the cancer cells can comprise an extracellular portion capable of recognition by the antibody construct of the conjugate. An antigen expressed by the cancer cells can be a tumor antigen. An antibody construct portion of the conjugate can recognize a tumor antigen. In certain embodiments, the tumor antigen is selected from MUC16, UPK1B, VTCN1, TMPRSS3, TMEM238, C1orf186, TMPRSS4, CLDN6, CLDN8 and STRA6. In certain embodiments, the tumor antigen is selected from MUC16, UPK1B, VTCN1, TMPRSS3, TMEM238, C1orf186 and TMPRSS4. In certain embodiments, the tumor antigen is MUC16. In certain embodiments, the tumor antigen is UPK1B. In certain embodiments, the tumor antigen is VTCN1. In certain embodiments, the tumor antigen is TMPRSS3. In certain embodiments, the tumor antigen is TMEM238. In certain embodiments, the tumor antigen is C1orf186. In certain embodiments, the tumor antigen is TMPRSS4. In certain embodiments, the tumor antigen is CLDN6. In certain embodiments, the tumor antigen is CLDN8. In certain embodiments, the tumor antigen is STRA6.

In certain embodiments, the tumor antigen is selected from ACSL5, AP1M2, AREG, CDH1, CDH17, CEACAM5, CEACAM6, CEACAM7, CLCA1, CLDN3, DPEP1, ERBB3, GPA33, GPRC5A, ITGA6, KRTCAP3, LSR, MUC13, NOX1, PLOD3, PLPP2, SLC12A2, SLC44A4, SLC52A2, SMIM22, ST14, TFRC, TMPRSS4, sLE(x) and TSPAN6. In certain embodiments, the tumor antigen is selected from CLDN4, CLDN7, EPCAM, PIGR, TMEM141, TMEM54, TSPAN1 LRG5, and TSPAN8. In certain embodiments, the tumor antigen is selected from ACSL5, AP1M2, AREG, CDH1, CDH17, CEACAM5, CEACAM6, CEACAM7, CLCA1, CLDN3, DPEP1, ERBB3, GPA33, GPRC5A, ITGA6, KRTCAP3, LSR, MUC13, NOX1, PLOD3, PLPP2, SLC12A2, SLC44A4, SLC52A2, SMIM22, ST14, TFRC, TMPRSS4, sLE(x), TSPAN6, CLDN4, CLDN7, EPCAM, PIGR, TMEM141, TMEM54, TSPAN1, LRG5 and TSPAN8. In certain embodiments, the tumor antigen is any one of ACSL5, AP1M2, AREG, CDH1, CDH17, CEACAM5, CEACAM6, CEACAM7, CLCA1, CLDN3, DPEP1, ERBB3, GPA33, GPRC5A, ITGA6, KRTCAP3, LSR, MUC13, NOX1, PLOD3, PLPP2, SLC12A2, SLC44A4, SLC52A2, SMIM22, ST14, TFRC, TMPRSS4, sLE(x), TSPAN6, CLDN4, CLDN7, EPCAM, PIGR, TMEM141, TMEM54, TSPAN1, LRG5 and TSPAN8.

In certain embodiment the tumor antigen is selected from LRRC15, ADAM12, MMP14, GPX8, PDPN, CDH11 and F2RL2.

In certain embodiments, the tumor antigen is selected from an antigen on myofibroblasts, such as LRRC15, ADAM12, FAP, and CDH11.

As described herein, an antigen binding domain portion of the conjugate may be configured to recognize an antigenexpressed by a cancer cell, such as for example, a disease antigen, tumor antigen or a cancer antigen. Often such antigens are known to those of ordinary skill in the art, or newly found to be associated with such a condition, to be commonly associated with, and/or, specific to, such conditions. For example, a disease antigen, tumor antigen, or a cancer antigen is, but is not limited to, MUC16, UPK1B, VTCN1, TMPRSS3, TMEM238, C1orf186, TMPRSS4, CLDN6, CLDN8 and STRA6. In certain embodiments, the tumor antigen is selected from MUC16, UPK1B, VTCN1, TMPRSS3, TMEM238, C1orf186 and TMPRSS4. In certain embodiments, the tumor antigen is MUC16. In certain embodiments, the tumor antigen is UPK1B. In certain embodiments, the tumor antigen is VTCN1. In certain embodiments, the tumor antigen is TMPRSS3. In certain embodiments, the tumor antigen is TMEM238. In certain embodiments, the tumor antigen is C1orf186. In certain embodiments, the tumor antigen is TMPRSS4. In certain embodiments, the tumor antigen is CLDN6. In certain embodiments, the tumor antigen is CLDN8. In certain embodiments, the tumor antigen is STRA6.

In certain embodiments, the tumor antigen is selected from ACSL5, AP1M2, AREG, CDH1, CDH17, CEACAM5, CEACAM6, CEACAM7, CLCA1, CLDN3, DPEP1, ERBB3, GPA33, GPRC5A, ITGA6, KRTCAP3, LSR, MUC13, NOX1, PLOD3, PLPP2, SLC12A2, SLC44A4, SLC52A2, SMIM22, ST14, TFRC, TMPRSS4, sLE(x) and TSPAN6. In certain embodiments, the tumor antigen is selected from CLDN4, CLDN7, EPCAM, PIGR, TMEM141, TMEM54, TSPAN1 LRG5, and TSPAN8. In certain embodiments, the tumor antigen is selected from ACSL5, AP1M2, AREG, CDH1, CDH17, CEACAM5, CEACAM6, CEACAM7, CLCA1, CLDN3, DPEP1, ERBB3, GPA33, GPRC5A, ITGA6, KRTCAP3, LSR, MUC13, NOX1, PLOD3, PLPP2, SLC12A2, SLC44A4, SLC52A2, SMIM22, ST14, TFRC, TMPRSS4, sLE(x), TSPAN6, CLDN4, CLDN7, EPCAM, PIGR, TMEM141, TMEM54, TSPAN1, LRG5 and TSPAN8. In certain embodiments, the tumor antigen is any one of ACSL5, AP1M2, AREG, CDH1, CDH17, CEACAM5, CEACAM6, CEACAM7, CLCA1, CLDN3, DPEP1, ERBB3, GPA33, GPRC5A, ITGA6, KRTCAP3, LSR, MUC13, NOX1, PLOD3, PLPP2, SLC12A2, SLC44A4, SLC52A2, SMIM22, ST14, TFRC, TMPRSS4, sLE(x), TSPAN6, CLDN4, CLDN7, EPCAM, PIGR, TMEM141, TMEM54, TSPAN1, LRG5 and TSPAN8.

In certain embodiment the tumor antigen is selected from LRRC15, ADAM12, MMP14, GPX8, PDPN, CDH11 and F2RL2.

In certain embodiments, the tumor antigen is selected from an antigen on myofibroblasts, such as LRRC15, ADAM12, FAP, and CDH11.

Additionally, such tumor antigens can be derived from the following specific conditions and/or families of conditions, including but not limited to, cancers such as brain cancers, skin cancers, lymphomas, sarcomas, lung cancer, liver cancer, leukemias, uterine cancer, breast cancer, ovarian cancer, cervical cancer, bladder cancer, kidney cancer, hemangiosarcomas, bone cancers, blood cancers, testicular cancer, prostate cancer, stomach cancer, intestinal cancers, pancreatic cancer, and other types of cancers as well as pre-cancerous conditions such as hyperplasia or the like. Non-limiting examples of cancers can include Acute lymphoblastic leukemia (ALL); Acute myeloid leukemia; Adrenocortical carcinoma; Astrocytoma, childhood cerebellar or cerebral; Basal-cell carcinoma; Bladder cancer; Bone tumor, osteosarcoma/malignant fibrous histiocytoma; Brain cancer; Brain tumors, such as, cerebellar astrocytoma, malignant glioma, ependymoma, medulloblastoma, visual pathway and hypothalamic glioma; Brainstem glioma; Breast cancer; Bronchial adenomas/carcinoids; Burkitt's lymphoma; Cerebellar astrocytoma; Cervical cancer; Cholangiocarcinoma; Chondrosarcoma; Chronic lymphocytic leukemia; Chronic myelogenous leukemia; Chronic myeloproliferative disorders; Colon cancer; Cutaneous T-cell lymphoma; Endometrial cancer; Ependymoma; Esophageal cancer; Eye cancers, such as, intraocular melanoma and retinoblastoma; Gallbladder cancer; Glioma; Hairy cell leukemia; Head and neck cancer; Heart cancer; Hepatocellular (liver) cancer; Hodgkin lymphoma; Hypopharyngeal cancer; Islet cell carcinoma (endocrine pancreas); Kaposi sarcoma; Kidney cancer (renal cell cancer); Laryngeal cancer; Leukemia, such as, acute lymphoblastic, acute myeloid, chronic lymphocytic, chronic myelogenous and, hairy cell; Lip and oral cavity cancer; Liposarcoma; Lung cancer, such as, non-small cell and small cell; Lymphoma, such as, AIDS-related, Burkitt; Lymphoma, cutaneous T-Cell, Hodgkin and Non-Hodgkin, Macroglobulinemia, Malignant fibrous histiocytoma of bone/osteosarcoma; Melanoma; Merkel cell cancer; Mesothelioma; Multiple myeloma/plasma cell neoplasm; Mycosis fungoides; Myelodysplastic syndromes; Myelodysplastic/myeloproliferative diseases; Myeloproliferative disorders, chronic; Nasal cavity and paranasal sinus cancer; Nasopharyngeal carcinoma; Neuroblastoma; Oligodendroglioma; Oropharyngeal cancer; Osteosarcoma/malignant fibrous histiocytoma of bone; Ovarian cancer; Pancreatic cancer; Parathyroid cancer; Pharyngeal cancer; Pheochromocytoma; Pituitary adenoma; Plasma cell neoplasia; Pleuropulmonary blastoma; Prostate cancer; Rectal cancer; Renal cell carcinoma (kidney cancer); Renal pelvis and ureter, transitional cell cancer; Rhabdomyosarcoma; Salivary gland cancer; Sarcoma, Ewing family of tumors; Sarcoma, Kaposi; Sarcoma, soft tissue; Sarcoma, uterine; Sézary syndrome; Skin cancer (non-melanoma); Skin carcinoma; Small intestine cancer; Soft tissue sarcoma; Squamous cell carcinoma; Squamous neck cancer with occult primary, metastatic; Stomach cancer; Testicular cancer; Throat cancer; Thymoma and thymic carcinoma; Thymoma,; Thyroid cancer; Thyroid cancer, childhood; Uterine cancer; Vaginal cancer; Waldenstrüm macroglobulinemia; Wilms tumor; and any combination thereof.

The invention provides any therapeutic compound or conjugate disclosed herein for use in a method of treatment of the human or animal body by therapy. Therapy may be by any mechanism disclosed herein, such as by stimulation of the immune system. The invention provides any therapeutic compound or conjugate disclosed herein for use in stimulation of the immune system, vaccination or immunotherapy, including for example enhancing an immune response. The invention further provides any therapeutic compound or conjugate disclosed herein for prevention or treatment of any condition disclosed herein, for example cancer, fibrosis, autoimmune disease, inflammation, sepsis, allergy, asthma, graft rejection, graft-versus-host disease, immunodeficiency or infectious disease (typically caused by an infectious pathogen). The invention also provides any therapeutic compound or conjugate disclosed herein for obtaining any clinical outcome disclosed herein for any condition disclosed herein, such as reducing tumour cells in vivo. The invention also provides use of any therapeutic compound or conjugate disclosed herein in the manufacture of a medicament for preventing or treating any condition disclosed herein.

### General Synthetic Schemes and Examples

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### General Scheme I for the Preparation of Exemplary ALK5 Inhibitors

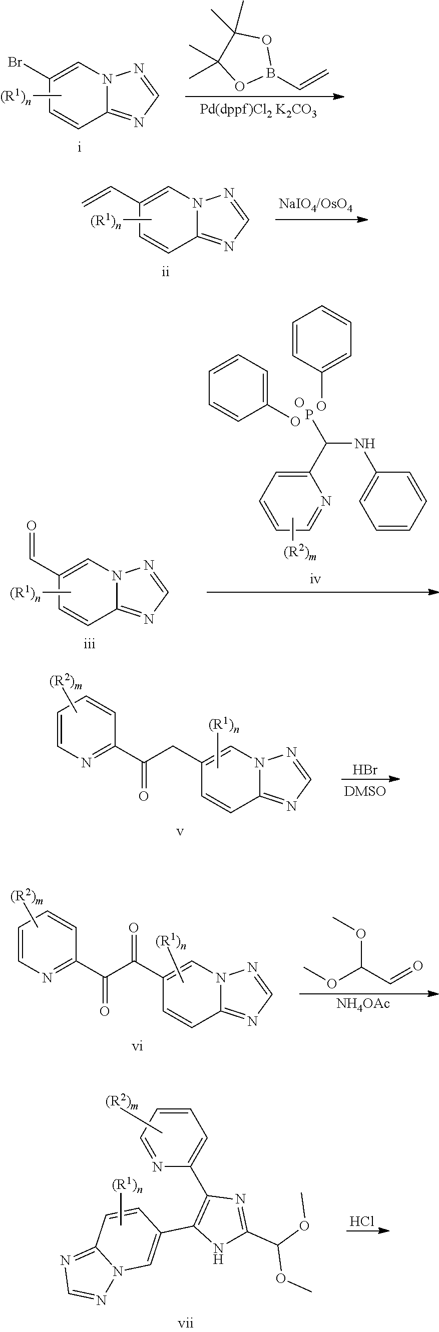

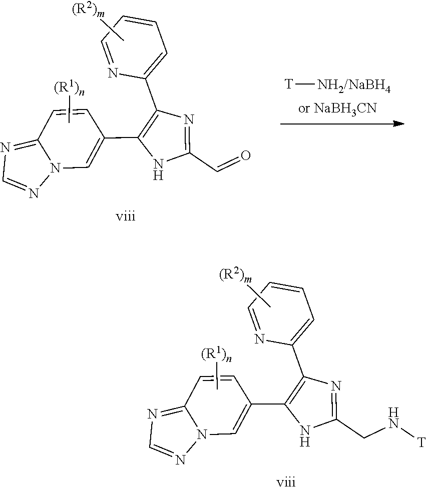

In one method, compounds of Formula (I) wherein Q is CH₂N are prepared according to Scheme 1. Specifically, R²-substituted pyridine-2-carbaldehydes are reacted with aniline and diphenyl phosphite to give N,P-acetal (iv), which can be further coupled with le substituted [1,2,4]triazolo[1,5-a]pyridine-6-carbaldehydes (iii) followed by hydrolysis in acidic condition to produce a monoketone (v). The monoketone (v) may be oxidized to a diketone (vi) with HBr in DMSO. Certain diketone analogs (vi) were commercially available (e.g. R¹=H and R²=CH³: CAS No. 356560-84-4) and were used as a starting point when appropriate. Diketones (vi) can be condensed with 2,2-dimethoxyacetaldehyde in the presence of ammonium acetate to yield an acetal-protected imidazole (vii), which can be hydrolyzed in acidic condition to produce an imidazole-2-carbaldehyde (viii). The imidazole-2-carbaldehyde (viii) can be reductively aminated in the presence of an amine and a reducing agent such as sodium borohydride or sodium cyanoborohydride to yield a compound of Formula (I).

### EXAMPLE 1

### Synthesis of N-((5-([1,2,4]triazolo[1,5-a]pyridin-6-yl)-4-(6-methylpyridin-2-yl)-1H-imidazol-2-yl)methyl)-1,2,3,4-tetrahydroisoquinolin-7-amine (Compound 1.1)

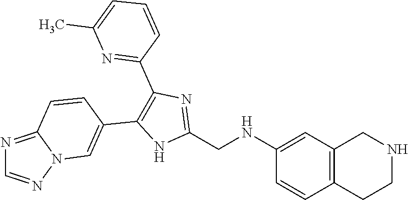 [SMILES: Cc1cccc(-c2nc(CNc3ccc4c(c3)CNCC4)[nH]c2-c2ccc3ncnn3c2)n1]

### Step A. Preparation of Int 1.1a

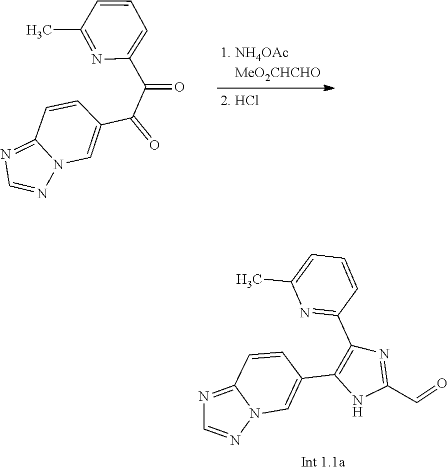 [SMILES: Cc1cccc(C(=O)C(=O)c2ccc3ncnn3c2)n1 ; Cc1cccc(-c2nc(C=O)[nH]c2-c2ccc3ncnn3c2)n1]

To a stirred solution of 1-([1,2,4]triazolo[1,5-a]pyridin-6-yl)-2-(6-methylpyridin-2-yl)ethane-1,2-dione (CAS 356560-84-4; 2.7 g, 10.1 mmol) in a 2:1 mixture of tert-butyl methyl ether and methanol (25 mL) were added 60% 2,2-dimethoxyacetaldehyde in H₂O (3.5 mL, 20 mmol) and NH₄OAc (1.95 g, 25.2 mmol). The mixture was stirred at room temperature for 5h before the solvent was removed. The pH of the reaction mixture was adjusted to 8 with saturated aqueous NaHCO₃ solution and extracted with CH₂Cl₂ (2×10 mL). The combined organic extracts were washed with brine (10 mL) and dried over anhydrous Na₂SO₄, filtered, and evaporated. The residue was purified on silica gel (ISCO gold, 40g; 0% to 20% CH₂Cl₂/MeOH over 15 minutes) to give the desired imidazole product which dissolved in 1 N HCl (20 mL) and heated at 70° C. for 4 h. The reaction mixture was allowed to cool to 0° C. and then it was neutralized with saturated aqueous NaHCO₃ solution. The precipitate was collected and washed with water (20 mL) and ether (40 mL) to give the desired product Int 1.1a as a yellow-brown solid. ¹H NMR (DMSO-d⁶) δ 10.0 (dd, J=1.6, 0.8 Hz, 1H), 9.56 (s, 1H), 8.42 (s, 1H), 8.27 (dd, J=9.2, 1.6Hz, 1H), 7.82 (br d, J=0.8 Hz, 1H), 7.72 (dd, J=9.2, 0.8Hz, 1H), 7.65 (t, J=7.8Hz, 1H), 7.05 (d, J=7.6Hz, 1H), 2.46 (s, 3H). LCMS (M+H)=305.1.

### Step B. Preparation of Int 1.1b

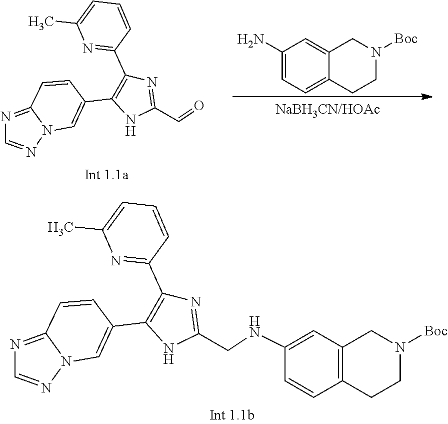 [SMILES: Cc1cccc(-c2nc(C=O)[nH]c2-c2ccc3ncnn3c2)n1 ; CC(C)(C)OC(=O)N1CCc2ccc(N)cc2C1 ; Cc1cccc(-c2nc(CNc3ccc4c(c3)CN(C(=O)OC(C)(C)C)CC4)[nH]c2-c2ccc3ncnn3c2)n1]

To a solution of 4-(6-methyl-2-pyridyl)-5-([1,2,4]triazolo[1,5-a]pyridin-6-yl)-1H-imidazole-2-carbaldehyde (200 mg, 0.66 mmol) in dichloroethane (30 mL) was added acetic acid (79 mg, 1.3 mmol, 75 uL) and tert-butyl 7-amino-3,4-dihydro-1H-isoquinoline-2-carboxylate (245 mg, 0.99 mmol). The mixture was stirred at 60° C. for 2 h then cooled to 0° C. Methanol (20 mL) and THF (10 mL) were added followed by NaBH₃CN (165 mg, 2.63 mmol, 4 eq) and then the reaction mixture was allowed to warm to 15° C. and stirred for an additional 3 h at which time LCMS showed the reaction to be complete. The reaction mixture was quenched by addition of 0.10 mL of water at 0° C. then concentrated under reduced pressure to give a residue that was purified by silica gel chromatography (DCM/MeOH=20/1 to 10/1) to afford 300 mg of tert-butyl 7-[[4-(6-methyl-2-pyridyl)-5-([1,2,4]triazolo[1,5-a]pyridin-6-yl)-1H-imidazol-2-yl]methyl-amino]-3,4-dihydro-1H-isoquinoline-2-carboxylate as a yellow solid. LCMS (M+H)=537.

### Step C. Preparation of Compound 1.1

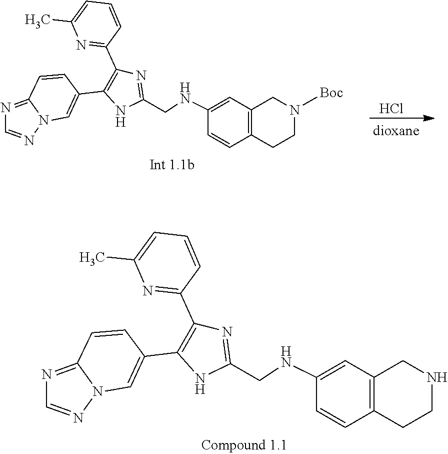 [SMILES: Cc1cccc(-c2nc(CNc3ccc4c(c3)CN(C(=O)OC(C)(C)C)CC4)[nH]c2-c2ccc3ncnn3c2)n1 ; Cl ; Cc1cccc(-c2nc(CNc3ccc4c(c3)CNCC4)[nH]c2-c2ccc3ncnn3c2)n1]

To a 0° C. mixture containing 108 mg (0.20 mmol) of compound Int 1.1b in 5 mL of dioxane was added 2 mL (8.0 mmol) of HCl in dioxane (4M). The reaction mixture was stirred for 2 h and the solvent was removed under reduced pressure. Saturated NaHCO₃ solution was added and the slurry was extracted with EtOAc (3×). The combine organic extracts were washed with brine then dried over Na₂SO₄. Evaporation of the solvent provided 77 mg of Compound 1.1 as an off-white solid. ¹H NMR (DMSO-d⁶) δ 9.51 (m, 3H), 8.65 (s, 1 H), 7.96 (d, J=9.6 Hz, 1H), 7.87 (m, 2H), 7.70 (d, J=8.0Hz, 1H), 7.37 (d, J=7.8 Hz, 1H), 6.97 (d, J=8.8 Hz, 1H), 6.73 (d, J=6.0Hz, 2H), 4.75 (s, 2H), 4.13-4.10 (m, 2H), 3.25 (s, 2H), 2.83 (t, J=6.0Hz, 2H), 2.46 (s, 3H). LCMS (M+H) =437.2.

The compounds in Table 1 were prepared in a manner similar to that described for compound 1.1 using the appropriately substituted amine.

TABLE 1
Compounds prepared according to the methods described herein.

| | | | MS |
|---|---|---|---|
| Cmpd | Structure and Name | ¹H NMR | (M + H) |
| 1.2 | 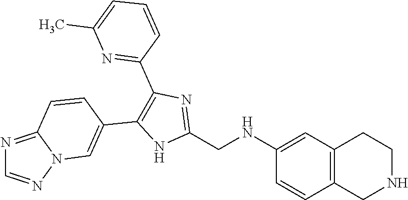 [SMILES: Cc1cccc(-c2nc(CNc3ccc4c(c3)CCNC4)[nH]c2-c2ccc3ncnn3c2)n1] | ¹H NMR (DMSO-d₆, 400 MHz) δ9.83 (s, 2H), 9.52 (s, 1H), 8.66 (s, 1H), 7.98-7.96 (m, 1H), 7.91-7.80 (m, 2H), 7.68 (d, J = 8.0 Hz, 1H), 7.37 (d, J = 8.0 Hz, 1H), 7.09 (t, J = 8.0 Hz, 1H), 6.76 (d, J = 8.0 Hz, 1H), 6.57 (d, J = 7.6 Hz, 1H), 4.84 (s, 2H), 4.21 (s, 2H), 3.27 (s, 2H), 2.94 (s, 2H), 2.46 (s, 3H) | 437.3 |
| | N-((5-([1,2,4]triazolo[1,5-a]pyridin-6-yl)- | | |
| | 4-(6-methylpyridin-2-yl)-1H-imidazol-2- | | |
| | yl)methyl)-1,2,3,4-tetrahydroisoquinolin- | | |
| | 6-amine | | |
| 1.3 | 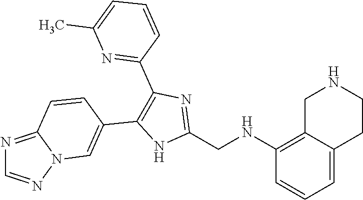 [SMILES: Cc1cccc(-c2nc(CNc3cccc4c3CNCC4)[nH]c2-c2ccc3ncnn3c2)n1] | ¹H NMR (DMSO-d₆, 400 MHz) δ 9.63 (s, 2H), 9.54 (s, 1H), 8.67 (s, 1H), 8.01-7.95 (m, 1H), 7.94-7.88 (m, 1H), 7.87-7.81 (m, 1H), 7.72 (d, J = 7.6 Hz, 1H), 7.36 (d, J = 7.6 Hz, 1H), 7.07 (t, J = 7.6 Hz, 1H), 6.84 (d, J = 8.0 Hz, 1H), 6.55 (d, J = 7.6 Hz, 1H), 4.87 (s, 2H), 4.14 (s, 2H), 3.37 (s, 2H), 2.93 (s, 2H), 2.47 (s, 3H) | 437.3 |
| | N-((5-([1,2,4]triazolo[1,5-a]pyridin-6-yl)- | | |
| | 4-(6-methylpyridin-2-yl)-1H-imidazol-2- | | |
| | yl)methyl)-1,2,3,4-tetrahydroisoquinolin- | | |
| | 8-amine | | |
| 1.4 | 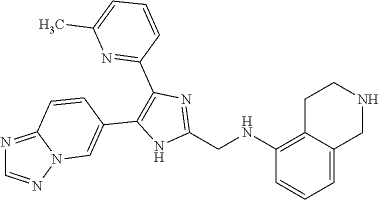 [SMILES: Cc1cccc(-c2nc(CNc3cccc4c3CCNC4)[nH]c2-c2ccc3ncnn3c2)n1] | ¹H NMR (DMSO-d₆, 400 MHz) δ 9.58 (s, 2H), 9.53 (s, 1H), 8.67 (s, 1H), 7.99-7.95 (m, 1H), 7.92-7.83 (m, 2H), 7.72 (d, J = 8.0 Hz, 1H), 7.37 (d, J = 8.0 Hz, 1H), 6.97 (d, J = 8.0 Hz, 1H), 6.78-6.68 (m, 2H), 4.77 (s, 2H), 4.05 (s, 2H), 3.24 (s, 2H), 2.91 (s, 2H), 2.48(s, 3H) | 437.3 |
| | N-((5-([1,2,4]triazolo[1,5-a]pyridin-6-yl)- | | |
| | 4-(6-methylpyridin-2-yl)-1H-imidazol-2- | | |
| | yl)methyl)-1,2,3,4-tetrahydroisoquinolin- | | |
| | 5-amine | | |
| 1.5 | 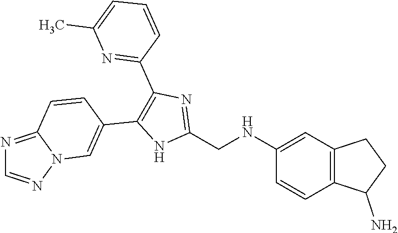 [SMILES: Cc1cccc(-c2nc(CNc3ccc4c(c3)CCC4N)[nH]c2-c2ccc3ncnn3c2)n1] | ¹H NMR (DMSO-d₆, 400 MHz) δ 9.54 (s, 1H), 8.49 (s, 1H), 7.98 (dd, J = 1.6, 9.4 Hz, 1H), 7.82 (d, J = 9.2 Hz, 1H), 7.70 (t, J = 7.8 Hz, 1H), 7.15 (d, J = 7.4 Hz, 1H), 7.05 (d, J = 8.6 Hz, 1H), 6.57-6.55 (m, 2H), 5.91 (t, J = 5.8 Hz, 1H), 4.33 (d, J = 5.8 Hz, 2H), 4.07 (t, J = 7.2 Hz, 1H), 2.75 (ddd, J = 3.2, 8.4, 15.6 Hz, 1H), 2.60-2.55 (m, 1H), 2.46 (s, 3H), 2.21-2.30 (m, 1H), 1.55-1.46 (m, 1H) | 459.2 |
| | N⁵-((5-([1,2,4]triazolo[1,5-a]pyridin-6- | | |
| | yl)-4-(6-methylpyridin-2-yl)-1H- | | |
| | imidazol-2-yl)methyl)-2,3-dihydro-1H- | | |
| | indene-1,5-diamine | | |
| 1.6 | 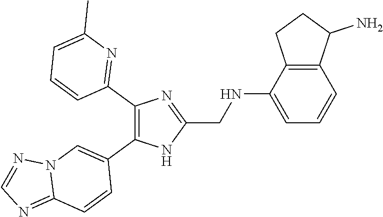 [SMILES: Cc1cccc(-c2nc(CNc3cccc4c3CCC4N)[nH]c2-c2ccc3ncnn3c2)n1] | ¹H NMR (DMSO-d₆, 400 MHz) δ 9.50 (s, 1H), 8.63 (s, 1H), 8.45 (s, 3H), 7.97 (d, J = 9.8 Hz, 1H), 7.87- 7.81 (m, 2H), 7.66 (d, J = 7.6 Hz, 1H), 7.36 (d, J = 7.6 Hz, 1H), 7.14- 7.10 (m, 1H), 6.96 (d, J = 7.6 Hz, 1H), 6.80 (d, J = 7.6 Hz, 1H), 4.82 (s, 2H), 4.62 (s, 1H), 3.06-3.00 (m, 1H), 2.86-2.78 (m, 1H), 2.49 (s, 3H), 2.03-1.98 (m, 1H) | 437.3 |
| | N⁴-((5-([1,2,4]triazolo[1,5-a]pyridin-6- | | |
| | yl)-4-(6-methylpyridin-2-yl)-1H- | | |
| | imidazol-2-yl)methyl)-2,3-dihydro-1H- | | |
| | indene-1,4-diamine | | |
| 1.7 | 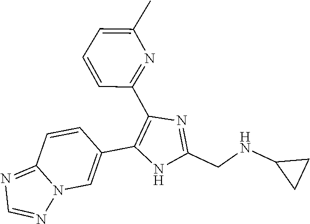 [SMILES: Cc1cccc(-c2nc(CNC3CC3)[nH]c2-c2ccc3ncnn3c2)n1] | ¹H NMR (DMSO-d₆, 400 MHz) δ 9.54 (s, 1H), 8.65 (s, 1H), 8.10-7.91 (m, 3H), 7.78-7.63 (m, 1H), 7.50 (d, J = 7.2 Hz, 1H), 4.48 (s, 2H), 3.00- 2.87 (m, 1H), 2.64 (s, 3H), 1.00 (s, 2H), 0.80 (d, J = 5.2 Hz, 2H) | 346.1 |
| | N-((5-([1,2,4]triazolo[1,5-a]pyridin-6-yl)- | | |
| | 4-(6-methylpyridin-2-yl)-1H-imidazol-2- | | |
| | yl)methyl)-cyclopropanamine | | |
| 1.8 | 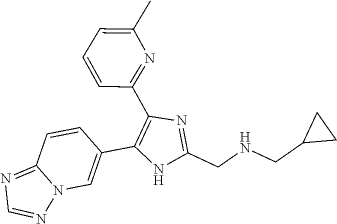 [SMILES: Cc1cccc(-c2nc(CNCC3CC3)[nH]c2-c2ccc3ncnn3c2)n1] | ¹H NMR (DMSO-d₆, 400 MHz) δ0.14 (s, 2H), 9.52 (s, 1H), 8.77 (s, 1H), 8.15-8.09 (m, 3H), 8.03 (d, J = 9.2 Hz, 3H), 8.00-7.95 (m, 2H), 7.76 (d, J = 8.0 Hz, 1H), 7.61 (d, J = 7.6 Hz, 1H), 4.49 (s, 2H), 3.05 (s, 2H), 2.68 (s, 3H), 1.20 (s, 1H), 0.58 (d, J = 7.6 Hz, 2H), 0.46 (d, J = 4.4 Hz, 2H) | 360.3 |
| | 1-(5-([1,2,4]triazolo[1,5-a]pyridin-6-yl)- | | |
| | 4-(6-methylpyridin-2-yl)-1H-imidazol-2- | | |
| | yl)-N-(cyclopropyl-methyl)methanamine | | |
| 1.9 | 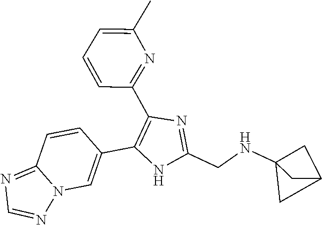 [SMILES: Cc1cccc(-c2nc(CNC34CC(C3)C4)[nH]c2-c2ccc3ncnn3c2)n1] | ¹¹H NMR (DMSO-d₆, 400 MHz) δ12.48 (s, 1H), 9.57 (s, 1H), 8.50 (s, 1H), 8.01 (s, 1H), 7.90-7.64 (m, 2H), 7.42 (s, 1H), 7.17-7.07 (m, 1H), 3.81 (s, 2H), 2.48-2.30 (m, 3H), 1.75-1.68 (m, 6H) | 372.3 |
| | N-((5-([1,2,4]triazolo[1,5-a]pyridin-6-yl)- | | |
| | 4-(6-methylpyridin-2-yl)-1H-imidazol-2- | | |
| | yl)methyl)bicyclo-[1.1.1]pentan-1-amine | | |
| 1.10 | 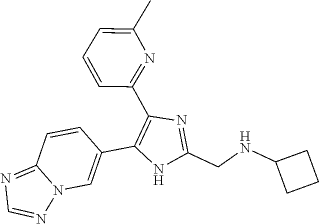 [SMILES: Cc1cccc(-c2nc(CNC3CCC3)[nH]c2-c2ccc3ncnn3c2)n1] | ¹H NMR (DMSO-d₆, 400 MHz) δ 9.56 (s, 1H), 8.49 (s, 1H), 8.00 (d, J = 9.2 Hz, 1H), 7.82 (d, J = 9.2 Hz, 1H), 7.69 (t, J = 7.6 Hz, 1H), 7.51 (d, J = 14.4 Hz, 1H), 7.13 (d, J = 7.2 Hz, 1H), 3.72 (s, 2H), 3.31-3.13 (m, 2H), 2.46 (s, 3H), 2.09-2.02 (m, 1H), 2.13-2.01 (m, 1H), 1.74-1.48 (m, 4H) | 360.2 |
| | 1-(5-([1,2,4]triazolo[1,5-a]pyridin-6-yl)- | | |
| | 4-(6-methylpyridin-2-yl)-1H-imidazol-2- | | |
| | yl)-N-(cyclobutyl)methanamine | | |
| 1.11 | 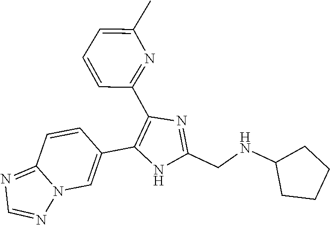 [SMILES: Cc1cccc(-c2nc(CNC3CCCC3)[nH]c2-c2ccc3ncnn3c2)n1] | ¹H NMR (DMSO-d₆, 400 MHz) δ 9.56 (br s, 1H), 8.50 (s, 1H), 7.99 (s, 1H), 7.83 (s, 1H), 7.73 (s, 1H), 7.52 (s, 1H), 7.15 (s, 1H), 3.80 (s, 2H), 3.08 (s, 1H), 2.46 (s, 3H), 1.83-1.29 (m, 10H) | 374.2 |
| | 1-(5-([1,2,4]triazolo[1,5-a]pyridin-6-yl)- | | |
| | 4-(6-methylpyridin-2-yl)-1H-imidazol-2- | | |
| | yl)-N-(cyclopentyl)methanamine | | |
| 1.12 | 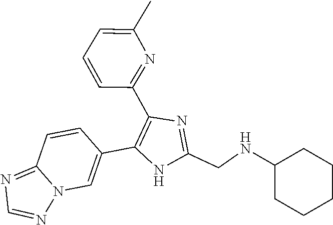 [SMILES: Cc1cccc(-c2nc(CNC3CCCCC3)[nH]c2-c2ccc3ncnn3c2)n1] | ¹H NMR (DMSO-d₆, 400 MHz) δ 9.55 (s, 1H), 9.65-9.49 (m, 1H), 8.49 (s, 1H), 7.99 (d, J = 6.8 Hz, 1H), 7.83 (s, 1H), 7.70 (s, 1H), 7.52 (s, 1H), 7.15 (s, 1H), 3.83 (s, 2H), 2.46 (s, 3H), 1.85 (s, 2H), 1.67 (s, 2H), 1.55 (s, 1H), 1.31-0.98 (m, 6H) | 388.3 |
| | 1-(5-([1,2,4]triazolo[1,5-a]pyridin-6-yl)- | | |
| | 4-(6-methylpyridin-2-yl)-1H-imidazol-2- | | |
| | yl)-N-(cyclohexyl)methanamine | | |
| 1.13 | 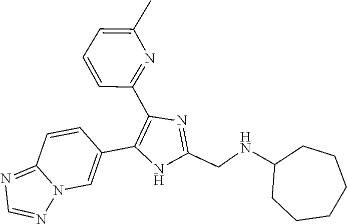 [SMILES: Cc1cccc(-c2nc(CNC3CCCCCC3)[nH]c2-c2ccc3ncnn3c2)n1] | ¹H NMR (DMSO-d₆, 400 MHz) δ 9.55 (br s, 1H), 8.49 (s, 1H), 7.99 (d, J = 8.8 Hz, 1H), 7.82 (d, J = 9.2 Hz, 1H), 7.70 (t, J = 7.6 Hz, 1H), 7.51 (s, 1H), 7.14 (d, J = 7.2 Hz, 1H), 3.79 (s, 2H), 2.75-2.58 (m, 1H), 2.47 (s, 3H), 1.81 (s, 2H), 1.62 (s, 2H), 1.49 (s, 4H), 1.37 (s, 4H) | 402.3 |
| | 1-(5-([1,2,4]triazolo[1,5-a]pyridin-6-yl)- | | |
| | 4-(6-methylpyridin-2-yl)-1H-imidazol-2- | | |
| | yl)-N-(cycloheptyl)methanamine | | |
| 1.14 | 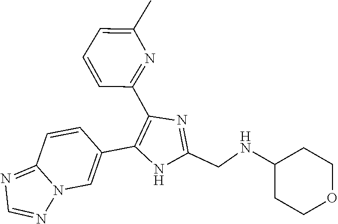 [SMILES: Cc1cccc(-c2nc(CNC3CCOCC3)[nH]c2-c2ccc3ncnn3c2)n1] | ¹H NMR (DMSO-d₆, 400 MHz) δ 9.55 (s, 1H), 8.50 (s, 1H), 7.99 (d, J = 9.2 Hz, 1H), 7.83 (d, J = 9.0 Hz, 1H), 7.71 (t, J = 7.8 Hz, 1H), 7.16 (s, 1H), 3.85 (s, 3H), 3.83 (d, J = 3.6 Hz, 1H), 3.42-3.19 (m, 4H), 2.75-2.63 (m, 1H), 2.46 (s, 3H), 1.81 (d, J = 12.0 Hz, 2H), 1.40-1.19 (m, 2H) | 390.2 |
| | 1-(5-([1,2,4]triazolo[1,5-a]pyridin-6-yl)- | | |
| | 4-(6-methylpyridin-2-yl)-1H-imidazol-2- | | |
| | yl)-N-(4-tetrahydropyranyl)methanamine | | |
| 1.15 | 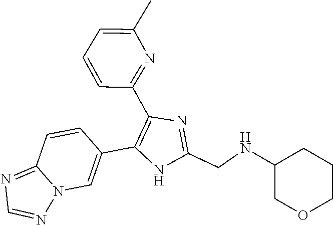 [SMILES: Cc1cccc(-c2nc(CNC3CCCOC3)[nH]c2-c2ccc3ncnn3c2)n1] | ¹H NMR (DMSO-d₆, 400 MHz) δ 9.54 (s, 1H), 8.49 (s, 1H), 7.98 (d, J = 9.2 Hz, 1H), 7.82 (d, J = 9.2 Hz, 1H), 7.70 (t, J = 8.0 Hz, 1H), 7.15 (s, 1H), 3.87-3.84 (m, 1H), 3.83 (d, J = 1.6 Hz, 2H), 3.71-3.64 (m, 1H), 3.30-3.23 (m, 3H), 3.04 (dd, J = 8.8, 10.8 Hz, 1H), 2.62-2.54 (m, 2H), 2.46 (s, 3H), 1.94 (d, J = 9.2 Hz, 1H), 1.68-1.55 (m, 1H), 1.51- 1.38 (m, 1H), 1.34-1.23 (m, 1H) | 390.3 |
| | 1-(5-([1,2,4]triazolo[1,5-a]pyridin-6-yl)- | | |
| | 4-(6-methylpyridin-2-yl)-1H-imidazol-2- | | |
| | yl)-N-(3-tetrahydropyranyl)methanamine | | |
| 1.16 | 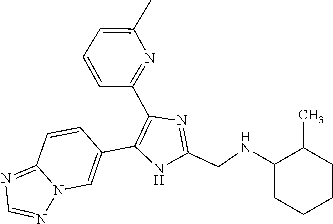 [SMILES: Cc1cccc(-c2nc(CNC3CCCCC3C)[nH]c2-c2ccc3ncnn3c2)n1] | ¹H NMR (DMSO-d₆, 400 MHz) δ 9.53 (s, 2H), 8.82 (s, 1H), 8.13-7.96 (m, 3H), 7.75 (d, J = 8.0 Hz, 2H), 7.58 (d, J = 8.0 Hz, 3H), 4.67-4.43 (m, 2H), 3.09-2.94 (m, 1H), 2.64 (s, 3H), 2.22 (d, J = 10.2 Hz, 1H), 1.75 (t, J = 13.2 Hz, 3H), 1.61-1.47 (m, 2H), 1.35 (s, 1H), 1.30-1.14 (m, 2H), 1.10 (d, J = 6.4 Hz, 3H), 1.04 (d, J = 7.2 Hz, 1H), 1.06-1.02 (m, 1H) | 402.3 |
| | 1-(5-([1,2,4]triazolo[1,5-a]pyridin-6-yl)- | | |
| | 4-(6-methylpyridin-2-yl)-1H-imidazol-2- | | |
| | yl)-N-(2-methylcyclohexyl)methanamine | | |
| 1.17 | 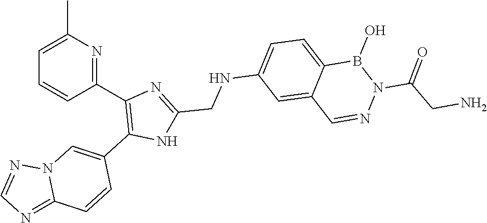 [SMILES: Cc1cccc(-c2nc(CNc3ccc4c(c3)C=NN(C(=O)CN)B4O)[nH]c2-c2ccc3ncnn3c2)n1] | | 507.2 |
| | 1-(6-(((5-([1,2,4]triazolo[1,5-a]pyridin-6-yl)-4-(6- | | |
| | methylpyridin-2-yl)-1H-imidazol-2- | | |
| | yl)methyl)amino)-1- | | |
| | hydroxybenzo[d][1,2,3]diazaborinin-2(1H)-yl)-2- | | |
| | aminoethan-1-one | | |
| 1.18 | 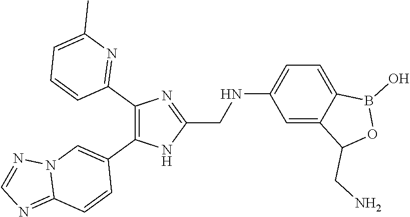 [SMILES: Cc1cccc(-c2nc(CNc3ccc4c(c3)C(CN)OB4O)[nH]c2-c2ccc3ncnn3c2)n1] | | 467.2 |
| | 5-(((5-([1,2,4]triazolo[1,5-a]pyridin-6-yl)-4-(6- | | |
| | methylpyridin-2-yl)-1H-imidazol-2- | | |
| | yl)methyl)amino)-3- | | |
| | (aminomethyl)benzo[c][1,2]oxaborol-1(3H)-ol | | |
| 1.19 | 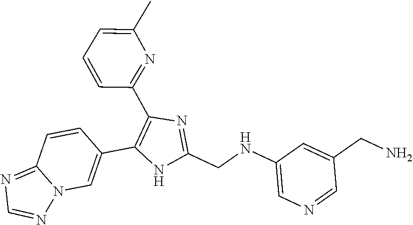 [SMILES: Cc1cccc(-c2nc(CNc3cncc(CN)c3)[nH]c2-c2ccc3ncnn3c2)n1] | ¹H NMR (DMSO-d₆, 400 MHz) δ 9.48 (s, 1H), 8.88 (br s, 3H), 8.64 (s, 1H), 8.42 (d, J = 2.4 Hz, 1H), 8.31 (s, 1H), 8.21 (s, 1H), 8.00-7.83 (m, 3H), 7.64 (d, J = 8.0 Hz, 1H), 7.43 (d, J = 8.0 Hz, 1H), 4.90 (s, 2H), 4.18 (d, J = 5.6 Hz, 1H), 4.21- 4.14 (m, 1H), 4.23-4.13 (m, 1H), 2.56 (s, 3H) | 412.1 |
| | N-((5-([1,2,4]triazolo[1,5-a]pyridin-6-yl)-4-(6- | | |
| | methylpyridin-2-yl)-1H-imidazol-2-yl)methyl)-5- | | |
| | (aminomethyl)pyridin-3-amine | | |
| 1.20 | 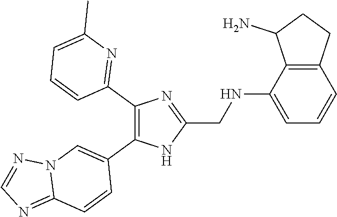 [SMILES: Cc1cccc(-c2nc(CNc3cccc4c3C(N)CC4)[nH]c2-c2ccc3ncnn3c2)n1] | ¹H NMR (DMSO-d₆, 400 MHz) δ 9.42 (s, 1H), 8.59 (s, 1H), 8.01 (s, 3H), 7.92 (d, J = 9.4 Hz, 1H), 7.80- 7.76 (m, 1H), 7.45 (d, J = 8.0 Hz, 1H), 7.27 (s, 1H), 7.31 (d, J = 7.6 Hz, 1H), 7.18 (t, J = 7.8 Hz, 1H), 6.66 (d, J = 7.6 Hz, 1H), 6.57 (d, J = 8.0 Hz, 1H), 4.89 (s, 1H), 4.66 (s, 2H), 3.12-3.16 (m, 1H), 2.78-2.83 (m, 1H), 2.53 (s, 3H), 2.42-2.32 (m, 1H), 2.12-2.06 (m, 1H) | 459.2 (M + Na) |
| | N7-((5-([1,2,4]triazolo[1,5-a]pyridin-6-yl)-4-(6- | | |
| | methylpyridin-2-yl)-1H-imidazol-2-yl)methyl)- | | |
| | 2,3-dihydro-1H-indene-1,7-diamine | | |
| 1.21 | 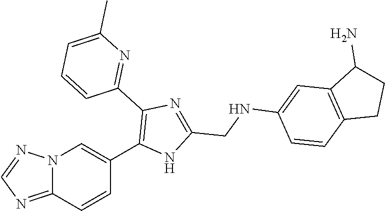 [SMILES: Cc1cccc(-c2nc(CNc3ccc4c(c3)C(N)CC4)[nH]c2-c2ccc3ncnn3c2)n1] | ¹H NMR (DMSO-d₆, 400 MHz) δ 9.54 (s, 1H), 8.49 (s, 1H), 7.99 (dd, J = 1.6, 9.4 Hz, 1H), 7.82 (d, J = 9.4 Hz, 1H), 7.70 (t, J = 7.8 Hz, 1H), 7.59-7.44 (m, 1H), 7.14 (d, J = 7.8 Hz, 1H), 6.92 (d, J = 8.0 Hz, 1H), 6.79 (s, 1H), 6.57 (dd, J = 1.8, 8.0 Hz, 1H), 5.89 (t, J = 5.8 Hz, 1H), 4.35 (d, J = 5.8 Hz, 2H), 4.12 (t, J = 7.4 Hz, 1H), 2.74-2.66 (m, 1H), 2.59-2.53 (m, 2H), 2.46 (s, 3H), 2.33-2.24 (m, 1H), 1.52-1.58 (m, 1H) | 459.1 (M + Na) |
| | N6-((5-([1,2,4]triazolo[1,5-a]pyridin-6-yl)-4-(6- | | |
| | methylpyridin-2-yl)-1H-imidazol-2-yl)methyl)- | | |
| | 2,3-dihydro-1H-indene-1,6-diamine | | |
| 1.22 | 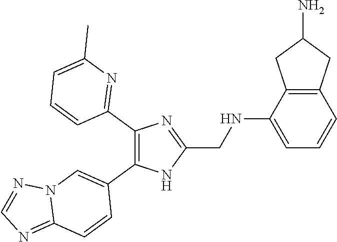 [SMILES: Cc1cccc(-c2nc(CNc3cccc4c3CC(N)C4)[nH]c2-c2ccc3ncnn3c2)n1] | ¹H NMR (DMSO-d₆, 400 MHz) δ 9.45 (s, 1H), 8.57 (s, 1H), 8.10 (s, 3H), 7.95-7.81 (m, 2H), 7.77 (t, J = 7.6 Hz, 1H), 7.47 (d, J = 8.0 Hz, 1H), 7.28 (d, J = 7.6 Hz, 1H), 7.04 (t, J = 7.6 Hz, 1H), 6.57 (dd, J = 7.6, 15.4 Hz, 2H), 4.58 (s, 3H), 4.01 (s, 2H), 3.29-3.13 (m, 2H), 2.92- 2.76 (m, 2H) | 437.2 |
| | N5-((5-([1,2,4]triazolo[1,5-a]pyridin-6-yl)-4-(6- | | |
| | methylpyridin-2-yl)-1H-imidazol-2-yl)methyl)- | | |
| | 2,3-dihydro-1H-indene-2,5-diamine | | |
| 1.23 | 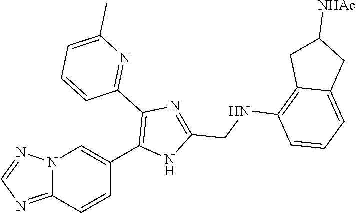 [SMILES: CC(=O)NC1Cc2cccc(NCc3nc(-c4cccc(C)n4)c(-c4ccc5ncnn5c4)[nH]3)c2C1] | ¹H NMR (MeOD, 400 MHz) δ 9.20 (s, 1H), 8.70-8.36 (m, 4H), 8.10- 7.93 (m, 2H), 7.85-7.71 (m, 1H), 7.85-7.71 (m, 1H), 7.38 (d, J = 7.6 Hz, 1H), 7.29 (br s, 1H), 5.38 (br d, J = 6.8 Hz, 1H), 5.32 (s, 2H), 3.98 (dd, J = 7.8, 15.6 Hz, 2H), 3.61-3.45 (m, 2H), 2.70 (s, 3H) | 479.3 |
| | N-(4-(((5-([1,2,4]triazolo[1,5-a]pyridin-6-yl)-4-(6- | | |
| | methylpyridin-2-yl)-1H-imidazol-2- | | |
| | yl)methyl)amino)-2,3-dihydro-1H-inden-2- | | |
| | yl)acetamide | | |
| 1.24 | 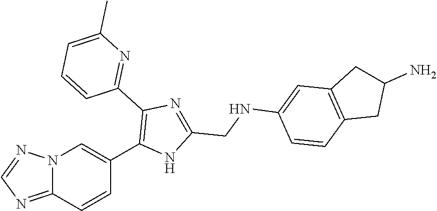 [SMILES: Cc1cccc(-c2nc(CNc3ccc4c(c3)CC(N)C4)[nH]c2-c2ccc3ncnn3c2)n1] | ¹H NMR (DMSO-d₆, 400 MHz) δ 9.41 (s, 1H), 8.61 (s, 1H), 8.06 (d, J = 3.2 Hz, 2H), 7.95 (dd, J = 0.8, 9.6 Hz, 1H), 7.83-7.78 (m, 2H), 7.46 (d, J = 8.0 Hz, 1H), 7.35 (d, J = 7.6 Hz, 1H), 7.03 (d, J = 8.0 Hz, 1H), 6.65 (s, 1H), 6.59 (dd, J = 2.4, 8.0 Hz, 1H), 4.62 (s, 2H), 3.94-3.92 (m, 1H), 3.19-3.11(m, 2H), 2.88- 2.73 (m, 2H), 2.52(s, 3H) | 437.2 |
| | N4-((5-([1,2,4]triazolo[1,5-a]pyridin-6-yl)-4-(6- | | |
| | methylpyridin-2-yl)-1H-imidazol-2-yl)methyl)- | | |
| | 2,3-dihydro-1H-indene-2,4-diamine | | |
| 1.25 | 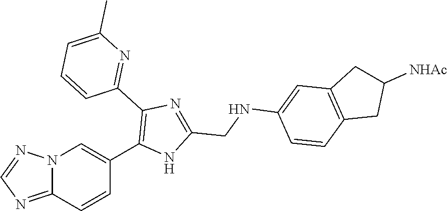 [SMILES: CC(=O)NC1Cc2ccc(NCc3nc(-c4cccc(C)n4)c(-c4ccc5ncnn5c4)[nH]3)cc2C1] | ¹H NMR (DMSO-d₆, 400 MHz) δ9.51 (br s, 1H), 8.52 (br s, 1H), 8.06 (br d, J = 6.0 Hz, 1H), 7.95 (d, J = 8.4 Hz, 1H), 7.89-7.79 (m, 1H), 7.79-7.67 (m, 1H), 7.51 (s, 1H), 7.20 (s, 2H), 7.08 (s, 1H), 6.99-6.88 (m, 2H), 6.70-6.49 (m, 2H), 4.36 (s, 3H), 3.11-2.93 (m, 2H), 2.68-2.56 (m, 3H), 2.46 (s, 3H), 1.77 (s, 3H) | 479.3 |
| | N-(5-(((5-([1,2,4]triazolo[1,5-a]pyridin-6-yl)-4-(6- | | |
| | methylpyridin-2-yl)-1H-imidazol-2- | | |
| | yl)methyl)amino)-2,3-dihydro-1H-inden-2- | | |
| | yl)acetamide | | |
| 1.26 | 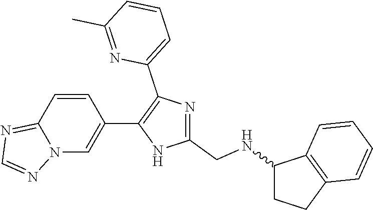 [SMILES: Cc1cccc(-c2nc(CNC3CCc4ccccc43)[nH]c2-c2ccc3ncnn3c2)n1] | 1H NMR (DMSO-d6, 400 MHz) δ 9.19 (s, 1H), 8.43 (s, 1H), 7.41 (s, 2H), 7.89 (d, J = 10.8 Hz, 1H), 7.78 (d, J = 9.2 Hz, 1H), 7.65 (t, J = 8.0 Hz, 1H), 7.44-7.41 (m, 1H), 7.21 (d, J = 2.0 Hz, 1H), 7.25-7.15 (m, 4H), 4.35 (t, J = 6.0 Hz, 1H), 4.05 (d, J = 2.0 Hz, 2H), 3.11-3.02 (m, 1H), 2.88-2.80 (m, 1H), 2.51 (s, 3H), 2.46-2.36 (m, 1H), 2.02-1.39 (m, 1H) | 422.2 |
| | rac-N-((5-([1,2,4]triazolo[1,5-a]pyridin-6-yl)-4- | | |
| | (6-methylpyridin-2-yl)-1H-imidazol-2-yl)methyl)- | | |
| | 2,3-dihydro-1H-inden-1-amine | | |
| 1.27 | 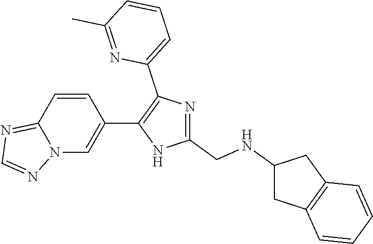 [SMILES: Cc1cccc(-c2nc(CNC3Cc4ccccc4C3)[nH]c2-c2ccc3ncnn3c2)n1] | 1H NMR (DMSO-d6, 400 MHz) δ 9.55 (s, 1H), 8.49 (s, 1H), 7.91 (d, J = 12.0 Hz, 1H), 7.82 (d, J = 8.8 Hz, 1H), 7.69 (t, J = 7.2 Hz, 1H), 7.65 (t, J = 8.0 Hz, 1H), 7.20-7.09 (m, 5H), 3.88 (s, 2H), 3.60 (t, J = 6.8 Hz, 1H), 3.10-3.03 (m, 2H), 2.75- 2.68 (m, 2H) | 422.2 |
| | N-((5-([1,2,4]triazolo[1,5-a]pyridin-6-yl)-4-(6- | | |
| | methylpyridin-2-yl)-1H-imidazol-2-yl)methyl)- | | |
| | 2,3-dihydro-1H-inden-2-amine | | |
| 1.30 | 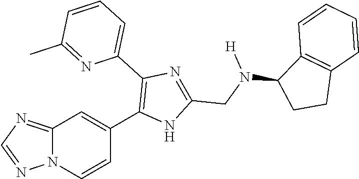 [SMILES: Cc1cccc(-c2nc(CN[C@@H]3CCc4ccccc43)[nH]c2-c2ccn3ncnc3c2)n1] | ¹H NMR (DMSO-d₆, 400 MHz) δ 9.56 (s, 1H), 8.50 (s, 1H), 8.01 (d, J = 8.0 Hz, 1H), 7.83 (d, J = 12.0 Hz, 1H), 7.72 (t, J = 7.2 Hz, 1H), 7.48-7.43 (m, 2H), 7.24-7.17 (m, 4H), 4.22 (t, J = 7.2 Hz, 1H), 3.91 (s, 2H), 3.01-2.93 (m, 1H), 2.77- 2.73 (m, 1H), 2.52 (s, 3H), 2.33- 2.29 (m, 1H), 1.82-1.79 (m, 1H) | 422.1 |
| | (R)-N-((5-([1,2,4]triazolo[1,5-a]pyridin-6-yl)-4- | | |
| | (6-methylpyridin-2-yl)-1H-imidazol-2-yl)methyl)- | | |
| | 2,3-dihydro-1H-inden-1-amine | | |
| 1.31 | 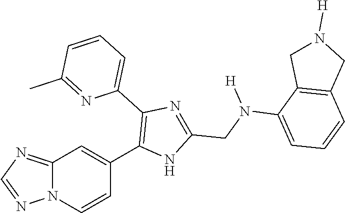 [SMILES: Cc1cccc(-c2nc(CNc3cccc4c3CNC4)[nH]c2-c2ccn3ncnc3c2)n1] | ¹H NMR (DMSO-d₆, 400 MHz) δ 9.43 (d, J = 1.0 Hz, 1H), 8.58 (s, 1H), 7.94-7.92 (m, 1H), 7.91-7.90 (m, 2H), 7.47 (d, J = 7.8 Hz, 1H), 7.31 (d, J = 7.8 Hz, 1H), 7.19 (t, J = 7.8 Hz, 1H), 6.68 (dd, J = 5.2, 7.6 Hz, 2H), 4.63 (s, 2H), 4.47 (t, J = 5.2 Hz, 2H), 4.42 (br t, J = 5.2 Hz, 2H), 2.53 (s, 3H) | 423.1 |
| | N-((5-([1,2,4]triazolo[1,5-a]pyridin-6-yl)-4-(6- | | |
| | methylpyridin-2-yl)-1H-imidazol-2- | | |
| | yl)methyl)isoindolin-4-amine | | |
| 1.32 | 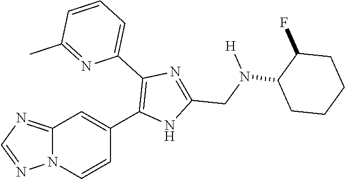 | ¹H NMR (DMSO-d₆, 400 MHz) δ 9.53 (s, 1H), 8.51 (s, 1H), 7.99 (d, J = 9.2 Hz, 1H), 7.84 (d, J = 9.2 Hz, 1H), 7.71 (t, J = 8.0 Hz, 1H), 7.44- 7.31 (m, 1H), 7.22-7.12 (m, 1H), 4.41-4.29 (m, 1H), 3.86 (d, J = 4.0 Hz, 2H), 2.68-2.59 (m, 1H), 2.56 (s, 3H), 2.03-1.95 (m, 3H), 1.69-1.55 (m, 2H), 1.18-1.13 (m, 4H) | 406.2 |
| | (1S,2S)-N-((5-([1,2,4]triazolo[1,5-a]pyridin-6-yl)- | | |
| | 4-(6-methylpyridin-2-yl)-1H-imidazol-2- | | |
| | yl)methyl)-2-fluorocyclohexan-1-amine | | |
| 1.33 | 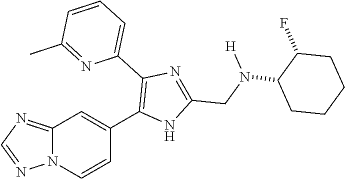 | ¹H NMR (MeOD, 400 MHz) δ 9.20 (s, 1H), 8.56 (s, 1H), 8.17 (t, J = 8.0 Hz, 1H), 7.97-7.81 (m, 2H), 7.68 (t, J = 8.6 Hz, 2H), 5.41-5.17 (m, 1H), 4.67-4.46 (m, 2H), 3.76-3.48 (m, 1H), 2.84 (s, 3H), 2.30-2.06 (m, 2H), 1.99-1.57 (m, 5H), 1.56-1.41 (m, 1H) | 406.1 |
| | (1S,2R)-N-((5-([1,2,4]triazolo[1,5-a]pyridin-6-yl)- | | |
| | 4-(6-methylpyridin-2-yl)-1H-imidazol-2- | | |
| | yl)methyl)-2-fluorocyclohexan-1-amine | | |
| 1.34 | 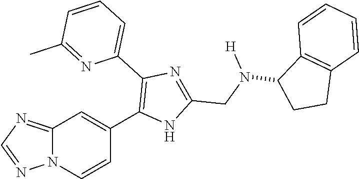 | | 422.1 |
| | (S)-N-((5-([1,2,4]triazolo[1,5-a]pyridin-6-yl)-4-(6- | | |
| | methylpyridin-2-yl)-1H-imidazol-2-yl)methyl)- | | |
| | 2,3-dihydro-1H-inden-1-amine | | |
| 1.35 | 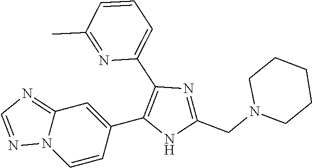 [SMILES: Cc1cccc(-c2nc(CN3CCCCC3)[nH]c2-c2ccn3ncnc3c2)n1] | | 374.2 |
| | 7-(4-(6-methylpyridin-2-yl)-2-(piperidin-1- | | |
| | ylmethyl)-1H-imidazol-5-yl)-[1,2,4]triazolo[1,5- | | |
| | a]pyridine | | |
| 1.36 | 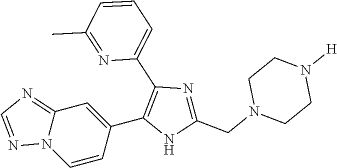 [SMILES: Cc1cccc(-c2nc(CN3CCNCC3)[nH]c2-c2ccn3ncnc3c2)n1] | | 375.2 |
| | 7-(4-(6-methylpyridin-2-yl)-2-(piperazin-1- | | |
| | ylmethyl)-1H-imidazol-5-yl)-[1,2,4]triazolo[1,5- | | |
| | a]pyridine | | |
| 1.37 | 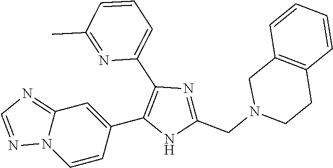 [SMILES: Cc1cccc(-c2nc(CN3CCc4ccccc4C3)[nH]c2-c2ccn3ncnc3c2)n1] | | 422.2 |
| | 2-((5-([1,2,4]triazolo[1,5-a]pyridin-7-yl)-4-(6- | | |
| | methylpyridin-2-yl)-1H-imidazol-2-yl)methyl)- | | |
| | 1,2,3,4-tetrahydroisoquinoline | | |
| 1.38 | 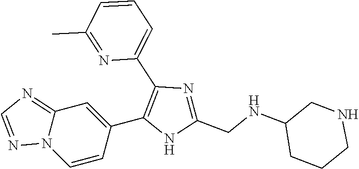 [SMILES: Cc1cccc(-c2nc(CNC3CCCNC3)[nH]c2-c2ccn3ncnc3c2)n1] | | 389.2 |
| | N-((5-([1,2,4]triazolo[1,5-a]pyridin-7-yl)-4-(6- | | |
| | methylpyridin-2-yl)-1H-imidazol-2- | | |
| | yl)methyl)piperidin-3-amine | | |
| 1.39 | 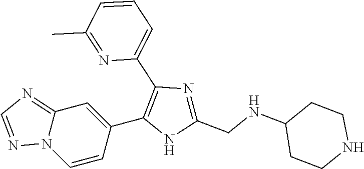 [SMILES: Cc1cccc(-c2nc(CNC3CCNCC3)[nH]c2-c2ccn3ncnc3c2)n1] | | 389.2 |
| | N-((5-([1,2,4]triazolo[1,5-a]pyridin-7-yl)-4-(6- | | |
| | methylpyridin-2-yl)-1H-imidazol-2- | | |
| | yl)methyl)piperidin-4-amine | | |
| 1.40 | 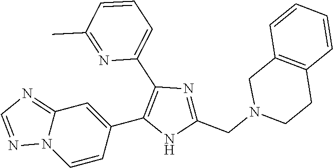 [SMILES: Cc1cccc(-c2nc(CN3CCc4ccccc4C3)[nH]c2-c2ccn3ncnc3c2)n1] | | 422.2 |
| | 2-((5-([1,2,4]triazolo[1,5-a]pyridin-7-yl)-4-(6- | | |
| | methylpyridin-2-yl)-1H-imidazol-2-yl)methyl)- | | |
| | 1,2,3,4-tetrahydroisoquinoline | | |
| 1.41 | 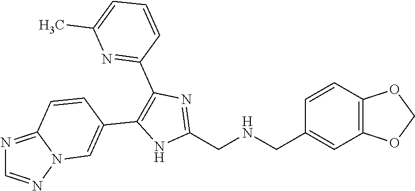 [SMILES: Cc1cccc(-c2nc(CNCc3ccc4c(c3)OCO4)[nH]c2-c2ccc3ncnn3c2)n1] | ¹H NMR (MeOD, 400 MHz) δ 9.20 (s, 1H), 8.53 (s, 1H), 8.10 (t, J = 8.0 Hz, 1H), 7.94-7.84 (m, 2H), 7.68- 7.59 (m, 2H), 7.06-7.03 (m, 2H), 6.91-6.87 (m, 1H), 6.00 (s, 2H), 4.46 (s, 2H), 4.39 (s, 2H), 2.79 (s, 3H) | 440.1 |
| | 1-(5-([1,2,4]triazolo[1,5-a]pyridin-6-yl)-4-(6- | | |
| | methylpyridin-2-yl)-1H-imidazol-2-yl)-N- | | |
| | (benzo[d][1,3]dioxol-5-ylmethyl)methanamine | | |
| 1.42 | 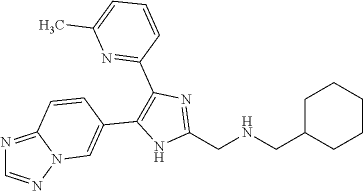 [SMILES: Cc1cccc(-c2nc(CNCC3CCCCC3)[nH]c2-c2ccc3ncnn3c2)n1] | ¹H NMR (MeOD, 400 MHz) δ 9.20 (s, 1H), 8.53 (s, 1H), 8.10 (t, J = 8.0 Hz, 1H), 7.93-7.89(m, 1H), 7.88- 7.84 (m, 1H), 7.62 (dd, J = 8.0, 19.3 Hz, 2H), 4.47 (s, 2H), 3.11 (d, J = 6.8 Hz, 2H), 2.79 (s, 3H), 1.92-1.77 (m, 5H), 1.73 (d, J = 12.0 Hz, 1H), 1.41-1.21 (m, 3H), 1.16-1.02 (m, 2H) | 402.1 |
| | 1-(5-([1,2,4]triazolo[1,5-a]pyridin-6-yl)-4-(6- | | |
| | methylpyridin-2-yl)-1H-imidazol-2-yl)-N- | | |
| | (cyclohexylmethyl)methanamine | | |
| 1.43 | 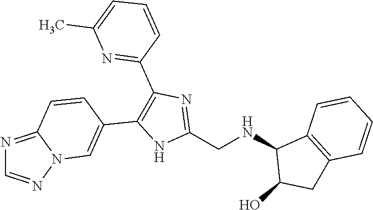 [SMILES: Cc1cccc(-c2nc(CN[C@H]3c4ccccc4C[C@H]3O)[nH]c2-c2ccc3ncnn3c2)n1] | ¹H NMR (MeOD, 400 MHz) δ 9.24- 9.14 (m, 1H), 8.55 (s, 1H), 8.15 (t, J = 8.0 Hz, 1H), 7.96-7.90 (m, 1H), 7.88-7.84 (m, 1H), 7.71-7.62 (m, 3H), 7.45-7.32 (m, 3H), 4.95 (dd, J = 4.8, 6.1 Hz, 2H), 4.65 (d, J = 1.2 Hz, 2H), 3.35 (d, J = 6.0 Hz, 1H), 3.30 (s, 1H), 3.18 (d, J = 4.8 Hz, 1H), 3.14 (d, J = 4.8 Hz, 1H), 2.82 (s, 3H) | 438.1 |
| | (1S,2R)-1-(((5-([1,2,4]triazolo[1,5-a]pyridin-6-yl)- | | |
| | 4-(6-methylpyridin-2-yl)-1H-imidazol-2- | | |
| | yl)methyl)amino)-2,3-dihydro-1H-inden-2-ol | | |
| 1.44 | 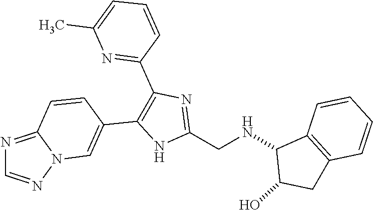 [SMILES: Cc1cccc(-c2nc(CN[C@@H]3c4ccccc4C[C@@H]3O)[nH]c2-c2ccc3ncnn3c2)n1] | ¹H NMR (MeOD, 400 MHz) δ 9.19 (s, 1H), 8.54 (s, 1H), 8.15 (t, J = 8.0 Hz, 1H), 7.96-7.81 (m, 2H), 7.71- 7.61 (m, 3H), 7.47-7.33 (m, 3H), 4.95-4.89 (s, 2H), 4.65 (s, 2H), 3.35 (d, J = 6.4 Hz, 1H), 3.16 (dd, J = 4.8, 16.4 Hz, 1H), 2.82 (s, 3H) | 438.1 |
| | (1R,2S)-1-(((5-([1,2,4]triazolo[1,5-a]pyridin-6-yl)- | | |
| | 4-(6-methylpyridin-2-yl)-1H-imidazol-2- | | |
| | yl)methyl)amino)-2,3-dihydro-1H-inden-2-ol | | |
| 1.45 | 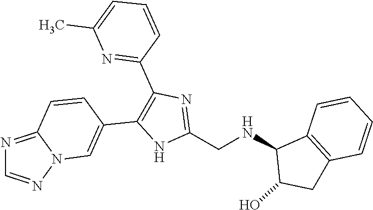 [SMILES: Cc1cccc(-c2nc(CN[C@H]3c4ccccc4C[C@@H]3O)[nH]c2-c2ccc3ncnn3c2)n1] | ¹H NMR (MeOD, 400 MHz) δ 9.21 (s, 1H), 8.54 (s, 1H), 8.09 (t, J = 8.0 Hz, 1H), 7.92-7.84 (m, 2H), 7.67- 7.64 (m, 2H), 7.59 (d, J = 7.8 Hz, 1H), 7.45-7.36 (m, 3H), 4.87-4.81 (m, 1H), 4.74 (q, J = 14.8 Hz, 2H), 3.57-3.48 (m, 1H), 3.07-2.96 (m, 1H), 2.78 (s, 3H) | 438.1 |
| | (1S,2S)-1-(((5-([1,2,4]triazolo[1,5-a]pyridin-6-yl)- | | |
| | 4-(6-methylpyridin-2-yl)-1H-imidazol-2- | | |
| | yl)methyl)amino)-2,3-dihydro-1H-inden-2-ol | | |
| 1.46 | 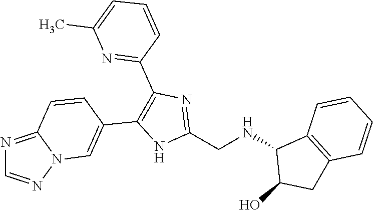 [SMILES: Cc1cccc(-c2nc(CN[C@@H]3c4ccccc4C[C@H]3O)[nH]c2-c2ccc3ncnn3c2)n1] | ¹H NMR (MeOD, 400 MHz) δ 9.25- 9.18 (m, 1H), 8.44 (s, 1H), 7.91 (dd, J = 1.6, 9.2 Hz, 1H), 7.78 (dd, J = 0.8, 9.2 Hz, 1H), 7.68 (t, J = 7.6 Hz, 1H), 7.46-7.38 (m, 2H), 7.25-7.22 (m, 3H), 7.19 (d, J = 7.6 Hz, 1H), 4.43 (td, J = 5.4, 6.4 Hz, 1H),4.20 (s, 2H), 4.16 (d, J = 4.6 Hz, 1H), 3.36 (d, J = 6.8 Hz, 1H), 3.32-3.30 (m, 1H), 2.81 (dd, J = 5.6, 15.8 Hz, 1H), 2.53 (s, 3H) | 438.2 |
| | (1R,2R)-1-(((5-([1,2,4]triazolo[1,5-a]pyridin-6- | | |
| | yl)-4-(6-methylpyridin-2-yl)-1H-imidazol-2- | | |
| | yl)methyl)amino)-2,3-dihydro-1H-inden-2-ol | | |
| 1.56 | 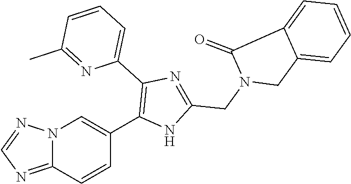 [SMILES: Cc1cccc(-c2nc(CN3Cc4ccccc4C3=O)[nH]c2-c2ccc3ncnn3c2)n1] | ¹H NMR (MeOD, 400 MHz) δ 9.18 (d, J = 1.6 Hz, 1H), 8.50 (s, 1H), 7.90 (t, J = 8.0 Hz 1H), 8.82 (s, 2H), 7.89-7.80 (m, 3H), 7.67-7.60 (m, 2H), 7.56-7.47 (m, 3H), 5.09 (s, 2H), 4.71 (s, 2H), 2.69 (s, 3H) | 422.2 |
| | 2-((5-([1,2,4]triazolo[1,5-a]pyridin-6-yl)-4-(6- | | |
| | methylpyridin-2-yl)-1H-imidazol-2- | | |
| | yl)methyl)isoindolin-1-one | | |
| 1.57 | 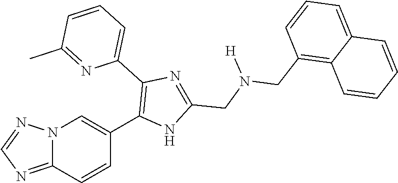 [SMILES: Cc1cccc(-c2nc(CNCc3cccc4ccccc34)[nH]c2-c2ccc3ncnn3c2)n1] | ¹H NMR (MeOD, 400 MHz): δ 9.14 (d, J = 1.2 Hz, 1H), 8.42 (s, 1H), 8.15 (d, J = 8.0 Hz, 1H), 7.87-7.81 (m, 2H), 7.78-7.73 (m, 2H), 7.69- 7.62 (m, 1H), 7.55-7.37 (m, 5H), 7.34-7.29 (m, 1H), 7.19-7.15 (m, 1H), 4.32 (s, 2H), 4.05 (s, 2H), 2.51 (s, 3H) | 446.2 |
| | 1-(5-([1,2,4]triazolo[1,5-a]pyridin-6-yl)-4-(6- | | |
| | methylpyridin-2-yl)-1H-imidazol-2-yl)-N- | | |
| | (naphthalen-1-ylmethyl)methanamine | | |
| 1.58 | 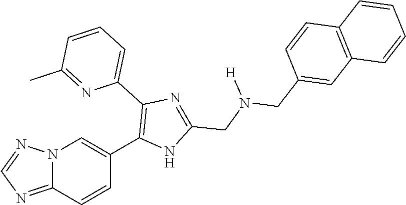 [SMILES: Cc1cccc(-c2nc(CNCc3ccc4ccccc4c3)[nH]c2-c2ccc3ncnn3c2)n1] | ¹H NMR (MeOD, 400 MHz) δ 9.16 (s, 1H), 8.51 (s, 3H), 8.06 (s, 2H), 7.97-7.85 (m, 4H), 7.85-7.84 (m, 2H), 7.64-7.58(m, 1H), 7.57-.55 (m, 3H), 7.55-7.53 (m, 1H), 4.65 (s, 2H), 4.51 (s, 2H), 2.70 (s, 3H) | 446.2 |
| | 1-(5-([1,2,4]triazolo[1,5-a]pyridin-6-yl)-4-(6- | | |
| | methylpyridin-2-yl)-1H-imidazol-2-yl)-N- | | |
| | (naphthalen-2-ylmethyl)methanamine | | |
| 1.59 | 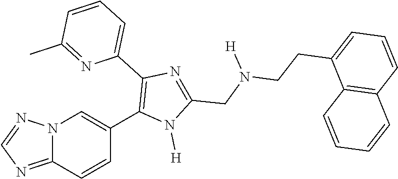 [SMILES: Cc1cccc(-c2nc(CNCCc3cccc4ccccc34)[nH]c2-c2ccc3ncnn3c2)n1] | ¹H NMR (MeOD, 400 MHz) δ 9.13 (s, 1H), 8.43 (s, 1H), 8.08 (d, J = 7.4 Hz, 1H), 7.83-7.81 (m, 2H), 7.76-7.70 (m, 2H), 7.65 (t, J = 7.8 Hz, 1H), 7.46-7.41 (m, 2H), 7.38 (d, J = 5.4 Hz, 2H), 7.32 (d, J = 7.8 Hz, 1H), 7.18 (d, J = 7.8 Hz, 1H), 3.98 (s, 1H), 3.35-3.33 (m, 2H), 3.04 (t, J = 7.4 Hz, 2H), 2.51 (s, 3H) | 460.3 |
| | N-((5-([1,2,4]triazolo[1,5-a]pyridin-6-yl)-4-(6- | | |
| | methylpyridin-2-yl)-1H-imidazol-2-yl)methyl)-2- | | |
| | (naphthalen-1-yl)ethan-1-amine | | |
| 1.60 | 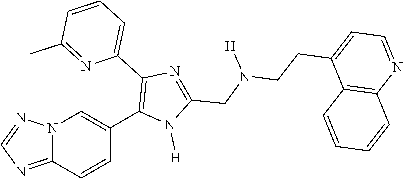 [SMILES: Cc1cccc(-c2nc(CNCCc3ccnc4ccccc34)[nH]c2-c2ccc3ncnn3c2)n1] | ¹H NMR (MeOD, 400 MHz) δ 9.15 (s, 1H), 8.73 (d, J = 4.4 Hz, 1H), 8.44 (s, 1H), 8.21 (d, J = 8.4 Hz, 1H), 8.01 (d, J = 8.4 Hz, 1H), 7.84 (dd, J = 1.6, 9.2 Hz, 1H), 7.78-7.58 (m, 4H), 7.46 (d, J = 4.4 Hz, 1H), 7.33 (d, J = 8.0 Hz, 1H), 7.18 (d, J = 7.6 Hz, 1H), 4.00 (s. 2H), 3.40 (t, J = 7.6 Hz, 2H), 3.12 (t, J = 7.6 Hz, 2H), 2.52 (s, 3H) | 461.3 |
| | N-((5-([1,2,4]triazolo[1,5-a]pyridin-6-yl)-4-(6- | | |
| | methylpyridin-2-yl)-1H-imidazol-2-yl)methyl)-2- | | |
| | (quinolin-4-yl)ethan-1-amine | | |
| 1.61 | 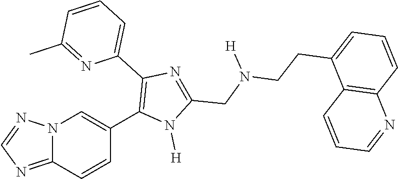 [SMILES: Cc1cccc(-c2nc(CNCCc3cccc4ncccc34)[nH]c2-c2ccc3ncnn3c2)n1] | ¹H NMR (DMSO-d6, 400 MHz) δ 9.21 (s, 1H), 8.84 (d, J = 2.8 Hz, 1H), 8.65 (d, J = 8.4 Hz, 1H), 8.45 (s, 1H), 7.96 (d, J = 8.4 Hz, 1H), 7.88 (dd, J = 1.6, 9.2 Hz, 1H), 7.79- 7.66 (m, 3H), 7.59 (d, J = 7.2 Hz, 1H), 7.55 (dd, J = 4.4, 8.4 Hz, 1H), 7.38 (d, J = 7.6 Hz, 1H), 7.21 (d, J = 7.6 Hz, 1H), 4.27 (s. 2H), 3.51 (t, J = 7.6 Hz, 2H), 3.36 (t, J = 7.6 Hz, 2H), 2.54 (s, 3H) | 461.3 |
| | N-((5-([1,2,4]triazolo[1,5-a]pyridin-6-yl)-4-(6- | | |
| | methylpyridin-2-yl)-1H-imidazol-2-yl)methyl)-2- | | |
| | (quinolin-5-yl)ethan-1-amine | | |
| 1.63 | 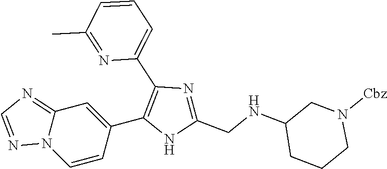 [SMILES: Cc1cccc(-c2nc(CNC3CCCN(C(=O)OCc4ccccc4)C3)[nH]c2-c2ccn3ncnc3c2)n1] | | 523.2 |
| | benzyl 3-(((5-([1,2,4]triazolo[1,5-a]pyridin-7-yl)- | | |
| | 4-(6-methylpyridin-2-yl)-1H-imidazol-2- | | |
| | yl)methyl)amino)piperidine-1-carboxylate | | |
| 1.64 | 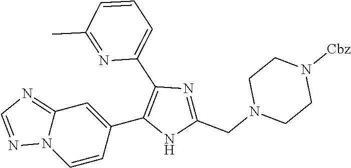 [SMILES: Cc1cccc(-c2nc(CN3CCN(C(=O)OCc4ccccc4)CC3)[nH]c2-c2ccn3ncnc3c2)n1] | | 509.2 |
| | benzyl 4-((5-([1,2,4]triazolo[1,5-a]pyridin-7-yl)-4- | | |
| | (6-methylpyridin-2-yl)-1H-imidazol-2- | | |
| | yl)methyl)piperazine-1-carboxylate | | |
| 1.65 | 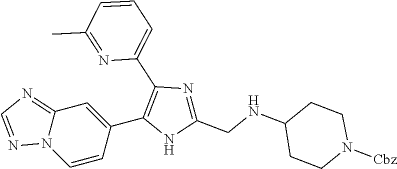 [SMILES: Cc1cccc(-c2nc(CNC3CCN(C(=O)OCc4ccccc4)CC3)[nH]c2-c2ccn3ncnc3c2)n1] | | 523.2 |
| | benzyl 4-(((5-([1,2,4]triazolo[1,5-a]pyridin-7-yl)- | | |
| | 4-(6-methylpyridin-2-yl)-1H-imidazol-2- | | |
| | yl)methyl)amino)piperidine-1-carboxylate | | |
| 1.66 | 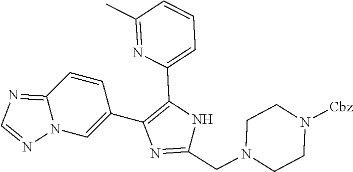 [SMILES: Cc1cccc(-c2[nH]c(CN3CCN(C(=O)OCc4ccccc4)CC3)nc2-c2ccc3ncnn3c2)n1] | | 509.2 |
| | benzyl 4-((4-([1,2,4]triazolo[1,5-a]pyridin-6-yl)-5- | | |
| | (6-methylpyridin-2-yl)-1H-imidazol-2- | | |
| | yl)methyl)piperazine-1-carboxylate | | |

### General Scheme 2 for the Preparation of exemplary ALK5 Inhibitors

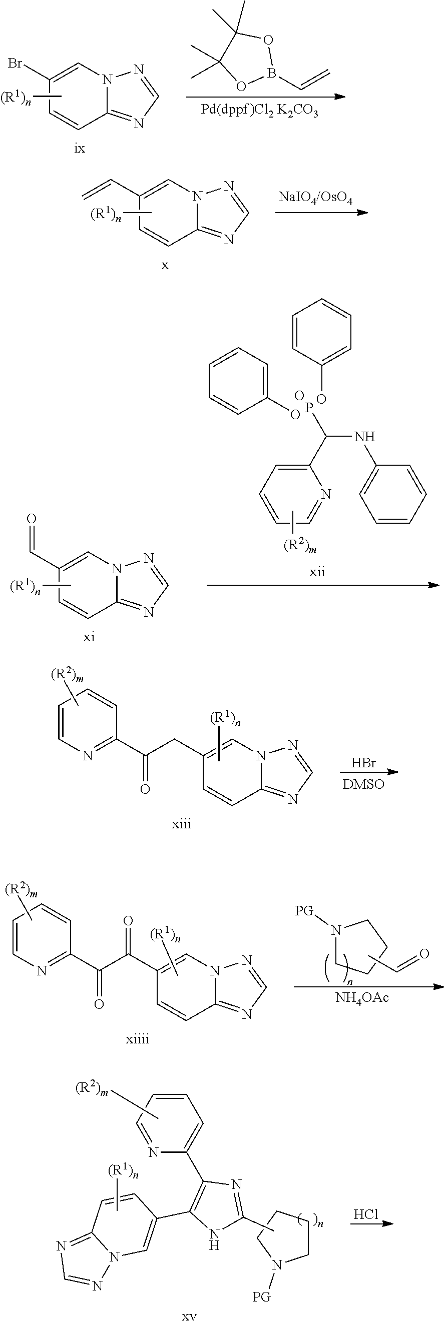

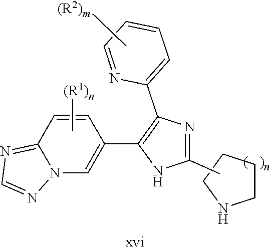

In one method, compounds of Formula (I) are prepared according to Scheme 2. Specifically, R²-substituted pyridine-2-carbaldehydes are reacted with aniline and diphenyl phosphite to give N,P-acetal (xii), which can be further coupled with le substituted [1,2,4]triazolo[1,5-a]pyridine-6-carbaldehydes (xi) followed by hydrolysis in acidic condition to produce a monoketone (xiii). The monoketone (xiii) may be oxidized to a diketone (xiv) with HBr in DMSO. Certain diketone analogs (vi) were commercially available (e.g. R¹=H and R²=CH³: CAS No. 356560-84-4) and were used as a starting point when appropriate. Diketones (vi) can be condensed with an appropriate protected-amine-containing aliphatic aldehydes in the presence of ammonium acetate to yield a compound of the structure illustrated as xv. Subsequent deprotection of the aliphatic amine yields compound (xvi).

### EXAMPLE 2

### Synthesis of 6-(4-(6-methylpyridin-2-yl)-2-(piperidin-4-yl)-1H-imidazol-5-yl)-[1,2,4]triazolo[1,5-a]pyridine (Compound 1.47)

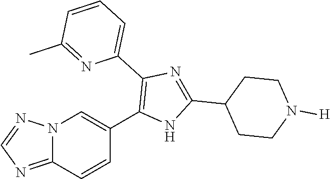 [SMILES: Cc1cccc(-c2nc(C3CCNCC3)[nH]c2-c2ccc3ncnn3c2)n1]

### Step A. Preparation of Int. 1.47a

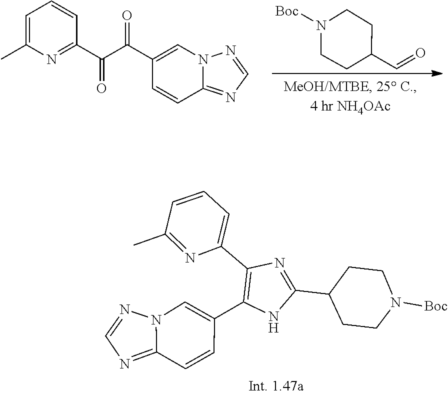 [SMILES: Cc1cccc(C(=O)C(=O)c2ccc3ncnn3c2)n1 ; CC(C)(C)OC(=O)N1CCC(C=O)CC1 ; Cc1cccc(-c2nc(C3CCN(C(=O)OC(C)(C)C)CC3)[nH]c2-c2ccc3ncnn3c2)n1]

To a solution of 1-(6-methyl-2-pyridyl)-2-([1,2,4]triazolo[1,5-a]pyridin-6-yl) ethane-1,2-dione (0.2 g, 751.16 umol, 1 eq) and tert-butyl 4-formylpiperidine-l-carboxylate (160.20 mg, 751.16 umol, 1 eq) in MTBE (5 mL) and MeOH (10 mL) was added NH₄OAc (289.51 mg, 3.76 mmol, 5 eq). The mixture was stirred at 25° C. for 4 hr. The reaction mixture was quenched by addition H₂O20 mL, extracted with EtOAc (20 mL×3). The combined organic layers were washed with brine (50mL×1), dried over Na₂SO₄, filtered and concentrated under reduced pressure to give a residue. Compound tert-butyl 4-(5-([1,2,4]triazolo[1,5-a]pyridin-6-yl)-4-(6-methylpyridin-2-yl)-1H-imidazol-2-yl)piperidine-1-carboxylate (0.3 g, crude) was obtained as a yellow solid

### Step B. Preparation of Compound 1.47

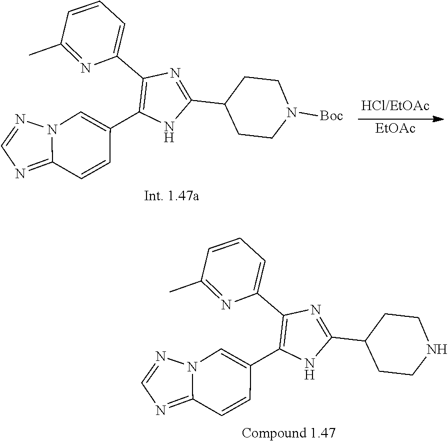 [SMILES: "EtOAc" ; Cc1cccc(-c2nc(C3CCN(C(=O)OC(C)(C)C)CC3)[nH]c2-c2ccc3ncnn3c2)n1 ; Cc1cccc(-c2nc(C3CCNCC3)[nH]c2-c2ccc3ncnn3c2)n1]

To a solution of tert-butyl 4-[4-(6-methyl-2-pyridyl)-5-([1,2,4]triazolo[1,5-a]pyridin-6-yl)-1H-imidazol-2-yl]piperidine-1-carboxylate (Int. 1.47a) (0.3 g, 652.82 umol, 1 eq) in DCM (10 mL) was added HCl/EtOAc (4 M, 326.41 uL, 2 eq). The mixture was stirred at 25° C. for 1 hr. The reaction mixture was concentrated under reduced pressure to give a residue. The residue was purified by prep-HPLC (column: Nano-micro Kromasil C18 100*30 mm 5 um; mobile phase: [water (0.1% TFA) -ACN]; B%: 1%-20%, 10 min) and freezen drying. Compound 6-[4-(6-methyl-2-pyridyl)-2-(4-piperidyl)-1H-imidazol-5-yl]-[1,2,4]triazolo[1,5-a]pyridine (Compound 1.47) (0.17 g, 359.07 umol, 55.00% yield, TFA) was obtained as a yellow solid. ¹H NMR (CDCl₃, 400 MHz) δ 9.44 (s, 1H), 8.94 (s, 1H), 8.65 (d, J=6.0 Hz, 1H), 8.60 (s, 1H), 7.94 (d, J=9.2 Hz, 1H), 7.87-7.80 (m, 3H), 7.52-7.47 (m, 1H), 7.36 (d, J=7.6 Hz, 1H), 3.86 (s, 1H), 3.46 (d, J=12.4 Hz, 2H), 3.41-3.33 (m, 1H), 3.17-3.04 (m, 2H), 2.56-2.53 (m, 3H), 2.27 (d, J=12.4 Hz, 2H), 2.11-2.00 (m, 2H); HPLC: 96.356% (220 nm), 98.486% (254 nm); MS (ESI): mass calcd. For C₂₀H₂₁N₇359.19, m/z found 360.0[M+H]⁺.

The compounds in Table 2 were prepared in a manner similar to that described for compound 1.47.

TABLE 2
Compounds prepared according to the methods described herein.

| | | | MS |
|---|---|---|---|
| Cmpd | Structure and Name | ¹H NMR | (M + H) |
| 1.47 | 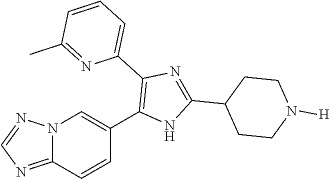 [SMILES: Cc1cccc(-c2nc(C3CCNCC3)[nH]c2-c2ccc3ncnn3c2)n1] | ¹H NMR (CDCl₃, 400 MHz) δ 9.44 (s, 1H), 8.94 (s, 1H), 8.65 (d, J = 6.0 Hz, 1H), 8.60 (s, 1H), 7.94 (d, J = 9.2 Hz, 1H), 7.87-7.80 (m, 3H), 7.52-7.47 (m, 1H), 7.36 (d, J = 7.6 Hz, 1H), 3.86 (s, 1H), 3.46 (d, J = 12.4 Hz, 2H), 3.41- 3.33 (m, 1H), 3.17-3.04 (m, 2H), 2.56- 2.53 (m, 3H), 2.27 (d, J = 12.4 Hz, 2H), 2.11-2.00 (m, 2H) | 360.0 |
| | 6-(4-(6-methylpyridin-2-yl)-2-(piperidin-4-yl)- | | |
| | 1H-imidazol-5-yl)-[1,2,4]triazolo[1,5-a]pyridine | | |
| 1.48 | 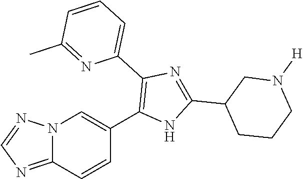 [SMILES: Cc1cccc(-c2nc(C3CCCNC3)[nH]c2-c2ccc3ncnn3c2)n1] | ¹H NMR (MeOD, 400 MHz) δ 9.20- 9.18 (m, 1H), 8.54 (s, 1H), 8.11-8.06 (m, 1H), 7.94-7.91 (m, 1H), 7.85-7.82 (m, 1H), 7.61 (t, J = 8.4 Hz, 2H), 4.00 (s, 1H), 3.70-3.49 (m, 3H), 3.40-3.34 (m, 1H), 3.27-3.18 (m, 1H), 2.80 (s, 3H), 2.27-2.32 (m, 1H), 2.08-2.00 (m, 2H), 2.00-1.83 (m, 1H) | 360.0 |
| | 6-(4-(6-methylpyridin-2-yl)-2-(piperidin-3-yl)- | | |
| | 1H-imidazol-5-yl)-[1,2,4]triazolo[1,5-a]pyridine | | |
| 1.49 | 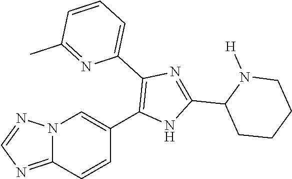 [SMILES: Cc1cccc(-c2nc(C3CCCCN3)[nH]c2-c2ccc3ncnn3c2)n1] | ¹H NMR (DMSO-d₆, 400 MHz) δ 9.50 (s, 1H), 9.46-9.08 (m, 1H), 8.55 (s, 1H), 7.96 (d, J = 9.6 Hz 1H), 7.92- 7.87 (m, 1H), 7.80 (t, J = 7.8 Hz, 1H), 7.55 (d, J = 8.0 Hz, 1H), 7.27 (d, J = 7.6 Hz, 1H), 4.95-4.36 (m, 1H), 3.87 (s, 1H), 3.43-3.39 (m, 1H), 3.22-3.10 (m, 1H), 2.53 (s, 3H), 2.33-2.30 (m, 1H), 1.94-1.71 (m, 5H) | 360.0 |
| | 6-(4-(6-methylpyridin-2-yl)-2-(piperidin-2-yl)- | | |
| | 1H-imidazol-5-yl)-[1,2,4]triazolo[1,5-a]pyridine | | |
| 1.50 | 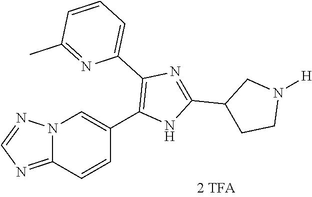 [SMILES: Cc1cccc(-c2nc(C3CCNC3)[nH]c2-c2ccc3ncnn3c2)n1] | ¹H NMR (DMSO-d₆, 400 MHz) δ 9.47 (s, 1H), 9.27 (s, 2H), 8.59 (s, 1H), 7.94-7.85 (m, 3H), 7.53 (d, J = 8.0 Hz, 2H), 7.38 (d, J = 7.6 Hz, 2H), 3.85- 3.69 (m, 1H), 3.58-3.28 (m, 4H), 2.57 (s, 3H), 2.48-2.42 (m, 1H), 2.36-2.26 (m, 1H) | 346.1 |
| | 6-(4-(6-methylpyridin-2-yl)-2-(pyrrolidin-3-yl)- | | |
| | 1H-imidazol-5-yl)-[1,2,4]triazolo[1,5-a]pyridine | | |
| | TFA salt | | |
| 1.51 | 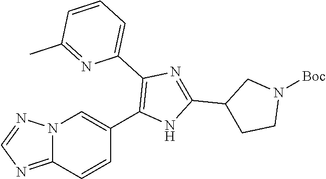 [SMILES: Cc1cccc(-c2nc(C3CCN(C(=O)OC(C)(C)C)C3)[nH]c2-c2ccc3ncnn3c2)n1] | | 468.2 (M + Na) |
| | tert-butyl 3-(5-([1,2,4]triazolo[1,5-a]pyridin-6- | | |
| | yl)-4-(6-methylpyridin-2-yl)-1H-imidazol-2- | | |
| | yl)pyrrolidine-1-carboxylate | | |
| 1.52 | 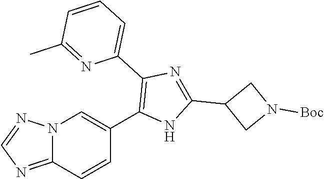 [SMILES: Cc1cccc(-c2nc(C3CN(C(=O)OC(C)(C)C)C3)[nH]c2-c2ccc3ncnn3c2)n1] | | 454.2 |
| | tert-butyl 3-(5-([1,2,4]triazolo[1,5-a]pyridin-6- | | |
| | yl)-4-(6-methylpyridin-2-yl)-1H-imidazol-2- | | |
| | yl)azetidine-1-carboxylate | | |
| 1.53 | 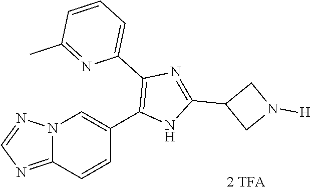 [SMILES: Cc1cccc(-c2nc(C3CNC3)[nH]c2-c2ccc3ncnn3c2)n1] | | 332.1 |
| | 6-(2-(azetidin-3-yl)-4-(6-methylpyridin-2-yl)- | | |
| | 1H-imidazol-5-yl)-[1,2,4]triazolo[1,5-a]pyridine | | |
| | TFA salt | | |
| 1.54 | 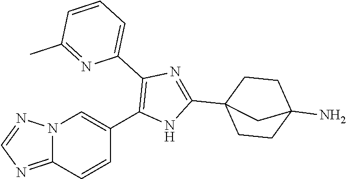 [SMILES: Cc1cccc(-c2nc(C34CCC(N)(CC3)C4)[nH]c2-c2ccc3ncnn3c2)n1] | | 386.2 |
| | 4-(5-([1,2,4]triazolo[1,5-a]pyridin-6-yl)-4-(6- | | |
| | methylpyridin-2-yl)-1H-imidazol-2- | | |
| | yl)bicyclo[2.2.1]heptan-1-amine | | |
| 1.55 | 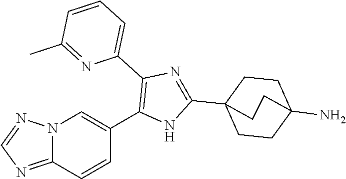 [SMILES: Cc1cccc(-c2nc(C34CCC(N)(CC3)CC4)[nH]c2-c2ccc3ncnn3c2)n1] | ¹H NMR (MeOD, 400 MHz) δ 9.22 (s, 1H), 8.55 (s, 1H), 7.97-7.88 (m, 2H), 7.80 (dd, J = 1.6, J = 9.2 Hz, 1H), 7.51-7.48 (m, 2H), 2.69 (s, 3H), 2.37- 2.26 (m, 6H), 2.09-1.97 (m, 6H) | 400.3 |
| | 4-(5-([1,2,4]triazolo[1,5-a]pyridin-6-yl)-4-(6- | | |
| | methylpyridin-2-yl)-1H-imidazol-2- | | |
| | yl)bicyclo[2.2.2]octan-1-amine | | |

### EXAMPLE 3

### Synthesis of Linker-Modified Payloads

Synthesis of 4-((S)-2-((S)-2-(6-(2,5-dioxo-2,5-dihydro-1H-pyrrol-1-yl)hexanamido)-3-methylbutanamido)-5-ureidopentanamido)benzyl 7-(((5-([1,2,4]triazolo[1,5-a]pyridin-6-yl)-4-(6-methylpyridin-2-yl)-1H-imidazol-2-yl)methyl)amino)-3,4-dihydroisoquinoline-2(1H)-carboxylate, trifluoroacetate salt (Compound 2.1)

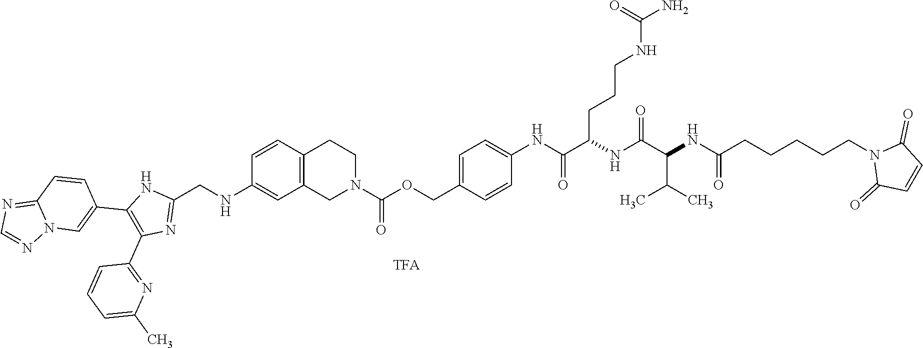 [SMILES: Cc1cccc(-c2nc(CNc3ccc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)[nH]c2-c2ccc3ncnn3c2)n1]

### Step A. Preparation of Compound 2.1

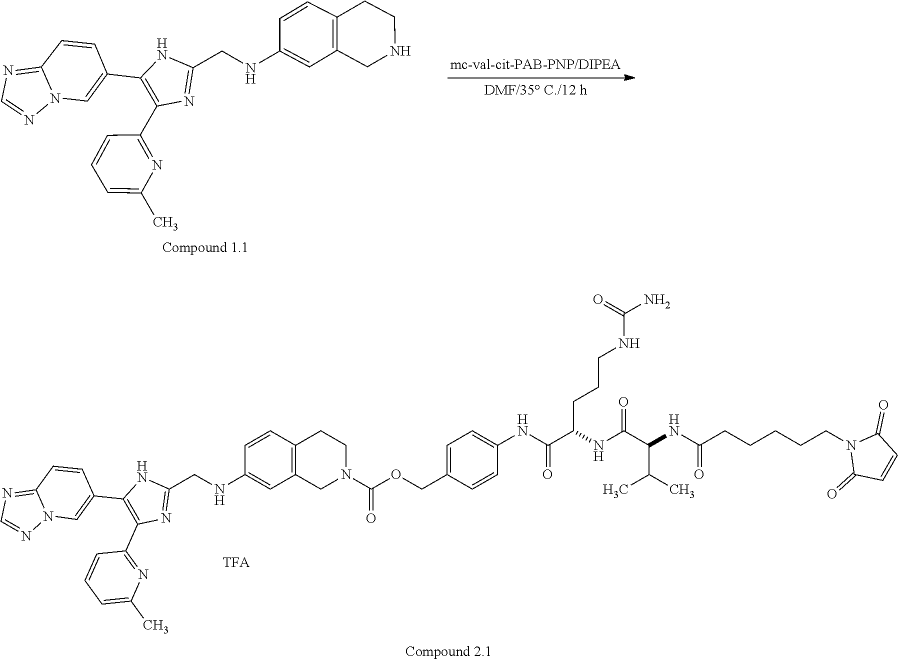 [SMILES: Cc1cccc(-c2nc(CNc3ccc4c(c3)CNCC4)[nH]c2-c2ccc3ncnn3c2)n1 ; Cc1cccc(-c2nc(CNc3ccc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)[nH]c2-c2ccc3ncnn3c2)n1]

To a solution containing 43.7 mg (0.10 mmol) of Compound 1.1 in 2 mL of DMF was added 73.7 mg (0.10 mmol) of mc-VC-PAB-PNP (Broad Pharm; CAS 159857-81-5) and 69 uL (0.40 mmol) of DIPEA. The reaction mixture was stirred at 35° C. for 12 then purified without work-up by HPLC (0% to 80% MeCN w/0.1% TFA/H₂O x/0.1% TFA). The product peak was collected and lyophilized to afford the TFA salt of Compound 2.1. ¹H NMR (DMSO-d⁶) δ 9.99 (s, 1H), 9.42 (s, 1H), 8.60 (s, 1H), 8.07 (d, J=7.6Hz, 1H), 7.93 (d, J=9.6Hz, 2H), 7.58 (d, J=8.4Hz, 2H), 7.43 (d, J=7.6Hz, 1H), 7.34-7.29 (m, 3H), 6.99 (s, 2H), 6.93 (d, J=8.4Hz, 1H), 6.58-6.53 (m, 2H), 6.00 (bs, 1H), 5.02 (s, 2H), 4.56 (s, 2H), 4.46 m, 2H), 4.36 (q, J=8.4Hz, 1H), 4.18 (t, J=8.4Hz, 1H), 3.55 (br t, 2H), 3.36 (t, J=7.2Hz, 2H), 3.30-2.92 (m, 2H), 2.54 (s, 3H), 2.20-2.08 (m, 2H), 1.98-1.93 (m, 1H), 1.68-1.57 (m, 2H), 1.51-1.33 (m, 7H), 1.19 (t, J=7.6Hz, 2H), 0.84 (d, J=6.8Hz, 3H), 0.81 (d, J=6.8Hz, 3H). LCMS (M+H)=1034.5.

The compounds in Table 3 and 4 were prepared in a manner similar to that described for compound 2.1 using the appropriately substituted amine.

TABLE 3
Compounds prepared according to the methods described herein.

| | | | MS |
|---|---|---|---|
| Cmpd | Structure and Name | ¹H NMR | (M + H) |
| 2.2 | 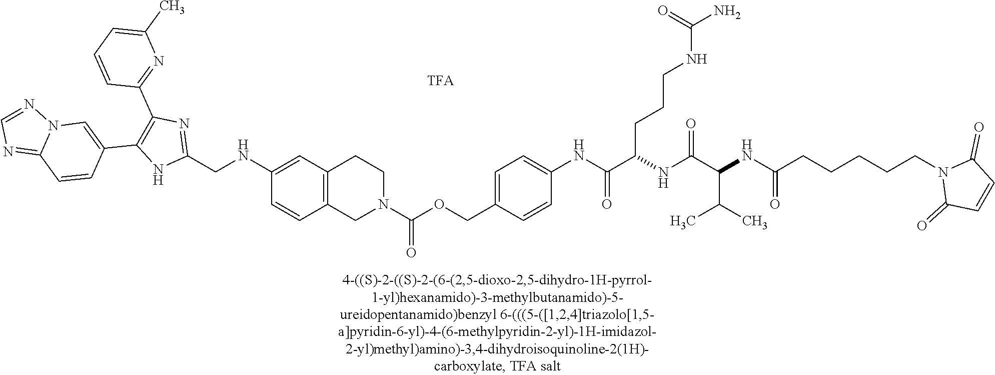 [SMILES: Cc1cccc(-c2nc(CNc3ccc4c(c3)CCN(C(=O)OCc3ccc(NC(=O)[C@H](CCCNC(N)=O)NC(=O)[C@@H](NC(=O)CCCCCN5C(=O)C=CC5=O)C(C)C)cc3)C4)[nH]c2-c2ccc3ncnn3c2)n1] | DMSO-d⁶ δ 11.1 (s, 1H), 9.99 (s, 1H), 9.42 (s, 1H), 8.58 (s, 1H), 8.13 (d, J = 7.6 Hz, 1H), 8.08 (d, J = 7.6 Hz, 2H), 7.92 (d, J = 9.2 Hz, 1H), 7.83-7.75 (m, 3H), 7.59 (d, J = 8.4 Hz, 2H), 7.43 (d, J = 7.6 Hz, 1H), 7.34-7.29 (m, 3H), 6.99 (s, 2H), 6.93 (d, J = 8.4 Hz, 1H), 6.51 (t, J = 7.6 Hz, 2H), 5.99 (bs, 1H), 5.08 (s, 2H), 4.62 (s, 2H), 4.46 m, 2H), 4.36 (q, J = 8.4 Hz, 1H), 4.18 (t, J = 8.4 Hz, 1H), 3.60 (br t, 2H), | 1034.5 |
| | | 3.36 (t, J = 7.2 Hz, | |
| | | 2H), 3.00-2.95 (m, | |
| | | 2H), 2.72 (m, 2H), | |
| | | 2.53 (s, 3H), 2.20- | |
| | | 2.08 (m, 2H), 1.98- | |
| | | 1.93 (m, 1H), 1.68- | |
| | | 1.57 (m, 2H), 1.51- | |
| | | 1.33 (m, 7H), 1.19 | |
| | | (t, J = 7.6 Hz, 2H), | |
| | | 0.84 (d, J = 6.8 Hz, | |
| | | 3H), 0.81 (d, | |
| | | J = 6.8 Hz, 3H). | |
| 2.3 | 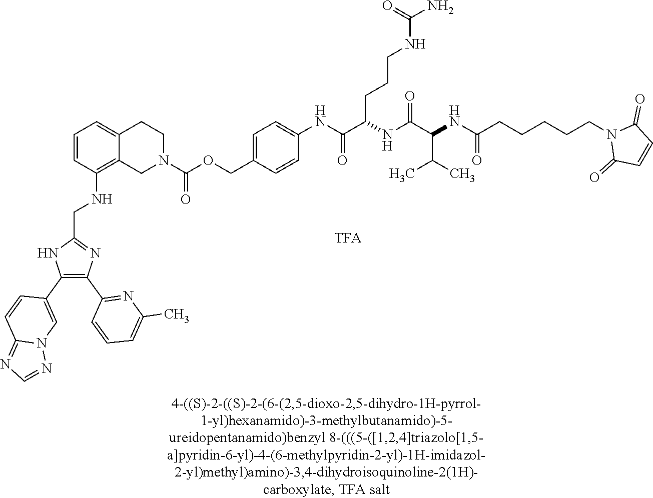 [SMILES: Cc1cccc(-c2nc(CNc3cccc4c3CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)[nH]c2-c2ccc3ncnn3c2)n1] | DMSO-d⁶ δ 9.99 (s, 1H), 9.42 (s, 1H), 8.59 (s, 1H), 8.07 (d, J = 7.6 Hz, 1H), 7.92 (d, J = 8.8 Hz, 1H), 7.82-7.75 (m, 3H), 7.59 (d, J = 8.0 Hz, 2H), 7.43 (d, J = 8.0 Hz, 1H), 7.33-7.31 (m, 3H), 7.02 (m, 1H), 7.00 (s, 2H), 6.51 (t, J = 7.6 Hz, 2H), 5.99 (bs, 1H), 5.03 (s, 2H), 4.62 (s, 2H), 4.50 (br s, 2H), 4.37 (q, J = 8.4 Hz, 1H), 4.18 (t, J = 8.4 Hz, 1H), 3.68 (br t, 2H), 3.36 (t, J = 7.2 Hz, 2H), 3.00-2.93 (m, 2H), 2.61 (m, 2H), 2.53 (s, 3H), 2.20-2.09 (m, 2H), 1.97-1.93 (m, 1H), 1.69-1.57 (m, 2H), 1.51-1.33 (m, 7H), 1.19 (t, J = 7.6 Hz, 2H), 0.84 | 1034.5 |
| | | (d, J = 6.8 Hz, 3H), | |
| | | 0.81 (d, J = 6.8 Hz, | |
| | | 3H). | |
| 2.4 | 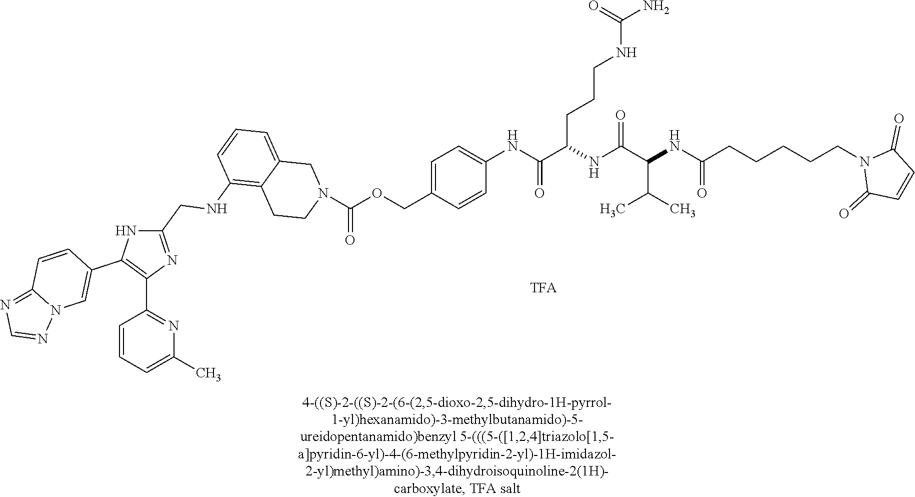 [SMILES: Cc1cccc(-c2nc(CNc3cccc4c3CCN(C(=O)OCc3ccc(NC(=O)[C@H](CCCNC(N)=O)NC(=O)[C@@H](NC(=O)CCCCCN5C(=O)C=CC5=O)C(C)C)cc3)C4)[nH]c2-c2ccc3ncnn3c2)n1] | DMSO-d⁶ δ 9.99 (s, 1H), 9.43 (s, 1H), 8.59 (s, 1H), 8.07 (d, J = 7.6 Hz, 1H), 7.93 (d, J = 6.8 Hz, 1H), 7.82-7.77 (m, 3H), 7.58 (d, J = 8.8 Hz, 2H), 7.43 (d, J = 8.0 Hz, 1H), 7.33-7.29 (m, 3H), 7.00 (s, 2H), 6.95 (m, 1H), 6.57-6.52 (m, 2H), 5.99 (bs, 1H), 5.03 (s, 2H), 4.55 (s, 2H), 4.39- 4.34 (m, 3H), 4.18 (q, J = 8.4 Hz, 1H), 3.55 (br t, 2H), 3.36 (t, J = 7.2 Hz, 2H), 3.00-2.93 (m, 2H), 2.69 (m, 2H), 2.54 (s, 3H), 2.20- 2.09 (m, 2H), 1.97- 1.93 (m, 1H), 1.69- 1.57 (m, 2H), 1.51- 1.33 (m, 7H), 1.19 (t, J = 7.6 Hz, 2H), 0.84 (d, J = 6.8 Hz, 3H), 0.81 (d, | 1034.5 |
| | | J = 6.8 Hz, 3H). | |
| 2.7 | 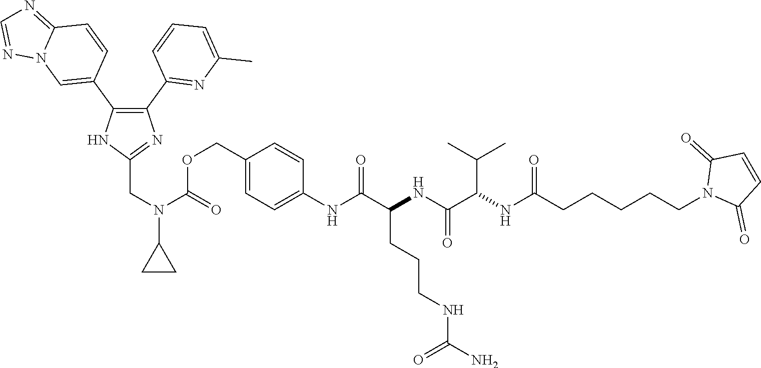 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)C3CC3)[nH]c2-c2ccc3ncnn3c2)n1] | | |
| | 4-((S)-2-((S)-2-(6-(2,5-dioxo-2,5-dihydro-1H-pyrrol- | | |
| | 1-yl)hexanamido)-3-methylbutanamido)-5- | | |
| | ureidopentanamido)benzyl ((5-([1,2,4]triazolo[1,5- | | |
| | a]pyridin-6-yl)-4-(6-methylpyridin-2-yl)-1H-imidazol- | | |
| | 2-yl)methyl)(cyclopropyl)carbamate | | |
| 2.9 | 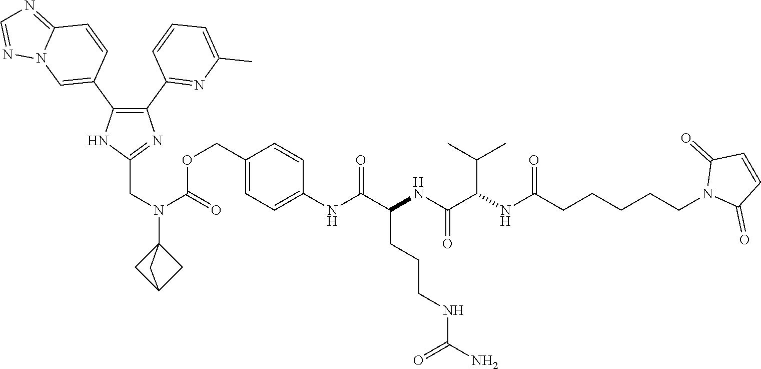 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)C34CC(C3)C4)[nH]c2-c2ccc3ncnn3c2)n1] | | |
| | 4-((S)-2-((S)-2-(6-(2,5-dioxo-2,5-dihydro-1H-pyrrol- | | |
| | 1-yl)hexanamido)-3-methylbutanamido)-5- | | |
| | ureidopentanamido)benzyl ((5-([1,2,4]triazolo[1,5- | | |
| | a]pyridin-6-yl)-4-(6-methylpyridin-2-yl)-1H-imidazol- | | |
| | 2-yl)methyl)(bicyclo[1.1.1]pentan-1-yl)carbamate | | |

TABLE 4
Compounds prepared according to the methods described herein.

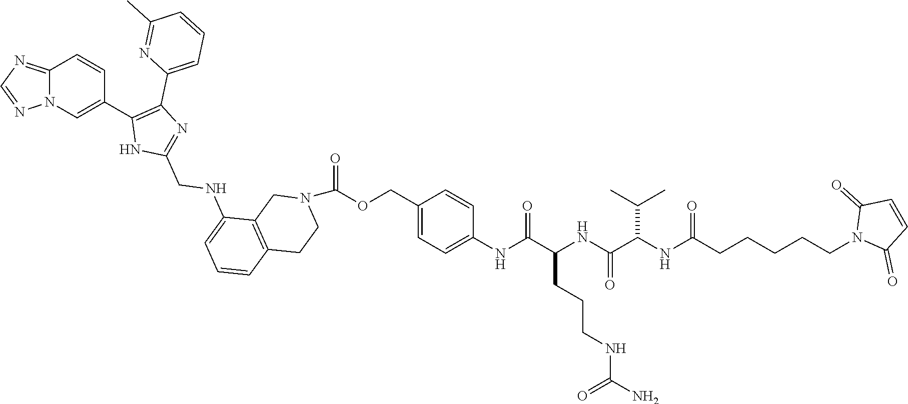 [SMILES: Cc1cccc(-c2nc(CNc3cccc4c3CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)[nH]c2-c2ccc3ncnn3c2)n1]

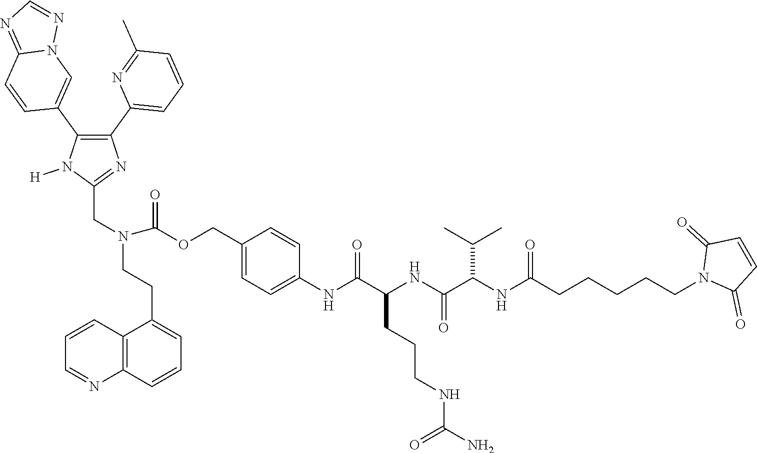 [SMILES: Cc1cccc(-c2nc(CN(CCc3cccc4ncccc34)C(=O)OCc3ccc(NC(=O)[C@H](CCCNC(N)=O)NC(=O)[C@@H](NC(=O)CCCCCN4C(=O)C=CC4=O)C(C)C)cc3)[nH]c2-c2ccc3ncnn3c2)n1]

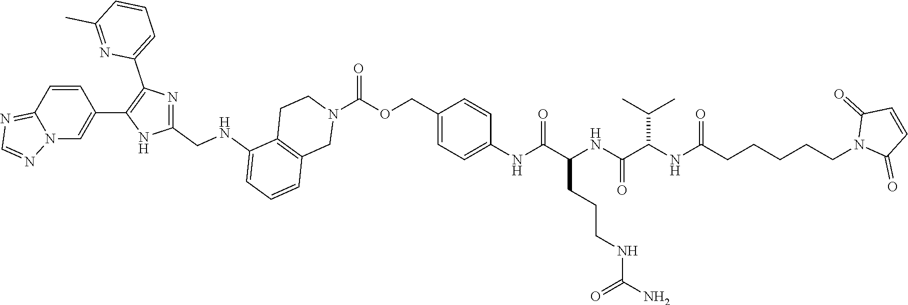 [SMILES: Cc1cccc(-c2nc(CNc3cccc4c3CCN(C(=O)OCc3ccc(NC(=O)[C@H](CCCNC(N)=O)NC(=O)[C@@H](NC(=O)CCCCCN5C(=O)C=CC5=O)C(C)C)cc3)C4)[nH]c2-c2ccc3ncnn3c2)n1]

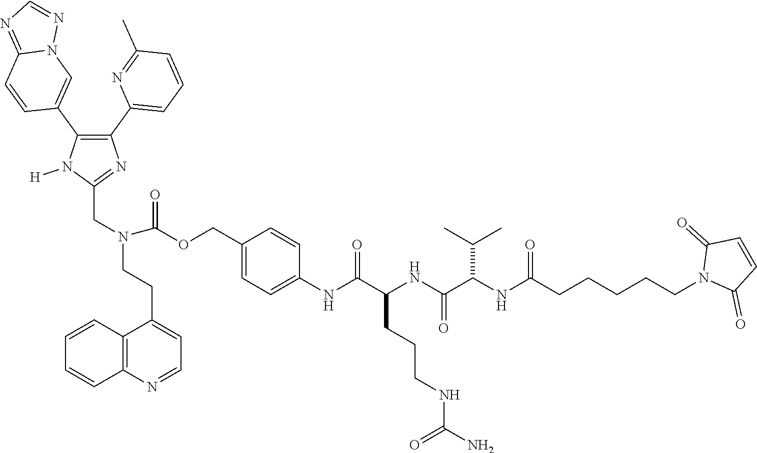 [SMILES: Cc1cccc(-c2nc(CN(CCc3ccnc4ccccc34)C(=O)OCc3ccc(NC(=O)[C@H](CCCNC(N)=O)NC(=O)[C@@H](NC(=O)CCCCCN4C(=O)C=CC4=O)C(C)C)cc3)[nH]c2-c2ccc3ncnn3c2)n1]

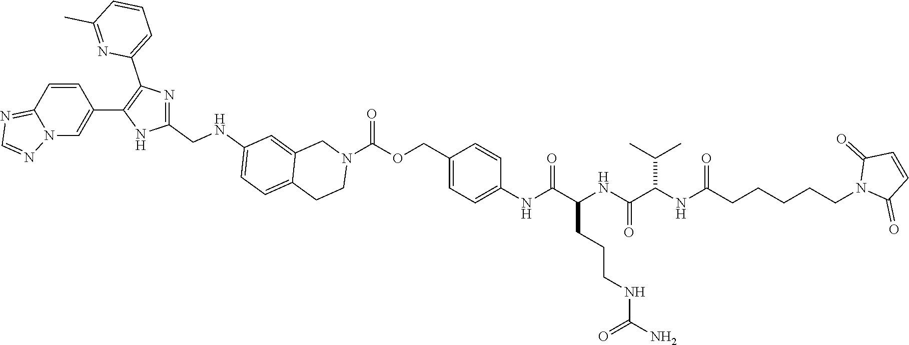 [SMILES: Cc1cccc(-c2nc(CNc3ccc4c(c3)CN(C(=O)OCc3ccc(NC(=O)[C@H](CCCNC(N)=O)NC(=O)[C@@H](NC(=O)CCCCCN5C(=O)C=CC5=O)C(C)C)cc3)CC4)[nH]c2-c2ccc3ncnn3c2)n1]

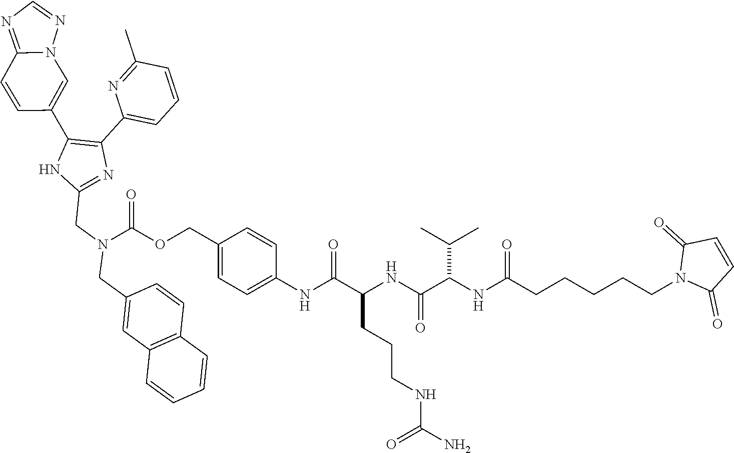 [SMILES: Cc1cccc(-c2nc(CN(Cc3ccc4ccccc4c3)C(=O)OCc3ccc(NC(=O)[C@H](CCCNC(N)=O)NC(=O)[C@@H](NC(=O)CCCCCN4C(=O)C=CC4=O)C(C)C)cc3)[nH]c2-c2ccc3ncnn3c2)n1]

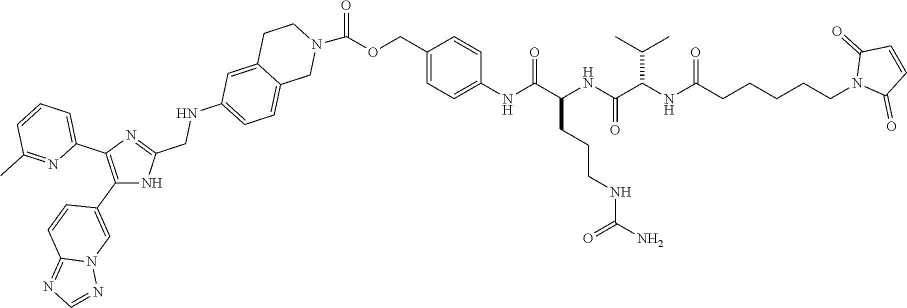 [SMILES: Cc1cccc(-c2nc(CNc3ccc4c(c3)CCN(C(=O)OCc3ccc(NC(=O)[C@H](CCCNC(N)=O)NC(=O)[C@@H](NC(=O)CCCCCN5C(=O)C=CC5=O)C(C)C)cc3)C4)[nH]c2-c2ccc3ncnn3c2)n1]

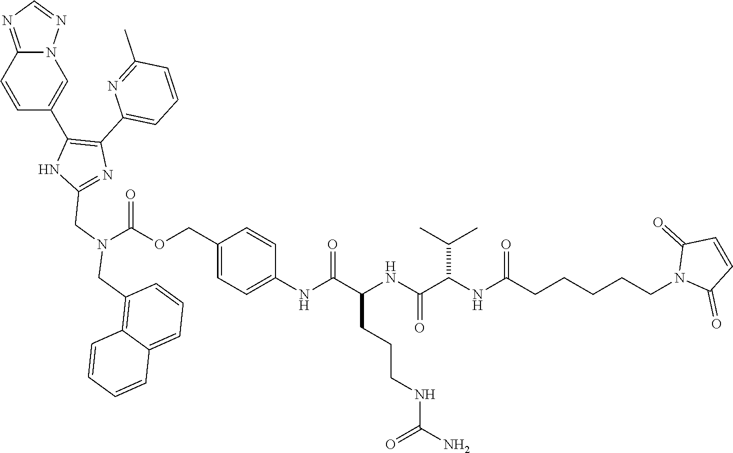 [SMILES: Cc1cccc(-c2nc(CN(Cc3cccc4ccccc34)C(=O)OCc3ccc(NC(=O)[C@H](CCCNC(N)=O)NC(=O)[C@@H](NC(=O)CCCCCN4C(=O)C=CC4=O)C(C)C)cc3)[nH]c2-c2ccc3ncnn3c2)n1]

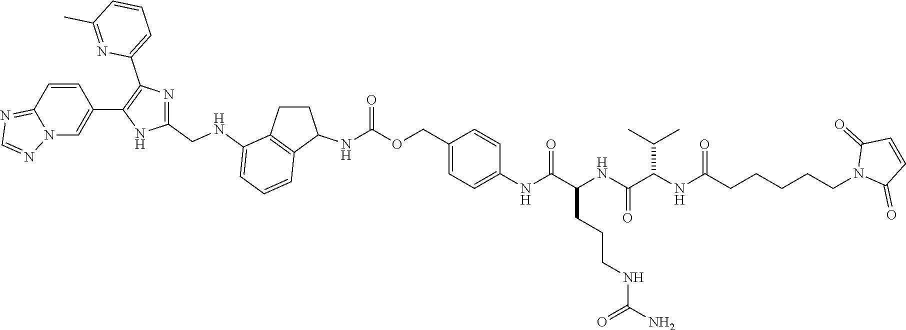 [SMILES: Cc1cccc(-c2nc(CNc3cccc4c3CCC4NC(=O)OCc3ccc(NC(=O)[C@H](CCCNC(N)=O)NC(=O)[C@@H](NC(=O)CCCCCN4C(=O)C=CC4=O)C(C)C)cc3)[nH]c2-c2ccc3ncnn3c2)n1]

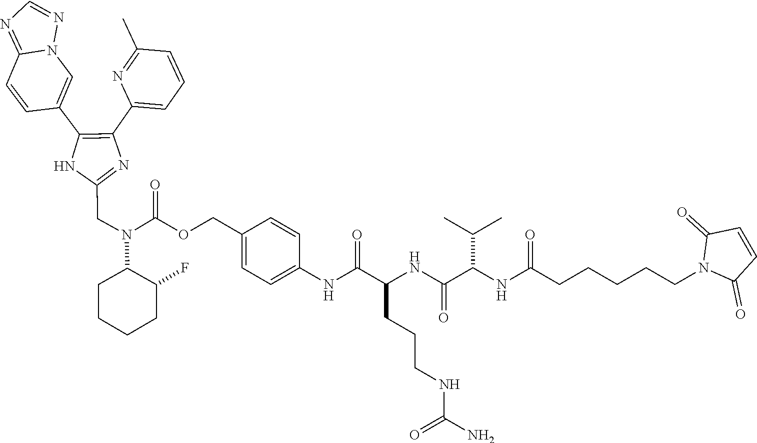 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)[C@H]3CCCC[C@H]3F)[nH]c2-c2ccc3ncnn3c2)n1]

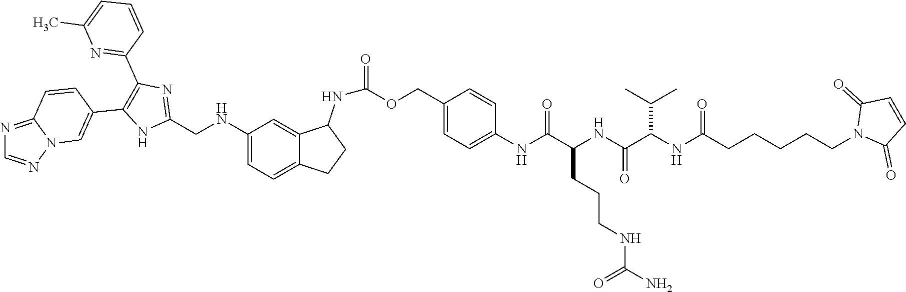 [SMILES: Cc1cccc(-c2nc(CNc3ccc4c(c3)C(NC(=O)OCc3ccc(NC(=O)[C@H](CCCNC(N)=O)NC(=O)[C@@H](NC(=O)CCCCCN5C(=O)C=CC5=O)C(C)C)cc3)CC4)[nH]c2-c2ccc3ncnn3c2)n1]

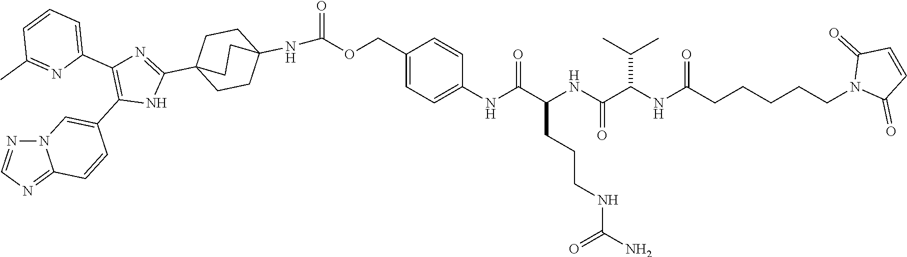 [SMILES: Cc1cccc(-c2nc(C34CCC(NC(=O)OCc5ccc(NC(=O)[C@H](CCCNC(N)=O)NC(=O)[C@@H](NC(=O)CCCCCN6C(=O)C=CC6=O)C(C)C)cc5)(CC3)CC4)[nH]c2-c2ccc3ncnn3c2)n1]

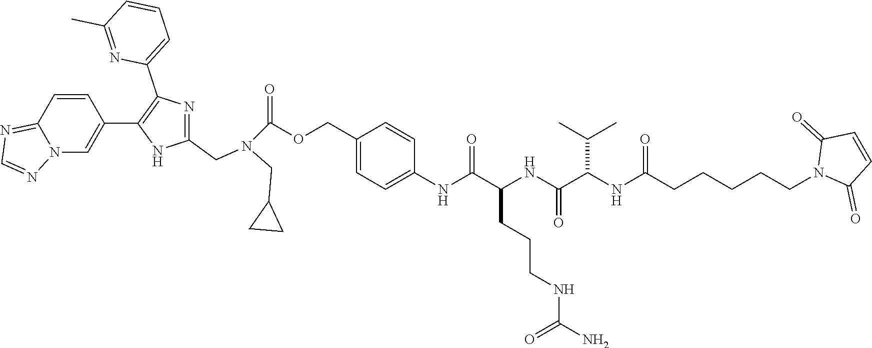 [SMILES: Cc1cccc(-c2nc(CN(CC3CC3)C(=O)OCc3ccc(NC(=O)[C@H](CCCNC(N)=O)NC(=O)[C@@H](NC(=O)CCCCCN4C(=O)C=CC4=O)C(C)C)cc3)[nH]c2-c2ccc3ncnn3c2)n1]

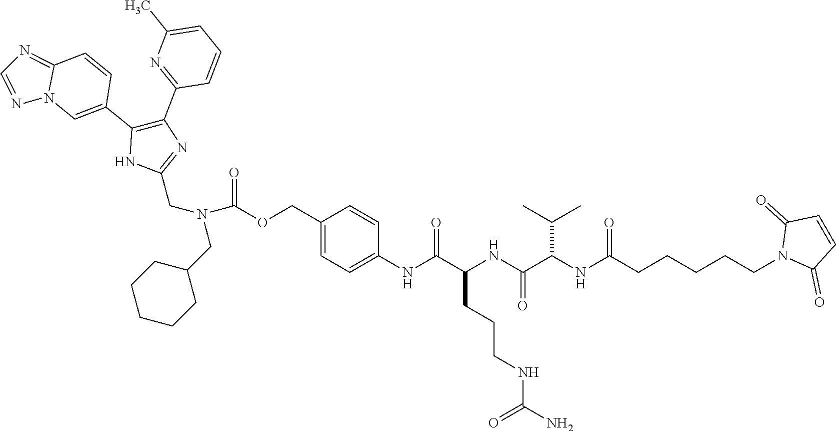 [SMILES: Cc1cccc(-c2nc(CN(CC3CCCCC3)C(=O)OCc3ccc(NC(=O)[C@H](CCCNC(N)=O)NC(=O)[C@@H](NC(=O)CCCCCN4C(=O)C=CC4=O)C(C)C)cc3)[nH]c2-c2ccc3ncnn3c2)n1]

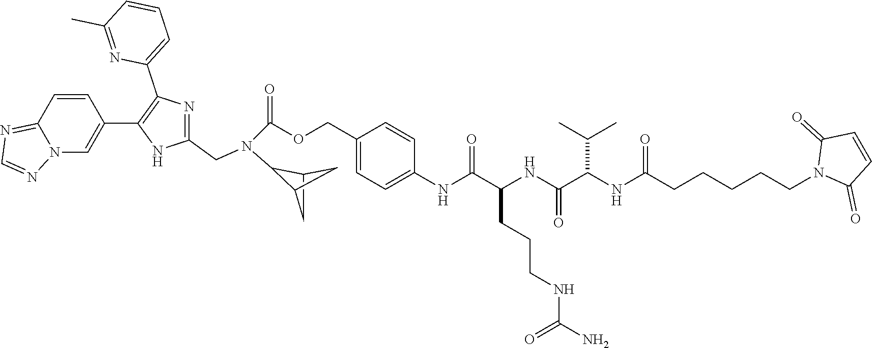 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)C3C4CC3C4)[nH]c2-c2ccc3ncnn3c2)n1]

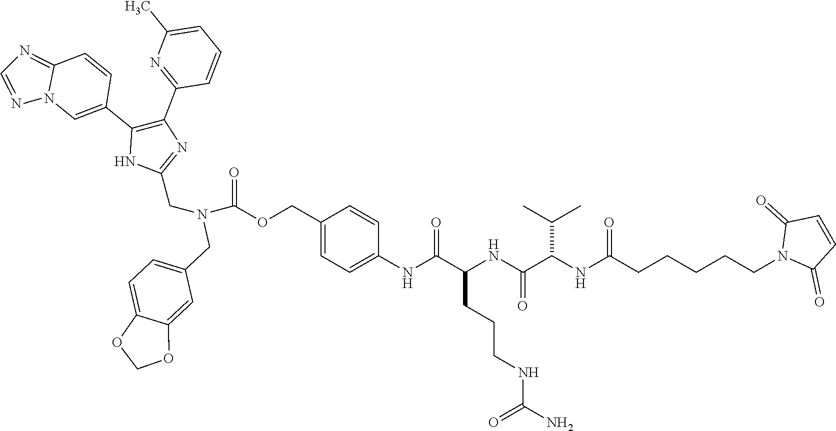 [SMILES: Cc1cccc(-c2nc(CN(Cc3ccc4c(c3)OCO4)C(=O)OCc3ccc(NC(=O)[C@H](CCCNC(N)=O)NC(=O)[C@@H](NC(=O)CCCCCN4C(=O)C=CC4=O)C(C)C)cc3)[nH]c2-c2ccc3ncnn3c2)n1]

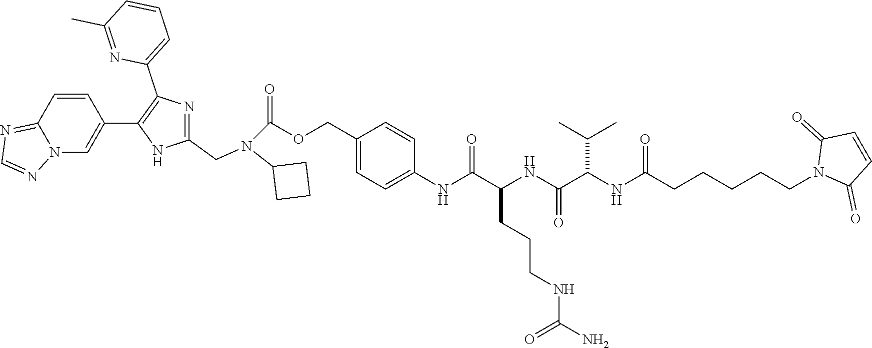 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)C3CCC3)[nH]c2-c2ccc3ncnn3c2)n1]

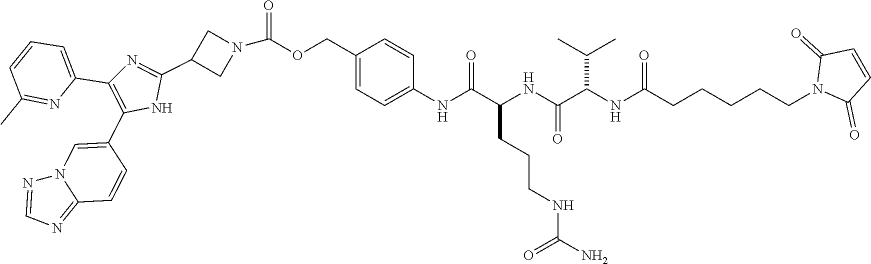 [SMILES: Cc1cccc(-c2nc(C3CN(C(=O)OCc4ccc(NC(=O)[C@H](CCCNC(N)=O)NC(=O)[C@@H](NC(=O)CCCCCN5C(=O)C=CC5=O)C(C)C)cc4)C3)[nH]c2-c2ccc3ncnn3c2)n1]

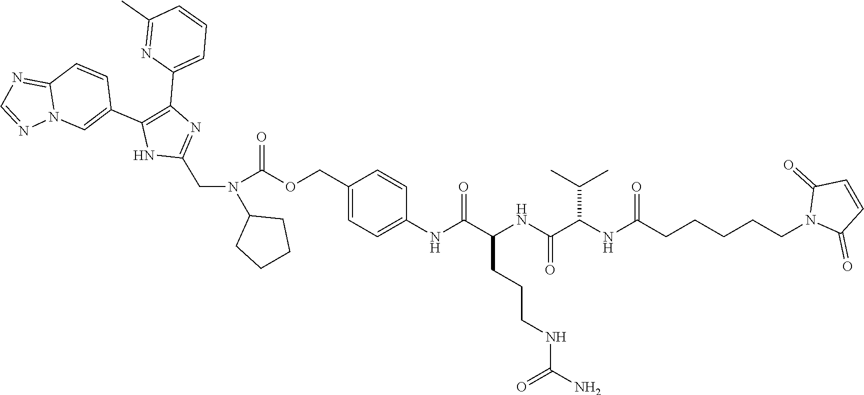 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)C3CCCC3)[nH]c2-c2ccc3ncnn3c2)n1]

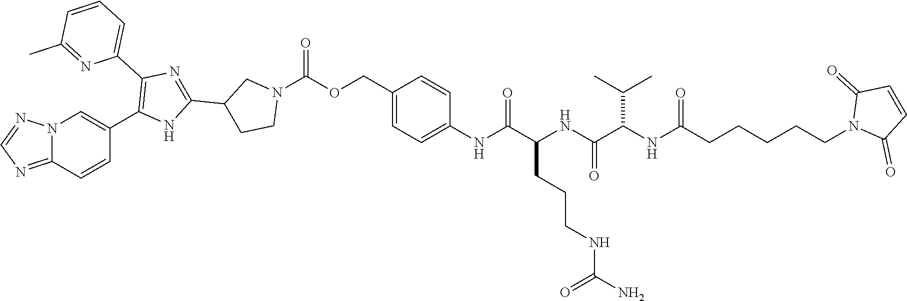 [SMILES: Cc1cccc(-c2nc(C3CCN(C(=O)OCc4ccc(NC(=O)[C@H](CCCNC(N)=O)NC(=O)[C@@H](NC(=O)CCCCCN5C(=O)C=CC5=O)C(C)C)cc4)C3)[nH]c2-c2ccc3ncnn3c2)n1]

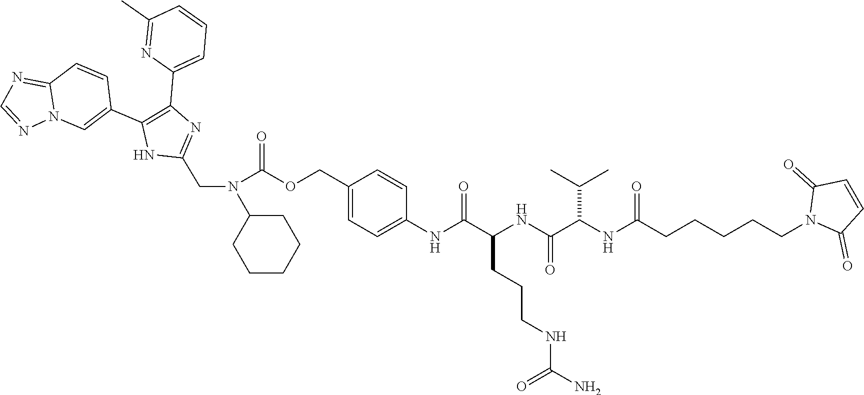 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)C3CCCCC3)[nH]c2-c2ccc3ncnn3c2)n1]

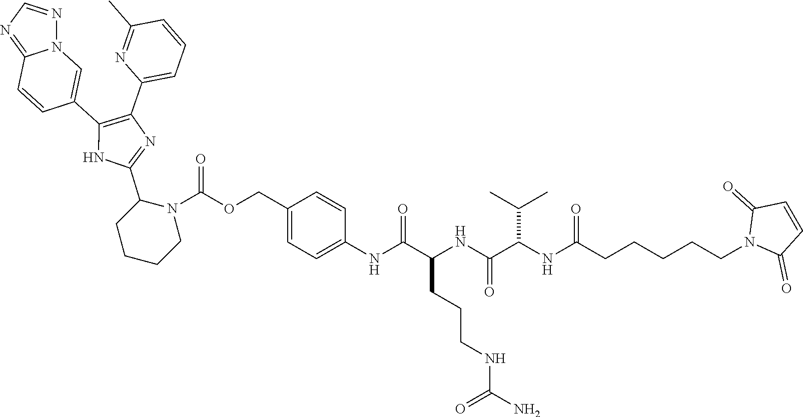 [SMILES: Cc1cccc(-c2nc(C3CCCCN3C(=O)OCc3ccc(NC(=O)[C@H](CCCNC(N)=O)NC(=O)[C@@H](NC(=O)CCCCCN4C(=O)C=CC4=O)C(C)C)cc3)[nH]c2-c2ccc3ncnn3c2)n1]

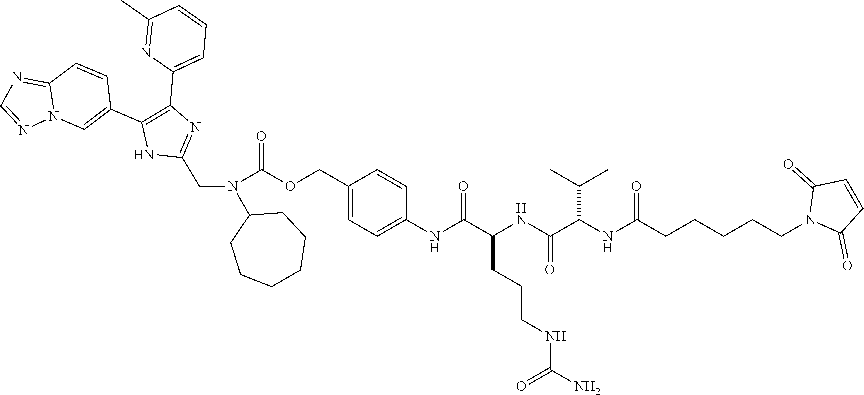 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)C3CCCCCC3)[nH]c2-c2ccc3ncnn3c2)n1]

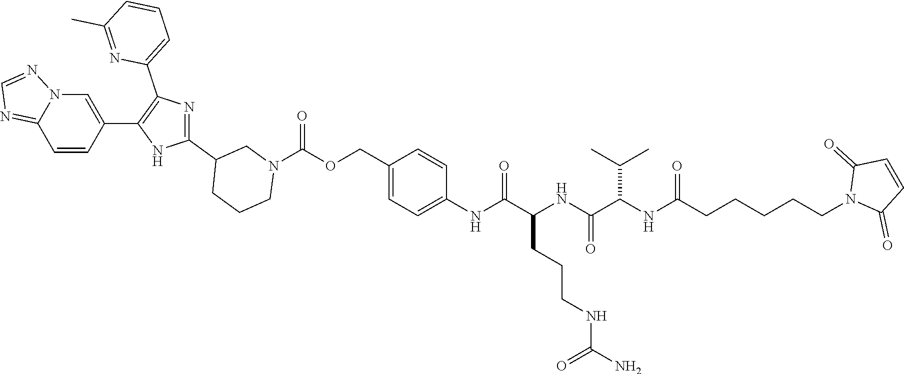 [SMILES: Cc1cccc(-c2nc(C3CCCN(C(=O)OCc4ccc(NC(=O)[C@H](CCCNC(N)=O)NC(=O)[C@@H](NC(=O)CCCCCN5C(=O)C=CC5=O)C(C)C)cc4)C3)[nH]c2-c2ccc3ncnn3c2)n1]

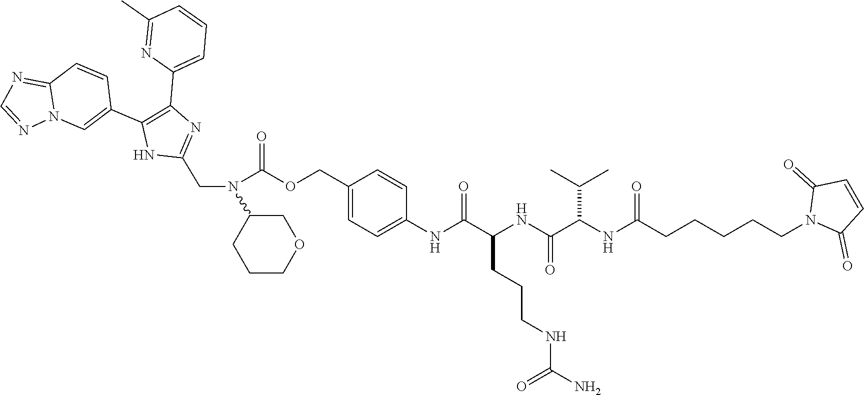 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)C3CCCOC3)[nH]c2-c2ccc3ncnn3c2)n1]

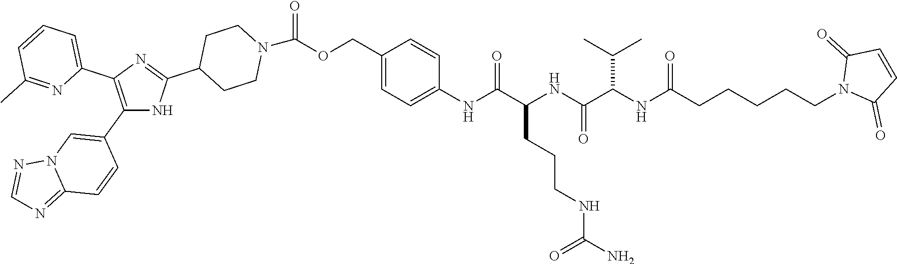 [SMILES: Cc1cccc(-c2nc(C3CCN(C(=O)OCc4ccc(NC(=O)[C@H](CCCNC(N)=O)NC(=O)[C@@H](NC(=O)CCCCCN5C(=O)C=CC5=O)C(C)C)cc4)CC3)[nH]c2-c2ccc3ncnn3c2)n1]

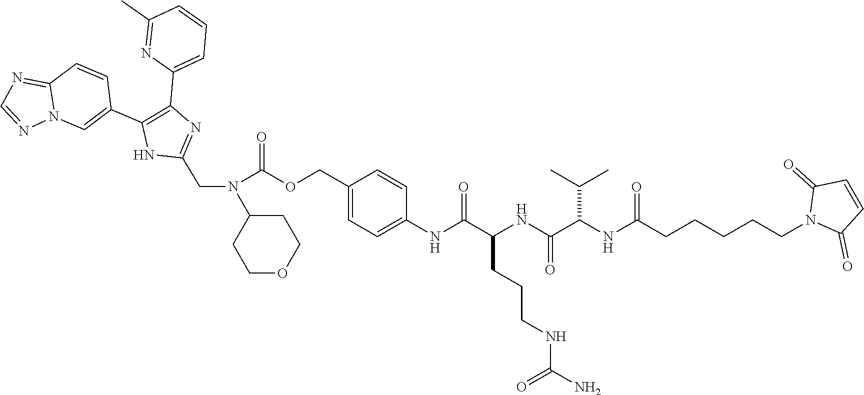 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)C3CCOCC3)[nH]c2-c2ccc3ncnn3c2)n1]

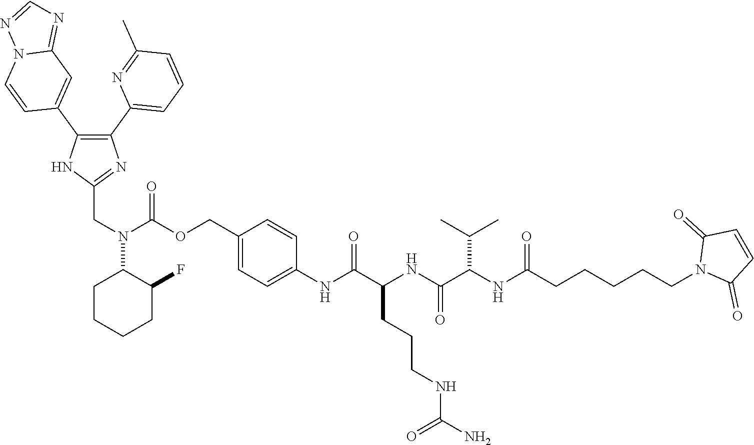 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)[C@H]3CCCC[C@@H]3F)[nH]c2-c2ccn3ncnc3c2)n1]

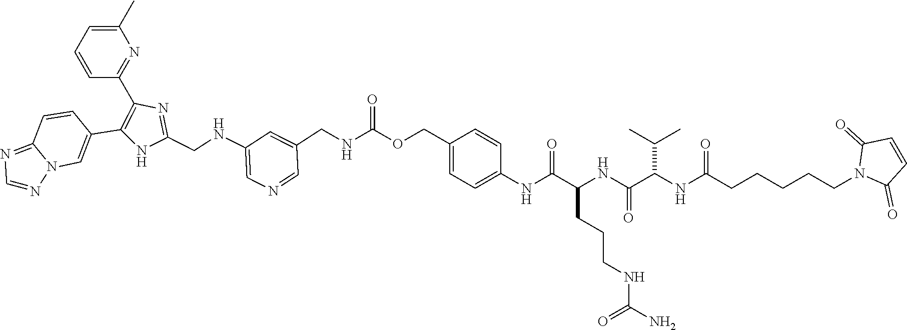 [SMILES: Cc1cccc(-c2nc(CNc3cncc(CNC(=O)OCc4ccc(NC(=O)[C@H](CCCNC(N)=O)NC(=O)[C@@H](NC(=O)CCCCCN5C(=O)C=CC5=O)C(C)C)cc4)c3)[nH]c2-c2ccc3ncnn3c2)n1]

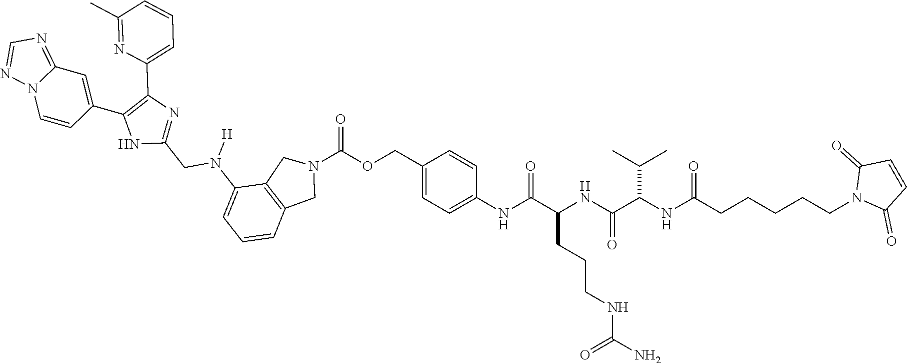 [SMILES: Cc1cccc(-c2nc(CNc3cccc4c3CN(C(=O)OCc3ccc(NC(=O)[C@H](CCCNC(N)=O)NC(=O)[C@@H](NC(=O)CCCCCN5C(=O)C=CC5=O)C(C)C)cc3)C4)[nH]c2-c2ccn3ncnc3c2)n1]

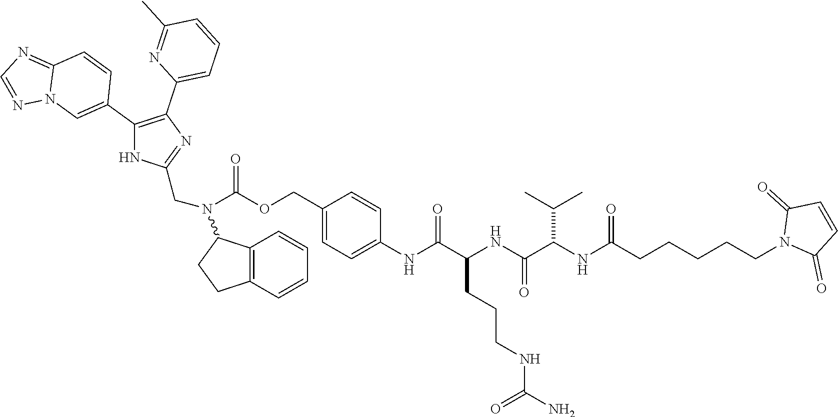 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)C3CCc4ccccc43)[nH]c2-c2ccc3ncnn3c2)n1]

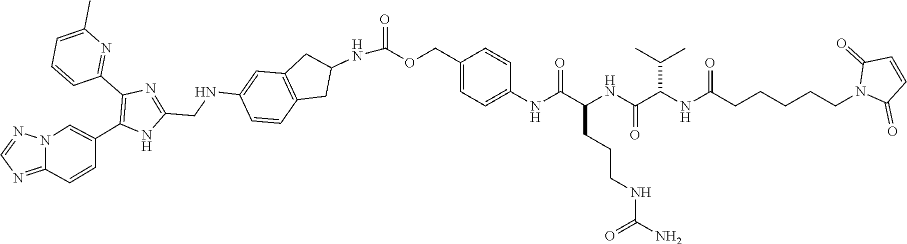 [SMILES: Cc1cccc(-c2nc(CNc3ccc4c(c3)CC(NC(=O)OCc3ccc(NC(=O)[C@H](CCCNC(N)=O)NC(=O)[C@@H](NC(=O)CCCCCN5C(=O)C=CC5=O)C(C)C)cc3)C4)[nH]c2-c2ccc3ncnn3c2)n1]

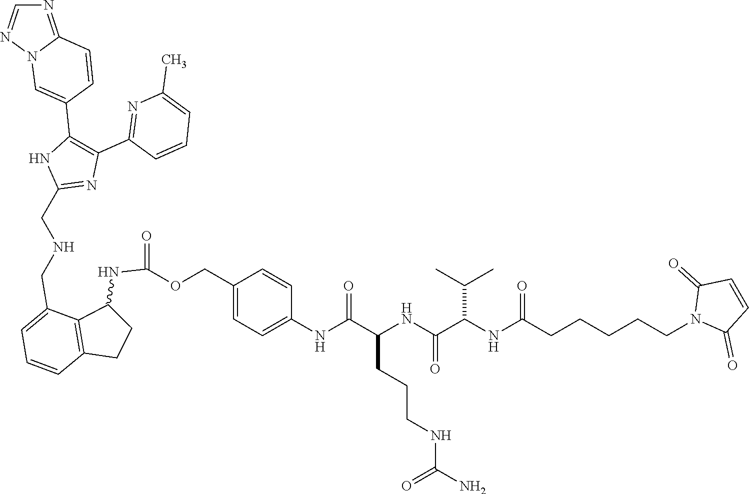 [SMILES: Cc1cccc(-c2nc(CNCc3cccc4c3C(NC(=O)OCc3ccc(NC(=O)[C@H](CCCNC(N)=O)NC(=O)[C@@H](NC(=O)CCCCCN5C(=O)C=CC5=O)C(C)C)cc3)CC4)[nH]c2-c2ccc3ncnn3c2)n1]

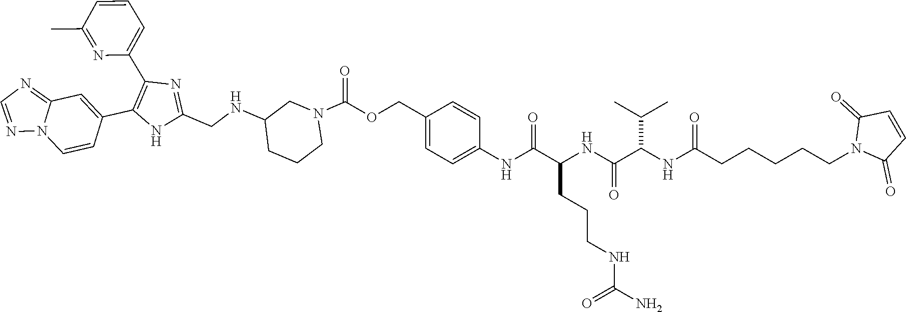 [SMILES: Cc1cccc(-c2nc(CNC3CCCN(C(=O)OCc4ccc(NC(=O)[C@H](CCCNC(N)=O)NC(=O)[C@@H](NC(=O)CCCCCN5C(=O)C=CC5=O)C(C)C)cc4)C3)[nH]c2-c2ccn3ncnc3c2)n1]

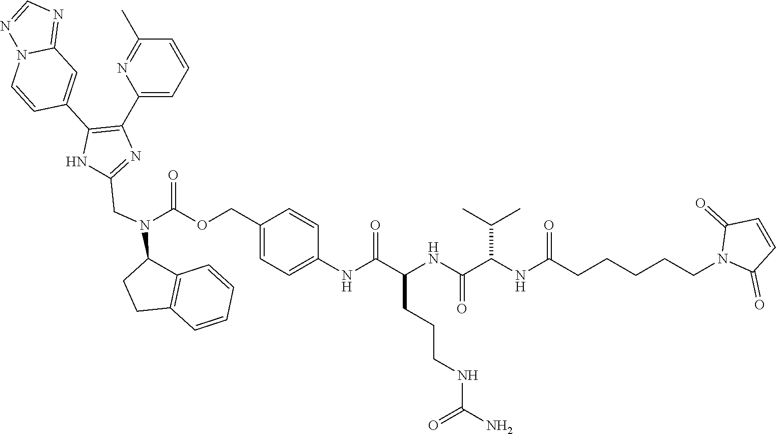 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)[C@@H]3CCc4ccccc43)[nH]c2-c2ccn3ncnc3c2)n1]

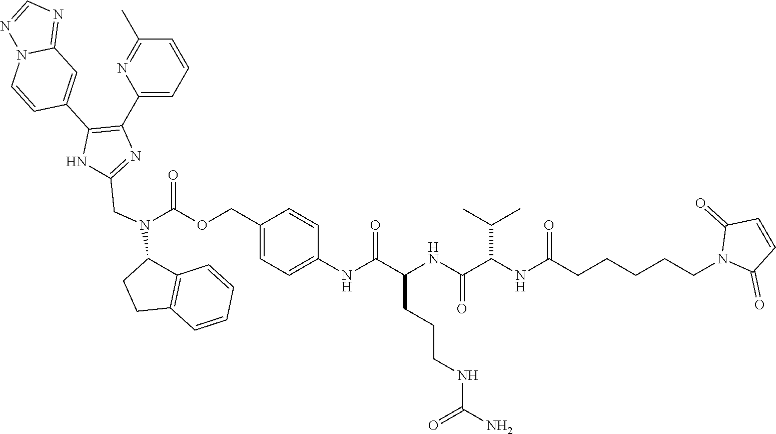 [SMILES: Cc1cccc(-c2nc(CN(C(=O)OCc3ccc(NC(=O)[C@H](CCCNC(N)=O)NC(=O)[C@@H](NC(=O)CCCCCN4C(=O)C=CC4=O)C(C)C)cc3)[C@H]3CCc4ccccc43)[nH]c2-c2ccn3ncnc3c2)n1]

### EXAMPLE 4

### Compounds of Formula (I) were assayed to measure their activity as ALK5 inhibitors.

### Example 4A

### Enzyme Inhibition Assay

ALK5 enzyme inhibition assays were performed by Reaction Biology Corp (Malvern, PA). 1 mg/mL of peptide subtrate (casein) and 10 uM ATP were prepared in a mixture of fresh reaction buffer. The kinase was delivered into the substrate solution which was gently mixed. Compounds in 100% DMSO were added to the kinase reaction mixture by Acoustic technology (Echo550; nanoliter range) and the mixture was incubated for 20 min at room temperature. ³³P-ATP (Specific activity 10 uCi/uL) was added into the reaction mixture to initiate the reaction and the reaction mixture was incubated for 2 hours at room temperature. Radioactivity was detected by filter-binding method and kinase activity data were expressed as the percent remaining kinase activity in test samples compared to vehicle (dimethyl sulfoxide) reactions. IC₅₀ values and curve fits were obtained using Prism (GraphPad Software). Compounds having an IC₅₀ value between 0.1 nM and 10 nM are denoted as ++++, 10 nM and 100 nM as +++, 100 nM and 1000 nM as ++, and 1000 nM to 10,000 nM as + in Table 5 below.

TABLE 5
Inhibition of ALK5 by compounds described herein.

| Compound | IC₅₀ |
|---|---|
| 1.1 | ++ |
| 1.2 | ++ |
| 1.3 | ++ |
| 1.4 | ++ |
| 1.5 | ++ |
| 1.6 | ++ |
| 1.7 | + |
| 1.8 | + |
| 1.9 | + |
| 1.10 | + |
| 1.11 | ++ |
| 1.12 | ++ |
| 1.13 | ++ |
| 1.14 | ++ |
| 1.15 | ++ |
| 1.16 | ++ |
| 1.19 | + |
| 1.20 | ++ |
| 1.21 | ++ |
| 1.22 | + |
| 1.23 | +++ |
| 1.24 | ++ |
| 1.25 | ++ |
| 1.26 | ++ |
| 1.27 | ++ |
| 1.30 | ++ |
| 1.31 | ++ |
| 1.32 | +++ |
| 1.33 | ++ |
| 1.34 | ++ |
| 1.35 | + |
| 1.32 | +++ |
| 1.33 | ++ |
| 1.40 | ++ |
| 1.41 | ++ |
| 1.42 | ++ |
| 1.43 | +++ |
| 1.44 | ++ |
| 1.45 | ++ |
| 1.46 | ++ |
| 1.48 | + |
| 1.51 | + |
| 1.52 | + |
| 1.55 | + |
| 1.56 | ++ |
| 1.57 | +++ |
| 1.58 | ++ |
| 1.59 | +++ |
| 1.60 | ++ |
| 1.61 | ++ |

### Example 4B

### TGFb Reporter Assay

TGFb/SMAD Signaling Pathway SBE reporter cell line was obtained from BPS Bioscience. Cells were passed/expanded/stored in liquid nitrogen per supplier's instruction with the exception that growth media was changed to DMEM-C with Geneticin (DMEM supplemented with 10% fetal bovine serum, 1× NEAA, 1 mM Pyruvate, 2 mM glutamine, 50 μg/mL penicillin, 50 U/mL streptomycin and 400 ug/mL of Geneticin). The assay media was MEM supplemented with 0.5% fetal bovine serum, 1× NEAA, 1 mM Pyruvate, 50 μg/mL penicillin and 50 U/mL streptomycin.

Reporter cells were harvested from the tissue culture flasks by incubation in small quantity of Versene at room temperature for three to five minutes after the media in the flask was removed and cells rinsed with PBS. Cells were counted and diluted in the assay media at ˜0.8×10⁶ cells/mL then 50 uL/well were added to 96-well assay plate. Test samples (at desired concentrations diluted in assay media) were added to assay plate containing the 50 uL/well of cells (or media only), 50 uL per well, and incubated for 5-6 hours at 37° C. in a 5% CO₂ humidified incubator. After that time, 15 uL of TGFb diluted to 12.5 ng/mL in the assay media was added to the plate. Controls included TGFb titration (from 25 to 0 ng/mL) without inhibitors, and media only (without cells, inhibitor or TGFb). Plates were incubated at 37° C. in a 5% CO₂ humidified incubator for 18 h. Luciferase substrate solution was subsequently added at 75 uL per well, incubated in dark with shaking at room temperature for 10 min, and luminescence was measured using a luminometer. Compounds having an IC₅₀ value between 0.1 nM and 10 nM are denoted as ++++, 10 nM and 100 nM as +++, 100 nM and 1000 nM as ++, and 1000 nM to 10,000 nM as + in Table 4 below.

TABLE 6
TGFb reporter activity by compounds described herein.

| Compound | IC50 |
|---|---|
| 1.1 | ++ |
| 1.2 | ++ |
| 1.3 | ++ |
| 1.4 | ++ |
| 1.5 | ++ |
| 1.6 | ++ |
| 1.7 | ++ |
| 1.8 | ++ |
| 1.9 | +++ |
| 1.10 | ++ |
| 1.11 | +++ |
| 1.12 | +++ |
| 1.13 | +++ |
| 1.14 | +++ |
| 1.15 | +++ |
| 1.16 | +++ |
| 1.19 | ++ |
| 1.20 | ++ |
| 1.21 | ++ |
| 1.22 | ++ |
| 1.23 | ++ |
| 1.24 | ++ |
| 1.25 | ++ |
| 1.26 | +++ |
| 1.27 | +++ |
| 1.30 | +++ |
| 1.31 | ++ |
| 1.32 | ++ |
| 1.33 | ++ |
| 1.34 | ++ |
| 1.35 | ++ |
| 1.38 | + |
| 1.39 | + |
| 1.40 | ++ |
| 1.41 | ++ |
| 1.42 | +++ |
| 1.43 | +++ |
| 1.44 | +++ |
| 1.45 | +++ |
| 1.46 | +++ |
| 1.48 | ++ |
| 1.49 | ++ |
| 1.50 | ++ |
| 1.51 | ++ |
| 1.52 | ++ |
| 1.53 | + |
| 1.55 | + |
| 1.56 | +++ |
| 1.57 | +++ |
| 1.58 | ++ |
| 1.59 | +++ |
| 1.60 | +++ |
| 1.61 | +++ |

### EXAMPLE 5

### Generation of Antibody ALK5 Inhibitor Conjugates through Partial Reduction of Native Disulfide Bonds of Non-Engineered Antibodies

The mAb (3-8 mg/mL in PBS) was exchanged into HEPES (100 mM, pH 7.0, 1 mM DTPA) via molecular weight cut-off centrifugal filtration (Millipore, 30 kDa). The resultant mAb solution was transferred to a tared 50 mL conical tube. The mAb concentration was determined to be 3-8 mg/mL by A₂₈₀. To the mAb solution was added TCEP (2.0-4.0 equivalents, 1 mM stock) at room temperature and the resultant mixture was incubated at 37° C. for 30-90 minutes, with gentle shaking. Upon being cooled to room temperature, a stir bar was added to the reaction tube. With stirring, DMA (5-15% v/v) was added dropwise to the reaction mixture. Next, the linker-payload from Example 3 (5.0-13.0 equivalents, 10 mM DMA) was added dropwise. The resultant reaction mixture was allowed to stir at ambient temperature for 30-60 minutes, at which point N-ethyl maleimide (3.0 equivalents, 100 mM DMA) was added. After an additional 15 minutes of stirring, cysteine (6.0-11.0 equivalents, 50 mM HEPES) was added. The crude ADC was then exchanged into PBS and purified by preparative SEC (e.g. HiLoad 26/600, Superdex 200pg) using PBS as the mobile phase. The pure fractions were concentrated via molecular weight cut-off centrifugal filtration (Millipore, 30 kDa), sterile filtered, and transferred to 15 mL conical tubes. Drug-antibody construct ratios (molar ratios) were determined by methods described in Example 7.

### EXAMPLE 6

### Lysine-based Bioconjugation

An antibody construct can be conjugated to a linker via lysine-based bioconjugation. An antibody construct described herein can be exchanged into an appropriate buffer, for example, phosphate, borate, histidine, PBS, Tris-Acetate at a concentration of about 2 mg/mL to about 10 mg/mL. An appropriate number of equivalents of a compound-linker, e.g., a compound or salt of Formulas (I), (Ia), (Ib), (Ic), (Id) and (Ie) attached to a linker can be added to a solution with stirring. Dependent on the physical properties of the compound-linker construct, a co-solvent can be introduced prior to the addition of the compound-linker construct to facilitate solubility. The reaction can be stirred at room temperature for 2 hours to about 12 hours depending on the observed reactivity. The progression of the reaction can be monitored by LC-MS. Once the reaction is deemed complete, the remaining compound-linker constructs can be removed by applicable methods and the antibody construct-compound conjugate can be exchanged into the desired formulation buffer. Lysine-linked conjugates can be synthesized starting with antibody (mAb) or bispecific antibody (bsAb) and compound-linker construct, e.g., 10 equivalents, following Scheme A below (Conjugate =antibody construct-compound conjugate). Monomer content and compound-antibody construct ratios (molar ratios) can be determined by methods described herein.

### EXAMPLE 7

### General Procedure for the Determination of the Drug-Antibody-Ratios

### Hydrophobic Interaction Chromatography

10 μL of a 6 mg/mL solution of the conjugate are injected into an HPLC system set-up with a TOSOH TSKgel Butyl-NPR TM hydrophobic interaction chromatography (HIC) column (2.5 μM particle size, 4.6 mm×35 mm) attached. Then, over the course of 18 minutes, a method is run in which the mobile phase gradient runs from 100% mobile phase A to 100% mobile phase B over the course of 12 minutes, followed by a six-minute re-equilibration at 100% mobile phase A. The flow rate is 0.8 mL/min and the detector is set at 280 nM. Mobile phase A was 1.5 M ammonium sulfate, 25 mM sodium phosphate (pH 7). Mobile phase B is 25% isopropanol in 25 mM sodium phosphate (pH 7). Post-run, the chromatogram is integrated and the molar ratio is determined by summing the weighted peak area.

### EXAMPLE 8

### Reporter Assay for Conjugates

The following reporter assay can be used to determine the activity of conjugates containing any of the compounds of Formulas (I), (Ia), (Ib), (Ic) (Id) or (Ie).

Materials and general procedures. A parental TGFb/SMAD Signaling Pathway SBE reporter cell line is maintained in DMEM 10% fetal bovine serum, 1× NEAA, 1 mM Pyruvate, 2 mM glutamine, 50 μg/mL penicillin, 50 U/mL streptomycin and 400 ug/mL of hygromycin B. Cells expressing a target antigen of interest are maintained according to standard method with the addition of 0.5 ug/mL puromycin. Assay media is MEM supplemented with 0.5% fetal bovine serum, 1× NEAA, 1 mM Pyruvate, 50 m/mL penicillin and 50 U/mL streptomycin.

General procedure for in vitro ALKS inhibitor conjugate screening. Reporter cells as described above, with and without the target antigen, are harvested from the tissue culture flasks by incubation in small quantity of Versene at room temperature for three to five minutes after the media in the flask is removed and cells rinsed with PBS. Cells are counted and diluted in the assay media at ˜0.8×10⁶ cells/mL then 50 μL/well is added to a 96-well assay plate. A volume of 50 μL/well of test samples (e.g., conjugate, antibody alone, etc., at desired concentrations diluted in assay media) is added to an assay plate containing the cells, and incubated for 24 hours at 37° C. in a 5% CO₂ humidified incubator. After that time, 15 μL of TGFβ diluted to 12.5 ng/mL in the assay media is added to the plate. Controls include TGFβ titration, cells with TGFβ treatment only, and media only (without cells, conjugate inhibitor or TGFβ). Plates are incubated at 37° C. in a 5% CO₂ humidified incubator for 18 h. Luciferase substrate solution is subsequently added at 75 μL per well, incubated in dark with shaking at room temperature for 10 min, and luminescence is measured using a luminometer.

### EXAMPLE 9

### Reporter Assay for LRRC15 Conjugates

The compound-linkers in Table 4 were conjugated to an anti-LRRC15 antibody as described in Example 5 to arrive at conjugates with an average drug load between 2 and 5. The reporter assay as described above in Example 8 was used to determine the activity of the LRRC15-conjugates. Briefly, parental reporter cells are plated in 6 well plates and the following day are transfected with a vector encoding LRRC15 using Lipofectamine 3000, per manufacturer's instructions. Twenty four hours post-transfection, cells are plated and treatments are added as described above for the parental cells. Cells and test samples, including an LRRC15 conjugate and an LRRC15 antibody alone, are incubated for 24 hours prior to adding TGFβ and then determining luciferase activity as described above. The conjugates reliably exhibited activity which demonstrated a high correlation (by rank order) with the activity demonstrated by the small molecule component in the analogous Reporter Assay (Table 6).

While aspects of the present disclosure have been shown and described herein, it will be apparent to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

What is claimed is:

1. A compound represented by Formula (I): 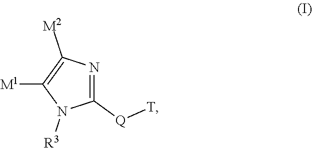
or a salt thereof, wherein: 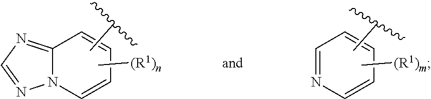
M¹ and M² are independently selected from
R¹ and R² are independently selected at each occurrence from:
a halogen —OR¹⁰, —SR¹⁰, —N(R¹⁰)₂, —C(O)N(R¹⁰)₂, —N(R¹⁰C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, and —CN; —C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, C(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle; and
a C₃-C₁₀ carbocycle and a 3- to 10-membered heterocycle, each of which is optionally substituted with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂), —OP(O)(OR¹⁰)₂, —NO₂, ═O, ═S, ═N(R¹⁰), —CN, —C₁-C₆ alkyl, —C₂-C₆ alkenyl, and —C₂-C₆ alkynyl;
R³ is selected from hydrogen; and —C₁-C₁₀ alkyl optionally substituted with one or more substituents independently selected from a halogen, —NO₂, ═O, ═S, ═N(R¹⁰), —CN, -OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, and —OC(O)R¹⁰;
n and m are independently selected from 0, 1, 2, 3, and 4;
Q is selected from a bond, —(CR¹⁰₂)ₚ—, —(CR¹⁰₂)_{q}C(═O)(CR¹⁰₂)_{q}—, —(CR¹⁰₂)_{q}C(═S)(CR¹⁰₂)_{q}—, —(CR¹⁰₂)_{q}C(═NR¹⁰)(CR¹⁰₂)_{q}, —(CR¹⁰₂)_{q}O(CR¹⁰₂)_{q}—, —(CR¹⁰₂)_{q}S(CR¹⁰₂)_{q}—, —(CR¹⁰₂)_{q}N(R¹⁰)(CR¹⁰₂)_{q}—, —(CR¹⁰₂)_{q}OC(═O)O(CR¹⁰₂)_{q}—, —(CR¹⁰₂)_{q}C(═O)N(R¹⁰)(CR¹⁰₂)_{q}—, —(CR¹⁰₂)_{q}N(R¹⁰)C(═O)(CR¹⁰₂)_{q}—, and —(CR¹⁰₂)_{q}N(R¹⁰)SO₂(CR¹⁰₂)_{q}—;
p is selected from 1, 2, 3, 4, and 5;
q is independently selected at each occurrence from 0, 1, 2, 3, 4, and 5;
T is selected from an optionally substituted saturated C₃-C₇ carbocycle, an optionally substituted C₅₋₁₂ bicyclic carbocycle, and an optionally substituted 4- to 12-membered heterocycle, wherein T is optionally substituted with one or more substituents independently selected at each occurrence from R¹³;
R¹³ is independently selected at each occurrence from:
a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), and —CN;
—C₁-C₁₀ alkyl, —C₂-C₁₀ alkenyl, and —C₂-C₁₀alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle; and
a C₃-C₁₀ carbocycle and a 3- to 10-membered heterocycle, each of which is optionally substituted with one or more substituents independently selected from a halogen, —OR¹⁰, —SR¹⁰, —N(R¹⁰)₂, —C(O)R¹⁰, —C(O)N(R¹⁰)₂), —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰₂), —OP(O)(OR¹⁰)₂, —NO₂, ═O, ═S, ═N(R¹⁰), —CN, —C₁-C₆ alkyl, —C₂-C₆ alkenyl, and —C₂-C₆ alkynyl; and
R¹⁰ is independently selected at each occurrence from:
hydrogen;
—C₁-C₁₀ alkyl, —C₂-C₁₀ alkenyl, and —C₂-C₁₀alkynyl, each of which is optionally substituted at each occurrence with one or more substituents independently selected from a halogen, —OH, —CN, —NO₂, —NH₂, ═O, ═S, —O-C₁-C₁₀ alkyl, C₃-C₁₂ carbocycle, and a 3- to 12-membered heterocycle; and
a C₃-C₁₂ carbocycle and a 3- to 12-membered heterocycle, each of which is optionally substituted at each occurrence with one or more substituents independently selected from a halogen, —OH, —CN, —NO₂, —NH₂, ═O, ═S, —C₁-C₁₀ alkyl, —O-C₁-C₁₀ alkyl, and —C₁-C₁₀ haloalkyl.

2. The compound or salt of claim 1, wherein one of M¹ and M² is 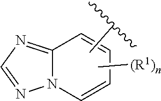
and the other of M¹ and M² is

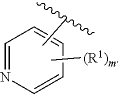

3. The compound or salt of claim 1 or 2, wherein Mⁱ is

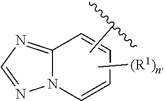

4. The compound of claim 3, wherein the compound of Fomula (I) is represented by Formula (Ia): 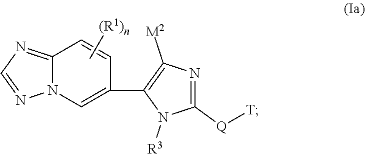
or a salt thereof.

5. The compound of claim 1 or 2, wherein the compound of Fomula (I) is represented by Formula (Ib): 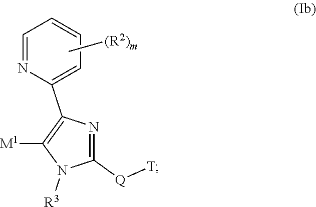
or a salt thereof.

6. The compound or salt of claim 2, wherein M¹ is 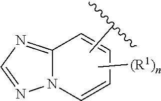
and M² is

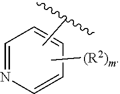

7. The compound or salt of claim 2, wherein M¹ is 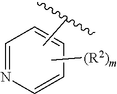
and M² is

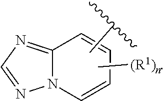

8. The compound of claim 6, wherein the compound of Fomula (I) is represented by Formula (Ic): 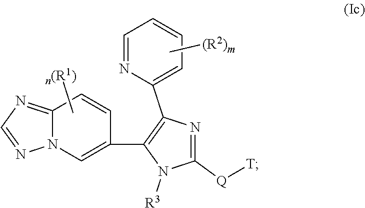
or a salt thereof.

9. The compound of claim 7, wherein the compound of Fomula (I) is represented by Formula (Id): 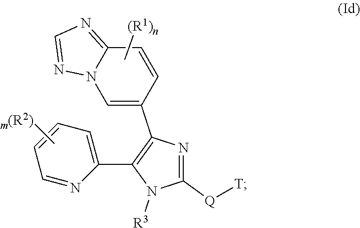
or a salt thereof.

10. The compound of salt of any one of claims 1 to 9, wherein R³ is selected from hydrogen and —C₁-C₁₀ alkyl optionally substituted with one or more substituents independently selected from a halogen, —NO₂, —CN, —OR¹⁰, —SR¹⁰, and —N(R¹⁰)₂.

11. The compound or salt of claim 10, wherein R³ is hydrogen.

12. The compound or salt of any one of claims 1 to 11, wherein n is 0.

13. The compound or salt of any one of claims 1 to 11, wherein n is selected from 1, 2, 3, and 4.

14. The compound or salt of claim 13, wherein each R¹ is independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰), —S(O)₂N(R¹⁰)₂, —P(O)(OR¹⁰)₂, —OP(O)(OR¹⁰)₂, —NO₂, and —CN; and —C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle.

15. The compound or salt of claim 14, wherein each le is independently selected from a halogen, —OR¹⁰, —SR¹⁰, —N(R¹⁰)₂, —NO₂, and —CN; and —C₁-C₁₀ alkyl optionally substituted at each occurrence with one or more substituents independently selected from a halogen, —OR¹⁰, —SR¹⁰, —N(R¹⁰)₂, —C(O)R¹⁰, —NO₂, and —CN.

16. The compound or salt of any one of claims 1 to 15, wherein m is 0.

17. The compound or salt of any one of claims 1 to 15, wherein m is selected from 1, 2, 3, and 4.

18. The compound or salt of claim 1 to 15, or 17, wherein each R² is independently selected from a halogen, —OR¹⁰, —SR¹⁰, N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, and —CN; and —C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle.

19. The compound or salt of claim 18, wherein each R² is independently selected from a halogen, —OR¹⁰, SR¹⁰, —N(R¹⁰)₂, —NO₂, and —CN; and —C₁-C₁₀ alkyl optionally substituted at each occurrence with one or more substituents independently selected from a halogen, —OR¹⁰, —SR¹⁰, —N(R¹⁰)₂, —C(O)R¹⁰, —NO₂, and —CN.

20. The compound or salt of claim 19, wherein each R² is independently selected from —OR¹⁰ and —C₁-C₁₀ alkyl optionally substituted with one or more substituents independently selected from a halogen, —OR¹⁰, —SR¹⁰, —N(R¹⁰)₂, —C(O)R¹⁰, —NO₂, and —CN.

21. The compound or salt of claim 20, wherein m is 1 and R² is —CH₃.

22. The compound or salt of claim 21, wherein M² is

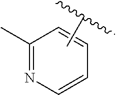

23. The compound or salt of claim 22, wherein M² is

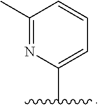 [SMILES: *c1cccc(C)n1]

24. The compound of claim 1 wherein the compound of Formula (I) is represented by Formula (Ie) 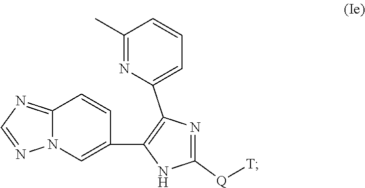
or a salt thereof.

25. The compound or salt of any one of claims 1 to 24, wherein Q is selected from a bond, —(CR¹⁰₂)ₚ—, —(CR¹⁰₂)_{q}O(CR¹⁰₂)_{q}—, —(CR¹⁰₂)_{q}S(CR¹⁰₂)_{q}—, and —(CR¹⁰₂)_{q}NR¹⁰(CR¹⁰₂)_{q}—.

26. The compound or salt of claim 25, wherein Q is selected from a bond, —(CR¹⁰₂)ₚ—, and —(CR¹⁰₂)_{q}NR¹⁰(CR¹⁰₂)_{q}—.

27. The compound or salt of claim 26, wherein Q is —(CR¹⁰₂)ₚ—.

28. The compound or salt of claim 27, wherein Q is —(CR¹⁰₂)—.

29. The compound or salt of claim 28, wherein Q is —CH₂—.

30. The compound or salt of claim 26, wherein Q is selected from —(CR¹⁰₂)_{q}NR¹⁰(CR¹⁰₂)_{q}—.

31. The compound or salt of claim 30, wherein Q is selected from —(CR¹⁰₂)_{q}NR¹⁰—.

32. The compound or salt of claim 31, wherein Q is —CH₂NH—.

33. The compound or salt of claim 30, wherein Q is —CR¹⁰₂NR¹⁰(CR¹⁰₂)₁₋₂—.

34. The compound or salt of claim 33, wherein Q is —CH₂NHCH₂— or —CH₂NHCH₂CH₂—.

35. The compound or salt of any one of claims 1 to 34, wherein each R¹⁰ is independently selected from hydrogen and —C₁-C₁₀ alkyl, —C₂-C₁₀ alkenyl, and —C₂-C₁₀ alkynyl, each of which is optionally substituted with one or more substituents independently selected from a halogen, —OH, —CN, —NO₂, and —NH₂.

36. The compound or salt of claim 35, wherein R¹⁰ is hydrogen at each occurrence.

37. The compound or salt of any one of claims 1 to 35 wherein the R¹⁰ of Q is hydrogen at each occurence.

38. The compound or salt of any one of claims 1 to 37, wherein T is selected from a saturated C₃-C₇ carbocycle optionally substituted with one or more substituents independently selected at each occurrence from R¹³.

39. The compound or salt of claim 38, wherein T is selected from a saturated monocyclic C₃-C₇ carbocycle optionally substituted with one or more substituents independently selected at each occurrence from R¹³.

40. The compound or salt of claim 38, wherein T is a saturated C₃ carbocycle optionally substituted with one or more substituents independently selected at each occurrence from R¹³.

41. The compound or salt of claim 38, wherein T is selected from 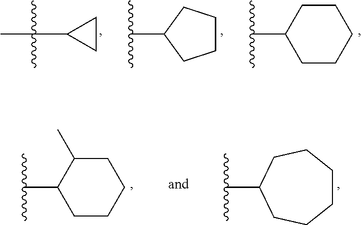
wherein T is optionally substituted with one or more substituents independently selected at each occurrence from R¹³.

42. The compound or salt of claim 39, wherein T is selected from

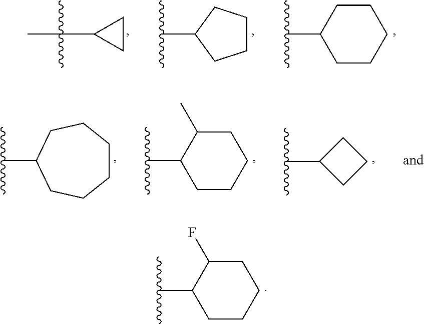

43. The compound or salt of any one of claims 1 to 37, wherein T is selected from a C₅₋₁₂ bicyclic carbocycle optionally substituted with one or more substituents independently selected at each occurrence from R¹³.

44. The compound or salt of claim 43, wherein T is selected from a saturated C₅₋₈ bridged carbocycle optionally substituted with one or more substituents independently selected at each occurrence from R¹³.

45. The compound or salt of claim 44, wherein T is selected from a saturated C₅ bridged carbocycle optionally substituted with one or more substituents independently selected at each occurrence from R¹³.

46. The compound or salt of claim 45, wherein T is selected from 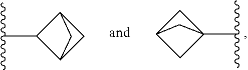
wherein T is optionally substituted with one or more substituents independently selected at each occurrence from R¹³.

47. The compound or salt of claim 43, wherein T is selected from C₈₋₁₁ bicyclic carbocycle optionally substituted with one or more substituents independently selected at each occurrence from R¹³.

48. The compound or salt of any one of claims 1 to 43 and 47, wherein T is a fused bicyclic ring.

49. The compound or salt of claim 47, wherein T is selected from: 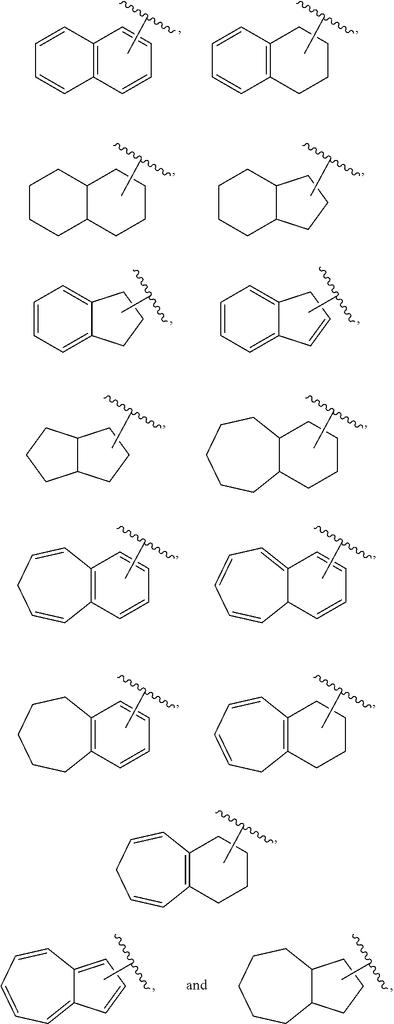
wherein T is optionally substituted with one or more substituents independently selected at each occurrence from R¹³.

50. The compound or salt of claim 49, wherein T is selected from 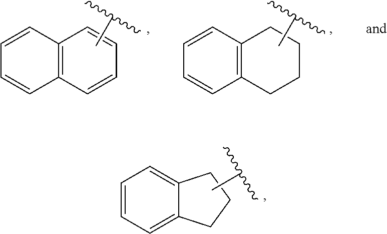
wherein T is optionally substituted with one or more substituents independently selected at each occurrence from R¹³.

51. The compound or salt of claim 49, wherein T is selected from:

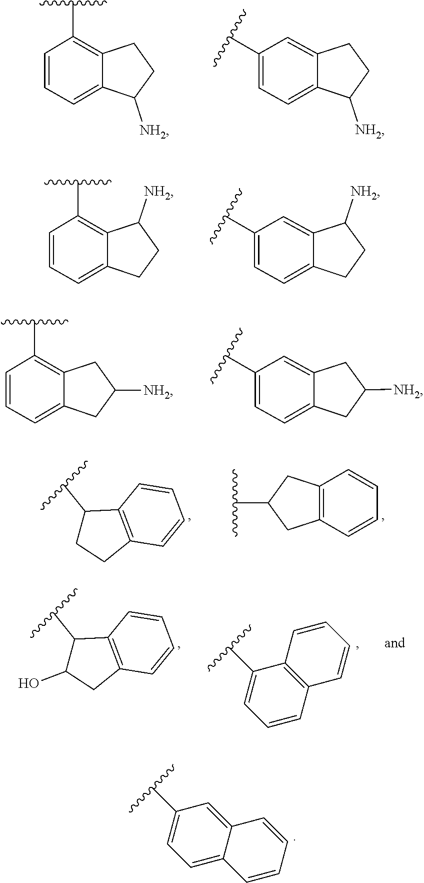

52. The compound or salt of claim 51, wherein T is 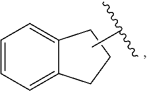
wherein T is optionally substituted with one or more substituents independently selected at each occurrence from R¹³.

53. The compound or salt of claim 51, wherein T is selected from naphthalene, 1,2,3,4-tetrahydronaphthalene, decahydronaphthalene, octahydro-1H-indene, 2,3-dihydro-1H-indene, 1H-indene, octahydropentalene, decahydro-1H-benzo[7]annulene, 7H-benzo[7]annulene, 4aH-benzo[7]annulene, 6,7,8,9-tetrahydro-5H-benzo[7]annulene, 2,3,4,5-tetrahydro-1H-benzo[7]annulene, 2,3,4,7-tetrahydro-1H-benzo[7]annulene, azulene, and decahydroazulene, and wherein T is optionally substituted with one or more substituents independently selected at each occurrence from R¹³.

54. The compound or salt of any one of claims 1 to 37, wherein T is selected from a 4- to 12-membered heterocycle optionally substituted with one or more substituents independently selected at each occurrence from R¹³.

55. The compound or salt of claim 54, wherein T is selected from 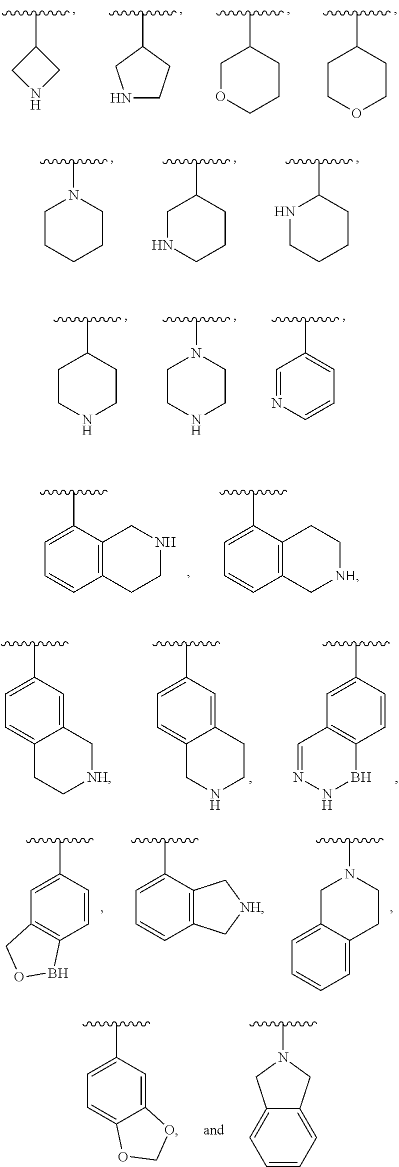
only one of which is optionally substituted with one or more substituents independently selected at each occurrence from R¹³.

56. The compound or salt of claim 54 wherein T is selected from

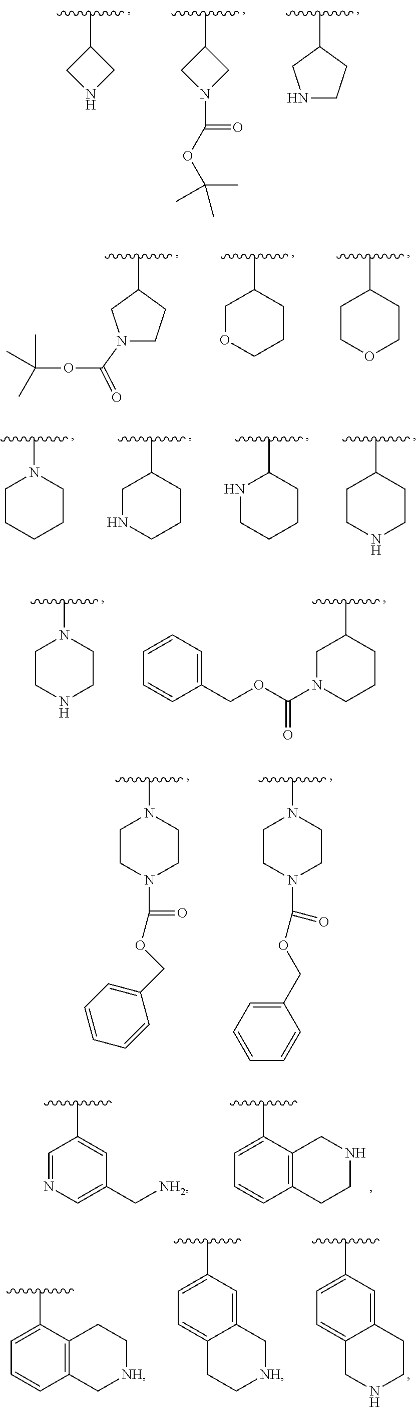

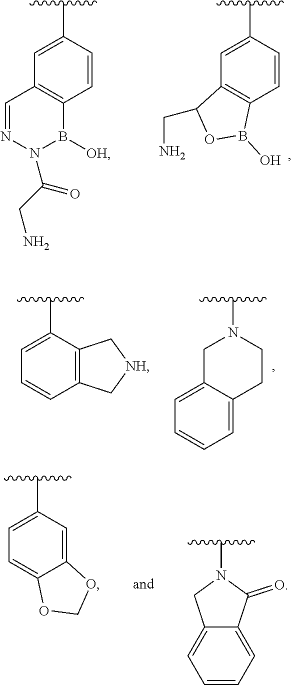

57. The compound or salt of claim 54, wherein T is selected from a 7- to 12-membered bicyclic heterocycle optionally substituted with one or more substituents independently selected at each occurrence from R¹³.

58. The compound or salt of claim 57, wherein T is selected from an 8- to 11-membered bicyclic heterocycle optionally substituted with one or more substituents independently selected at each occurrence from R¹³.

59. The compound or salt of claim 58, wherein T is selected from an 8- to 11-membered bicyclic heteroaryl group optionally substituted with one or more substituents independently selected at each occurrence from R¹³.

60. The compound or salt of any one of claims 57 to 59, wherein T has one to four ring heteroatoms independently selected from N, O, S, and B.

61. The compound or salt of claim 60, wherein T has at least one ring heteroatom that is B.

62. The compound or salt of claim 60, wherein T is selected from 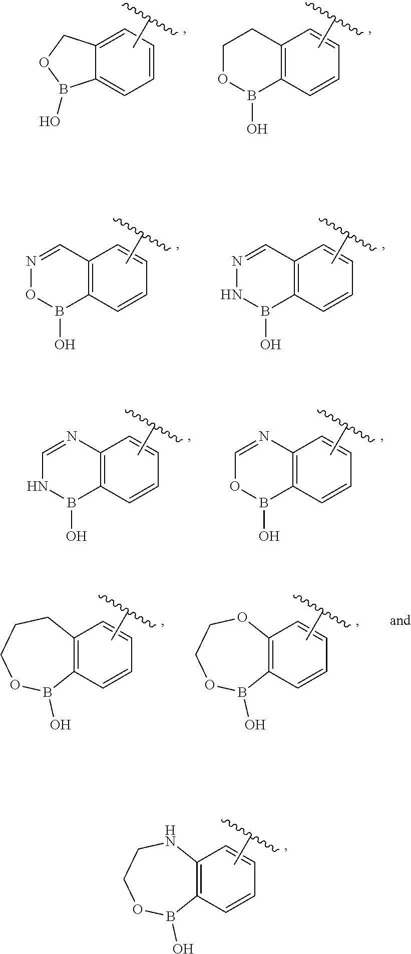
wherein T is optionally substituted with one or more substituents independently selected at each occurrence from R¹³.

63. The compound or salt of claim 60, wherein T is selected from 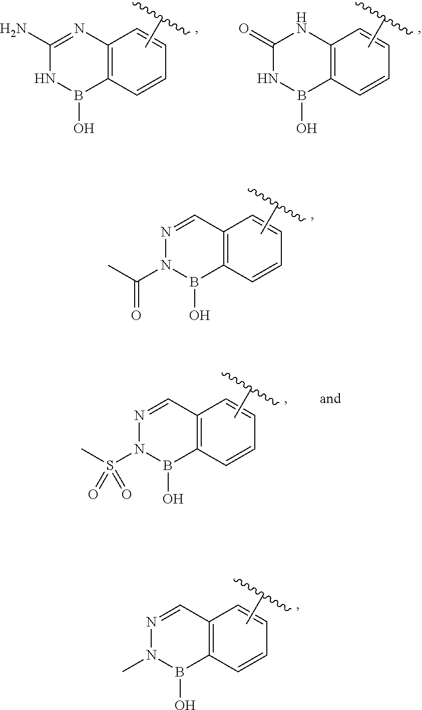
wherein T is optionally substituted with one or more substituents independently selected at each occurrence from R¹³.

64. The compound or salt of claim 54, wherein T is selected from 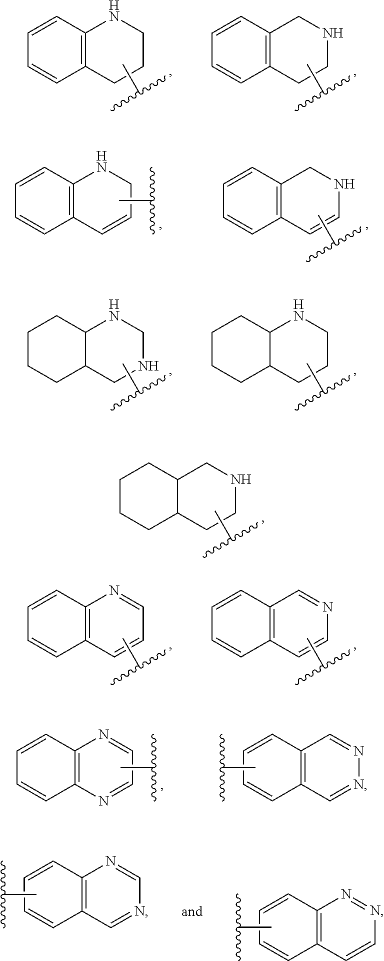
wherein T is optionally substituted with one or more substituents independently selected at each occurrence from R¹³.

65. The compound or salt of claim 54, wherein T is selected from: 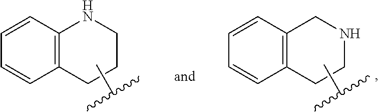
wherein T is optionally substituted with one or more substituents independently selected at each occurrence from R¹³.

66. The compound or salt of claim 54, wherein T is selected from: 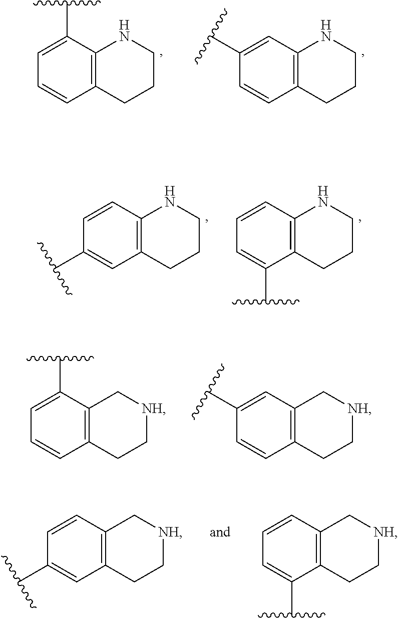
wherein T is optionally substituted with one or more substituents independently selected at each occurrence from R¹³.

67. The compound or salt of claim 54, wherein T is selected from 1,2,3,4-tetrahydroquinoline, 1,2,3,4-tetrahydroisoquinoline, 1,2-dihydroquinoline, 1,2-dihydroisoquinoline, 1,2,3,4-tetrahydroquinazoline, decahydroquinoline, decahydroisoquinoline, quinoline, isoquinoline, quinoxaline, phthalazine, quinazoline, and cinnoline, wherein T is optionally substituted with one or more substituents independently selected at each occurrence from R¹³.

68. The compound or salt of claim 54, wherein T is selected from

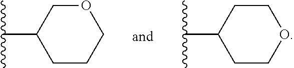 [SMILES: *C1CCCOC1 ; *C1CCOCC1]

69. The compound or salt of claims 1 to 24 wherein -Q-T is selected from 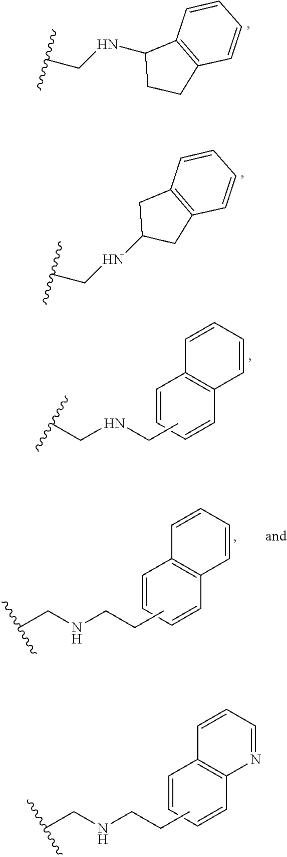
wherein T is optionally substituted with one or more substituents independently selected at each occurrence from R¹³.

70. The compound or salt of claim 69 wherein -Q-T is selected from

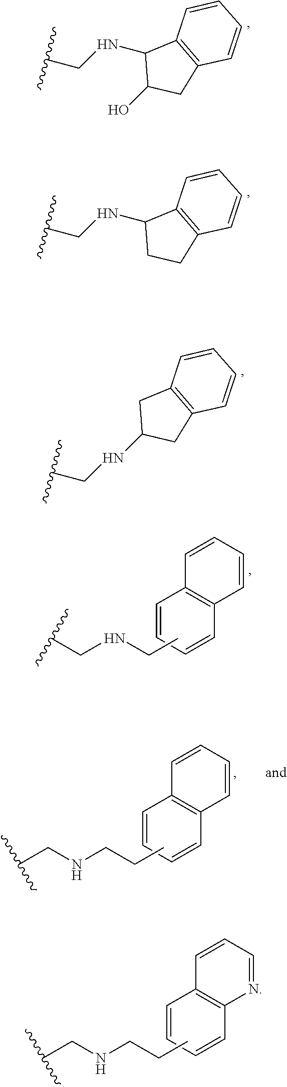

71. The compound or salt of any one of claims 1 to 67 and 69, wherein R¹³ is independently selected at each occurrence from: a halogen —OR¹⁰, —SR¹⁰, —N(R¹⁰)₂, —C(O)R¹⁰, —C(O)N(R¹⁰)₂, —N(R¹⁰)C(O)R¹⁰, —C(O)OR¹⁰, —OC(O)R¹⁰, —S(O)R¹⁰, —S(O)₂R¹⁰, —S(O)₂N(R¹⁰)₂, -P(O)(OR¹⁰)₂, —OP(O)(OR¹⁰)₂, —NO₂, ═O, ═S, ═N(R¹⁰), and —CN; —C₁-C₁₀ alkyl, —C₂-C₁₀ alkenyl, and —C₂-C₁₀ alkynyl, each of which is optionally substituted at each occurrence with one or more substituents independently selected from a halogen, —OR¹⁰, —SR¹⁰, —N(R¹⁰)₂, —C(O)R¹⁰, —C(O)N(R¹⁰)₂, —N(R¹⁰)C(O)OR¹⁰, —C(O)OR¹⁰, —OC(O)R¹⁰, —S(O)R¹⁰, —S(O)₂R¹⁰, —S(O)₂N(R¹⁰)₂, —P(O)(OR¹⁰)₂, —OP(O)(OR¹⁰)₂, —NO₂, ═O, ═S, ═N(R¹⁰), —CN, a C₃-C₁₀ carbocycle, and a 3- to 10-membered heterocycle.

72. The compound or salt of claim 71, wherein R¹³ is independently selected at each occurrence from a halogen, —OR¹⁰, —SR¹⁰, —N(R¹⁰)₂, —NO₂, and —CN; and —C₁-C₁₀ alkyl optionally substituted at each occurrence with one or more substituents independently selected from a halogen, —OR¹⁰, —SR¹⁰, —N(R¹⁰)₂, —C(O)R¹⁰, —NO₂, and —CN.

73. The compound or salt of claim 71, wherein R¹³ is independently selected at each occurrence from halogen, —OR¹⁰, —N(R¹⁰)₂, —C(O)R¹⁰, —C(O)OR¹⁰, —N(R¹⁰)C(O)R¹⁰, and —C₁-C₃ alkyl optionally substituted with one or more substituents independently selected from a halogen, —OR¹⁰ and —N(R¹⁰)₂.

74. The compound of claim 1, wherein the compound is selected from: 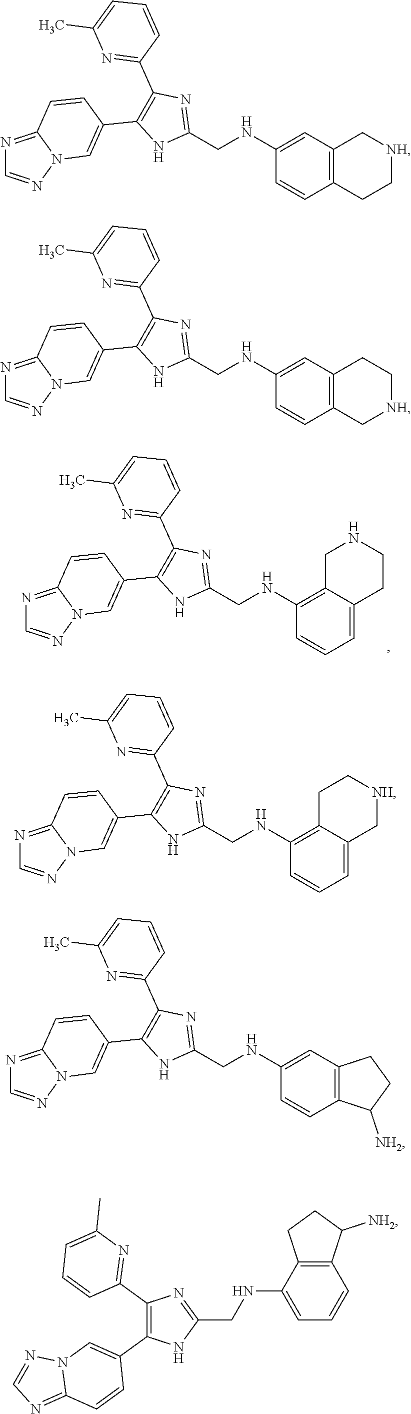 [SMILES: Cc1cccc(-c2nc(CNc3ccc4c(c3)CNCC4)[nH]c2-c2ccc3ncnn3c2)n1 ; Cc1cccc(-c2nc(CNc3ccc4c(c3)CCNC4)[nH]c2-c2ccc3ncnn3c2)n1 ; Cc1cccc(-c2nc(CNc3cccc4c3CNCC4)[nH]c2-c2ccc3ncnn3c2)n1 ; Cc1cccc(-c2nc(CNc3cccc4c3CCNC4)[nH]c2-c2ccc3ncnn3c2)n1 ; Cc1cccc(-c2nc(CNc3ccc4c(c3)CCC4N)[nH]c2-c2ccc3ncnn3c2)n1 ; Cc1cccc(-c2nc(CNc3cccc4c3CCC4N)[nH]c2-c2ccc3ncnn3c2)n1] 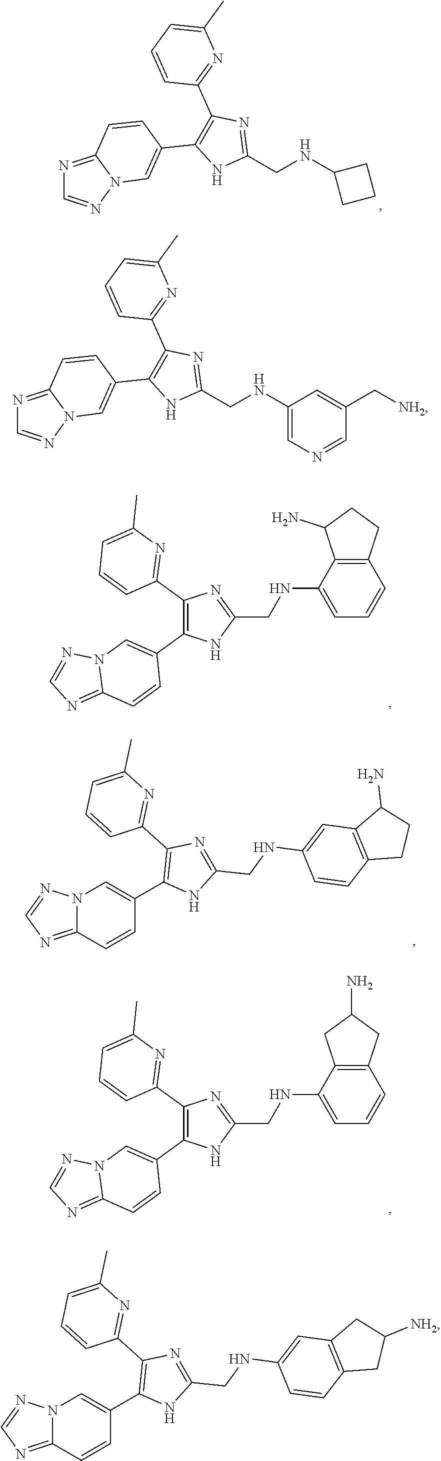 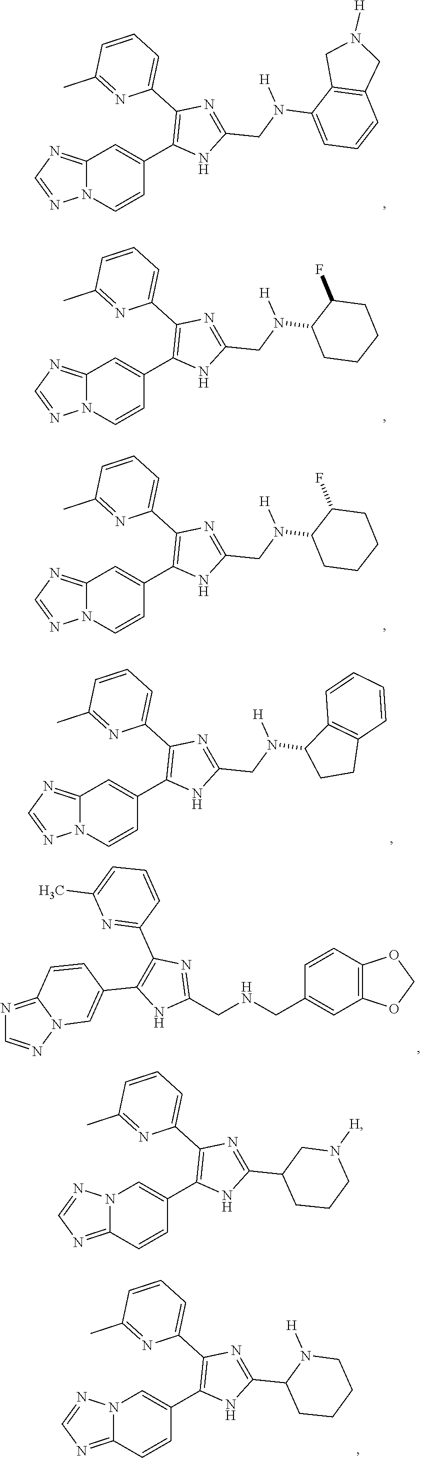 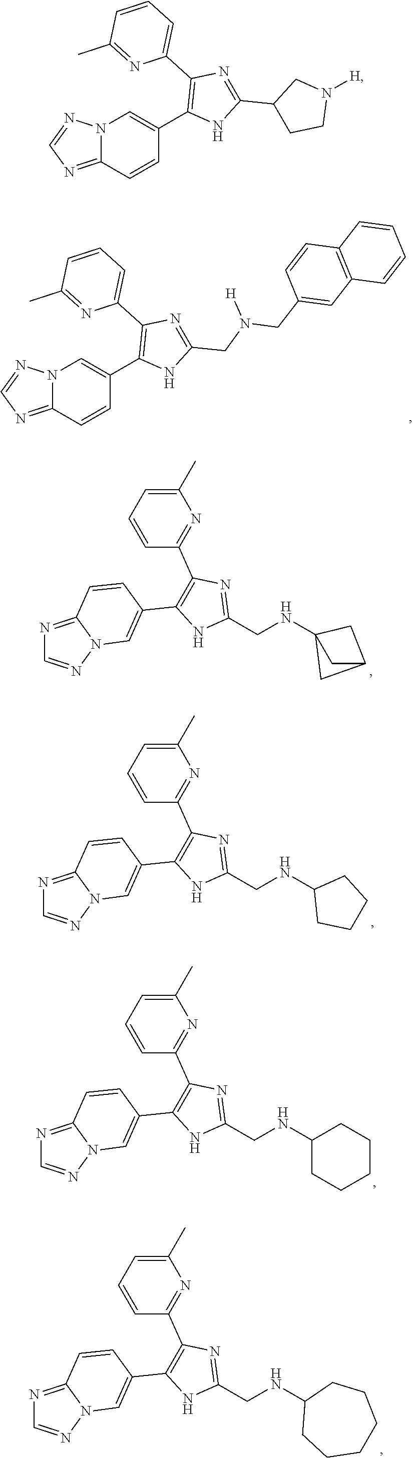 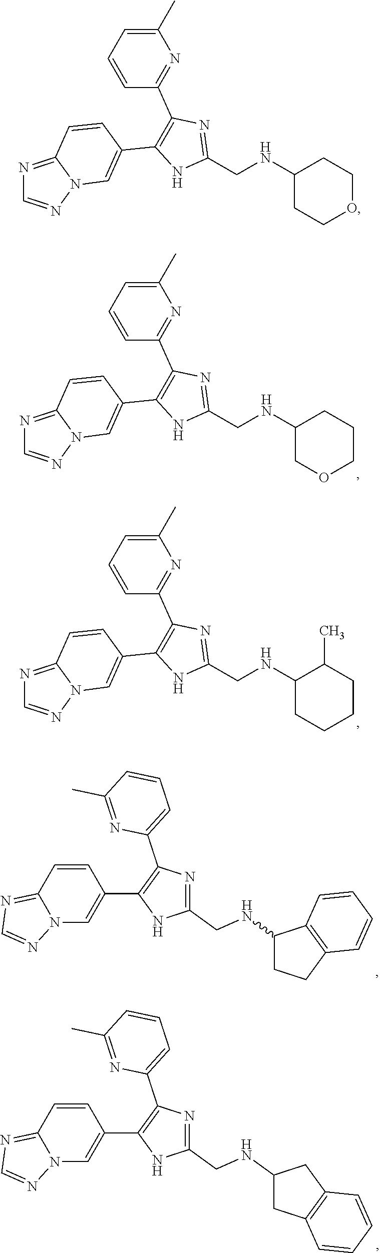 [SMILES: Cc1cccc(-c2nc(CNC3CCOCC3)[nH]c2-c2ccc3ncnn3c2)n1 ; Cc1cccc(-c2nc(CNC3CCCOC3)[nH]c2-c2ccc3ncnn3c2)n1 ; Cc1cccc(-c2nc(CNC3CCCCC3C)[nH]c2-c2ccc3ncnn3c2)n1 ; Cc1cccc(-c2nc(CNC3CCc4ccccc43)[nH]c2-c2ccc3ncnn3c2)n1 ; Cc1cccc(-c2nc(CNC3Cc4ccccc4C3)[nH]c2-c2ccc3ncnn3c2)n1] 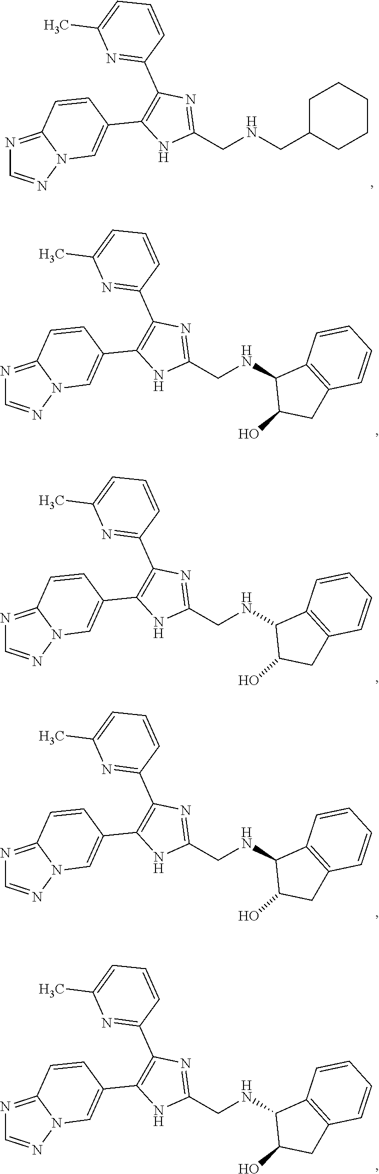 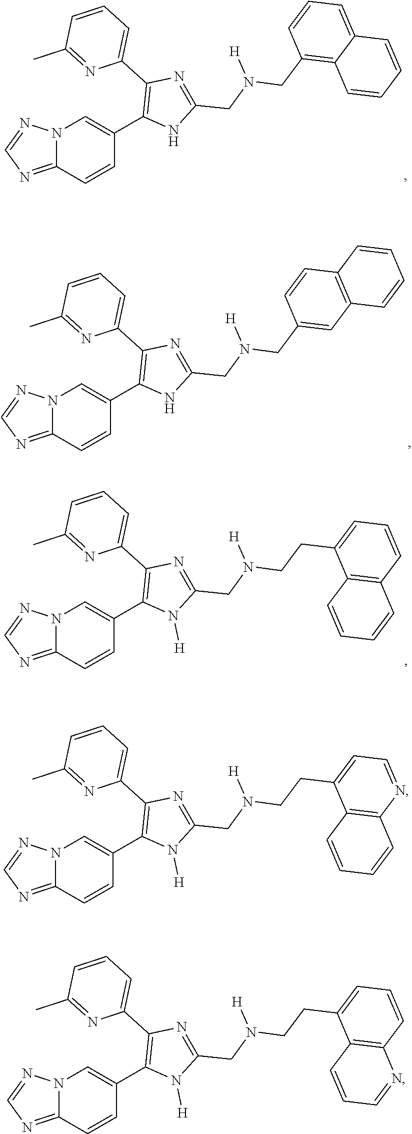
and a salt of any one thereof.

75. The compound of claim 1, wherein the compound is selected from 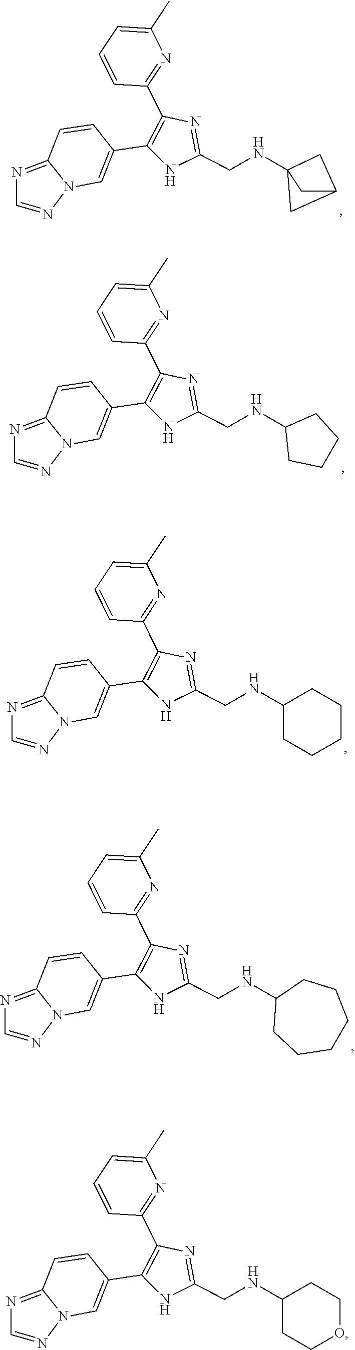 [SMILES: Cc1cccc(-c2nc(CNC34CC(C3)C4)[nH]c2-c2ccc3ncnn3c2)n1 ; Cc1cccc(-c2nc(CNC3CCCC3)[nH]c2-c2ccc3ncnn3c2)n1 ; Cc1cccc(-c2nc(CNC3CCCCC3)[nH]c2-c2ccc3ncnn3c2)n1 ; Cc1cccc(-c2nc(CNC3CCCCCC3)[nH]c2-c2ccc3ncnn3c2)n1 ; Cc1cccc(-c2nc(CNC3CCOCC3)[nH]c2-c2ccc3ncnn3c2)n1] 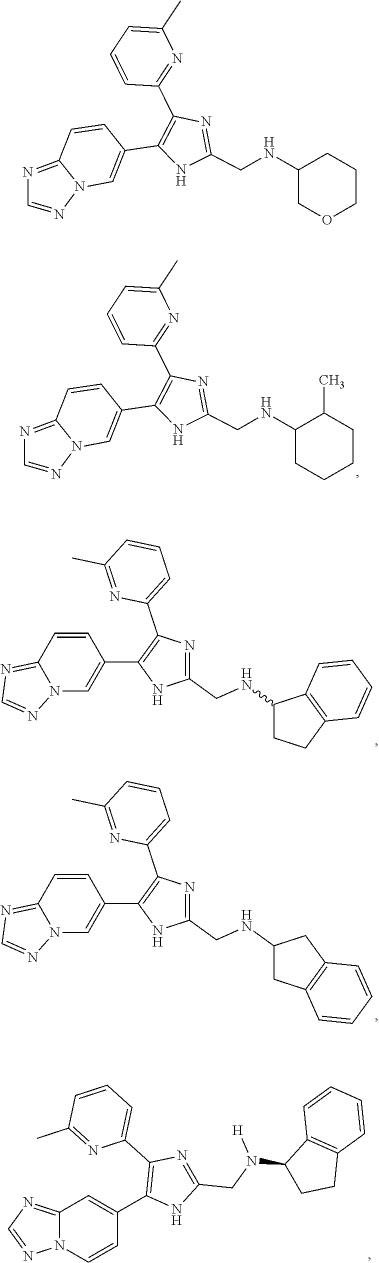 [SMILES: Cc1cccc(-c2nc(CNC3CCCOC3)[nH]c2-c2ccc3ncnn3c2)n1 ; Cc1cccc(-c2nc(CNC3CCCCC3C)[nH]c2-c2ccc3ncnn3c2)n1 ; Cc1cccc(-c2nc(CNC3CCc4ccccc43)[nH]c2-c2ccc3ncnn3c2)n1 ; Cc1cccc(-c2nc(CNC3Cc4ccccc4C3)[nH]c2-c2ccc3ncnn3c2)n1 ; Cc1cccc(-c2nc(CN[C@@H]3CCc4ccccc43)[nH]c2-c2ccn3ncnc3c2)n1] 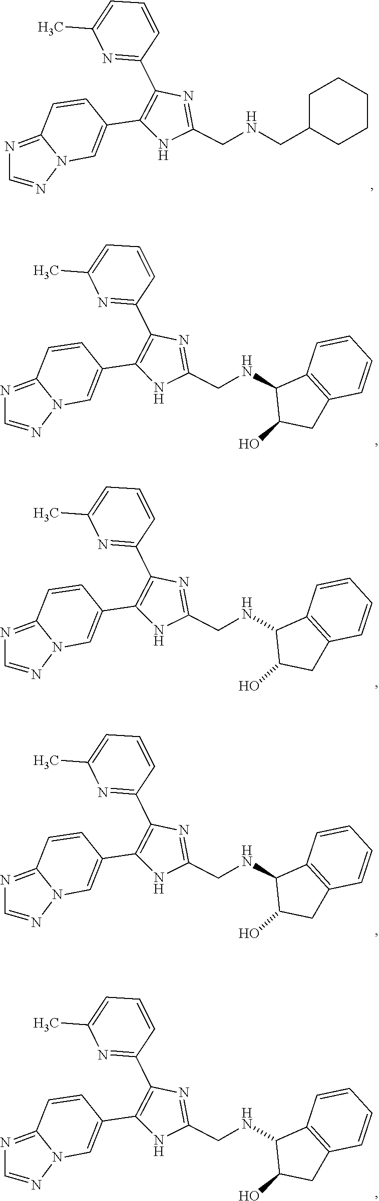 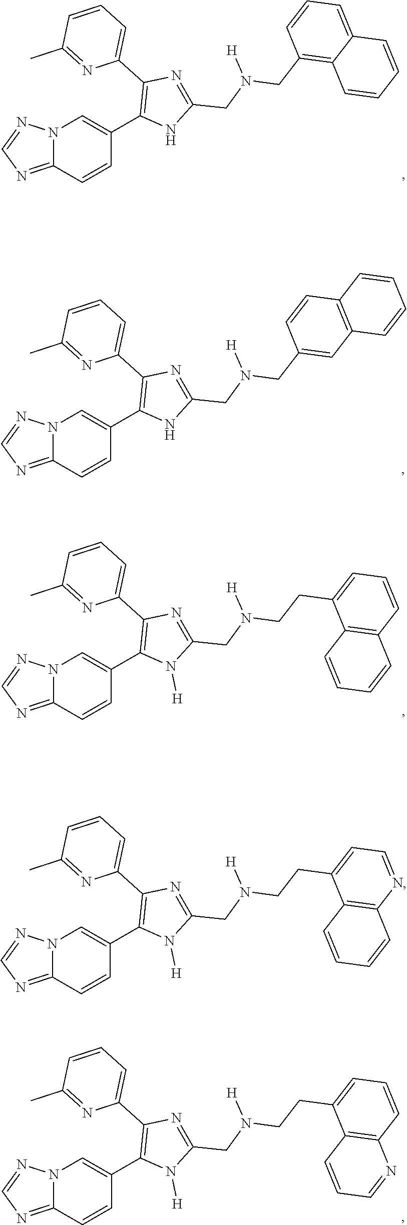 [SMILES: Cc1cccc(-c2nc(CNCc3cccc4ccccc34)[nH]c2-c2ccc3ncnn3c2)n1 ; Cc1cccc(-c2nc(CNCc3ccc4ccccc4c3)[nH]c2-c2ccc3ncnn3c2)n1 ; Cc1cccc(-c2nc(CNCCc3cccc4ccccc34)[nH]c2-c2ccc3ncnn3c2)n1 ; Cc1cccc(-c2nc(CNCCc3ccnc4ccccc34)[nH]c2-c2ccc3ncnn3c2)n1 ; Cc1cccc(-c2nc(CNCCc3cccc4ncccc34)[nH]c2-c2ccc3ncnn3c2)n1]
and a salt of any one thereof.

76. The compound of claim 1, wherein the compound is selected from 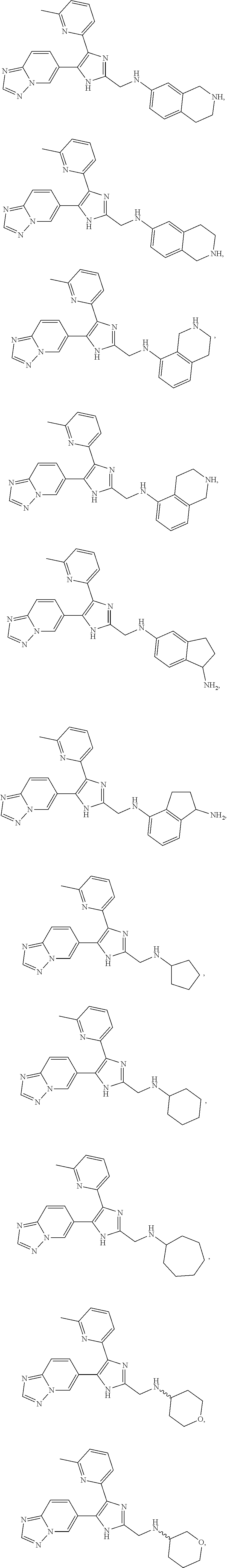 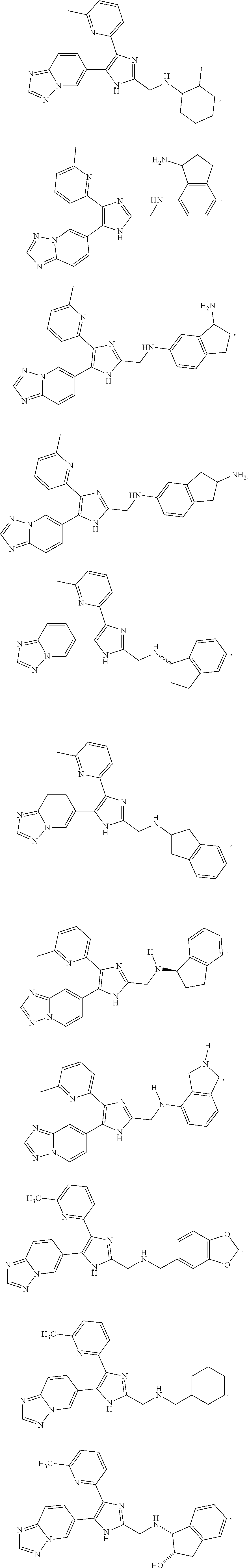 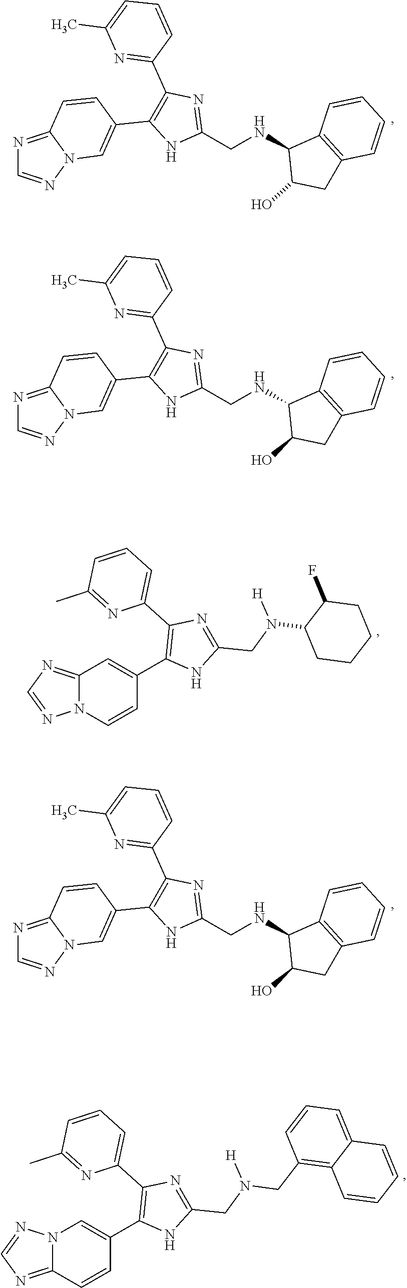
and a salt of any one thereof.

77. The compound of claim 1, wherein the compound is selected from 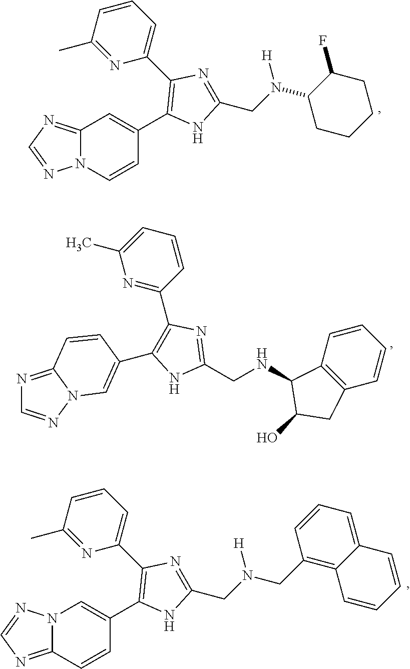
and a salt of any one thereof.

78. The compound of claim 1, wherein the compound is selected from 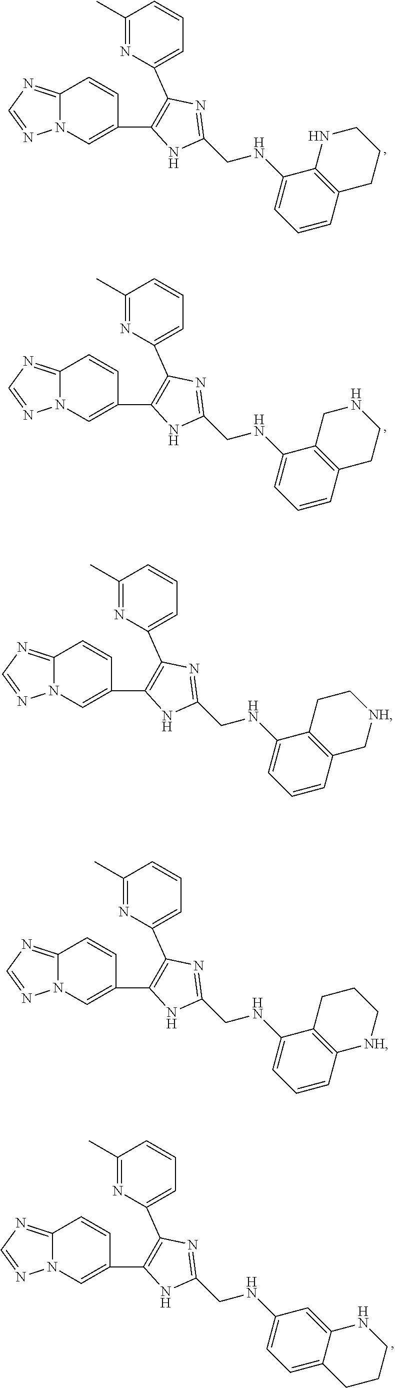 [SMILES: Cc1cccc(-c2nc(CNc3cccc4c3NCCC4)[nH]c2-c2ccc3ncnn3c2)n1 ; Cc1cccc(-c2nc(CNc3cccc4c3CNCC4)[nH]c2-c2ccc3ncnn3c2)n1 ; Cc1cccc(-c2nc(CNc3cccc4c3CCNC4)[nH]c2-c2ccc3ncnn3c2)n1 ; Cc1cccc(-c2nc(CNc3cccc4c3CCCN4)[nH]c2-c2ccc3ncnn3c2)n1 ; Cc1cccc(-c2nc(CNc3ccc4c(c3)NCCC4)[nH]c2-c2ccc3ncnn3c2)n1] 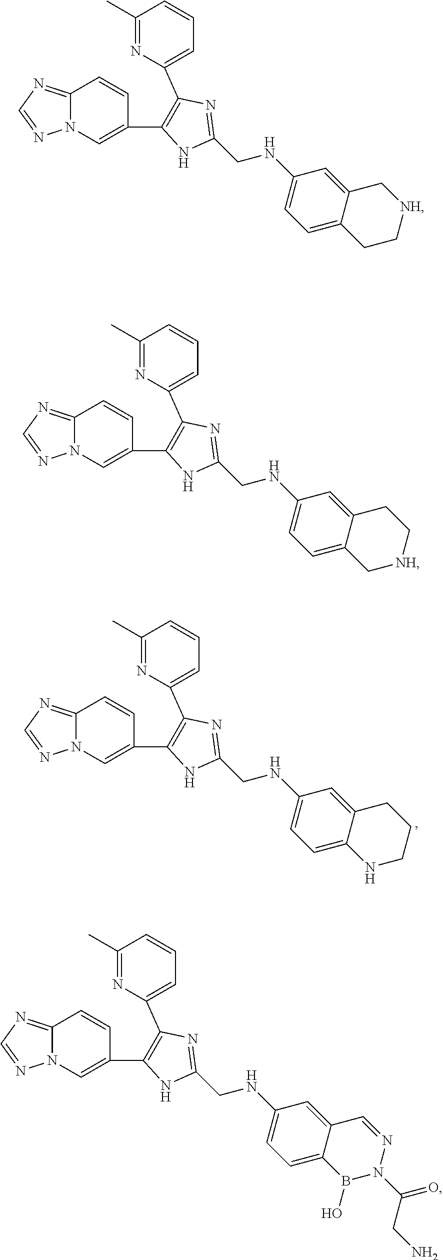 [SMILES: Cc1cccc(-c2nc(CNc3ccc4c(c3)CNCC4)[nH]c2-c2ccc3ncnn3c2)n1 ; Cc1cccc(-c2nc(CNc3ccc4c(c3)CCNC4)[nH]c2-c2ccc3ncnn3c2)n1 ; Cc1cccc(-c2nc(CNc3ccc4c(c3)CCCN4)[nH]c2-c2ccc3ncnn3c2)n1 ; Cc1cccc(-c2nc(CNc3ccc4c(c3)C=NN(C(=O)CN)B4O)[nH]c2-c2ccc3ncnn3c2)n1] 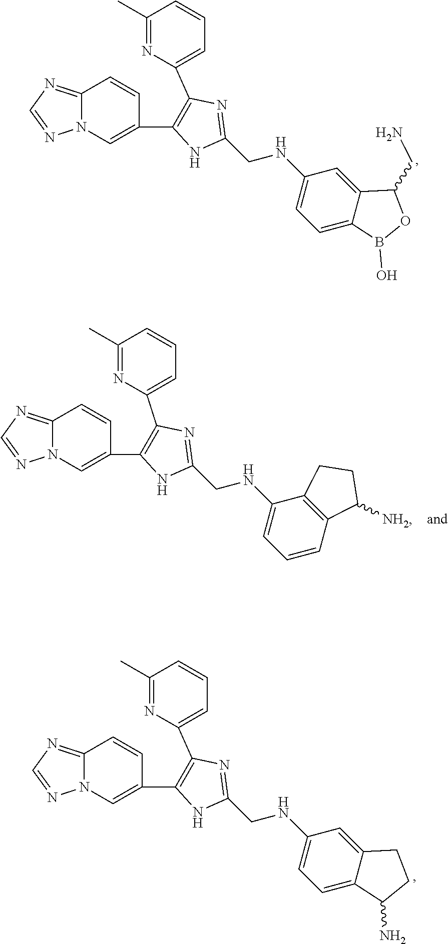 [SMILES: Cc1cccc(-c2nc(CNc3ccc4c(c3)C(CN)OB4O)[nH]c2-c2ccc3ncnn3c2)n1 ; Cc1cccc(-c2nc(CNc3cccc4c3CCC4N)[nH]c2-c2ccc3ncnn3c2)n1 ; Cc1cccc(-c2nc(CNc3ccc4c(c3)CCC4N)[nH]c2-c2ccc3ncnn3c2)n1]
or a salt of any one thereof.

79. The compound of claim 1, wherein the compound is selected from 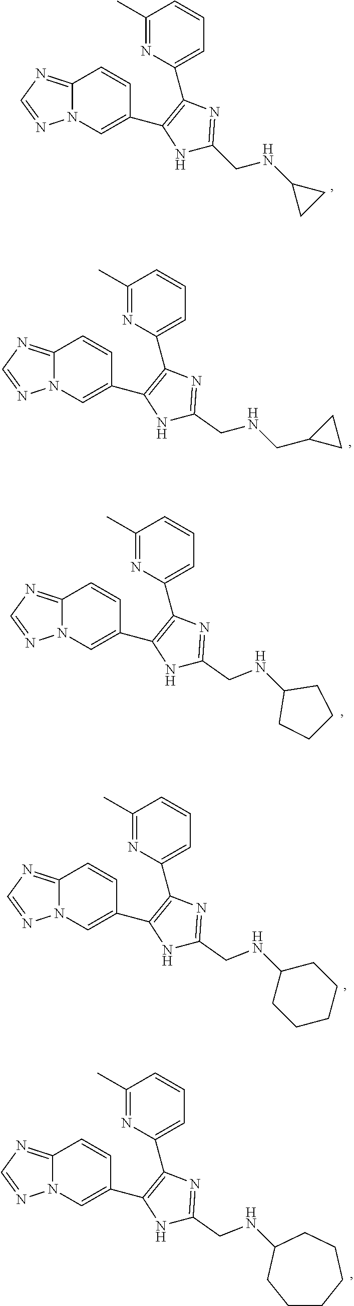 [SMILES: Cc1cccc(-c2nc(CNC3CC3)[nH]c2-c2ccc3ncnn3c2)n1 ; Cc1cccc(-c2nc(CNCC3CC3)[nH]c2-c2ccc3ncnn3c2)n1 ; Cc1cccc(-c2nc(CNC3CCCC3)[nH]c2-c2ccc3ncnn3c2)n1 ; Cc1cccc(-c2nc(CNC3CCCCC3)[nH]c2-c2ccc3ncnn3c2)n1 ; Cc1cccc(-c2nc(CNC3CCCCCC3)[nH]c2-c2ccc3ncnn3c2)n1] 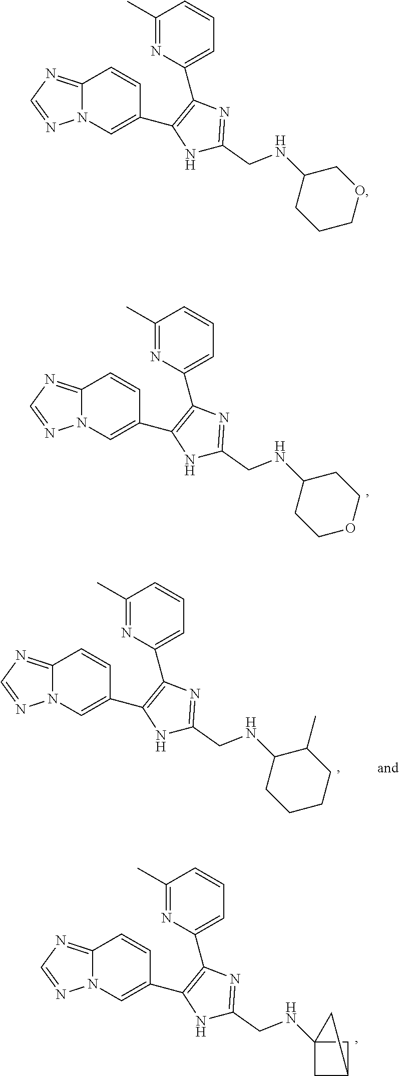 [SMILES: Cc1cccc(-c2nc(CNC3CCCOC3)[nH]c2-c2ccc3ncnn3c2)n1 ; Cc1cccc(-c2nc(CNC3CCOCC3)[nH]c2-c2ccc3ncnn3c2)n1 ; Cc1cccc(-c2nc(CNC3CCCCC3C)[nH]c2-c2ccc3ncnn3c2)n1 ; Cc1cccc(-c2nc(CNC34CC(C3)C4)[nH]c2-c2ccc3ncnn3c2)n1]
or a salt of any one thereof.

80. The compound of claim 1, wherein the compound is selected from 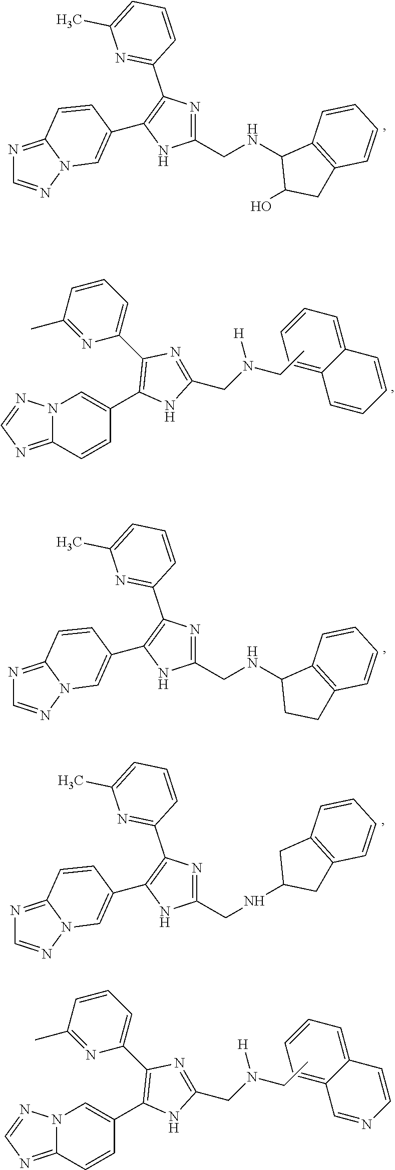
and a salt of any one thereof.

81. The compound or salt of any one of claims 1 to 80, wherein the compound is covalently bound to a linker -L³.

82. The compound or salt of claim 81, wherein L³ is covalently bound to a substitutable nitrogen or substitutable oxygen of the compound.

83. The compound or salt of claim 82, wherein L³ is covalently bound to a substitutable nitrogen of the compound.

84. The compound or salt of claim 81-83, wherein -L³ is a non-cleavable linker.

85. The compound or salt of claim 81-83, wherein -L³ is a cleavable linker.

86. The compound or salt of claim 84, wherein -L³ is cleavable by a lysosomal enzyme.

87. The compound or salt of any one of claims 81-86, wherein the linker is attached to an atom of T.

88. The compound of claim 87, wherein the compound covalently bound to a linker -L³ is represented by: 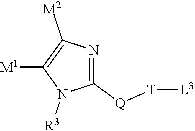
or a salt thereof.

89. The compound or salt of claim 87 or 88, wherein the linker L³ is attached to an endocyclic nitrogen atom.

90. The compound or salt of any one of claims 81 to 86, wherein the linker is attached to a substituent that is attached to an atom of T.

91. The compound of claim 90, wherein the compound covalently bound to a linker -L³ is represented by: 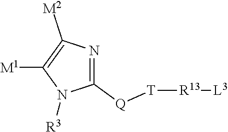
or salt thereof.

92. The compound or salt of claim 90-91, wherein the linker L³ is attached to an exocyclic nitrogen or oxygen atom.

93. The compound or salt of any one of claims 81 to 86, wherein the linker is attached to an atom of Q.

94. The compound of claim 93, wherein the compound covalently bound to a linker -L³ is represented by: 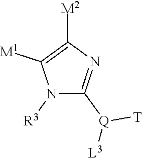
or salt thereof.

95. The compound or salt of claims 93 to 94, wherein the linker L³ is attached to a nitrogen atom of Q.

96. The compound or salt of any one of claims 81-95, wherein -L³ comprises a reactive moiety.

97. The compound or salt of claim 96, wherein -L³ is represented by the formula: 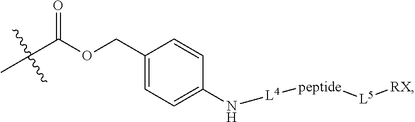
wherein L⁴ represents the C-terminus of the peptide and L⁵ is selected from a bond, alkylene and heteroalkylene, wherein L⁵ is optionally substituted with one or more groups independently selected from R³², and RX is the reactive moiety; and R³² is independently selected at each occurrence from halogen, —OH, —CN, —O-alkyl, —SH, ═O, ═S, —NH₂, —NO₂;
and C₁₋₁₀ alkyl, C₂₋₁₀ alkenyl, C₂₋₁₀ alkynyl, each of which is independently optionally substituted at each occurrence with one or more substituents selected from halogen, —OH, —CN, —O-alkyl, —SH, ═O, ═S, —NH₂, —NO₂, and 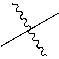
represents the point of attachment to the compound or salt of any one of claims 1 to 80.

98. The compound or salt of claim 96 or 97, wherein RX comprises a leaving group.

99. The compound or salt of claim 98 wherein RX is a maleimide.

100. The compound or salt of any one of claims 97 to 100, wherein the peptide of -L³ comprises Val-Cit or Val-Ala.

101. The compound or salt of claim 100, wherein -L³ is represented by the formula:

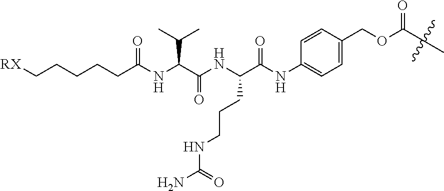

102. The compound or salt of claim 96, wherein -L³ is represented by the formula: 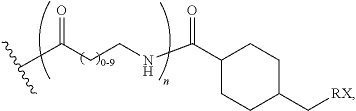
wherein RX comprises the reactive moiety, and n =0-9.

103. The compound or salt of claim 102, wherein RX comprises a leaving group.

104. The compound or salt of claim 102, wherein RX is a maleimide.

105. The compound or salt of any one of claims 81-104, wherein -L³ is further covalently bound to an antibody construct.

106. The compound or salt of claim 105, wherein -L³ is represented by the formula: 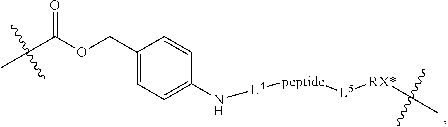
wherein L⁴ represents the C-terminal of the peptide and L⁵ is selected from a bond, alkylene and heteroalkylene, wherein L⁵ is optionally substituted with one or more groups independently selected from R³²; RX* is a bond, a succinimide moiety, or a hydrolyzed succinimide moiety bound to a residue of an antibody construct, wherein 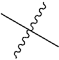
on RX* represents the point of attachment to the residue of the antibody construct; and, R³² is independently selected at each occurrence from halogen, —OH, —CN, —O-alkyl, —SH, ═O, ═S, —NH₂, —NO₂; and C₁₋₁₀ alkyl, C₂₋₁₀ alkenyl, C₂₋₁₀ alkynyl, each of which is independently optionally substituted at each occurrence with one or more substituents selected from halogen, —OH, —CN, —O-alkyl, —SH, ═O, ═S, —NH₂, —NO₂.

107. The compound or salt of claim 106, wherein the peptide of -L³ comprises Val-Cit or Val-Ala.

108. The compound or salt of claim 107, wherein -L³ is represented by the formula:

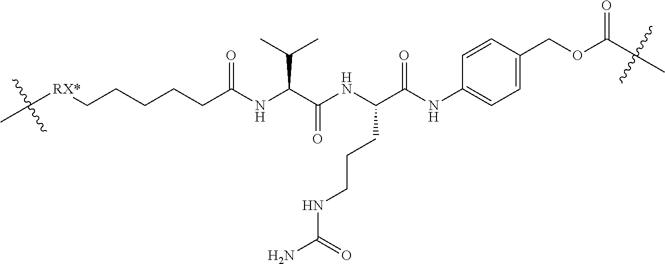

109. The compound or salt of claim 105, wherein -L³ is represented as follows: 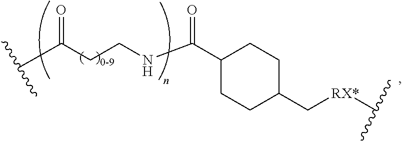
wherein RX* is a bond, a succinimide moiety, or a hydrolyzed succinimide moiety bound to a residue of an antibody construct, wherein 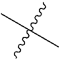
on RX* represents the point of attachment to the residue of the antibody construct, and n=0-9.

110. The compound or salt of any one of claims 105-109, wherein the antibody construct comprises an antigen binding domain that specifically binds to LRRC15, ADAM12, MMP14, GPX8, PDPN, CDH11 or F2RL2.

111. The compound or salt of any one of claims 105-109, wherein the antibody construct comprises an antibody or an antigen binding fragment thereof that specifically binds to MUC16, UPK1B, VTCN1, TMPRSS3, TMEM238, C1orf186, TMPRSS4, CLDN6, CLDN8 or STRA6.

112. The compound of claim 81, wherein the compound bound to a linker is a compound-linker as set forth in Table 4 or a salt of any one thereof.

113. The compound or salt of any one of claims 1 to 112, wherein the compound is covalently attached to an antibody construct.

114. The compound or salt of any of claims 81 to 112, wherein the compound is covalently attached to a targeting moiety through the linker.

115. The compound or salt of claim 113-114, wherein the targeting moiety or antibody construct specifically binds to a tumor antigen.

116. The compound or salt of any one of claims 113-115, wherein the antibody construct or targeting moiety further comprises a target binding domain.

117. A conjugate represented by the formula: 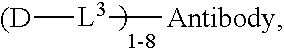
wherein Antibody is an antibody construct, D is a compound or salt selected from any one of claims 1 to 80 and L³ is a linker moiety.

118. A conjugate represented by the formula: 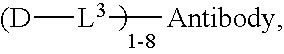
wherein Antibody is an antibody construct and D-L³ is a compound or salt selected from any of claims 81 to 112.

119. The conjugate of claim 117-119, wherein the Fc domain of the antibody construct is an Fc null.

120. The conjugate of claim 117-119, wherein the antibody construct has a functioning Fc domain.

121. A pharmaceutical composition comprising the compound or salt of any one of claims 1 to 80 and at least one pharmaceutically acceptable excipient.

122. A pharmaceutical composition, comprising the conjugate of any one of claims 105 to 120 and at least one pharmaceutically acceptable excipient.

123. The pharmaceutical composition of claim 122, wherein the average drug-to-antibody ratio of the conjugate is from about 2 to about 8, or about 1 to about 3, or about 3 to about 5.

124. A method of killing tumor cells in vivo, comprising contacting a tumor cell population with a conjugate of any one of claims 105 to 120 or a compound of any one of claims 1 to 80.

125. A method for treatment, comprising administering to a subject a conjugate of any one of claims 105 to 120 or a compound of any one of claims 1 to 80.

126. A method for treatment, comprising administering to a subject in need thereof a compound or salt of any one of claims 1 to 80.

127. A method for treating cancer, comprising administering to a subject in need thereof a conjugate of any one of claims 105 to 120.

128. A method for treating cancer, comprising administering to a subject in need thereof a compound or salt of any one of claims 1 to 80.

129. A compound or salt of any one of claims 1 to 80 for use in a method of treatment of a subject's body by therapy.

130. A compound or salt of any one of claims 1 to 80 for use in a method of treating cancer.

131. A conjugate of any one of claims 105 to 120 for use in a method of treatment of a subject's body by therapy.

132. A conjugate of any one of claims 105 to 120 for use in a method of treating cancer.

133. A method for treating fibrosis comprising administering to a subject in need thereof a compound or salt of any one of claims 1 to 80.

134. A method of treating fibrosis comprising administering to a subject in need thereof a conjugate of any one of claims 105 to 120.

135. The method of claim 134 wherein, the targeting moiety or antibody construct specifically binds to LRRC15.

136. The method of any one of claims 105-120, wherein the targeting moiety or antibody construct specifically binds to a tumor antigen on non-cancerous cells associated with fibrosis, autoimmune disease or inflammatory disease.

137. The method of claim 136 for the treatment of fibrosis, autoimmune disease, or inflammatory disease.

138. The method of any one of claims 105 to 137 wherein the antibody construct comprises a chimeric, human or humanized antibody.

139. A method of preparing an antibody conjugate comprising a targeting moiety, at least one linker, L³, and at least one compound, D, wherein L³-D is selected from a compound-linker or salt thereof of any one of claims 81-104, comprising contacting L³-D with an antibody construct.

140. A method of preparing an antibody conjugate of the formula 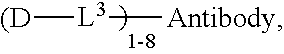
wherein Antibody is an antibody construct, L³ is a linker and D is a compound or salt, wherein L³-D is selected from a compound-linker or salt thereof of any one of claims 81-104 comprising contacting L³-D with an antibody construct.

141. The compound of claim 1 wherein the compound is selected from: 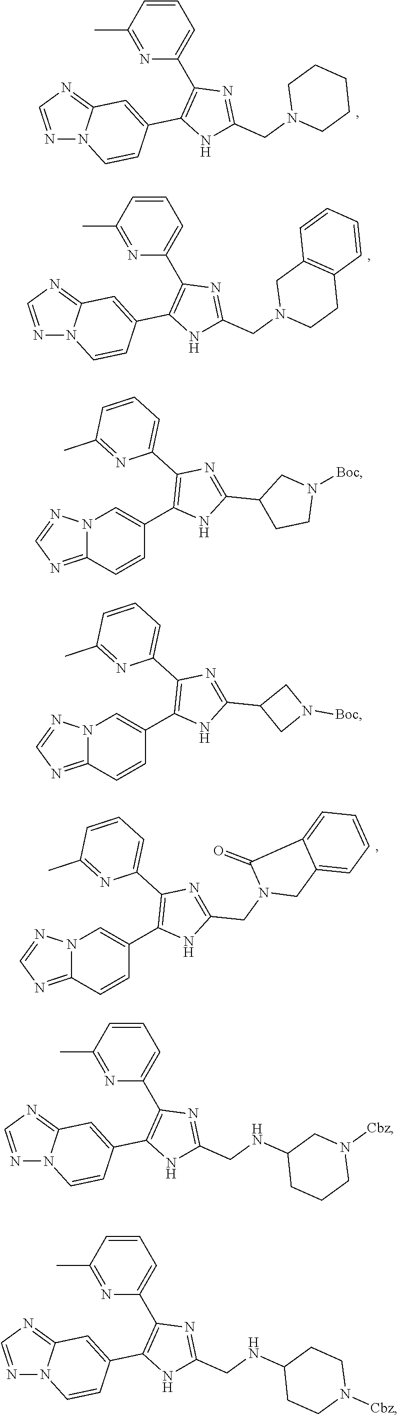 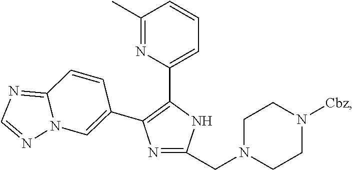 [SMILES: Cc1cccc(-c2[nH]c(CN3CCN(C(=O)OCc4ccccc4)CC3)nc2-c2ccc3ncnn3c2)n1]
and a salt of any one thereof.
